SEQUENCE LISTING (92 sequences)

SEQ ID NO: 1-2 - skipped (2 entries)

SEQ ID NO: 3 - DNA, 12 bp, synthetic construct
  source 1..12: mol_type = other DNA; note = Kozak consensus sequence
gcggccgccatg

SEQ ID NO: 4 - skipped

SEQ ID NO: 5 - DNA, 660 bp, synthetic construct
  source 1..660: mol_type = other DNA; note = Human IL-12A (p35) ("wild-type" no CpG modification)
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctga

SEQ ID NO: 6 - DNA, 660 bp, synthetic construct
  source 1..660: mol_type = other DNA; note = Human IL-12A (p35) (with CpG modification)
atgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttcctga

SEQ ID NO: 7 - DNA, 660 bp, synthetic construct
  source 1..660: mol_type = other DNA; note = Human IL-12A (p35) (with CpG modification)
atgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttcctga

SEQ ID NO: 8 - DNA, 987 bp, synthetic construct
  source 1..987: mol_type = other DNA; note = Human IL-12B (p40) ("wild-type" no CpG modification and with stop codon)
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttga

SEQ ID NO: 9 - DNA, 986 bp, synthetic construct
  source 1..986: mol_type = other DNA; note = Human IL-12B (p40) (with CpG modification)
tgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttga

SEQ ID NO: 10 - DNA, 987 bp, synthetic construct
  source 1..987: mol_type = other DNA; note = Human IL-12B (p40) (with CpG modification)
atgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttga

SEQ ID NO: 11 - DNA, 489 bp, synthetic construct
  source 1..489: mol_type = other DNA; note = IL-15 ("wild-type" no CpG modification)
atgagaatttcgaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 12 - DNA, 489 bp, synthetic construct
  source 1..489: mol_type = other DNA; note = IL-15(with CpG modification)
atgagaattagcaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatactgaaagtgatgttcaccccagttgcaaagtaacagcaatgaagtgctttctcttggagttacaagttatttcacttgagagcggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 13 - DNA, 489 bp, synthetic construct
  source 1..489: mol_type = other DNA; note = IL-15 (with CpG modification)
atgagaattagcaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatactgaaagtgatgttcaccccagttgcaaagtaacagcaatgaagtgctttctcttggagttacaagttatttcacttgagtcaggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 14 - DNA, 4554 bp, synthetic construct
  source 1..4554: mol_type = other DNA; note = Human EF-1a -wild-type IL-12A/IL-12B/IL-15 - IRES (no CpG modification)
gtcgaccacacaaaaaaccaacacacagatctaatgaaaataaagatcttttattggcgcgcctcaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacttgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatcacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaatttcgaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 15 - DNA, 4062 bp, synthetic construct
  source 1..4062: mol_type = other DNA; note = Human EF-1a- wild-type IL-12A/IL-12B/IL-15 - F2A (no CpG modification)
gtcgaccacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctccggactcaagtgaaataacttgtaactccaagagaaagcacttcattgctgttactttgcaactggggtgaacatcactttccgtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttcgaaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 16 - DNA, 4554 bp, synthetic construct
  source 1..4554: mol_type = other DNA; note = Human EF-1a - modified IL-12A/IL-12B/IL-15 - IRES (with CpG modification)
gtcgaccacacaaaaaaccaacacacagatctaatgaaaataaagatcttttattggcgcgcctcaactgcagggcacagatgcccattctgaccaagatgagctatagtagcggtcctgggcccgcactgaaatgctggcatttttgcggcagatgactgtggctgaggtcttgtcagtgaagactctatctttcttttctctcttgctcttgccctggacttgaacacagaatgtcagggagaagtaggaatgtggagtactccaggtgtcagggtactcccagctgacctccacctgcctagaattctttaatggcttcagctgcaagttcttgggtgggtcaggtttgatgatgtccctgatgaagaagctgctggtgtagttttcatacttgagcttgtgaaccgcatccaccatcacctcaatgggcagactctcctcagcagctgggcaggcactgtcctcctggcactccactgagtactcatactccttgttgtcccctctgactctctctgcagagagtgtagcagctccgcatgtcaccccttgggggtcagaagagcctctgctgcttttgacactgaatgtcaaatcagtactgattgtagtcagccaccagcaggtgaaccttccagaataattcttggcctcacatcttagaaaggtcttatttttgggttctttctggtcctttaaaatatcagtggaccaaattccatcttcctttttgtgaagcagcaggagggaatggcttagaacctcccctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatcaggataccaatccaattctaccacataaacatctttcttcagttcccatatggccacaaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaattagcaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatactgaaagtgatgttcaccccagttgcaaagtaacagcaatgaagtgctttctcttggagttacaagttatttcacttgagagcggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 17 - DNA, 4062 bp, synthetic construct
  source 1..4062: mol_type = other DNA; note = Human EF1a- modified IL-12A/IL-12B/IL-15 - F2A (with CpG modification)
gtcgaccacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctccgctctcaagtgaaataacttgtaactccaagagaaagcacttcattgctgttactttgcaactggggtgaacatcactttcagtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttgctaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 18 - DNA, 4554 bp, synthetic construct
  source 1..4554: mol_type = other DNA; note = Human EF1a - modified IL-12A/IL-12B/IL-15 - IRES (with CpG modification)
gtcgaccacacaaaaaaccaacacacagatctaatgaaaataaagatcttttattggcgcgcctcaactgcagggcacagatgcccattctgaccaagatgagctatagtatctgtcctgggccctcactgaaatgctggcatttttcctgcagatgactgtggctgaggtcttgtcagtgaagactctatctttcttttctctcttgctcttgccctggacttgaacacagaatgtcagggagaagtaggaatgtggagtactccaggtgtcagggtactcccagctgacctccacctgcctagaattctttaatggcttcagctgcaagttcttgggtgggtcaggtttgatgatgtccctgatgaagaagctgctggtgtagttttcatacttgagcttgtgaactgcatccaccatcacctcaatgggcagactctcctcagcagctgggcaggcactgtcctcctggcactccactgagtactcatactccttgttgtcccctctgactctctctgcagagagtgtagcagctccacatgtcaccccttgggggtcagaagagcctctgctgcttttgacactgaatgtcaaatcagtactgattgtagtcagccaccagcaggtgaaccttccagaataattcttggcctcacatcttagaaaggtcttatttttgggttctttctggtcctttaaaatatcagtggaccaaattccatcttcctttttgtgaagcagcaggagggaatggcttagaacctcccctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatcaggataccaatccaattctaccacataaacatctttcttcagttcccatatggccacaaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaattagcaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatactgaaagtgatgttcaccccagttgcaaagtaacagcaatgaagtgctttctcttggagttacaagttatttcacttgagtcaggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 19 - DNA, 4062 bp, synthetic construct
  source 1..4062: mol_type = other DNA; note = Human EF-1a - modified IL-12A/IL-12B/IL-15 - F2A (with CpG modification)
gtcgaccacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctcctgactcaagtgaaataacttgtaactccaagagaaagcacttcattgctgttactttgcaactggggtgaacatcactttcagtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttgctaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 20 - DNA, 4602 bp, synthetic construct
  source 1..4602: mol_type = other DNA; note = Human EF-1a- wild-type IL-12A/IL-12B/IL-15 - ODN-IRES (no CpG modification)
gtcgactcgtcgttttgtcgttttgtcgttcacacaaaaaaccaacacacagatctaatgaaaataaagatcttttattggcgcgcctcaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacttgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatcacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaatttcgaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtcgtcgttttgtcgttttgtcgttgagctcgag

SEQ ID NO: 21 - DNA, 4110 bp, synthetic construct
  source 1..4110: mol_type = other DNA; note = Human EF-1a - wild-type IL-12A/IL-12B/IL-15 - ODN - F2A (no CpG modification)
gtcgactcgtcgttttgtcgttttgtcgttcacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctccggactcaagtgaaataacttgtaactccaagagaaagcacttcattgctgttactttgcaactggggtgaacatcactttccgtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttcgaaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtcgtcgttttgtcgttttgtcgttgagctcgag

SEQ ID NO: 22 - DNA, 4408 bp, synthetic construct
  source 1..4408: mol_type = other DNA; note = Human UbC - wild-type IL-12A/IL-12B/IL-15 - IRES (no CpG modification)
gtcgaccacacaaaaaaccaacacacagatctaatgaaaataaagatcttttattggcgcgcctcaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacttgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatcacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaatttcgaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 23 - DNA, 3916 bp, synthetic construct
  source 1..3916: mol_type = other DNA; note = Human UbC - wild-type IL-12A/IL-12B/IL-15 - F2A (no CpG modification)
gtcgaccacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctccggactcaagtgaaataacttgtaactccaagagaaagcacttcattgctgttactttgcaactggggtgaacatcactttccgtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttcgaaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 24 - DNA, 4408 bp, synthetic construct
  source 1..4408: mol_type = other DNA; note = Human UbC - modified IL-12A/IL-12B/IL-15 - IRES (with CpG modification)
gtcgaccacacaaaaaaccaacacacagatctaatgaaaataaagatcttttattggcgcgcctcaactgcagggcacagatgcccattctgaccaagatgagctatagtagcggtcctgggcccgcactgaaatgctggcatttttgcggcagatgactgtggctgaggtcttgtcagtgaagactctatctttcttttctctcttgctcttgccctggacttgaacacagaatgtcagggagaagtaggaatgtggagtactccaggtgtcagggtactcccagctgacctccacctgcctagaattctttaatggcttcagctgcaagttcttgggtgggtcaggtttgatgatgtccctgatgaagaagctgctggtgtagttttcatacttgagcttgtgaaccgcatccaccatcacctcaatgggcagactctcctcagcagctgggcaggcactgtcctcctggcactccactgagtactcatactccttgttgtcccctctgactctctctgcagagagtgtagcagctccgcatgtcaccccttgggggtcagaagagcctctgctgcttttgacactgaatgtcaaatcagtactgattgtagtcagccaccagcaggtgaaccttccagaataattcttggcctcacatcttagaaaggtcttatttttgggttctttctggtcctttaaaatatcagtggaccaaattccatcttcctttttgtgaagcagcaggagggaatggcttagaacctcccctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatcaggataccaatccaattctaccacataaacatctttcttcagttcccatatggccacaaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaattagcaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatactgaaagtgatgttcaccccagttgcaaagtaacagcaatgaagtgctttctcttggagttacaagttatttcacttgagagcggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 25 - DNA, 3916 bp, synthetic construct
  source 1..3916: mol_type = other DNA; note = Human UbC -modified IL-12A/IL-12B/IL-15 - F2A (with CpG modification)
gtcgaccacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctccgctctcaagtgaaataacttgtaactccaagagaaagcacttcattgctgttactttgcaactggggtgaacatcactttcagtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttgctaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 26 - DNA, 4408 bp, synthetic construct
  source 1..4408: mol_type = other DNA; note = Human UbC - modified IL-12A/IL-12B/IL-15 - IRES (with CpG modification)
gtcgaccacacaaaaaaccaacacacagatctaatgaaaataaagatcttttattggcgcgcctcaactgcagggcacagatgcccattctgaccaagatgagctatagtatctgtcctgggccctcactgaaatgctggcatttttcctgcagatgactgtggctgaggtcttgtcagtgaagactctatctttcttttctctcttgctcttgccctggacttgaacacagaatgtcagggagaagtaggaatgtggagtactccaggtgtcagggtactcccagctgacctccacctgcctagaattctttaatggcttcagctgcaagttcttgggtgggtcaggtttgatgatgtccctgatgaagaagctgctggtgtagttttcatacttgagcttgtgaactgcatccaccatcacctcaatgggcagactctcctcagcagctgggcaggcactgtcctcctggcactccactgagtactcatactccttgttgtcccctctgactctctctgcagagagtgtagcagctccacatgtcaccccttgggggtcagaagagcctctgctgcttttgacactgaatgtcaaatcagtactgattgtagtcagccaccagcaggtgaaccttccagaataattcttggcctcacatcttagaaaggtcttatttttgggttctttctggtcctttaaaatatcagtggaccaaattccatcttcctttttgtgaagcagcaggagggaatggcttagaacctcccctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatcaggataccaatccaattctaccacataaacatctttcttcagttcccatatggccacaaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaattagcaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatactgaaagtgatgttcaccccagttgcaaagtaacagcaatgaagtgctttctcttggagttacaagttatttcacttgagtcaggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 27 - DNA, 3916 bp, synthetic construct
  source 1..3916: mol_type = other DNA; note = Human UbC - modified IL-12A/IL-12B/IL-15 - F2A - (with CpG modification)
gtcgaccacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctcctgactcaagtgaaataacttgtaactccaagagaaagcacttcattgctgttactttgcaactggggtgaacatcactttcagtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttgctaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 28 - DNA, 4456 bp, synthetic construct
  source 1..4456: mol_type = other DNA; note = Human UbC- wild-type IL-12A/IL-12B/IL-15 -ODN-IRES (no CpG modification)
gtcgactcgtcgttttgtcgttttgtcgttcacacaaaaaaccaacacacagatctaatgaaaataaagatcttttattggcgcgcctcaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacttgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatcacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgagaatttcgaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtcgtcgttttgtcgttttgtcgttgagctcgag

SEQ ID NO: 29 - DNA, 3964 bp, synthetic construct
  source 1..3964: mol_type = other DNA; note = Human UbC- wild-type IL-12A/IL-12B/IL-15 -ODN-F2A (no CpG modification)
gtcgactcgtcgttttgtcgttttgtcgttcacacaaaaaaccaacacacagatctaatgaaaataaagatcttttattggcgcgcctcaagaagtgttgatgaacatttggacaatatgtacaaaactctgcaaaaattctttaatatttttttcctccagttcctcacattctttgcatccagattctgttacattcccattagaagacaaactgttgtttgctaggatgatcagattttctactgtatcatgaatacttgcatctccggactcaagtgaaataacttgtaactccaagagaaagcacttcattgctgttactttgcaactggggtgaacatcactttccgtatataaagtagcatcaatatgcatagattgaataagatcttcaatttttttcaaatcacttattacattcacccagttggcttctgttttaggaagccctgcagagaaacagcccaaaatgaagacatgaatgccagcttcagttagaaaatgactgtttagaagtaaacacaagtagcactggatggaaatacttctcaaatgtggtttcgaaattctcatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcagaactgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtcgtcgttttgtcgttttgtcgttgagctcgag

SEQ ID NO: 30 - DNA, 2782 bp, synthetic construct
  source 1..2782: mol_type = other DNA; note = Human CMV- wild-type IL-12A/IL-12B - -F2A (no CpG modification)
gtcgacttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 31 - DNA, 1531 bp, synthetic construct
  source 1..1531: mol_type = other DNA; note = Human CMV- wild-type IL-15 (no CpG modification)
gtcgacttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatgagaatttcgaaaccacatttgagaagtatttccatccagtgctacttgtgtttacttctaaacagtcattttctaactgaagctggcattcatgtcttcattttgggctgtttctctgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 32-33 - skipped (2 entries)

SEQ ID NO: 34 - DNA, 4509 bp, synthetic construct
  source 1..4509: mol_type = other DNA; note = Human CAG - wild-type IL-12A/IL-12B - IRES (no CpG modification) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccttgttcatgccttcttctctttcctacagctcctgggcaacgtgctggttgttgtgctgtctcatcattttggcaaagaattcaagcttccagctagcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 35 - DNA, 2250 bp, synthetic construct
  source 1..2250: mol_type = other DNA; note = Human CAG - wild-type IL-12A/IL-12B - IRES (no CpG modification) expression sequence
gctagcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgacaattg

SEQ ID NO: 36 - DNA, 2766 bp, synthetic construct
  source 1..2766: mol_type = other DNA; note = Human CAG - wild-type IL-15 (no CpG modification) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgagaatttcgaaaccacatttgagaagtatttccatccagtgctacttgtgtttacttctaaacagtcattttctaactgaagctggcattcatgtcttcattttgggctgtttctctgctgggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 37 - DNA, 508 bp, synthetic construct
  source 1..508: mol_type = other DNA; note = Human CAG - wild-type IL-15 (no CpG modification) expression sequence
agctagcgccaccatgagaatttcgaaaccacatttgagaagtatttccatccagtgctacttgtgtttacttctaaacagtcattttctaactgaagctggcattcatgtcttcattttgggctgtttctctgctgggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgacaattg

SEQ ID NO: 38 - DNA, 4912 bp, synthetic construct
  source 1..4912: mol_type = other DNA; note = Mouse EF1a - wild-type IL-15 (no CpG modification) and modified IL-12B (with CpG modification) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgaccacacaaaaaaccaacacacagatctaatgaaaataaagatcttttattggcgcgcctcaggatcggactctgcagggaacacaggcccacttgctgcaggaggaattgtaatagcgatcctgagcttgcacgcagacattcccgcctttgcactggacttcggtagatgtcttctctacgaggaacgctcctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 39 - DNA, 4626 bp, synthetic construct
  source 1..4626: mol_type = other DNA; note = Mouse EF1a - wild-type IL-15 (no CpG modification) and modified IL-12B (with CpG modification) expression sequence
gtcgaccacacaaaaaaccaacacacagatctaatgaaaataaagatcttttattggcgcgcctcaggatcggactctgcagggaacacaggcccacttgctgcaggaggaattgtaatagcgatcctgagcttgcacgcagacattcccgcctttgcactggacttcggtagatgtcttctctacgaggaacgctcctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 40 - DNA, 4912 bp, synthetic construct
  source 1..4912: mol_type = other DNA; note = Mouse EF1a - modified IL-12B/IL-15 (CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgaccacacaaaaaaccaacacacagatctaatgaaaataaagatcttttattggcgcgcctcaggacctgactctgcagggaacacatgcccacttgctgcaggaggaattgtaatatctatcctgagcttgcacacagacattcccacctttgcattggacttctgtagatgtcttctctacaaggaaggcacctttctggttacacccctcctctgtctccttcatcttttctttcttcctctggattctaacaaagaacttgagggagaagtaggaatggggagtgctccaggagtcagggtactcccagctgacctccacctgtgagttcttcaaaggcttcatctgcaagttcttgggggggtctggtttgatgatgtccctgatgaagaagctggtgctgtagttctcatatttattctgctgccttgcttccaaggccagttcaatgggcagggtctcctcagcagttgggcaggtgacatcctcctggcaggacactgaatacttctcatagtccctttggtccagtgtgaccttctctgcagacagagaagccattccacatgtcactgctctagagtcaggggaactgctactgctcttgatgttgaacttcaagtccatgtttctttgcaccagccatgagcatgtgaatcttccactgtaatttggtgcttcacacttcaggaaagtcttgtttttgaaattttttaaaatttcagtggaccaaattccattttccttcttgtggagcagcagatgtgagtggctcagagtctcccctcctttgtggcaggtgtactggccagcatctagaaactctttgacagtgatggtcagggtctttccagagcctatgactccatgtctctggtctgaggtccaggtgatgtcatcttcttcaggtgtgtcacaggtgaggttcactgtttctccaggggcatctggagtccagtccacctctacaacataaacatctttctccagctcccacatggccatgagtggagacaccagcaaaacaatggcaaaccaggagatggttagcttctgaggacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatggtcagtgttccaacagcctcaccctcagcatccagcagctcctctcagtgcaggtccagcatgtgtcaatcaagatacctcctctttttggccacccttgccctcctaaaccacctcagtttggccagggtcattccagtctctggacctgccaggtgtcttagccagtccaggaacctgctgaagaccacagatgacatggtgaagactgccagagaaaaactgaaacattattcctgcactgctgaagacatagatcatgaagacatcacaagagaccaaaccagcacattgaagacctgtttaccactggaactacacaagaatgagagttgcctggctactagagagacttcttccacaacaagagggagctgcctgcccccacagaagacctctttgatgatgaccctgtgccttggtagcatctatgaggacttgaagatgtaccagacagagttccaggccatcaatgcagcacttcagaatcacaaccatcagcagatcattctagacaagggcatgctggtggccattgatgagctgatgcagtctctgaatcataatggggagactctgaggcagaaacctcctgtgggagaagcagacccttacagagtgaaaatgaagctctgcatcctgcttcatgccttcagcaccagagtagtgaccatcaacagggtgatgggctatctgagcagtgcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgaaaattttgaaaccatatatgaggaatacatccatctcatgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaatgtgctctaccttgcaaacagcactctgtcttctaacaagaatgtagcagaatctggctgcaaggaatgtgaggagctggaggagaaaaccttcacagagtttttgcaaagctttataaggattgtccaaatgttcatcaacacctcctgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 41 - DNA, 4626 bp, synthetic construct
  source 1..4626: mol_type = other DNA; note = Mouse EF1a - modified IL-12B/IL-15 (CpG modified) expression sequence
gtcgaccacacaaaaaaccaacacacagatctaatgaaaataaagatcttttattggcgcgcctcaggacctgactctgcagggaacacatgcccacttgctgcaggaggaattgtaatatctatcctgagcttgcacacagacattcccacctttgcattggacttctgtagatgtcttctctacaaggaaggcacctttctggttacacccctcctctgtctccttcatcttttctttcttcctctggattctaacaaagaacttgagggagaagtaggaatggggagtgctccaggagtcagggtactcccagctgacctccacctgtgagttcttcaaaggcttcatctgcaagttcttgggggggtctggtttgatgatgtccctgatgaagaagctggtgctgtagttctcatatttattctgctgccttgcttccaaggccagttcaatgggcagggtctcctcagcagttgggcaggtgacatcctcctggcaggacactgaatacttctcatagtccctttggtccagtgtgaccttctctgcagacagagaagccattccacatgtcactgctctagagtcaggggaactgctactgctcttgatgttgaacttcaagtccatgtttctttgcaccagccatgagcatgtgaatcttccactgtaatttggtgcttcacacttcaggaaagtcttgtttttgaaattttttaaaatttcagtggaccaaattccattttccttcttgtggagcagcagatgtgagtggctcagagtctcccctcctttgtggcaggtgtactggccagcatctagaaactctttgacagtgatggtcagggtctttccagagcctatgactccatgtctctggtctgaggtccaggtgatgtcatcttcttcaggtgtgtcacaggtgaggttcactgtttctccaggggcatctggagtccagtccacctctacaacataaacatctttctccagctcccacatggccatgagtggagacaccagcaaaacaatggcaaaccaggagatggttagcttctgaggacacatggtggcggcgcgccatttaaatctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttcgatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcgatcttaattaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgtttaaacttctgctctgcggccgcgccaccatggtcagtgttccaacagcctcaccctcagcatccagcagctcctctcagtgcaggtccagcatgtgtcaatcaagatacctcctctttttggccacccttgccctcctaaaccacctcagtttggccagggtcattccagtctctggacctgccaggtgtcttagccagtccaggaacctgctgaagaccacagatgacatggtgaagactgccagagaaaaactgaaacattattcctgcactgctgaagacatagatcatgaagacatcacaagagaccaaaccagcacattgaagacctgtttaccactggaactacacaagaatgagagttgcctggctactagagagacttcttccacaacaagagggagctgcctgcccccacagaagacctctttgatgatgaccctgtgccttggtagcatctatgaggacttgaagatgtaccagacagagttccaggccatcaatgcagcacttcagaatcacaaccatcagcagatcattctagacaagggcatgctggtggccattgatgagctgatgcagtctctgaatcataatggggagactctgaggcagaaacctcctgtgggagaagcagacccttacagagtgaaaatgaagctctgcatcctgcttcatgccttcagcaccagagtagtgaccatcaacagggtgatgggctatctgagcagtgcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgaaaattttgaaaccatatatgaggaatacatccatctcatgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaatgtgctctaccttgcaaacagcactctgtcttctaacaagaatgtagcagaatctggctgcaaggaatgtgaggagctggaggagaaaaccttcacagagtttttgcaaagctttataaggattgtccaaatgttcatcaacacctcctgagcggccgcgctagccaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgag

SEQ ID NO: 42 - DNA, 4581 bp, synthetic construct
  source 1..4581: mol_type = other DNA; note = Mouse CAG - wild-type IL-12B - IRES (not CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggagcgttcctcgtagagaagacatctaccgaagtccaatgcaaaggcgggaatgtctgcgtgcaagctcaggatcgctattacaattcctcatgcagcaagtgggcatgtgttccctgcagggtccgatcctgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 43 - DNA, 2322 bp, synthetic construct
  source 1..2322: mol_type = other DNA; note = Mouse CAG - wild-type IL-12B - IRES (not CpG modified) expression sequence
gctagcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggagcgttcctcgtagagaagacatctaccgaagtccaatgcaaaggcgggaatgtctgcgtgcaagctcaggatcgctattacaattcctcatgcagcaagtgggcatgtgttccctgcagggtccgatcctgacaattg

SEQ ID NO: 44 - DNA, 2766 bp, synthetic construct
  source 1..2766: mol_type = other DNA; note = Mouse CAG - wild-type IL-15 (not CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 45 - DNA, 507 bp, synthetic construct
  source 1..507: mol_type = other DNA; note = Mouse CAG - wild-type IL-15 (not CpG modified) expression sequence
gctagcgccaccatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgacaattg

SEQ ID NO: 46 - DNA, 4089 bp, synthetic construct
  source 1..4089: mol_type = other DNA; note = Mouse CAG - wild-type IL-12B - F2A (not CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccagaaagagaaggagtggctcaggagcccctgtgaaacagaccctgaactttgacctcttgaagcttgctggggatgtggagtctaatcctggtccaatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggagcgttcctcgtagagaagacatctaccgaagtccaatgcaaaggcgggaatgtctgcgtgcaagctcaggatcgctattacaattcctcatgcagcaagtgggcatgtgttccctgcagggtccgatcctgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 47 - DNA, 1830 bp, synthetic construct
  source 1..1830: mol_type = other DNA; note = Mouse CAG - wild-type IL-12B - F2A (not CpG modified) expression sequence
gctagcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccagaaagagaaggagtggctcaggagcccctgtgaaacagaccctgaactttgacctcttgaagcttgctggggatgtggagtctaatcctggtccaatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggagcgttcctcgtagagaagacatctaccgaagtccaatgcaaaggcgggaatgtctgcgtgcaagctcaggatcgctattacaattcctcatgcagcaagtgggcatgtgttccctgcagggtccgatcctgacaattg

SEQ ID NO: 48 - DNA, 4017 bp, synthetic construct
  source 1..4017: mol_type = other DNA; note = Human CAG - modified IL-12A/IL-12B - F2A (CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 49 - DNA, 1758 bp, synthetic construct
  source 1..1758: mol_type = other DNA; note = Human CAG - modified IL-12A/IL-12B - F2A (CpG modified) expression sequence
gctagcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttgacaattg

SEQ ID NO: 50 - DNA, 4017 bp, synthetic construct
  source 1..4017: mol_type = other DNA; note = Human CAG - modified IL-12A/IL-12B - F2A (CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 51 - DNA, 1758 bp, synthetic construct
  source 1..1758: mol_type = other DNA; note = Human CAG - modified IL-12A/IL-12B - F2A (CpG modified) expression sequence
gctagcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttgacaattg

SEQ ID NO: 52 - DNA, 4509 bp, synthetic construct
  source 1..4509: mol_type = other DNA; note = Human CAG - modified IL-12A/IL-12B - IRES (CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 53 - DNA, 2250 bp, synthetic construct
  source 1..2250: mol_type = other DNA; note = Human CAG - modified IL-12A/IL-12B - IRES (CpG modified) expression sequence
gctagcgccaccatgtgtccagctaggagcctcctccttgtggctaccctggtcctcctggaccacctcagtttggccagaaacctccctgtggccactccagacccaggaatgttcccatgccttcaccactcccaaaacctgctgagggcagtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattagggcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgtggagctgctacactctctgcagagagagtcagaggggacaacaaggagtatgagtactcagtggagtgccaggaggacagtgcctgcccagctgctgaggagagtctgcccattgaggtgatggtggatgcagttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgcaggaaaaatgccagcatttcagtgagggcccaggacagatactatagctcatcttggtcagaatgggcatctgtgccctgcagttgacaattg

SEQ ID NO: 54 - DNA, 4509 bp, synthetic construct
  source 1..4509: mol_type = other DNA; note = Human CAG - modified IL-12A/IL-12B - IRES (CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 55 - DNA, 2250 bp, synthetic construct
  source 1..2250: mol_type = other DNA; note = Human CAG - modified IL-12A/IL-12B - IRES (CpG modified) expression sequence
gctagcgccaccatgtgtccagcgcgcagcctcctccttgtggctaccctggtcctcctggaccacctcagtttggccagaaacctccctgtggccactccagacccaggaatgttcccatgccttcaccactcccaaaacctgctgagggctgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaacctgatttttataaaactaaaatcaagctctgcatacttcttcatgctttccggattcgcgcagtgactattgatagagtgatgagctatctgaatgcttcctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgtgtcaccagcagttggtcatctcttggttttccctggtttttctggcatctccccttgtggccatatgggaactgaagaaagatgtttatgtggtagaattggattggtatcctgatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggggaggttctaagccattccctcctgctgcttcacaaaaaggaagatggaatttggtccactgatattttaaaggaccagaaagaacccaaaaataagacctttctaagatgtgaggccaagaattattctggaaggttcacctgctggtggctgactacaatcagtactgatttgacattcagtgtcaaaagcagcagaggctcttctgacccccaaggggtgacatgcggagctgctacactctctgcagagagagtcagaggggacaacaaggagtatgagtactcagtggagtgccaggaggacagtgcctgcccagctgctgaggagagtctgcccattgaggtgatggtggatgcggttcacaagctcaagtatgaaaactacaccagcagcttcttcatcagggacatcatcaaacctgacccacccaagaacttgcagctgaagccattaaagaattctaggcaggtggaggtcagctgggagtaccctgacacctggagtactccacattcctacttctccctgacattctgtgttcaagtccagggcaagagcaagagagaaaagaaagatagagtcttcactgacaagacctcagccacagtcatctgccgcaaaaatgccagcatttcagtgcgggcccaggaccgctactatagctcatcttggtcagaatgggcatctgtgccctgcagttgacaattg

SEQ ID NO: 56 - DNA, 4017 bp, synthetic construct
  source 1..4017: mol_type = other DNA; note = Human CAG - wild-type IL-12A/IL-12B - F2A (CpG modified) construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgacaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 57 - DNA, 1758 bp, synthetic construct
  source 1..1758: mol_type = other DNA; note = Human CAG - wild-type IL-12A/IL-12B - F2A (CpG modified) expression sequence
gctagcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgacaattg

SEQ ID NO: 58 - DNA, 489 bp, synthetic construct
  source 1..489: mol_type = other DNA; note = IL-15 (with CpG modification)
atgagaatttcgaaaccacatttgagaagtatttccatccagtgctacttgtgtttacttctaaacagtcattttctaactgaagctggcattcatgtcttcattttgggctgtttctctgctgggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 59 - DNA, 585 bp, synthetic construct
  source 1..585: mol_type = other DNA; note = IRES
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaca

SEQ ID NO: 60 - DNA, 24 bp, synthetic construct
  source 1..24: mol_type = other DNA; note = Furin cleaveage site
agaaagagaaggagtggctcagga

SEQ ID NO: 61 - DNA, 72 bp, synthetic construct
  source 1..72: mol_type = other DNA; note = 2A site
gcccctgtgaaacagaccctgaactttgacctcttgaagcttgctggggatgtggagtctaatcctggtcca

SEQ ID NO: 62 - DNA, 60 bp, synthetic construct
  source 1..60: mol_type = other DNA; note = IL-2 leader
atgtacaggatgcaactcctgtcttgcattgcactaagtcttgcacttgtcacaaactcg

SEQ ID NO: 63 - DNA, 54 bp, synthetic construct
  source 1..54: mol_type = other DNA; note = IL-10 leader
atgcacagctcagcactgctctgttgcctggtcctcctgactggggtgagggcc

SEQ ID NO: 64 - DNA, 23 bp, synthetic construct
  source 1..23: mol_type = other DNA; note = miR-142 binding site
tccataaagtaggaaacactaca

SEQ ID NO: 65 - DNA, 104 bp, synthetic construct
  source 1..104: mol_type = other DNA; note = 4x miR-142 binding site
tccataaagtaggaaacactacactattccataaagtaggaaacactacatcactccataaagtaggaaacactacaagtctccataaagtaggaaacactaca

SEQ ID NO: 66 - DNA, 97 bp, synthetic construct
  source 1..97: mol_type = other DNA; note = SV40 intron
gtaagtttagtctttttgtcttttatttcaggtcccggatccggtggtggtgcaaatcaaagaactgctcctcagtggatgttgcctttacttctag

SEQ ID NO: 67 - DNA, 230 bp, synthetic construct
  source 1..230: mol_type = other DNA; note = EF-1alpha
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 68 - DNA, 380 bp, synthetic construct
  source 1..380: mol_type = other DNA; note = CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 69 - DNA, 218 bp, synthetic construct
  source 1..218: mol_type = other DNA; note = CMVp
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 70 - DNA, 598 bp, synthetic construct
  source 1..598: mol_type = other DNA; note = CMV ehancer and promoter (CMVe/p)
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 71 - DNA, 1725 bp, synthetic construct
  source 1..1725: mol_type = other DNA; note = CAG
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaa

SEQ ID NO: 72 - DNA, 49 bp, synthetic construct
  source 1..49: mol_type = other DNA; note = Synthetic poly(A)
aataaaagatctttattttcattagatctgtgtgttggttttttgtgtg

SEQ ID NO: 73 - DNA, 224 bp, synthetic construct
  source 1..224: mol_type = other DNA; note = BGH poly(A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 74 - DNA, 92 bp, synthetic construct
  source 1..92: mol_type = other DNA; note = Pause element
aacatacgctctccatcaaaacaaaacgaaacaaaacaaactagcaaaataggctgtccccagtgcaagtgcaggtgccagaacatttctct

SEQ ID NO: 75 - DNA, 269 bp, synthetic construct
  source 1..269: mol_type = other DNA; note = 5' LTR
ggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 76 - DNA, 133 bp, synthetic construct
  source 1..133: mol_type = other DNA; note = Intron (chimera of human betaglobin intron and immunoglobulin heavy chain intron)
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 77 - DNA, 657 bp, synthetic construct
  source 1..657: mol_type = other DNA; note = Human IL-12A (p35) ("wild-type" no CpG modification and no stop codon)
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttcc

SEQ ID NO: 78 - protein, 236 aa, synthetic construct
  source 1..236: note = Mouse IL-12A (p35) NP_001152896
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 79 - protein, 335 aa, synthetic construct
  source 1..335: note = Mouse IL-12B (p40) NP_001290173
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 80 - protein, 253 aa, synthetic construct
  source 1..253: note = Human IL-12A (p35) NP_000873
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 81 - protein, 328 aa, synthetic construct
  source 1..328: note = Human IL-12B (p40) NP_002178
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 82 - DNA, 4280 bp, synthetic construct
  source 1..4280: mol_type = other DNA; note = CMV enhancer -CBA promoter- WT human IL-12A (no stop codon) - F2A - WT human IL-12B (with stop codon) -hGH polyA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttccggattcgggcagtgactattgatagagtgatgagctatctgaatgcttccagaaagagaaggagtggctcaggagcccctgtgaaacagaccctgaactttgacctcttgaagcttgctggggatgtggagtctaatcctggtccaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttgacaattggatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 83 - DNA, 4352 bp, synthetic construct
  source 1..4352: mol_type = other DNA; note = CMV enhancer -CBA promoter- WT mouse IL-12A (no stop codon) - F2A - WT mouse IL-12B (with stop codon) -hGH polyA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccagaaagagaaggagtggctcaggagcccctgtgaaacagaccctgaactttgacctcttgaagcttgctggggatgtggagtctaatcctggtccaatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggagcgttcctcgtagagaagacatctaccgaagtccaatgcaaaggcgggaatgtctgcgtgcaagctcaggatcgctattacaattcctcatgcagcaagtgggcatgtgttccctgcagggtccgatcctgacaattggatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 84 - DNA, 130 bp, synthetic construct
  source 1..130: mol_type = other DNA; note = AAV2 5' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 85 - DNA, 276 bp, synthetic construct
  source 1..276: mol_type = other DNA; note = Chicken Beta Actin (CBA) Promoter
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 86 - DNA, 1064 bp, synthetic construct
  source 1..1064: mol_type = other DNA; note = CAG Intron
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaa

SEQ ID NO: 87 - skipped

SEQ ID NO: 88 - DNA, 487 bp, synthetic construct
  source 1..487: mol_type = other DNA; note = Human growth hormone (hGH) poly(A)
gatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctg

SEQ ID NO: 89 - DNA, 141 bp, synthetic construct
  source 1..141: mol_type = other DNA; note = AAV2 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 90 - DNA, 708 bp, synthetic construct
  source 1..708: mol_type = other DNA; note = WT mouse IL-12A (p35)(no stop codon)
atggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 91 - DNA, 1008 bp, synthetic construct
  source 1..1008: mol_type = other DNA; note = WT mouse IL-12B (p40)
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggagcgttcctcgtagagaagacatctaccgaagtccaatgcaaaggcgggaatgtctgcgtgcaagctcaggatcgctattacaattcctcatgcagcaagtgggcatgtgttccctgcagggtccgatcctga

SEQ ID NO: 92 - DNA, 984 bp, synthetic construct
  source 1..984: mol_type = other DNA; note = Human IL-12B (p40) ("wild-type" no CpG modification and without stop codon)
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtgatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaagtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

(19) United States
(12) Patent Application Publication
(10) Pub. No.: US 2025/0059564 A1
(43) Pub. Date: Feb. 20, 2025

(54) VIRAL VECTOR CONSTRUCTS FOR DELIVERY OF NUCLEIC ACIDS ENCODING CYTOKINES AND USES THEREOF FOR TREATING CANCER

(71) Applicant: KRIYA THERAPEUTICS, INC., Morrisville, NC (US)

(72) Inventors: Brian FURMANSKI, Morrisville, NC (US); Nachiketa GUPTA, Morrisville, NC (US); Bruce SCHNEPP, Morrisville, NC (US); Michele STONE, Morrisville, NC (US)

(21) Appl. No.: 18/785,522

(22) Filed: Jul. 26, 2024

Related U.S. Application Data
(62) Division of application No. 17/500,898, filed on Oct. 13, 2021, now Pat. No. 12,071,633.
(60) Provisional application No. 63/091,270, filed on Oct. 13, 2020.
(60) Provisional application No. 63/108,838, filed on Nov. 2, 2020.
(60) Provisional application No. 63/141,921, filed on Jan. 26, 2021.
(60) Provisional application No. 63/182,632, filed on Apr. 30, 2021.

(51) Int. Cl. C12N 15/86; A61K 38/20; A61K 39/395; A61P 35/00

(52) U.S. Cl. CPC C12N 15/86; A61K 38/208; A61K 39/3955; A61P 35/00; C12N 2750/14132; C12N 2750/14143; C12N 2750/14171; C12N 2830/008; C12N 2840/203

(57) ABSTRACT

The present disclosure provides the gene therapy compositions comprising vectors (e.g., viral vectors) suitable for delivery of nucleic acids encoding immunomodulatory proteins or functional fragments thereof, and methods of using the same. Certain aspects of the disclosure are directed to an adeno-viral vector (AAV) delivery of nucleic acids encoding two or more immunomodulatory proteins or functional fragments thereof to a tumor.

### CROSS REFERENCE TO RELATED APPLICATIONS

The present application claims the priority benefit of U.S. Provisional Application No. 63/182,632, filed Apr. 30, 2021; U.S. Provisional Application No. 63/141,921, filed Jan. 26, 2021; U.S. Provisional Application No. 63/108,838, filed Nov. 2, 2020; and U.S. Provisional Application No. 63/091,270, filed Oct. 13, 2020, each of which is hereby incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4525_0320005_Seqlisting_ST25.txt; Size: 254,500 bytes; and Date of Creation: Oct. 9, 2023), filed with the application, is incorporated herein by reference in its entirety.

### FIELD OF DISCLOSURE

The present disclosure pertains to the medical field including oncology and gene therapy. Certain aspects of the disclosure relate to compositions and methods for expressing immunomodulatory proteins (e.g., cytokines) or functional fragments thereof and uses thereof.

### BACKGROUND

Cytokines participate in regulation of the immune system. When used in cancer therapy, cytokines can act as immunomodulatory agents that have anti-tumor effects and can increase the immune response towards different types of tumors. However, rapid blood clearance and lack of tumor specificity require systemic administration of high doses of a cytokine in order to achieve a concentration of the cytokine at the tumor site and/or other relevant tissues (e.g., lymph nodes and spleen) sufficient to activate an immune response or have an antitumor effect. However, high levels of systemic cytokine can lead to severe toxicity and adverse reactions clinically.

### BRIEF SUMMARY

In some aspects, provided herein are compositions (e.g., gene therapy compositions) and methods comprising a combination of (a) a viral vector (e.g., AAV capsid) comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 or PDL-1 inhibitor). In some aspects, the viral vector (e.g., AAV capsid comprising a polynucleotide, also referred to herein as an AAV particle) and the checkpoint inhibitor are delivered in combination (e.g., simultaneously or sequentially). In some aspects, the viral vector (e.g., AAV particle) described herein can be administered prior to, at the same time, or after the administration of a checkpoint inhibitor. In some aspects, the polynucleotide comprises: (i) a first nucleic acid encoding a first cytokine subunit or functional fragment thereof; and (ii) a second nucleic acid encoding a second cytokine subunit or functional fragment thereof. In some aspects, the first nucleic acid comprises a nucleic acid encoding an IL-12 p35 subunit and/or a nucleic acid encoding an IL-12 p40 subunit. In some aspects, the second nucleic acid comprises a nucleic acid encoding an IL-12 p35 subunit and/or a nucleic acid encoding an IL-12 p40 subunit.

In some aspects, the first nucleic acid encodes a polypeptide having at least 85%, 90%, 95%, 96%, 97%, 98%, 99%, or 100% identity to the amino acid sequence of any of SEQ ID NO: 80 or 81, or any amino acid sequence in Table 3. In some aspects, the nucleic acid encoding the first nucleic acid (e.g., a nucleic acid encoding an IL-12 p35 subunit and/or a nucleic acid encoding an IL-12 p40 subunit) does not include an in-frame stop codon (e.g., TGA, TAG, and TAA). In some aspects, the first nucleic acid and/or the second nucleic acid comprises a nucleic acid sequence having at least 85%, 90%, 95%, 96%, 97%, 98%, 99%, or 100% identity to any of SEQ ID NO: 5-10, 77, 92, or any nucleic acid sequence in Table 2. In some aspects, the first nucleic acid and/or the second nucleic acid comprises a nucleic acid sequence having at least 85%, 90%, 95%, 96%, 97%, 98%, 99%, or 100% identity to a reverse complement of any of SEQ ID NO: 5-10, 77, 92, or any nucleic acid sequence in Table 2. In some aspects, the first nucleic acid comprises a nucleic acid sequence having at least 95%, 96%, 97%, 98%, 99%, or 100% identity to SEQ ID NO: 77, and the second nucleic acid comprises a nucleic acid sequence having at least 95%, 96%, 97%, 98%, 99%, or 100% identity to SEQ ID NO: 8.

In some aspects, provided herein are compositions and methods comprising one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof. In some aspects, each delivery vector (e.g., viral vector) comprises a polynucleotide comprising the first nucleic acid and the second nucleic acid. In some aspects, each delivery vector (e.g., viral vector) comprises either the first nucleic acid or the second nucleic acid, wherein two or more delivery vectors are delivered in combination (e.g., simultaneously or sequentially). In some aspects, the delivery vector comprises constructs comprising nucleic acids encoding a first immunomodulatory protein and the subunits of a second immunomodulatory protein.

In some aspects, a delivery vector (e.g., viral vector) for delivery of (i) a first nucleic acid encoding a first immunomodulatory protein, one or more subunits thereof, or functional fragment thereof and/or (ii) a second nucleic acid encoding a second immunomodulatory protein, one or more subunits thereof, or functional fragment thereof is administered in combination with a checkpoint inhibitor. In some aspects, the delivery vector described herein can be administered prior to, at the same time, or after the administration of a checkpoint inhibitor. In some aspects, the viral vector comprises constructs comprising nucleic acids encoding a first immunomodulatory protein and the subunits of a second immunomodulatory protein.

In some aspects, two or more delivery vectors (e.g., viral vectors) described herein are delivered in combination (e.g., simultaneously or sequentially) and further in combination with a checkpoint inhibitor. In some aspects, the two or more delivery vectors described herein can be administered prior to, at the same time, or after the administration of a checkpoint inhibitor.

In some aspects, provided herein is a composition comprising: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector).

In some aspects, provided herein is a polynucleotide comprising: (a) a first nucleic acid encoding a first cytokine, one or more subunits thereof, or functional fragment thereof; and (b) a second nucleic acid encoding a second cytokine, one or more subunits thereof, or functional fragment thereof.

In some aspects, the first immunomodulatory protein or functional fragment thereof and/or the second immunomodulatory protein or functional fragment thereof is a cytokine or functional fragment thereof.

In some aspects, the cytokine (e.g., the first cytokine and/or the second cytokine) is selected from the group consisting of tumor necrosis factor alpha (TNF-α), a type I interferon (INF), a type II IFN, interleukin (IL)-2, IL-12, IL-15, IL-21, IL-23, IL-27, IL-18, IL-1, IL-3, IL-4, IL-5, IL-6, IL-7, IL-8, IL-9, IL-10, IL17, IL33, TL1A, CD40L, granulocyte-macrophage colony-stimulating factor (GM-CSF), any functional fragment thereof, and any combination thereof.

In some aspects, the first immunomodulatory and the second immunomodulatory proteins are cytokines.

In some aspects, the first immunomodulatory protein (e.g., the cytokine) is IL-12 or a functional fragment thereof. In some aspects, the first nucleic acid comprises an IL-12 p35 gene and/or an IL-12 p40 gene. In some aspects, the first immunomodulatory protein (e.g., the cytokine) is IL-12 or a functional fragment thereof. In some aspects, the first nucleic acid comprises a nucleic acid encoding an IL-12 p35 subunit and/or a nucleic acid encoding an IL-12 p40 subunit. In some aspects, the first nucleic acid encodes a polypeptide having at least 85%, 90%, 95%, 96%, 97%, 98%, 99%, or 100% identity to the amino acid sequence of any of SEQ ID NO: 80 or 81, or any amino acid sequence in Table 3. In some aspects, the nucleic acid encoding the first nucleic acid (e.g., a nucleic acid encoding an IL-12 p35 subunit and/or a nucleic acid encoding an IL-12 p40 subunit) does not include an in-frame stop codon (e.g., TGA, TAG, and TAA). In some aspects, the first nucleic acid comprises a nucleic acid sequence having at least 85%, 90%, 95%, 96%, 97%, 98%, 99%, or 100% identity to any of SEQ ID NO: 5-10, 77, or any nucleic acid sequence in Table 2. In some aspects, the first nucleic acid comprises a nucleic acid sequence having at least 85%, 90%, 95%, 96%, 97%, 98%, 99%, or 100% identity to a reverse complement of any of SEQ ID NO: 5-10, 77, 92, or any nucleic acid sequence in Table 2.

In some aspects, the second immunomodulatory protein (e.g., the cytokine) comprises IL-15 or a functional fragment thereof. In some aspects, the second nucleic acid comprises an IL-15 gene. In some aspects, the second nucleic acid comprises a nucleic acid sequence having at least 85%, 90%, 95%, 96%, 97%, 98%, 99%, or 100% identity to any of SEQ ID NO: 11-13, 58, or any nucleic acid sequence in Table 4. In some aspects, the second nucleic acid comprises a nucleic acid sequence having at least 85%, 90%, 95%, 96%, 97%, 98%, 99%, or 100% identity to a reverse complement of any of SEQ ID NO: 11-13, 58, or any nucleic acid sequence in Table 4.

In some aspects, provided herein are compositions and methods comprising one or more delivery vectors (e.g., viral vectors) for delivery of one or more polynucleotides encoding the combination of IL-12, one or more subunits thereof, or a functional fragment thereof and IL-15 or a functional fragment thereof. In some apects, a delivery vector (e.g., a viral vector, a non-viral vectors, a plasmid, a lipid, protein particle, a bacterial vector, a lysosome, a virus-like particle, a polymeric particle, an exosome, or a vault particle). In some aspects, each delivery vector is an AAV capsid (e.g., an AAV2 capsid) which comprises a polynucleotide encoding IL-12, one or more subunits thereof, or a functional fragment thereof and a polynucleotide encoding IL-15 or a functional fragment thereof.

In some aspects, each delivery vector is an AAV capsid (e.g., an AAV2 capsid) comprising either a polynucleotide encoding IL-12, one or more subunits thereof, or a functional fragment thereof or a polynucleotide encoding IL-15 or a functional fragment thereof, wherein two or more of the AAV capsids are delivered in combination (e.g., simultaneously or sequentially), e.g., intratumorally, intravenously, intrasplenically, intrathecally, intrahepatically, via intraosseous infusion, intradermal, intraparenchymal, via delivery to salivary gland, or via delivery into a lymph node. In some aspects, the AAV capsid (e.g., an AAV2 capsid) comprising a polynucleotide encoding IL-12, one or more subunits thereof, or a functional fragment thereof is administered intratumorally.

In some aspects, provided herein are compositions and methods comprising one or more delivery vectors (e.g., viral vectors) for delivery of one or more polynucleotides encoding the combination of IL-18 or a functional fragment thereof and IL-15 or a functional fragment thereof. In some aspects, each delivery vector is an AAV capsid (e.g., an AAV2 capsid) which comprises a polynucleotide encoding IL-18 or a functional fragment thereof and a polynucleotide encoding IL-15 or a functional fragment thereof. In some aspects, each delivery vector is an AAV capsid (e.g., an AAV2 capsid) comprising either a polynucleotide encoding IL-18 or a functional fragment thereof or a polynucleotide encoding IL-15 or a functional fragment thereof, wherein two or more of the AAV capsids are delivered in combination (e.g., simultaneously or sequentially), e.g., intratumorally, intrasplenically, intrathecally, intrahepatically, via intraosseous infusion, intradermal, intraparenchymal, via delivery to salivary gland, or via delivery into a lymph node. In some aspects, the AAV capsid (e.g., an AAV2 capsid) comprising a polynucleotide encoding IL-18 or a functional fragment thereof or a polynucleotide encoding IL-15 or a functional fragment thereof is administered intratumorally.

In some aspects, provided herein are compositions and methods comprising one or more delivery vectors (e.g., viral vectors) for delivery of one or more polynucleotides encoding the combination of IL-12, one or more subunits thereof, or a functional fragment thereof and IL-2 or a functional fragment thereof. In some aspects, each delivery vector is an AAV capsid (e.g., an AAV2 capsid) which comprises a polynucleotide encoding IL-12, one or more subunits thereof, or a functional fragment thereof and a polynucleotide encoding IL-2 or a functional fragment thereof. In some aspects, each delivery vector is an AAV capsid (e.g., an AAV2 capsid) comprising either a polynucleotide encoding IL-12, one or more subunits thereof, or a functional fragment thereof or a polynucleotide encoding IL-2 or a functional fragment thereof, wherein two or more of the AAV capsids are delivered in combination (e.g., simultaneously or sequentially), e.g., intratumorally, intravenously, intrasplenically, intrathecally, intrahepatically, via intraosseous infusion, intradermal, intraparenchymal, via delivery to salivary gland, or via delivery into a lymph node. In some aspects, the AAV capsid (e.g., an AAV2 capsid) comprising a polynucleotide encoding IL-12 or a functional fragment thereof or a polynucleotide encoding IL-2 or a functional fragment thereof is administered intratumorally.

In some aspects, provided herein are compositions and methods comprising one or more delivery vectors (e.g., viral vectors) for delivery of one or more polynucleotides encoding the combination of IL-18 or a functional fragment thereof and IL-2 or a functional fragment thereof. In some aspects, each delivery vector is an AAV capsid (e.g., an AAV2 capsid) which comprises a polynucleotide encoding IL-18 or a functional fragment thereof and a polynucleotide encoding IL-2 or a functional fragment thereof. In some aspects, each delivery vector is an AAV capsid (e.g., an AAV2 capsid) comprising either a polynucleotide encoding IL-18 or a functional fragment thereof or a polynucleotide encoding IL-2 or a functional fragment thereof, wherein two or more of the AAV capsids are delivered in combination (e.g., simultaneously or sequentially), e.g., intratumorally, intravenously, intrasplenically, intrathecally, intrahepatically, via intraosseous infusion, intradermal, intraparenchymal, via delivery to salivary gland, or via delivery into a lymph node. In some aspects, the AAV capsid (e.g., an AAV2 capsid) comprising a polynucleotide encoding IL-18 or a functional fragment thereof or a polynucleotide encoding IL-2 or a functional fragment thereof is administered intratumorally.

In some aspects, provided herein are compositions and methods comprising one or more delivery vectors (e.g., viral vectors) for delivery of one or more polynucleotides encoding the combination of IL-21 or a functional fragment thereof and IL-15 or a functional fragment thereof. In some aspects, each delivery vector is an AAV capsid (e.g., an AAV2 capsid) which comprises a polynucleotide encoding IL-21 or a functional fragment thereof and a polynucleotide encoding IL-15 or a functional fragment thereof. In some aspects, each delivery vector is an AAV capsid (e.g., an AAV2 capsid) comprising either a polynucleotide encoding IL-21 or a functional fragment thereof or a polynucleotide encoding IL-15 or a functional fragment thereof, wherein two or more of the AAV capsids are delivered in combination (e.g., simultaneously or sequentially), e.g., intratumorally, intravenously, intrasplenically, intrathecally, intrahepatically, via intraosseous infusion, intradermal, intraparenchymal, via delivery to salivary gland, or via delivery into a lymph node. In some aspects, the AAV capsid (e.g., an AAV2 capsid) comprising a polynucleotide encoding IL-21 or a functional fragment thereof or a polynucleotide encoding IL-15 or a functional fragment thereof is administered intratumorally.

In some aspects, the first immunomodulatory protein or functional fragment thereof or the second immunomodulatory protein or functional fragment thereof is a soluble natural killer (NK), B-cell, T-cell, neutrophil and/or macrophage ligand.

In some aspects, the first immunomodulatory protein or functional fragment thereof or the second immunomodulatory protein or functional fragment thereof is selected from the group consisting of soluble CD40 ligand (CD40L), CD19 ligand (CD19L), CD48 ligand (CD48L), CD20 ligand (CD20L), any functional fragment thereof, and any combination thereof.

In some aspects, the polynucleotides disclosed herein further comprise a promoter.

In some aspects, the promoter comprises a first promoter which is operably linked to the first nucleic acid.

In some aspects, the promoter comprises a second promoter which is operably linked to the second nucleic acid.

In some aspects, the promoter is a constitutively active promoter, a cell-type specific promoter, or an inducible promoter.

In some aspects, the promoter comprises a CBA promoter, a CMV promoter, an EF-1a (Elongation Factor 1a) promoter, aRSV (Rous Sarcoma Virus) promoter, an Ubiquitin (UbC promoter, or any combination thereof.

In some aspects, the polynucleotides disclosed herein further comprise an internal ribosomal entry site (IRES) sequence, a furin cleavage sequence, a 2A self-processing peptide sequence (including an F2A, E2A, P2A or T2A self-processing peptide), or a combination thereof. In some aspects, the polynucleotide comprises a furin cleavage sequence and a 2A self-processing peptide sequence (e.g., F2A).

In some aspects, the polynucleotides disclosed herein further comprise an enhancer sequence, an intron sequence, or a combination thereof.

In some aspects, the polynucleotides disclosed herein further comprises a poly(A) (pA) sequence (e.g., a growth hormone pA).

In some aspects, the polynucleotide of the disclosure comprises a promoter operably linked to an IL-12 p35 gene or functional fragment thereof (e.g., corresponding to SEQ ID NO: 77), a furin cleavage sequence and/or a 2A self-processing peptide sequence (including an F2A, E2A, P2A or T2A self-processing peptide), and an IL-12 p40 gene or a functional fragment thereof (e.g., corresponding to SEQ ID NO: 8); and a pA sequence (e.g., a growth hormone pA).

In some aspects, the first nucleic acid comprises a promoter operably linked to an IL-12 p35 gene or functional fragment thereof, an IRES sequence, and an IL-15 gene or a functional fragment thereof; and the second nucleic acid comprises a promoter operably linked to an IL-12 p40 gene or functional fragment thereof. In some aspects, the first nucleic acid comprises a promoter operably linked to a nucleic acid encoding an IL-12 p35 subunit or functional fragment thereof, an IRES sequence, and an IL-15 gene or a functional fragment thereof; and the second nucleic acid comprises a promoter operably linked to a nucleic acid encoding an IL-12 p40 subunit or functional fragment thereof.

In some aspects, the first nucleic acid comprises a promoter operably linked to an IL-12 p35 gene or functional fragment thereof, a furin cleavage sequence and/or a 2A self-processing peptide sequence (including an F2A, E2A, P2A or T2A self-processing peptide), and an IL-12 p40 gene or a functional fragment thereof, and the second nucleic acid comprises a promoter operably linked to an IL-15 gene or functional fragment thereof. In some aspects, the first nucleic acid comprises a promoter operably linked to a nucleic acid encoding an IL-12 p35 subunit or functional fragment thereof, a furin cleavage sequence and/or a 2A self-processing peptide sequence (including an F2A, E2A, P2A or T2A self-processing peptide), and a nucleic acid encoding an IL-12 p40 subunit or a functional fragment thereof, and the second nucleic acid comprises a promoter operably linked to an IL-15 gene or functional fragment thereof.

In some aspects, the first nucleic acid further comprises an intron sequence, a pA sequence, an enhancer sequence, or any combination thereof.

In some aspects, the first nucleic acid further comprises a human growth hormone (hGH) pA sequence.

In some aspects, the first nucleic acid further comprises an intron sequence and a bovine growth hormone (BGH) pA sequence.

In some aspects, the second nucleic acid further comprises an intron sequence, a pA sequence, an enhancer sequence, or any combination thereof.

In some aspects, the second nucleic acid further comprises an SV40 intron sequence and a synthetic (SYN) pA sequence.

In some aspects, the promoter of the first nucleic acid comprises a CMV promoter, a CBA promoter, an EF-1a (Elongation Factor 1a) promoter, aRSV (Rous Sarcoma Virus) promoter, or an Ubiquitin (UbC) promoter.

In some aspects, the promoter of the second nucleic acid comprises a CMV promoter, a CBA promoter, an EF-1a (Elongation Factor 1a) promoter, aRSV (Rous Sarcoma Virus) promoter, or an Ubiquitin (UbC) promoter.

Certain aspects of the disclosure are directed to an expression construct comprising a formula selected from:

X-I1-P1-E-P2-I2-X′-T-Y; (a)

Y-I1-P1-E-P2-I2-X-T-X′; (b)

pA-X-I1-L-P1-E-P2-I2-X-T-Y-pA; (c)

pA-Y-I1-L-P1-E-P2-I2-X-T-X′-pA; (d)

P1-X-T-X′-pA; or (e)

E-P1-I1-X-T-X′-pA; (f)
wherein X and X′ encode subunits of a first immunomodulatory molecule; I1 and I2 each is an intron; P and P2 each is a promoter; E is an enhancer; T is a translation modification sequence; Y encodes a second immunomodulatory molecule; L is a long-terminal repeat; and pA is a polyA sequence.

In some aspects, X is a nucleic acid encoding an IL-12 p35 subunit or a functional fragment thereof or a nucleic acid encoding an IL-12 p40 subunit or a functional fragment thereof. In some aspects, X′ is a nucleic acid encoding an IL-12 p35 subunit or a functional fragment thereof or a nucleic acid encoding an IL-12 p40 subunit or a functional fragment thereof. In some aspects, X (e.g., the nucleic acid encoding an IL-12 p35 subunit and/or an IL-12 p40 subunit) does not include an in-frame stop codon (e.g., TGA, TAG, and TAA).

In some aspects, P1 or P2 comprises a CBA promoter, a CMV promoter, an EF-1a (Elongation Factor 1a) promoter, aRSV (Rous Sarcoma Virus) promoter, an Ubiquitin (UbC) promoter, or any combination thereof. In some aspects, P1 or P2 comprises a CBA promoter or fragment thereof.

In some aspects, E comprises a CMV enhancer sequence.

In some aspects, I1 or I2 comprises a SV40 intron sequence or a CAG intron sequence.

In some aspects, T is an internal ribosomal entry site (IRES) sequence, a furin cleavage sequence, a 2A self-processing peptide sequence (including an F2A, E2A, P2A or T2A self-processing peptide), or any combination thereof. In some aspects, T is a furin cleavage sequence followed by a 2A self-processing peptide sequence (F2A).

In some aspects, the expression construct further comprises a Kozak sequence.

In some aspects, the expression construct further comprising a microRNA binding site.

In some aspects, X comprises a gene encoding an IL-12 subunit (e.g., IL-12 p35 or IL-12 p40) or a functional fragment thereof and X′ comprises a gene encoding an IL-12 subunit (e.g., IL-12 p40 or IL-12 p35) or functional fragment thereof. In some aspects, the gene encoding an IL-12 subunit is an IL-12 p35 gene or an IL-12 p40 gene. In some aspects, X comprises a nucleic acid encoding an IL-12 p35 subunit or a functional fragment thereof and X′ comprises a nucleic acid encoding an IL-12 p40 subunit or functional fragment thereof, P1 comprises a CBA promoter or fragment thereof; T comprises a furin cleavage sequence followed by a 2A self-processing peptide (F2A); pA comprises a human growth hormone (hGH) pA sequence; and optionally, E comprises a CMV enhancer sequence and I1 comprises a CAG intron.

In some aspects, X is a gene encoding an IL-12 subunit or a functional fragment thereof, X′ a gene encoding an IL-12 subunit or functional fragment thereof, and Y is IL-15 gene or a functional fragment thereof. In some aspects, the gene encoding an IL-12 subunit is an IL-12 p35 gene or an IL-12 p40 gene. In some aspects, X is a nucleic acid encoding an IL-12 subunit or a functional fragment thereof; X′ a nucleic acid encoding an IL-12 subunit or functional fragment thereof, and Y is IL-15 gene or a functional fragment thereof. In some aspects, the nucleic acid encoding an IL-12 subunit is a nucleic acid encoding an IL-12 p35 subunit or a nucleic acid encoding an IL-12 p40 subunit.

In some aspects, X is a nucleic acid encoding an IL-12 subunit or a functional fragment thereof and X′ is a nucleic acid encoding an IL-12 subunit or functional fragment thereof.

In some aspects, one or more of the pA comprises a human growth hormone (hGH) pA sequence, a bovine growth hormone (BGH) pA sequence, or a synthetic (SYN) pA sequence.

In some aspects, I1, I2, or both comprises a SV40 intron sequence. In some aspects, I1, I2, or both comprises a CAG intron sequence.

In some aspects, E comprises a CMV enhancer sequence.

In some aspects, the polynucleotides disclosed herein further comprise two inverted terminal repeats (ITRs). In some aspects, the polynucleotide comprises an expression cassette comprising the first nucleic acid and/or the second nucleic acid flanked by ITR sequences.

In some aspects, the disclosure is directed to a polynucleotide comprising a nucleic acid sequence having at least 80%, 85%, 90%, 95% 96%, 97%, 98%, 99%, or 100% identity to any of SEQ ID NOs: 5-37, 48-57, 77, 82, 83, 92, any nucleic acid shown in Table 2, Table 4, or Table 5, or any combinations thereof.

In some aspects, a polynucleotide of the disclosure comprises a nucleic acid sequence having at least 80%, 85%, 90%, 95% 96%, 97%, 98%, 99%, or 100% identity to any of SEQ ID NOs: 5-37, 48-57, 60, 61, 68, 71, 77, 82, 83, 85, 86, 88, 92, any nucleic acid sequence shown in Table 2, Table 4, Table 5, Table 6, or any combinations thereof.

In some aspects, provided herein is a composition comprising a polynucleotide disclosed herein and a delivery vector. In some aspects, the composition is suitable for an intratumoral delivery. In some aspects, the composition is suitable for an intravenous delivery. In some aspects, the composition is suitable for an intrathecal delivery. In some aspects, the composition is suitable for an intrahepatic delivery. In some aspects, the composition is suitable for a delivery to a lymphoid organ (e.g., a spleen, a lymph node, or a bone marrow).

In some aspects, the delivery vector is selected from the group consisting of a viral vector, a plasmid, a lipid, a protein particle, a bacterial vector, a lysosome, a virus-like particle, a polymeric particle, an exosome, and a vault particle.

In some aspects, the delivery vector is selected from the group consisting of an adeno-associated viral (AAV) vector, an adenoviral vector, a lentiviral vector, or a retroviral vector.

In some aspects, wherein the delivery vector is a recombinant AAV (rAAV) vector comprising an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, the rAAV vector is an AAV2 serotype. In some aspects, the rAAV is modified relative to the wild-type serotype.

In some aspect, provided herein is an adeno-associated virus (AAV) capsid comprising a polynucleotide disclosed herein (e.g., a polynucleotide comprising an expression cassette comprising the first nucleic acid and/or the second nucleic acid flanked by ITR sequences), wherein the AAV capsid is suitable for an intratumoral delivery of the nucleic acids disclosed herein.

In some aspect, provided herein is an adeno-associated virus (AAV) capsid comprising a polynucleotide disclosed herein (e.g., a polynucleotide comprising an expression cassette comprising the first nucleic acid and/or the second nucleic acid flanked by ITR sequences), wherein the AAV capsid is suitable for an intravenous delivery of the nucleic acids disclosed herein.

In some aspect, provided herein is an adeno-associated virus (AAV) capsid comprising a polynucleotide disclosed herein (e.g., a polynucleotide comprising an expression cassette comprising the first nucleic acid and/or the second nucleic acid flanked by ITR sequences), wherein the AAV capsid is suitable for a delivery of the nucleic acids disclosed herein to a lymphoid organ (e.g., a spleen, a lymph node, or a bone marrow).

In some aspects, the AAV capsid comprises an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, the AAV serotype is AAV2. In some aspects, the AAV is modified relative to the wild-type serotype.

In some aspects, the immunomodulatory proteins or functional fragments thereof are immunostimulatory proteins or functional fragments thereof.

In some aspects, provided herein is a method of expressing two or more immunomodulatory proteins or functional fragments thereof in a subject in need thereof comprising administering an effective amount of a combination of the first nucleic acid and the second nucleic acid disclosed herein using one or more delivery vectors disclosed herein, the polynucleotides disclosed herein, the compositions, or the AAV capsids (e.g., AAV particle) disclosed herein to the subject. In some aspects, the administration is intratumoral. In some aspects, the administration is intravenous. In some aspects, the administration is intrasplenic. In some aspects, the administration is intrathecal. In some aspects, the administration is intrahepatic. In some aspects, the administration is intraosseous. In some aspects, the administration is into a lymph node. In some aspects, the administration is intradermal. In some aspects, the administration is intraparenchymal. In some aspects, the administration is via delivery to salivary gland. In some aspects, the AAV capsid (e.g., AAV particle) has an AAV2 serotype.

In some aspects, provided herein is a method of delivering two or more immunomodulatory proteins functional fragments thereof to a tumor comprising administering a combination of the first nucleic acid and the second nucleic acid disclosed herein using one or more delivery vectors disclosed herein, the polynucleotides disclosed herein, the polynucleotides, the compositions, or the AAV capsids (e.g., AAV2 particles) disclosed herein to the tumor by direct injection.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intratumoral.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., rAAV2) disclosed herein to the subject, wherein the administration is intravenous.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intrasplenic.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intrathecal.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intrahepatic.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intraosseous.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is into a lymph node.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intradermal.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is intraparenchymal.

In some aspects, provided herein is a method of treating or reducing symptoms in a subject suffering from a tumor comprising administering a combination of the first nucleic acid and the second nucleic acid disclosed herein using one or more delivery vectors disclosed herein, the polynucleotides disclosed herein, the compositions, or the AAV capsids (e.g., AAV2 particles) disclosed herein to the subject, wherein the administration is via delivery to salivary gland.

In some aspects, the subject suffers from a solid tumor cancer. In some aspects, the solid tumor is a sarcoma, a carcinoma, or a lymphoma. In some aspects, the solid tumor is a surface lesion. In some aspects, the solid tumor is a visceral tumor.

In some aspects, the subject suffers from a cancer selected from the group consisting of skin cancer, gastrointestinal cancer, breast cancer, brain cancer, bone cancer, thoracic cancer, head and neck cancer, gynecologic cancer, urologic cancer, ocular cancer, and any combination thereof.

In some aspects, cancer is selected from a group consisting of:
skin cancer (e.g., metastatic melanoma, cutaneous malignant melanoma, cutaneous squamous cell carcinoma, basal cell carcinoma);
breast cancer (e.g., invasive breast cancer, triple-negative breast cancer, inflammatory breast cancer);
brain cancer (e.g., glioblastoma multiforme, medulloblastoma, pituitary carcinoma, brain stem gliomas, astrocytomas, oligodendrogliomas, hemangiopericytomas, germ cell tumors, pineal tumors);
bone cancer (e.g., chordomas, chondrosarcomas, osteosarcomas, Ewing sarcomas, fibrosarcomas, adamantiomas, giant cell tumors);
head and neck cancer (e.g., head and neck squamous cell carcinoma (HNSCC); salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer);
gynecologic cancer (e.g., ovarian cancer, uterine cancer, fallopian tube cancer, endometrial cancer, cervical cancer, vaginal cancer, vulvar cancer);
urologic cancer (e.g., renal cancer, urethral cancer, urothelial cancer, bladder cancer, cancer of the kidney or ureter, cancer of the renal pelvis, testicular cancer, penile cancer, cancer of the adrenal gland);
gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer);
ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors);
thoracic cancer (e.g., non-small cell lung cancer, small cell lung cancer, mesothelioma, thymic malignancies, tracheal tumors, some esophageal cancer),
leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)),
lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma),
other blood cancer (e.g., multiple myeloma)
cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination thereof.

In some aspects, the solid tumor is a surface lesion. In some aspects, the solid tumor is a visceral tumor.

In some aspects, the method of treating or reducing symptoms in a subject in need thereof comprises administering the composition, the gene therapy, the polynucleotide, the expression cassette, or the AAV capsid of the present disclosure to the subject in combination with a checkpoint inhibitor.

In some aspects, the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

In some aspects, the checkpoint inhibitor is a PD-1 inhibitor. In some aspects, the checkpoint inhibitor is a PD-L1 inhibitor. In some aspects, the checkpoint inhibitor is a CTLA-4 inhibitor. In some aspects, the checkpoint inhibitor is a combination of checkpoint inhibitors, including a combination of PD-1 inhibitors, a combination of PD-L1 inhibitors, a combination of CTLA-4 inhibitors, a combination of LAG-3 inhibitors, a combination of TIGIT inhibitors, a combination of TIM-3 inhibitors, a combination of VISTA inhibitors, a combination of BTLA inhibitors, a combination of ICOS inhibitors, a combination of B7-H3 inibitors, or a combination of any combination thereof.

In some aspects, the checkpoint inhibitor comprises pembrolizumab, nivolumab, cemiplimab, toripalimab, spartalizumab, dostarlimab-gxly, tislelizumab, balstilimab, BI-754091, zimberelimab, INCMGA00012, camrelizumab, or any combination thereof. In some aspects, the checkpoint inhibitor comprises pembrolizumab or nivolumab.

In some aspects, the checkpoint inhibitor comprises durvalumab, atezolizumab, avelumabor, or any combination thereof.

In some aspects, the checkpoint inhibitor comprises ipilimumab, tremelimumab (formerly ticilimumab), zalifrelimab, quavonlimab, BA3071, YH001, or any combination thereof.

In some aspects, the checkpoint inhibitor comprises relatlimab, BI-754111, favezelimab, eftilagimod alpha, LAG525, fianlimab, TSR-033, Sym022, FS118, MGD013, mavezelimab, INCAGN02385 or any combination thereof.

In some aspects, the checkpoint inhibitor comprises BMS-986207, domvanalimab, AB308, tiragolumab, AGEN1327, vibostolimab (MK-7864), etigalimab, or any combination thereof.

In some aspects, the checkpoint inhibitor comprises cobolimab, BMS-986258, TSR-022, MBG453, Sym023, INCAGN2390, LY3321367, SHR-1702, INCAGN02390, sabotolimab, R07121661, or any combination thereof.

In some aspects, the checkpoint inhibitor comprises JNJ-61610588 (CI-8993), CA-170, or any combination thereof. In some aspects, the checkpoint inhibitor comprises INBRX-106, PF-04518600, cudarolimab (IBI101), TAB004 (JS004), or any combination thereof.

In some aspects, the checkpoint inhibitor comprises GSK3359609, vopratelimab (JTX-2011), MEDI-570, alomfilimab (KY1044), or any combination thereof.

In some aspects, the checkpoint inhibitor comprises enoblituzumab, DS-7300a, orlotamab, or any combination thereof.

In some aspects, the gene therapy, the composition, the polynucleotide, the expression cassette, or the AAV capsid of the present disclosure can be administered prior to, at the same time, or after the administration of a checkpoint inhibitor.

In some aspects, provided herein is a composition comprising: (a) a polynucleotide comprising a nucleic acid encoding a cytokine, one or more subunits thereof, or functional fragment thereof, and (b) a viral vector (e.g., AAV capsid, e.g., AAV2 serotype). In some aspects, the polynucleotide comprises: (i) a first nucleic acid encoding a first cytokine subunit or functional fragment thereof; and (ii) a second nucleic acid encoding a second cytokine subunit or functional fragment thereof.

In some aspects, the cytokine is selected from the group consisting of IL-12, tumor necrosis factor alpha (TNF-α), a type I interferon (INF), a type II IFN, IL-2, IL-15, IL-21, IL-23, IL-27, IL-18, IL-1, IL-3, IL-4, IL-5, IL-6, IL-7, IL-8, IL-9, IL-10, IL17, IL33, TL1A, CD40L, granulocyte-macrophage colony-stimulating factor (GM-CSF), and any combination thereof. In some aspects, the cytokine is IL-12.

In some aspects, the polynucleotide comprises a nucleic acid encoding an IL-12 p35 subunit and/or a nucleic acid encoding an IL-12 p40 subunit. In some aspects, the nucleic acid (e.g., the first nucleic acid) encoding an IL-12 p35 subunit and/or the nucleic acid encoding encoding an IL-12 p40 subunit does not include an in-frame stop codon (e.g., TGA, TAG, and TAA).

In some aspects, the polynucleotide further comprises one or more promoters. In some aspects, the construct can comprise a promoter for the nucleic acid encoding a first cytokine subunit (e.g., an IL-12 p35 subunit) and a promoter for the nucleic acid sequence encoding a second cytokine subunit (e.g., an IL-12 p40 subunit). In some aspects, the two or more promoters are the same. In some aspects, the two or more promoters are different. In some aspects, one promoter can drive the expression of both the first cytokine subunit (e.g., an IL-12 p35 subunit) and the second cytokine subunit (e.g., an IL-12 p40 subunit). In some aspects, the nucleic acid encoding an IL-12 p35 subunit and/or the nucleic acid encoding an IL-12 p40 subunit does not include an in-frame stop codon (e.g., TGA, TAG, and TAA).

In some aspects, the promoter is a constitutively active promoter, a cell-type specific promoter, or an inducible promoter. In some aspects, the promoter comprises a CBA promoter, a CMV promoter, an EF-1a (Elongation Factor 1a) promoter, a RSV (Rous Sarcoma Virus) promoter, an Ubiquitin (UbC) promoter, or any combination thereof. In some aspects, the promoter comprises a CBA promoter. In some aspects, the promoter comprises a CMV enhancer, a CBA promoter, and a CAG intron sequence (e.g., SEQ ID NO: 71).

In some aspects, the polynucleotide further comprises translation modification sequence, e.g., a furin cleavage sequence, a 2A self-processing peptide sequence (including an F2A, E2A, P2A or T2A self-processing peptide), an internal ribosomal entry site (IRES) sequence or any combination thereof. In some aspects, the nucleic acid comprises the combination of a furin cleavage sequence followed by a nucleic acid encoding a 2A self-processing peptide sequence (F2A). In some aspects, the furin cleavage sequence followed by the 2A self-processing peptide sequence (F2A) is located between the first nucleic acid encoding an IL-12 p35 subunit or a functional fragment thereof or an IL-12 p40 subunit or a functional fragment thereof and the second nucleic acid encoding an IL-12 p35 subunit or a functional fragment thereof or an IL-12 p40 subunit or a functional fragment thereof. In some aspects, the first nucleic acid (e.g., nucleic acid encoding an IL-12 p35 subunit and/or the nucleic acid encoding encoding an IL-12 p40 subunit) does not include an in-frame stop codon (e.g., TGA, TAG, and TAA).

In some aspects, the polynucleotide further comprises a poly(A) (pA) sequence. In some aspects, the pA sequence is a human growth hormone (HGH) pA sequence. In some aspects, the pA sequence is a bovine growth hormone (BGH) pA sequence.

In some aspects, provided herein is a composition comprsing (a) a polynucleotide comprising a CMV enhancer, a CBA promoter or fragment thereof operably linked to a first nucleic acid encoding an IL-12 p35 subunit or a functional fragment thereof, a furin cleavage sequence followed by a 2A self-processing peptide sequence (F2A), a second nucleic acid encoding an IL-12 p40 subunit or a functional fragment thereof, and a human growth hormone (HGH) pA sequence; and (b) a viral vector (e.g., an AAV capsid). In some aspects, the polynucleotide further comprise an intron sequence (e.g., a CAG intron).

In some aspects, provided herein is a composition comprsing (a) a polynucleotide comprising a CMV enhancer, a CBA promoter or fragment thereof operably linked to a first nucleic acid encoding an IL-12 p35 subunit or a functional fragment thereof, a furin cleavage sequence followed by a 2A self-processing peptide (F2A), a second nucleic acid encoding an IL-12 p40 subunit or a functional fragment thereof, and a bovine growth hormone (BGH) pA sequence; and (b) a viral vector (e.g., an AAV capsid).

In some aspects, the nucleic acid encoding the IL-12 p35 subunit comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77. In some aspects, the nucleic acid encoding the IL-12 p40 subunit comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92. In some aspects, the nucleic acid encoding an IL-12 p35 subunit does not include an in-frame stop codon (e.g., TGA, TAG, and TAA). In some aspects, the nucleic acid encoding the IL-12 p35 subunit comprises a sequence at least 85%, at least 90%, at least 96%, at least 97%, at least 98%, at least 99%, or 100% identical to SEQ ID NO: 77. In some aspects, the nucleic acid encoding an IL-12 p40 subunit does not include an in-frame stop codon (e.g., TGA, TAG, and TAA). In some aspects, the nucleic acid encoding the IL-12 p40 subunit comprises a sequence at least 85%, at least 90%, at least 96%, at least 97%, at least 98%, at least 99%, or 100% identical to SEQ ID NO: 92.

In some aspects, provided herein is a method of treating or reducing symptoms in a subject suffering from a tumor, comprising administering to the subject a composition comprising (a) a polynucleotide comprising (i) a first nucleic acid encoding an IL-12 p35 subunit or functional fragment thereof and (ii) a second nucleic acid encoding an IL-12 p40 subunit or functional fragment thereof, and (b) an AAV vector (e.g., AAV2 serotype).

In some aspects, provided herein is a method of reducing the size of a tumor in a subject in need thereof comprising administering to the subject a composition comprising (a) a polynucleotide comprising (i) a first nucleic acid encoding an IL-12 p35 subunit or functional fragment thereof and (ii) a second nucleic acid encoding an IL-12 p40 subunit or functional fragment thereof, and (b) an AAV vector (e.g., AAV2 serotype).

In some aspects, the nucleic acid encoding an IL-12 p35 subunit and/or IL-12 p40 subunit does not include an in-frame stop codon (e.g., TGA, TAG, and TAA).

In some aspects, the administration is intratumoral, intravenous, intraperitoneal, intrasplenic, intrathecal, intrahepatic, intraosseous, intradermal, intraparenchymal, via delivery to salivary gland, or into a lymph node.

In some aspects, the tumor is derived from a cancer selected from the group consisting of hepatocellular carcinoma, colon carcinoma, melanoma, and any combination thereof. In some aspects, the subject suffers from a cancer selected from the group consisting of hepatocellular carcinoma, colon carcinoma, melanoma, and any combination thereof.

In some aspects, the tumor is derived from a cancer selected from the group consisting of skin cancer (e.g., metastatic melanoma, cutaneous malignant melanoma, cutaneous squamous cell carcinoma, basal cell carcinoma); breast cancer (e.g., invasive breast cancer, triple-negative breast cancer, inflammatory breast cancer); brain cancer (e.g., glioblastoma multiforme, medulloblastoma, pituitary carcinoma, brain stem gliomas, astrocytomas, oligodendrogliomas, hemangiopericytomas, germ cell tumors, pineal tumors); bone cancer (e.g., chordomas, chondrosarcomas, osteosarcomas, Ewing sarcomas, fibrosarcomas, adamantiomas, giant cell tumors); head and neck cancer (e.g., head and neck squamous cell carcinoma; salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer); gynecologic cancer (e.g., ovarian cancer, uterine cancer, fallopian tube cancer, endometrial cancer, cervical cancer, vaginal cancer, vulvar cancer); urologic cancer (e.g., renal cancer, urethral cancer, urothelial cancer, bladder cancer, cancer of the kidney or ureter, cancer of the renal pelvis, testicular cancer, penile cancer, cancer of the adrenal gland); gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer); ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors); thoracic cancer (e.g., non-small cell lung cancer, small cell lung cancer, mesothelioma, thymic malignancies, tracheal tumors, some esophageal cancer), leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)), lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma), other blood cancer (e.g., multiple myeloma), cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination thereof.

In some aspects, the composition further comprises a checkpoint inhibitor.

In some aspects, the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

In some aspects, the checkpoint inhibitor is a combination of checkpoint inhibitors, including a combination of PD-1 inhibitors, a combination of PD-L1 inhibitors, a combination of CTLA-4 inhibitors, a combination of LAG-3 inhibitors, a combination of TIGIT inhibitors, a combination of TIM-3 inhibitors, a combination of VISTA inhibitors, a combination of BTLA inhibitors, a combination of ICOS inhibitors, a combination of B7-H3 inibitors, or a combination of any combination thereof.

In some aspects, the checkpoint inhibitor is a PD-1 inhibitor. In some aspects, the PD-1 inhibitor is an anti-PD-1 antibody. In some aspects, the checkpoint inhibitor comprises pembrolizumab, nivolumab, cemiplimab, toripalimab, spartalizumab, dostarlimab-gxly, tislelizumab, balstilimab, BI-754091, zimberelimab, INCMGA00012, camrelizumab, or any combination thereof. In some aspects, the checkpoint inhibitor comprises pembrolizumab or nivolumab.

In some aspects, the checkpoint inhibitor is a PD-L1 inhibitor. In some aspects, the checkpoint inhibitor comprises durvalumab, atezolizumab, avelumabor, or any combination thereof.

In some aspects, the checkpoint inhibitor is a CTLA-4 inhibitor. In some aspects, the checkpoint inhibitor comprises ipilimumab, tremelimumab (formerly ticilimumab), zalifrelimab, quavonlimab, BA3071, YH001, or any combination thereof.

In some aspects, the composition disclosed herein, the polynucleotide disclosed herein, or the AAV capsid disclosed herein is administered prior to, at the same time, or after the administration of the checkpoint inhibitor.

### BRIEF DESCRIPTION OF DRAWINGS

FIG. 1A shows an exemplary AAV construct designed to include a backbone, flanking ITRs, a promoter, polyA, and open reading frame (ORF).

FIG. 1B shows an exemplary AAV construct designed with an expression cassette comprising a promoter, an open reading frame (ORF) (comprising a sequence encoding a first cytokine, a linker, and a sequence encoding a second cytokine), an optional miRNA, and a poly(A) signal sequence, which is flanked by ITR sequences. The construct further comprises a backbone.

FIG. 2A shows an exemplary expression cassette comprising: a synthetic (SYN) poly(A) (pA) (SYNpA) sequence; gene of interest (GOI) (e.g., an IL-12 p40 gene); an intron sequence; 5′ long terminal repeat (LTR), a promoter (Promoter 1); an enhancer sequence; a second promoter (Promoter 2); intron; gene of interest (GOI) (e.g., an IL-12 p35 gene); an internal ribosomal entry site (IRES) sequence; gene of interest (GOI) (e.g., an IL-15 gene); and a polyA (e.g., a bovine growth hormone (BGH) pA (BGHpA)) sequence.

FIG. 2B shows an exemplary expression cassette comprising: a synthetic (SYN) poly(A) (pA) (SYNpA) sequence; gene of interest (GOI) (an IL-15 gene); an intron sequence; 5′ long terminal repeat (LTR), a promoter (Promoter 1); an enhancer sequence; a second promoter (Promoter 2); an intron; gene of interest (GOI) (an IL-12 p35 gene); a furin cleavage sequence followed by the 2A self-processing peptide (F2A); gene of interest (GOI) (an IL-12 p40 gene); and a polyA (e.g., a bovine growth hormone (BGH) pA (BGHpA)) sequence.

FIG. 2C and FIG. 2D show an exemplary multicistronic expression cassettes including two promoters and three genes of interest (e.g., IL-15, IL-12A, or IL-12B).

FIG. 3 is a graph showing luciferase expression two and five days after in vitro transduction with an AAV2-luciferase vector at MOIs ranging from 2000 to 40500 vp/cell, in the five different cancer cell lines (MC38, B16F10, CloudmanS91, CT26, and A20) used to create syngeneic mouse cancer models.

FIG. 4A and FIG. 4B show bioluminescence imaging showing luciferase expression in MC38 (FIG. 4A) and CloudmanS91 (FIG. 4B) tumors after intratumoral injection with an AAV2-luciferase vector. Luciferase activity was measured over 21 days.

FIG. 5A-FIG. 5E show bioluminescence imaging showing luciferase expression co-localized with tumor mass after intratumoral injection for CT26 (FIG. 5A), B16F10 (FIG. 5B), A20 (FIG. 5C), MC38 (FIG. 5D), and CloudmanS91 (FIG. 5E) after intratumoral injection with an AAV2-luciferase vector.

FIG. 6A and FIG. 6B show bioluminescence imaging showing luciferase expression in A20 (FIG. 6A) and CloudmanS91 (FIG. 6B) after intratumoral injection with an AAV2-luciferase vector.

FIG. 7A-FIG. 7D show tumor regression as measured by tumor volume (mm³) in a syngeneic H22 hepatocellular cancer mouse model after treatment with vehicle (FIG. 7A), AAV2-IL12 (FIG. 7B), an anti-PD-1 antibody (FIG. 7C), or AAV2-IL12 and an anti-PD-1 antibody (FIG. 7D). Each line represents an individual mouse. Horizontal dashed line (-----) represents a maximum tumor volume (animals are euthanized once this volume is exceeded). The x-axis represents the number of days after treatment (0, 5, 10, 15, 20, 25, 30, 35, 40, 45, 50, 55, 60, 65, 70, and 75 days).

FIG. 8A and FIG. 8B show tumor regression as measured by mean area under the curve (AUC) (FIG. 8A) or by median area under the curve (AUC) (FIG. 8B) of tumor growth in a syngeneic H22 hepatocellular cancer mouse model after treatment with vehicle, AAV2-IL12, AAV2-IL12 and an anti-PD-1 antibody, or an anti-PD-1 antibody. In FIG. 8A, data are expressed as the mean values±standard error of the mean (SEM). (ns=not significant; * p<0.05; ***<0.0001; 2-sided t-test).

FIG. 9A-FIG. 9E show tumor regression as measured by tumor volume (mm³) in a syngeneic MC38 colorectal cancer mouse model after treatment with vehicle (FIG. 9A), AAV2-IL12 (FIG. 9B and FIG. 9E), an anti-PD-1 antibody (FIG. 9C), or AAV2-IL12 (1×10¹¹ viral genomes) and an anti-PD-1 antibody (FIG. 9D). AAV2-IL12 was administered at dosages of 1×10¹¹ viral genomes (FIG. 9B) and 3×10¹⁰ viral genomes (FIG. 9E). Each line represents an individual mouse. Horizontal dashed line (-----) represents a maximum tumor volume (animals are euthanized once this volume is exceeded). The x-axis represents the number of days after treatment (0, 10, 20, 30, 40, 50, and 60 days).

FIG. 10A and FIG. 10B show tumor regression as measured by mean area under the curve (AUC) (FIG. 10A) or by median area under the curve (AUC) (FIG. 10B) of tumor growth in a syngeneic MC38 colorectal cancer mouse model after treatment with vehicle, AAV2-IL12 at a dose of 1×10¹¹ viral genomes, AAV2IL12 at a dose of 3×10¹⁰ viral genomes, AAV2-IL12 and an anti-PD-1 antibody, or an anti-PD-1 antibody. In FIG. 10A, data are expressed as the mean values±standard error of the mean (SEM). (ns=not significant; * p<0.05; ***<0.0001; 2-sided t-test).

FIG. 11A-FIG. 11E show tumor regression as measured by tumor volume (mm³) in a syngeneic Cloudman S91 melanoma cancer mouse models after treatment with vehicle (FIG. 11A), AAV2-IL12 (FIG. 11B and FIG. 11E), an anti-PD-1 antibody (FIG. 11C), or AAV2-IL12 (1×10¹¹ viral genomes) and an anti-PD-1 antibody (FIG. 11D). AAV2-IL12 was administered at dosages of 1×10¹¹ viral genomes (FIG. 11B) and 3×10¹⁰ viral genomes (FIG. 11E). Each line represents an individual mouse. Horizontal dashed line (-----) represents a maximum tumor volume (animals are euthanized once this volume is exceeded). The x-axis represents the number of days after treatment (0, 5, 10, 15, 20, 25, 30, 35, 40, and 45 days).

FIG. 12A and FIG. 12B show tumor regression as measured by mean area under the curve (AUC) (FIG. 12A) or by median area under the curve (AUC) (FIG. 12B) of tumor growth in a syngeneic Cloudman S91 melanoma cancer model a syngeneic after treatment with vehicle, AAV2-IL12 at a dose of 1×10¹¹ viral genomes, AAV2-IL12 at a dose of 3×101° viral genomes, AAV2-IL12 and an anti-PD-1 antibody, or an anti-PD-1 antibody. In FIG. 12A, data are expressed as the mean values±standard error of the mean (SEM). (ns=not significant; * p<0.05; ***<0.0001; 2-sided t-test).

FIG. 13A-13D show tumor regression as measured by tumor volume (mm³) in a syngeneic Hepa1-6 hepatocellular cancer mouse model after treatment with vehicle (FIG. 13A), AAV2-IL12 (FIG. 13B), an anti-PD-1 antibody (FIG. 13C), or AAV2-IL12 and an anti-PD-1 antibody (FIG. 13D). Each line represents an individual mouse. Horizontal dashed line (-----) represents a maximum tumor volume (animals are euthanized once this volume is exceeded). The x-axis represents the number of days after treatment (0, 5, 10, 15, 20, 25, and 30 days).

FIG. 13E-13F show tumor regression as measured by mean area under the curve (AUC) (FIG. 13E) or by median area under the curve (AUC) (FIG. 13F) of tumor growth in a syngeneic Hepa1-6 hepatocellular cancer mouse model after treatment with control (vehicle), AAV2-IL12, AAV2-IL12 and an anti-PD-1 antibody, or an anti-PD-1 antibody. In FIG. 13E, data are expressed as the mean values±standard error of the mean (SEM). (ns=not significant).

FIG. 14A-14D show tumor regression as measured by tumor volume (mm³) in a syngeneic B16F10 melanoma cancer mouse model after treatment with vehicle (FIG. 14A), AAV2-IL12 (FIG. 14B), an anti-PD-1 antibody (FIG. 14C), or AAV2-IL12 and an anti-PD-1 antibody (FIG. 14D). Each line represents an individual mouse. Horizontal dashed line (-----) represents a maximum tumor volume (animals are euthanized once this volume is exceeded). The x-axis represents the number of days after treatment (0, 5, 10, 15, 20, 25, and 30 days).

FIG. 15A and FIG. 15B show tumor regression as measured by mean area under the curve (AUC) (FIG. 15A) or by median area under the curve (AUC) (FIG. 15B) of tumor growth in a syngeneic B16F10 melanoma cancer mouse model after treatment with vehicle, AAV2-IL12, AAV2-IL12 and an anti-PD-1 antibody, or an anti-PD-1 antibody. In FIG. 15A, data are expressed as the mean values±standard error of the mean (SEM). (ns=not significant; * p<0.05; ***<0.0001; 2-sided t-test).

FIG. 16 shows overall survival (% survival) of a syngeneic H22 hepatocellular cancer mouse model after treatment with vehicle, AAV2-IL12, AAV2-IL12 and an anti-PD-1 antibody, or an anti-PD-1 antibody. The x-axis represents the number of days after treatment (0, 5, 10, 15, 20, 25, 30, 35, 50, 45, and 50 days). Horizontal dashed line (-----) indicates 50% survival.

FIG. 17 shows overall survival (% survival) of a syngeneic MC38 colorectal cancer mouse model after treatment with vehicle, AAV-IL12, AAV-IL12 and an anti-PD-1 antibody, or an anti-PD-1 antibody. The x-axis represents the number of days after treatment (0, 5, 10, 15, 20, 25, 30, 35, 50, 45, and 50 days). Horizontal dashed line (-----) indicates 50% survival.

FIG. 18 shows overall survival (% survival) of a syngeneic Cloudman S91 melanoma cancer mouse model after treatment with vehicle, AAV2-IL12, AAV2-IL12 and an anti-PD-1 antibody, or an anti-PD-1 antibody. The x-axis represents the number of days after treatment (0, 5, 10, 15, 20, 25, 30, 35, 50, 45, and 50 days). Horizontal dashed line (-----) indicates 50% survival.

FIG. 19 shows overall survival (% survival) of a syngeneic B16F10 melanoma cancer mouse model after treatment with vehicle, AAV2-IL12, AAV2-IL12 and an anti-PD-1 antibody, or an anti-PD-1 antibody. The x-axis represents the number of days after treatment (0, 5, 10, 15, 20, 25, 30, 35, 50, 45, and 50 days).

FIG. 20A-FIG. 20E show mean body weight change relative to day 1(%) of syngeneic H22 hepatocellular (FIG. 20A), B16F10 melanoma (FIG. 20B), Cloudman S91 melanoma (FIG. 20C) MC38 colorectal (FIG. 20D), and Hepa1-6 hepatocellular cancer mouse models (FIG. 20E) after treatment with vehicle, AAV2-IL2 (1×10¹¹ viral genomes or 3×10¹⁰ viral genomes), AAV2-IL12 (1×10¹¹ viral genomes) and an anti-PD-1 antibody, or an anti-PD-1 antibody.

FIG. 21 shows body weight (grams) of normal mice that were intravenously administered AAV2-IL12 in escalated doses in escalated doses (1×10⁹ viral genomes, 1×10¹⁰ viral genomes, or 1×10¹¹ viral genomes) or control buffer.

FIG. 22 shows IL12 expression (ng/mL) in vitro after administration of an AAV2-IL12 construct (either AAV2 vector #1 (AAV2-CAG-mIL12a-IRES-mIL12b-bgh PA) or AAV2 vector #2 (AAV2-CAG-mIL12a-F2A-mIL12b-hgh PA).

FIG. 23A shows an AAV2 construct designed with an expression cassette comprising a CMV enhancer, a CBA promoter, a CAG intron, a Kozak sequence, a first nucleic acid encoding a mouse native IL-12 p35 subunit, a furin cleavage sequence followed by a 2A self-processing peptide (F2A), a second nucleic acid encoding a mouse native IL-12 p40 subunit, and a human growth hormone (hGH) polyA signal sequence, which is flanked by ITR sequences (“AAV2-CAG-mIL12a-F2A-mIL12b-hgh PA”).

FIG. 23B shows an AAV2 construct designed with an expression cassette comprising a CMV enhancer, a CBA promoter, a CAG intron, a Kozak sequence, a first nucleic acid encoding a human native IL-12 p35 subunit, a furin cleavage sequence followed by a 2A self-processing peptide (F2A), a second nucleic acid encoding a human native IL-12 p40 subunit, and a human growth hormone (hGH) polyA signal sequence, which is flanked by ITR sequences (“AAV2-CAG-hIL12a-F2A-hIL12b-hgh PA”).

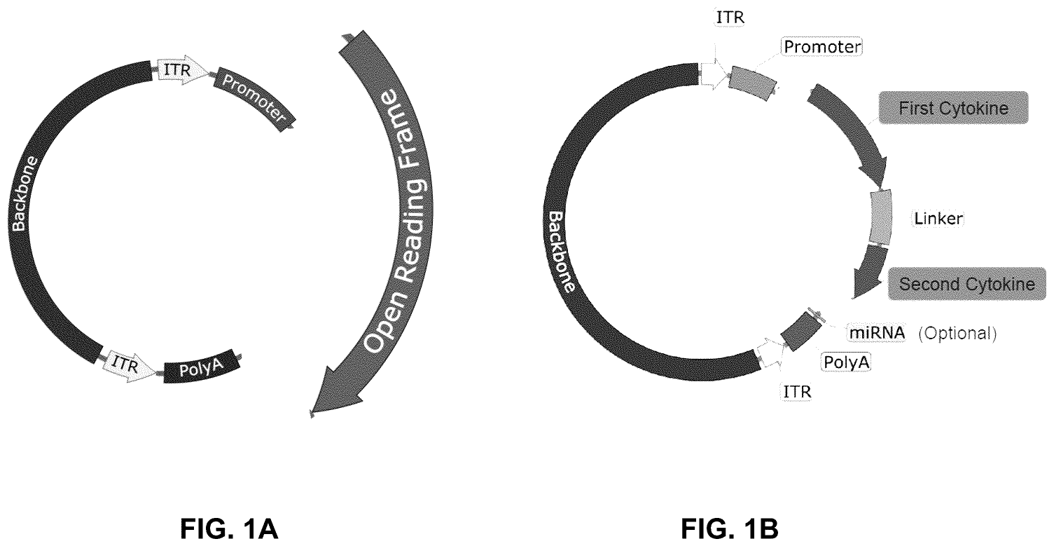

### DETAILED DESCRIPTION OF THE DISCLOSURE

Some aspects of the present disclosure are directed to compositions (e.g., gene therapy compositions) and/or combinations and methods comprising one or more viral vectors (e.g., an AAV vector) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof. In some aspects, each viral vector (e.g., AAV vector) comprises a polynucleotide (e.g., an expression cassette) comprising the first nucleic acid and the second nucleic acid. In some aspects, each delivery vector (e.g., viral vector) comprises either the first nucleic acid or the second nucleic acid, wherein two or more viral vectors (e.g., AAV vector) are delivered in combination (e.g., simultaneously or sequentially). In some aspects, the delivery vector comprises constructs comprising nucleic acids encoding a first immunomodulatory protein and the subunits of a second immunomodulatory protein.

In some aspects, the disclosure is directed to a composition comprising: (a) a polynucleotide (e.g., an expression cassette) comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector). In some aspects, the expression cassette comprises nucleic acids encoding a first immunomodulatory protein and the subunits of a second immunomodulatory protein.

In some aspects, the disclosure is directed to a polynucleotide (e.g., an expression cassette) comprising: (a) a first nucleic acid encoding a first cytokine, one or more subunits thereof, or functional fragment thereof, and (b) a second nucleic acid encoding a second cytokine, one or more subunits thereof, or functional fragment thereof.

In some aspects, the disclosure is directed to a composition comprising: (a) a polynucleotide comprising a first nucleic acid encoding a first immunomodulatory protein (e.g., a cytokine), one or more subunits thereof, or functional fragment thereof and a second nucleic acid encoding a second immunomodulatory protein (e.g., a cytokine), one or more subunits thereof, or functional fragment thereof; and (b) a delivery vector (e.g., a viral vector). Some aspects of the present disclosure are directed to a polynucleotide comprising: (a) a first nucleic acid encoding a first cytokine, one or more subunits thereof, or functional fragment thereof, and (b) a second nucleic acid encoding a second cytokine, one or more subunits thereof, or functional fragment thereof.

In some aspects, the disclosure is directed to an adeno-associated virus (AAV) capsid comprising one or more of the polynucleotides disclosed herein, wherein the AAV capsid is suitable for an intratumoral delivery of the nucleic acid(s) encoding immunomodulatory proteins (e.g., cytokines) disclosed herein.

In some aspects, the disclosure is directed to an adeno-associated virus (AAV) capsid comprising one or more of the polynucleotides disclosed herein, wherein the AAV capsid is suitable for an intravenous delivery of the nucleic acid(s) encoding immunomodulatory proteins (e.g., cytokines) disclosed herein.

In some aspects, the disclosure is directed to an adeno-associated virus (AAV) capsid comprising one or more of the polynucleotides disclosed herein, wherein the AAV capsid is suitable for a delivery of the nucleic acid(s) encoding immunomodulatory proteins (e.g., cytokines) disclosed herein to a lymphoid organ (e.g., a spleen, a lymph node, or a bone marrow).

In some aspects, the AAV capsid comprises an AAV serotype, wherein the AAV serotype is AAV2. In some aspects, the AAV is modified relative to the wild-type serotype.

Some aspects of the disclosure are directed to a method of expressing two or more immunomodulatory proteins, the subunits thereof, or functional fragments thereof in a subject in need thereof comprising administering an effective amount of the compositions (e.g., gene therapy compositions) or the AAV capsids disclosed herein to the subject. In some aspects, the administration is intratumoral. In some aspects, the administration is intravenous. In some aspects, the administration is intrasplenic. In some aspects, the administration is intrathecal. In some aspects, the administration is intrahepatic. In some aspects, the administration is intraosseous. In some aspects, the administration is into a lymph node. In some aspects, the administration is intradermal. In some aspects, the administration is intraparenchymalv. In some aspects, the administration is a delivery to salivary gland.

In some aspects, the expression is intratumoral. In some aspects, the composition is administered intratumorally by direct injection. In some aspects, the direct injection comprises multiple injections of the composition to the tumor. In some aspects, the administration is a single dose comprising one or more injections.

Some aspects of the disclosure are directed to a method of delivering nucleic acids encoding two or more immunomodulatory proteins, the subunits thereof, or functional fragments thereof to a tumor comprising administering the compositions (e.g., gene therapy compositions), the polynucleotides, or the AAV capsids disclosed herein, wherein the administration is to the tumor by direct injection.

Some aspects of the disclosure are directed to a method of treating or reducing symptoms in a subject suffering from a tumor comprising administering a composition (e.g., gene therapy composition), a polynucleotide, an expression cassette, or an AAV capsid (e.g., AAV2 particles) disclosed herein to the subject, wherein the administration is intratumoral, intravenous, intrasplenic, intrathecal, intrahepatic, intraosseous, intradermal, intraparenchymal, via delivery to salivary gland, or into a lymph node.

In some aspects, the subject suffers from a cancer selected from the group consisting of a skin cancer, a gastrointestinal cancer, breast cancer, brain cancer, or any combination thereof. In some aspects, cancer is selected from a group consisting of melanoma, colorectal cancer, breast cancer, glioblastoma multiforme, and any combination thereof.

In some aspects, the subject suffers from a cancer selecte from the group consisting of skin cancer (e.g., metastatic melanoma, cutaneous malignant melanoma, cutaneous squamous cell carcinoma, basal cell carcinoma); breast cancer (e.g., invasive breast cancer, triple-negative breast cancer, inflammatory breast cancer); brain cancer (e.g., glioblastoma multiforme, medulloblastoma, pituitary carcinoma, brain stem gliomas, astrocytomas, oligodendrogliomas, hemangiopericytomas, germ cell tumors, pineal tumors); bone cancer (e.g., chordomas, chondrosarcomas, osteosarcomas, Ewing sarcomas, fibrosarcomas, adamantiomas, giant cell tumors); head and neck cancer (e.g., head and neck squamous cell carcinoma; salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer); gynecologic cancer (e.g., ovarian cancer, uterine cancer, fallopian tube cancer, endometrial cancer, cervical cancer, vaginal cancer, vulvar cancer); urologic cancer (e.g., renal cancer, urethral cancer, urothelial cancer, bladder cancer, cancer of the kidney or ureter, cancer of the renal pelvis, testicular cancer, penile cancer, cancer of the adrenal gland); gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer); ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors); thoracic cancer (e.g., non-small cell lung cancer, small cell lung cancer, mesothelioma, thymic malignancies, tracheal tumors, some esophageal cancer), leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)), lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma), other blood cancer (e.g., multiple myeloma), cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination thereof.

In some aspects, the constructs, polynucleotides, expression cassettes, delivery vectors, and compositions disclosed herein are designed to (a) introduce, eliminate, or reduce immunostimulatory motifs, (b) utilize optimal genome length, (c) reduce reverse packaging, (d) enhance expression in target tissues and/or cells, (e) reduce or increase CpG motifs, or (f) any combination thereof.

Non-limiting examples of the various aspects are shown in the present disclosure.

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “immune response” refers to a biological response within an organism against a foreign agent or abnormal cell (e.g., a tumor cell), wherein the response protects the organism against such agents/cells and diseases caused by them. An immune response is mediated by the action of a cell of the immune system (e.g., a T lymphocyte (T cell), B lymphocyte (B cell), natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the organism's body of invading pathogens, cells or tissues infected with pathogens, cancerous or other abnormal cells, or, in cases of autoimmunity or pathological inflammation, normal human cells or tissues. In some aspects, an immune reaction includes, e.g., activation or inhibition of a T cell, e.g., an effector T cell or a Th cell, such as a CD4⁺ or CD8⁺ T cell, or the inhibition of a regulatory T cell (Treg cell).

As used herein, the term an “immunomodulatory protein,” “immune modulator,” “immunoregulatory protein,” or “immunoregulator” refers to an agent, e.g., a component of a signaling pathway, that can be involved in stimulating, modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some aspects, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets can include, for example, receptors on the surface of a cell (“immunomodulatory receptors”) and receptor ligands (“immunomodulatory ligands”). In some aspects, the immunomodulatory protein is a cytokine. In some aspects, the immunomodulatory protein is a soluble natural killer (NK) cell, B lymphocyte cell, T lymphocyte cell, neutrophil, or macrophage ligand.

As used herein, the term “functional fragment thereof” refers to a fragment or portion of a protein, e.g., an immunomodulatory protein, that is still capable of one or functions associated with the full protein (e.g., stimulating, modulating, regulating, or modifying an immune response).

As used herein, the term “checkpoint inhibitor” refers to an inhibitor for a negative checkpoint regulator or an inhibitor for a binding partner of a negative checkpoint regulator. In some aspects, In certain aspects, the negative checkpoint regulator comprises programmed cell death protein 1 (PD-1), programmed death ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

As used herein, the term “cytokine” refers to one or more factors that exert effects on cells, for example, influencing growth or proliferation. In some aspects, cytokines can possess one or more of the following properties: ability to mediate and/or regulate immune defense functions by acting as messengers between the various immune cells; functioning over short distances with a brief half-life; produced by a variety of cells types; ability to act on diverse cell targets within the immune system and/or on organs; ability to stimulate and/or inhibit growth; and/or directly or indirectly causing a cytokine cascade. In some aspects, cytokines can include interleukins, interferons, colony stimulating factors and tumour necrosis factor. Non-limiting examples of cytokines which can be used alone or in combination in the practice of the present disclosure include, tumor necrosis factor-α (TNFα), a type I interferon (IFN), a type II IFN, interleukin-2 (IL-2), interleukin-12 (IL-12), interleukin-15 (IL-15), interleukin-21 (IL-21), interleukin-23 (IL-23), interleukin-27 (IL-27), interleukin-18 (IL-18), IL-1, IL-3, IL-4, IL-5, IL-6, IL-7, IL-8, IL-9, IL-10, IL17, IL33, TL1A, CD40L, stem cell factor (SCF), interleukin-3 (IL-3), interleukin-4 (IL-4), interleukin-6 (IL-6), interleukin-11 (IL-11), interleukin-13 (IL-13), granulocyte-colony stimulating factor (G-CSF), granulocyte macrophage-colony stimulating factor (GM-CSF), interleukin-1 beta (IL-10), prostaglandin E₂ (PGE₂), MIP-11, leukemia inhibitory factor (LIF), c-kit ligand, thrombopoietin (TPO) and flt3 ligand. It is intended, although not always explicitly stated, that molecules having similar biological activity as wild-type or purified cytokines (e.g., recombinantly produced cytokines, functional fragments, or muteins thereof) are intended to be used within the scope of the disclosure.

The term “subunit” refers to a polypeptide that can assemble with another polypeptide to form a complex (e.g., a heterodimer). In some aspects, the cytokine can comprise a single encoded polypeptide or multiple encoded polypeptide subunits (e.g., p35 and p40 subunits). For example, p35 subunit can be subunit alpha of interleukin 12 (also known as IL-12A, cytotoxic lymphocyte maturation factor p35, or interleukin-12 subunit p35). For example, p40 subunit can be subunit beta of interleukin 12 (also known as IL-12B, natural killer cell stimulatory factor 2, cytotoxic lymphocyte maturation factor p40, or interleukin-12 subunit p40).

A “heterodimer” refers to a polypeptide composed of two polypeptide chains differing in composition. For example, interleukin 12 is a heterodimer consisting of one p35 subunit and one p40 subunit.

The term “immunotherapy” refers to the treatment of a disease by inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease (e.g., cancer).

As used herein, the term “toll-like receptor 9 (TLR9)” a protein that in humans is encoded by the TLR9 gene. TLR9 has also been designated as CD289 (cluster of differentiation 289). It is a member of the toll-like receptor (TLR) family. TLR9 is a receptor expressed in immune system cells including dendritic cells, macrophages, natural killer cells, and other antigen presenting cells. TLR9 recognizes unmethylated bacterial CpG DNA and initiates a signaling cascade leading to the production of proinflammatory cytokines. See e.g., Martinez-Campos C, et al., Viral Immunology. 30(2):98-105 (2017); Notley C A et al., Scientific Reports. 7: 42204 (2017). The stimulatory effect of CpG DNA is conferred by unmethylated CpG dinucleotides in particular base contexts (CpG motifs) that also determine the species-specific activity of CpG DNA. CpG motifs containing the core sequence GACGTT highly stimulate mouse TLR9, whereas CpG motifs containing more than one CpG and the core sequence GTCGTT are optimal inducers of human TLR9. Cancer, infection, and tissue damage can all modulate TLR9 expression and activation. In some aspects, the nucleic acids, the constructs, polynucleotides, and/or expression cassettes disclosed herein comprise sequences with reduced or eliminated toll-like receptor 9 (TLR9) stimulatory motifs.

“Immunostimulating therapy” or “immunomodulatory therapy” refers to a therapy that results in increasing (inducing or enhancing) an immune response in a subject, e.g., for treating cancer.

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a protein particle, a bacterial vector, a lysosome, a virus-like particle, a polymeric particle, an exosome, or a vault particle.

Some aspects of the disclosure are directed to biological vectors, which can include viruses, particularly attenuated and/or replication-deficient viruses.

As used herein, the term “promoter” refers to a DNA sequence recognized by the machinery of the cell, or introduced synthetic machinery, required to initiate the transcription of a gene. The term “promoter” is also meant to encompass those nucleic acid elements sufficient for promoter-dependent gene expression controllable for cell-type specific, tissue-specific or inducible by external signals or agents; such elements can be located in the 5′ or 3′ regions of the native gene. In some aspects, the promoter is a constitutively active promoter, a cell-type specific promoter, or an inducible promoter.

In some aspects, microRNA targeting sequences are included to increase specificity of vector-mediated transgene expression. See e.g., Anja Geisler and Henry Fechner, World J Exp Med., 20; 6(2):37-54 (2016).

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

As used herein, the term “regulatable promoter” is any promoter whose activity is affected by a cis or trans acting factor (e.g., an inducible promoter, such as an external signal or agent).

As used herein, the term “constitutive promoter” is any promoter that directs RNA production in many or all tissue/cell types at most times, e.g., the human CMV immediate early enhancer/promoter region that promotes constitutive expression of cloned DNA inserts in mammalian cells.

The terms “transcriptional regulatory protein,” “transcriptional regulatory factor,” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to, or is bound to a DNA response element.

As used herein, the term “termination signal sequence” can be any genetic element that causes RNA polymerase to terminate transcription, such as for example a polyadenylation (polyA or pA) signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

As used herein, the term “multicistronic” or “multicistronic vector” refers to a nucleic acid sequence having two or more open reading frames (e.g., genes). An open reading frame in this context is a sequence of codons that is translatable into a polypeptide or protein (e.g. protein subunits, e.g., cytokine subunits). “Bicistronic” or “bicistronic vector” refers to a nucleic acid sequence having two open reading frames (e.g., genes). An open reading frame in this context is a sequence of codons that is translatable into a polypeptide or protein (e.g. protein subunits, e.g., cytokine subunits). In some aspects, the construct of the disclosure is a multicistronic (e.g., bicistronic) construct (e.g., comprising cytokine (e.g., IL-12) subunits).

As used herein, the term “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). “Under translational control of an IRES” as used herein means that translation is associated with the IRES and proceeds in a cap-independent manner.

The term “self-processing cleavage site” or “self-processing cleavage sequence,” as used herein refers to a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. Suitable 2A sites include the F2A, E2A, P2A or T2A self-processing sites. The term “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

As used herein, the term “additional proteolytic cleavage site,” refers to a sequence that is incorporated into an expression construct of the disclosure adjacent a self-processing cleavage site, such as a 2A or 2A like sequence, and provides a means to remove additional amino acids that remain following cleavage by the self-processing cleavage sequence. Exemplary “additional proteolytic cleavage sites” are described herein and include, but are not limited to, furin cleavage sites with the consensus sequence RXK(R)R (SEQ ID NO: 1).

Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. In some aspects, the term “operably linked” means that a DNA sequence and a regulatory sequence(s) are connected in such a way as to permit gene expression when the appropriate molecules (e.g., transcriptional activator proteins) are bound to the regulatory sequence(s). In some aspects, the term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

The term “expression vector”, “expression construct” or “expression cassette” means any type of genetic construct containing a nucleic acid in which part or all of the nucleic acid encoding sequence is capable of being transcribed.

A “viral vector” refers to a sequence that comprises one or more polynucleotide regions encoding or comprising a molecule of interest, e.g., a protein, a peptide, and an oligonucleotide or a plurality thereof. Viral vectors can be used to deliver genetic materials into cells. Viral vectors can be modified for specific applications. In some aspects, the delivery vector of the disclosure is a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome”, “AAV capsid”, and “AAV vector” can be used interchangeably. In some aspects, the AAV vector is modified or mutated relative to the wild-type AAV serotype sequence.

As used herein, an “AAV particle” is an AAV virus that comprises an AAV vector having at least one payload region (e.g., a polynucleotide encoding a cytokine) and at least one inverted terminal repeat (ITR) region. In some aspects, the terms “AAV vectors of the present disclosure” or “AAV vectors” refer to AAV vectors comprising a polynucleotide encoding a cytokine, e.g., encapsulated in an AAV particle.

A “coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an AAV vector) that is derived from a second nucleic acid sequence (e.g., another AAV vector) can include a nucleotide sequence that is identical or substantially similar to the nucleotide sequence of the second nucleic acid sequence.

In the case of a polynucleotide disclosed herein, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each. The mutagenesis of a polynucleotide to create a different polynucleotide derived from the first can be a random event (e.g., caused by polymerase infidelity) and the identification of the derived polynucleotide can be made by appropriate screening methods.

As used herein, the term “mutation” refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

As used herein, the term “administration” refers to the administration of a composition of the present disclosure (e.g., an AAV vector or a composition (e.g., gene therapy composition) disclosed herein) to a subject or system. Administration to an animal subject (e.g., to a human) can be by any appropriate route, such as direct injection into a tumor.

As used herein, the terms “re-administration,” “repeat administration,” and “redosing” refer to an administration of one or more additional dose(s) of a therapeutic agent, e.g., a gene therapy, a delivery construct, an AAV capsid, or a composition of the disclosure, following a first administration.

As used herein, the term “modified” refers to a changed state or structure of a molecule of the disclosure. Molecules can be modified in many ways including chemically, structurally, and functionally.

As used herein, the term “synthetic” means produced, prepared, and/or manufactured by the hand of man. Synthesis of polynucleotides or polypeptides or other molecules of the present disclosure can be chemical or enzymatic.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “mRNA,” as used herein, refers to a single stranded RNA that encodes the amino acid sequence of one or more polypeptide chains.

The term “antisense,” as used herein, refers to a nucleic acid that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA, e.g., an shRNA, that includes a region that is substantially complementary to a target sequence, e.g., mRNA. The antisense strand has sequence sufficiently complementary to the desired target mRNA sequence to direct target-specific silencing, e.g., complementarity sufficient to trigger the destruction of the desired target mRNA by the RNAi machinery or process.

The terms “sense strand” and “passenger strand,” as used herein, refer to the strand of a dsRNA, e.g., an shRNA, that includes a region that is substantially complementary to a region of the antisense strand as that term is defined herein. The antisense and sense strands of a dsRNA, e.g., an shRNA, are hybridized to form a duplex structure.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

The term a “therapeutic agent” or “therapeutic molecule” includes a compound or molecule that, when present in an effective amount, produces a desired therapeutic effect, pharmacologic and/or physiologic effect on a subject in need thereof. It includes any compound, e.g., a small molecule drug, or a biologic (e.g., a polypeptide drug or a nucleic acid drug) that when administered to a subject has a measurable or conveyable effect on the subject, e.g., it alleviates or decreases a symptom of a disease, disorder or condition. In some aspects, the therapeutic molecule is a immunomodulatory protein (e.g., a cytokine) or functional fragment thereof.

As used herein, the term “inhibitor” refers to any agent that reduces the level and/or activity of a protein. Non-limiting examples of inhibitors include dsRNA, e.g., siRNA or shRNA, and vectors, e.g., AAV, comprising polynucleotides encoding dsRNA, e.g., siRNA or shRNA. The term “reducing,” as used herein, is used interchangeably with “silencing,” “downregulating,” “suppressing,” and other similar terms, and includes any level of reduction.

The phrase “contacting a cell” (e.g., contacting a cell with an AAV vector, an AAV capsid, or the composition (e.g., gene therapy composition) of the disclosure) as used herein, includes contacting a cell directly or indirectly. In some aspects, contacting a cell with an AAV vector, an AAV capsid, or the composition (e.g., gene therapy composition) includes contacting a cell in vitro with the composition (e.g., gene therapy composition), the AAV vector, or the AAV capsid or contacting a cell in vivo with the AAV vector, the AAV capsid, or the composition (e.g., gene therapy composition). Thus, for example, the AAV vector, the AAV capsid, or the composition (e.g., gene therapy composition) can be put into physical contact with the cell by the individual performing the method, or alternatively, the AAV vector, the AAV capsid, or the composition (e.g., gene therapy composition) can be put into a situation that will permit or cause it to subsequently come into contact with the cell.

In some aspects, contacting a cell in vitro can be done, for example, by incubating the cell with the AAV vector. In some aspects, contacting a cell in vivo can be done, for example, by injecting the AAV vector, the AAV capsid, or the composition (e.g., gene therapy composition) of the disclosure into or near the tissue where the cell is located (e.g., a tumor), or by injecting the AAV vector, the AAV capsid, or the composition (e.g., gene therapy composition) into another area, e.g., the bloodstream or the subcutaneous space, such that the agent will subsequently reach the tissue where the cell to be contacted is located. For example, the AAV vector can be encapsulated and/or coupled to a ligand that directs the AAV vector to a site of interest. Combinations of in vitro and in vivo methods of contacting are also possible. For example, a cell can be contacted in vitro with an AAV vector, an AAV capsid, or the composition (e.g., gene therapy composition) and subsequently transplanted into a subject.

In some aspects, contacting a cell with an AAV vector, an AAV capsid, or the composition (e.g., gene therapy composition) of the present disclosure includes “introducing” or “delivering” (directly or indirectly) the AAV vector, the AAV capsid, or the composition (e.g., gene therapy composition) into the cell by facilitating or effecting uptake or absorption into the cell. Introducing an AAV vector, an AAV capsid, or the composition (e.g., gene therapy composition) into a cell can be in vitro and/or in vivo. For example, for in vivo introduction, an AAV vector, an AAV capsid, the composition (e.g., gene therapy composition) can be injected into a specific tissue site (e.g., the locus where a therapeutic effect is desired) or administered systemically (e.g., administering a AAV vector targeted to a locus where a therapeutic effect is desired). In vitro introduction into a cell includes methods known in the art such as electroporation and lipofection.

As used herein, the terms “effective amount,” “therapeutically effective amount,” and a “sufficient amount” of, e.g., an AAV vector, an AAV capsid, or the composition (e.g., gene therapy composition) disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., an AAV vector, an AAV capsid, the composition (e.g., gene therapy composition) disclosed herein) is an amount that results in a beneficial or desired result in a subject as compared to a control.

The amount of a given agent (e.g., an AAV vector, an AAV capsid, or the composition (e.g., gene therapy composition) disclosed herein) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “gene therapy” is the insertion of nucleic acid sequences (e.g., a polynucleotide comprising a promoter operably linked to a nucleic acid encoding an immunomodulatory protein (e.g., a cytokine or subunit thereof) or functional fragment thereof as disclosed herein) into an individual's cells and/or tissues to treat, reduce the symptoms of, or reduce the likelihood of a disease. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases are amenable to gene therapy.

The term “prophylactically effective amount,” as used herein, includes the amount of an agent, (e.g., an AAV vector, an AAV capsid, or the composition (e.g., gene therapy composition) disclosed (herein) that, when administered to a subject having or predisposed to have a disease or disorder (e.g., a cancer), is sufficient to prevent, reduce the symptoms of, or ameliorate the disease or disorder or one or more symptoms of the disease or disorder. Ameliorating the disease or disorder includes slowing the course of the disease or disorder or reducing the severity of later-developing disease or disorder. The “prophylactically effective amount” can vary depending on the characteristics of the agent, e.g., an AAV vector, an AAV capsid, or the composition (e.g., gene therapy composition), how the agent is administered, the degree of risk of disease, and the history, age, weight, family history, genetic makeup, the types of preceding or concomitant treatments, if any, and other individual characteristics of the patient to be treated.

As used herein, “off target” refers to any unintended effect on any one or more target, gene, or cellular transcript.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

By “level” is meant a level or activity of a protein, or mRNA encoding the protein, optionally as compared to a reference. The reference can be any useful reference, as defined herein. By a “decreased level” or an “increased level” of a protein is meant a decrease or increase in protein level, as compared to a reference.

A level of a protein can be expressed in mass/vol (e.g., g/dL, mg/mL, g/mL, ng/mL) or percentage relative to total protein or mRNA in a sample.

The term “pharmaceutical composition,” as used herein, represents a composition comprising a compound or molecule described herein, e.g., an AAV vector disclosed herein, formulated with a pharmaceutically acceptable excipient, and can be manufactured or sold with the approval of a governmental regulatory agency as part of a therapeutic regimen for the treatment of disease in a mammal.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration.

As used herein, the term “subject” refers to any organism to which a composition disclosed herein, e.g., an AAV vector or composition (e.g., gene therapy composition) of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

In some aspects, the human can be immunocompromised or immunodeficient. In some aspects, the human can be elderly. In some aspects, the human can be immunocompromised or imminodeficient and elderly.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. In some aspects, the methods of the present disclosure can be used to reduce the tumor size of a tumor derived from, for example, skin cancer (e.g., metastatic melanoma, cutaneous malignant melanoma, cutaneous squamous cell carcinoma, basal cell carcinoma); breast cancer (e.g., invasive breast cancer, triple-negative breast cancer, inflammatory breast cancer); brain cancer (e.g., glioblastoma multiforme, medulloblastoma, pituitary carcinoma, brain stem gliomas, astrocytomas, oligodendrogliomas, hemangiopericytomas, germ cell tumors, pineal tumors); bone cancer (e.g., chordomas, chondrosarcomas, osteosarcomas, Ewing sarcomas, fibrosarcomas, adamantiomas, giant cell tumors); head and neck cancer (e.g., head and neck squamous cell carcinoma; salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer); gynecologic cancer (e.g., ovarian cancer, uterine cancer, fallopian tube cancer, endometrial cancer, cervical cancer, vaginal cancer, vulvar cancer); urologic cancer (e.g., renal cancer, urethral cancer, urothelial cancer, bladder cancer, cancer of the kidney or ureter, cancer of the renal pelvis, testicular cancer, penile cancer, cancer of the adrenal gland); gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer); ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors); thoracic cancer (e.g., non-small cell lung cancer, small cell lung cancer, mesothelioma, thymic malignancies, tracheal tumors, some esophageal cancer), leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)), lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma), other blood cancer (e.g., multiple myeloma), cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination of said cancers.

The term “tumor” as used herein refers to any mass of tissue that results from excessive cell growth or proliferation, either benign (non-cancerous) or malignant (cancerous), including pre-cancerous lesions.

The term a “lymphoid organ” as used herein refers to primary lymphoid organs, which include the bone marrow and the thymus and secondary lymphoid organs, which include the lymph nodes, the spleen, the tonsils and certain tissue in various mucous membrane layers in the body (for instance in the bowel).

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. In some aspects, treating reduces or lessens the symptoms associated with a disease or disorder. In some aspects, the treating results in a beneficial or desired clinical result.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years.

### II. Immunomodulatory Proteins and Polynucleotides of Interest

The present disclosure provides compositions, e.g., gene therapy compositions, comprising a polynucleotide comprising a nucleic acid encoding an immunomodulatory protein, one or more subunits thereof, or functional fragment thereof. In some aspects, the immunomodulatory protein is a cytokine, or a soluble natural killer (NK) cell, B cell, T cell, neutrophil, or macrophage ligand, or any combination thereof. In some aspects, the immunomodulatory protein is a cytokine. In some aspects, the immunomodulatory protein is a soluble natural killer (NIK) cell, B cell, T cell neutrophil, or macrophage ligand.

### Cytokines

Cytokines are proteinaceous signaling compounds that are mediators of the immune response. They control many different cellular functions including proliferation, differentiation and cell survival/apoptosis. Cytokines are also involved in several pathophysiological processes including viral infections and autoimmune diseases. Cytokines are synthesized under various stimuli by a variety of cells, including those of both the innate (monocytes, macrophages, dendritic cells) and adaptive (T and B cells) immune systems.

Table 1 shows a classification of cytokine receptor families.

TABLE 1
Classification of Cytokine Receptor Families

| Receptor Family | Ligands | Structure/Function |
|---|---|---|
| Type I Cytokine | IL-2 | Composed of multimeric chains. |
| Receptors | IL-3 | Signals through JAK-STAT |
| | IL-4 | pathway using common |
| | IL-5 | signaling chain. Contains |
| | IL-6 | cytokine binding chains. |
| | IL-7 | |
| | IL-9 | |
| | IL-11 | |
| | IL-12 | |
| | IL-13 | |
| | IL-15 | |
| | IL-21 | |
| | IL-23 | |
| | IL-27 | |
| | IL-33 | |
| Type I Cytokine | Erythropoietin | Composed of multimeric chains. |
| Receptors | GM-CSF | Signals through JAK-STAT |
| | G-CSF | pathway using common |
| | Growth hormone | signaling chain. Contains |
| | Prolactin | cytokine binding chains. |
| | Oncostatin M | |
| | Leukemia | |
| | inhibitory factor | |
| Type II Cytokine | IFN-α/β | Immunoglobulin-like domains. |
| Receptors | IFN-ψ | Uses heterodimer and multimeric |
| | IL-10 | chains. Signals through |
| | IL-20 | JAK-STAT. |
| | IL-22 | |
| | IL-28 | |
| Immunoglobulin | IL-1 | Shares homology with |
| Superfamily | CSF1 | immunoglobulin structures. |
| Receptors | c-kit | |
| | IL-18 | |
| IL-17 Receptor | IL-17 | |
| | IL-17B | |
| | IL-17C | |
| | IL-17D | |
| | IL-17E | |
| | IL-17F | |
| G Protein-Coupled | IL-8 | Functions to mediate cell |
| Receptors (GPCR) | CC chemokines | activation and migration. |
| | CXC chemokine | |
| TGF-β receptors ½ | TGF-β | |
| Tumor Necrosis | CD27 | Functions as co-stimulatory |
| Factor Receptors | CD30 | and co-inhibitory receptors. |
| (TNFR) | CD40 | |
| | CD120 | |
| | Lymphotoxin-β | |

CD—cluster of differentiation;
c-kit—mast/stem cell growth factor receptor;
CSF—colony-stimulating factor;
G-CSF—granulocyte-colony stimulating factor;
GM-CSF—granulocyte-macrophage colony stimulating factor;
IL—interleukin;
JAK—janus kinase;
STAT—signal transducer and activator of transcription;
TGF—transforming growth factor. See e.g., Lee S. et al., Cancers, 3:3856-3893 (2011).

In some aspects, the present disclosure is directed to a nucleic acid encoding a cytokine selected from any of the cytokines show in Table 1 or a nucleic acid encoding one or more subunits of a cytokine selected from any of the multi-subunit (e.g., heterodimeric) cytokines show in Table 1.

The present disclosure provides compositions (e.g., gene therapy compositions) and AAV vectors comprising a polynucleotide comprising a first nucleic acid encoding a first cytokine, one or more subunits thereof, or functional fragment thereof and/or a second nucleic acid encoding a second cytokine, one or more subunits thereof, or functional fragment thereof. In some aspects, the composition (e.g., gene therapy composition) comprises a multicistronic (e.g., bicistronic) construct (e.g., comprising cytokine (e.g., IL-12) subunits).

In some aspects, provided herein is a composition (e.g., a gene therapy composition) comprising: (a) a polynucleotide comprising a nucleic acid encoding an immunomodulatory protein, one or more subunits thereof, or functional fragment thereof (e.g., a cytokine or one or more subunits thereof); and (b) a delivery vector (e.g., an AAV capsid).

In some aspects, the cytokine is full-length, a functional fragment of a cytokine, or a functional variant of a cytokine, e.g., a cytokine comprising one or more mutations. In some aspects, the cytokine is a heterodimeric cytokine composed of more than one subunit. In some aspects, the cytokine molecule can be a monomer or a dimer. In some aspects, the cytokine molecule further comprises a receptor domain, e.g., a cytokine receptor domain.

In some aspects, the cytokine is selected from the group consisting of tumor necrosis factor alpha (TNF-α), a type I interferon (INF), a type II IFN, interleukin (IL)-2, IL-12, IL-15, IL-21, IL-23, IL-27, IL-18, IL-1, IL-3, IL-4, IL-5, IL-6, IL-7, IL-8, IL-9, IL-10, IL17, IL33, TL1A, CD40L, granulocyte-macrophage colony-stimulating factor (GM-CSF), any functional fragment thereof, and any combination thereof.

IFN-γ can promote Treg fragility, and can reduce suppression in the tumor microenvironment. IL-21 and IL-23 can induce Tregs to produce pro-inflammatory IL-17 and/or convert Tregs to T helper 17 cells (Th17) T cell subset. IL-12 promotes IFN-γ production in Tregs, leading Treg fragility and a general pro-immunogenic environment. TNF-α both impairs Treg development and reduces the function of existing Tregs. Thus, these cytokines can impair Treg development, reduce Treg function, or induce Treg trans-differentiation into the immune activating cells. In the context of cancer, it is desired to reduce Treg activity. See e.g., Int'l Publ. No. WO2019/010219.

The present disclosure also provides a polynucleotide comprising: (a) a first nucleic acid encoding a first cytokine subunit or functional fragment thereof, and (b) a second nucleic acid encoding a second cytokine subunit or functional fragment thereof.

The present disclosure also provides a polynucleotide comprising: (a) a first nucleic acid encoding a first cytokine, one or more subunits thereof, or functional fragment thereof, and (b) a second nucleic acid encoding a second cytokine, one or more subunits thereof, or functional fragment thereof.

In some aspects, the first cytokine is IL-12, IL-18, IL-21, or a functional fragment thereof. In some aspects, the first nucleic acid comprises an IL-12 p35 gene and/or an IL-12 p40 gene. In some aspects, the first cytokine is IL-12 or a functional fragment thereof. In some aspects, the first nucleic acid comprises a nucleic acid encoding an IL-12 p35 subunit and/or a nucleic acid encoding an IL-12 p40 subunit.

In some aspects, the first nucleic acid (e.g., nucleic acid encoding an IL-12 p35 subunit and/or the nucleic acid encoding an IL-12 p40 subunit) does not include an in-frame stop codon (e.g., TGA, TAG, and TAA).

In some aspects, the first nucleic acid comprises a nucleic acid sequence having at least 85%, 90%, 95%, 96%, 97%, 98%, 99%, or 100% identity to SEQ ID NOs: 5-10, 77, 92, or any nucleic acid sequence shown in Table 2, or any combination thereof. In some aspects, the first nucleic acid comprises a nucleic acid sequence having at least 85%, 90%, 95%, 96%, 97%, 98%, 99%, or 100% identity to a reverse complement of SEQ ID NOs: 5-10, 77, 92, or any nucleic acid sequence shown in Table 2, or any combination thereof.

In some aspects, the polynucleotide of the disclosure comprises one or more nucleic acids comprising a nucleic acid sequence having at least 85%, 90%, 95% 96%, 97%, 98%, 99%, or 100% identity to SEQ ID NOs: 5-10, 77, 92, or any nucleic acid sequence shown in Table 2, or any combination thereof. In some aspects, the polynucleotide of the disclosure comprises one or more nucleic acids comprising a nucleic acid sequence having at least 85%, 90%, 95% 96%, 97%, 98%, 99%, or 100% identity to a reverse complement of SEQ ID NOs: 5-10, 77, 92, or any nucleic acid sequence shown in Table 2, or any combination thereof.

TABLE 2
IL-12 Nucleic Acid Sequences

| | IL-12 Nucleic Acid |
|---|---|
| IL-12 | Sequence (SEQ ID NO) |
| Human IL-12A | ATGTGTCCAGCGCGC |
| (p35) (“wild- | AGCCTCCTCCTTGTG |
| type” no CpG | GCTACCCTGGTCCTC |
| modification) | CTGGACCACCTCAGT |
| | TTGGCCAGAAACCTC |
| | CCCGTGGCCACTCCA |
| | GACCCAGGAATGTTC |
| | CCATGCCTTCACCAC |
| | TCCCAAAACCTGCTG |
| | AGGGCCGTCAGCAAC |
| | ATGCTCCAGAAGGCC |
| | AGACAAACTCTAGAA |
| | TTTTACCCTTGCACT |
| | TCTGAAGAGATTGAT |
| | CATGAAGATATCACA |
| | AAAGATAAAACCAGC |
| | ACAGTGGAGGCCTGT |
| | TTACCATTGGAATTA |
| | ACCAAGAATGAGAGT |
| | TGCCTAAATTCCAGA |
| | GAGACCTCTTTCATA |
| | ACTAATGGGAGTTGC |
| | CTGGCCTCCAGAAAG |
| | ACCTCTTTTATGATG |
| | GCCCTGTGCCTTAGT |
| | AGTATTTATGAAGAC |
| | TTGAAGATGTACCAG |
| | GTGGAGTTCAAGACC |
| | ATGAATGCAAAGCTT |
| | CTGATGGATCCTAAG |
| | AGGCAGATCTTTCTA |
| | GATCAAAACATGCTG |
| | GCAGTTATTGATGAG |
| | CTGATGCAGGCCCTG |
| | AATTTCAACAGTGAG |
| | ACTGTGCCACAAAAA |
| | TCCTCCCTTGAAGAA |
| | CCGGATTTTTATAAA |
| | ACTAAAATCAAGCTC |
| | TGCATACTTCTTCAT |
| | GCTTTCcggATTCGG |
| | GCAGTGACTATTGAT |
| | AGAGTGATGAGCTAT |
| | CTGAATGCTTCCTGA |
| | (SEQ ID NO: 5) |
| Human IL-12A | ATGTGTCCAGCGCGC |
| (p35) (“wild- | AGCCTCCTCCTTGTG |
| type” no CpG | GCTACCCTGGTCCTC |
| modification | CTGGACCACCTCAGT |
| and no | TTGGCCAGAAACCTC |
| in-frame stop | CCCGTGGCCACTCCA |
| codon) | GACCCAGGAATGTTC |
| | CCATGCCTTCACCAC |
| | TCCCAAAACCTGCTG |
| | AGGGCCGTCAGCAAC |
| | ATGCTCCAGAAGGCC |
| | AGACAAACTCTAGAA |
| | TTTTACCCTTGCACT |
| | TCTGAAGAGATTGAT |
| | CATGAAGATATCACA |
| | AAAGATAAAACCAGC |
| | ACAGTGGAGGCCTGT |
| | TTACCATTGGAATTA |
| | ACCAAGAATGAGAGT |
| | TGCCTAAATTCCAGA |
| | GAGACCTCTTTCATA |
| | ACTAATGGGAGTTGC |
| | CTGGCCTCCAGAAAG |
| | ACCTCTTTTATGATG |
| | GCCCTGTGCCTTAGT |
| | AGTATTTATGAAGAC |
| | TTGAAGATGTACCAG |
| | GTGGAGTTCAAGACC |
| | ATGAATGCAAAGCTT |
| | CTGATGGATCCTAAG |
| | AGGCAGATCTTTCTA |
| | GATCAAAACATGCTG |
| | GCAGTTATTGATGAG |
| | CTGATGCAGGCCCTG |
| | AATTTCAACAGTGAG |
| | ACTGTGCCACAAAAA |
| | TCCTCCCTTGAAGAA |
| | CCGGATTTTTATAAA |
| | ACTAAAATCAAGCTC |
| | TGCATACTTCTTCAT |
| | GCTTTCcggATTCGG |
| | GCAGTGACTATTGAT |
| | AGAGTGATGAGCTAT |
| | CTGAATGCTTCC |
| | (SEQ ID NO: 77) |
| Human IL-12A | ATGTGTCCAGCGCGC |
| (p35) (with CpG | AGCCTCCTCCTTGTG |
| modification) | GCTACCCTGGTCCTC |
| | CTGGACCACCTCAGT |
| | TTGGCCAGAAACCTC |
| | CCTGTGGCCACTCCA |
| | GACCCAGGAATGTTC |
| | CCATGCCTTCACCAC |
| | TCCCAAAACCTGCTG |
| | AGGGCTGTCAGCAAC |
| | ATGCTCCAGAAGGCC |
| | AGACAAACTCTAGAA |
| | TTTTACCCTTGCACT |
| | TCTGAAGAGATTGAT |
| | CATGAAGATATCACA |
| | AAAGATAAAACCAGC |
| | ACAGTGGAGGCCTGT |
| | TTACCATTGGAATTA |
| | ACCAAGAATGAGAGT |
| | TGCCTAAATTCCAGA |
| | GAGACCTCTTTCATA |
| | ACTAATGGGAGTTGC |
| | CTGGCCTCCAGAAAG |
| | ACCTCTTTTATGATG |
| | GCCCTGTGCCTTAGT |
| | AGTATTTATGAAGAC |
| | TTGAAGATGTACCAG |
| | GTGGAGTTCAAGACC |
| | ATGAATGCAAAGCTT |
| | CTGATGGATCCTAAG |
| | AGGCAGATCTTTCTA |
| | GATCAAAACATGCTG |
| | GCAGTTATTGATGAG |
| | CTGATGCAGGCCCTG |
| | AATTTCAACAGTGAG |
| | ACTGTGCCACAAAAA |
| | TCCTCCCTTGAAGAA |
| | CCTGATTTTTATAAA |
| | ACTAAAATCAAGCTC |
| | TGCATACTTCTTCAT |
| | GCTTTCcggATTCGC |
| | GCAGTGACTATTGAT |
| | AGAGTGATGAGCTAT |
| | CTGAATGCTTCCTGA |
| | (SEQ ID NO: 6) |
| Human IL-12A | ATGTGTCCAGCTAGG |
| (p35) (with CpG | AGCCTCCTCCTTGTG |
| modification) | GCTACCCTGGTCCTC |
| | CTGGACCACCTCAGT |
| | TTGGCCAGAAACCTC |
| | CCTGTGGCCACTCCA |
| | GACCCAGGAATGTTC |
| | CCATGCCTTCACCAC |
| | TCCCAAAACCTGCTG |
| | AGGGCAGTCAGCAAC |
| | ATGCTCCAGAAGGCC |
| | AGACAAACTCTAGAA |
| | TTTTACCCTTGCACT |
| | TCTGAAGAGATTGAT |
| | CATGAAGATATCACA |
| | AAAGATAAAACCAGC |
| | ACAGTGGAGGCCTGT |
| | TTACCATTGGAATTA |
| | ACCAAGAATGAGAGT |
| | TGCCTAAATTCCAGA |
| | GAGACCTCTTTCATA |
| | ACTAATGGGAGTTGC |
| | CTGGCCTCCAGAAAG |
| | ACCTCTTTTATGATG |
| | GCCCTGTGCCTTAGT |
| | AGTATTTATGAAGAC |
| | TTGAAGATGTACCAG |
| | GTGGAGTTCAAGACC |
| | ATGAATGCAAAGCTT |
| | CTGATGGATCCTAAG |
| | AGGCAGATCTTTCTA |
| | GATCAAAACATGCTG |
| | GCAGTTATTGATGAG |
| | CTGATGCAGGCCCTG |
| | AATTTCAACAGTGAG |
| | ACTGTGCCACAAAAA |
| | TCCTCCCTTGAAGAA |
| | CCTGATTTTTATAAA |
| | ACTAAAATCAAGCTC |
| | TGCATACTTCTTCAT |
| | GCTTTCcggATTAGG |
| | GCAGTGACTATTGAT |
| | AGAGTGATGAGCTAT |
| | CTGAATGCTTCCTGA |
| | (SEQ ID NO: 7) |
| Human IL-12B | ATGTGTCACCAGCAG |
| (p40) (“wild- | TTGGTCATCTCTTGG |
| type” no CpG | TTTTCCCTGGTTTTT |
| modification | CTGGCATCTCCCCTC |
| and with | GTGGCCATATGGGAA |
| stop codon) | CTGAAGAAAGATGTT |
| | TATGTCGTAGAATTG |
| | GATTGGTATCCGGAT |
| | GCCCCTGGAGAAATG |
| | GTGGTCCTCACCTGT |
| | GACACCCCTGAAGAA |
| | GATGGTATCACCTGG |
| | ACCTTGGACCAGAGC |
| | AGTGAGGTCTTAGGC |
| | TCTGGCAAAACCCTG |
| | ACCATCCAAGTCAAA |
| | GAGTTTGGAGATGCT |
| | GGCCAGTACACCTGT |
| | CACAAAGGAGGCGAG |
| | GTTCTAAGCCATTCG |
| | CTCCTGCTGCTTCAC |
| | AAAAAGGAAGATGGA |
| | ATTTGGTCCACTGAT |
| | ATTTTAAAGGACCAG |
| | AAAGAACCCAAAAAT |
| | AAGACCTTTCTAAGA |
| | TGCGAGGCCAAGAAT |
| | TATTCTGGACGTTTC |
| | ACCTGCTGGTGGCTG |
| | ACGACAATCAGTACT |
| | GATTTGACATTCAGT |
| | GTCAAAAGCAGCAGA |
| | GGCTCTTCTGACCCC |
| | CAAGGGGTGACGTGC |
| | GGAGCTGCTACACTC |
| | TCTGCAGAGAGAGTC |
| | AGAGGGGACAACAAG |
| | GAGTATGAGTACTCA |
| | GTGGAGTGCCAGGAG |
| | GACAGTGCCTGCCCA |
| | GCTGCTGAGGAGAGT |
| | CTGCCCATTGAGGTg |
| | ATGGTGGATGCCGTT |
| | CACAAGCTCAAGTAT |
| | GAAAACTACACCAGC |
| | AGCTTCTTCATCAGG |
| | GACATCATCAAACCT |
| | GACCCACCCAAGAAC |
| | TTGCAGCTGAAGCCA |
| | TTAAAGAATTCTCGG |
| | CAGGTGGAGGTCAGC |
| | TGGGAGTACCCTGAC |
| | ACCTGGAGTACTCCA |
| | CATTCCTACTTCTCC |
| | CTGACATTCTGCGTT |
| | CAaGTCCAGGGCAAG |
| | AGCAAGAGAGAAAAG |
| | AAAGATAGAGTCTTC |
| | ACGGACAAGACCTCA |
| | GCCACGGTCATCTGC |
| | CGCAAAAATGCCAGC |
| | ATTAGCGTGCGGGCC |
| | CAGGACCGCTACTAT |
| | AGCTCATCTTGGAGC |
| | GAATGGGCATCTGTG |
| | CCCTGCAGTTGA |
| | (SEQ ID NO: 8) |
| Human IL-12B | TGTGTCACCAGCAGT |
| (p40) | TGGTCATCTCTTGGT |
| (with CpG | TTTCCCTGGTTTTTC |
| modification) | TGGCATCTCCCCTTG |
| | TGGCCATATGGGAAC |
| | TGAAGAAAGATGTTT |
| | ATGTGGTAGAATTGG |
| | ATTGGTATCCTGATG |
| | CCCCTGGAGAAATGG |
| | TGGTCCTCACCTGTG |
| | ACACCCCTGAAGAAG |
| | ATGGTATCACCTGGA |
| | CCTTGGACCAGAGCA |
| | GTGAGGTCTTAGGCT |
| | CTGGCAAAACCCTGA |
| | CCATCCAAGTCAAAG |
| | AGTTTGGAGATGCTG |
| | GCCAGTACACCTGTC |
| | ACAAAGGAGGGGAGG |
| | TTCTAAGCCATTCCC |
| | TCCTGCTGCTTCACA |
| | AAAAGGAAGATGGAA |
| | TTTGGTCCACTGATA |
| | TTTTAAAGGACCAGA |
| | AAGAACCCAAAAATA |
| | AGACCTTTCTAAGAT |
| | GTGAGGCCAAGAATT |
| | ATTCTGGAAGGTTCA |
| | CCTGCTGGTGGCTGA |
| | CTACAATCAGTACTG |
| | ATTTGACATTCAGTG |
| | TCAAAAGCAGCAGAG |
| | GCTCTTCTGACCCCC |
| | AAGGGGTGACATGCG |
| | GAGCTGCTACACTCT |
| | CTGCAGAGAGAGTCA |
| | GAGGGGACAACAAGG |
| | AGTATGAGTACTCAG |
| | TGGAGTGCCAGGAGG |
| | ACAGTGCCTGCCCAG |
| | CTGCTGAGGAGAGTC |
| | TGCCCATTGAGGTgA |
| | TGGTGGATGCGGTTC |
| | ACAAGCTCAAGTATG |
| | AAAACTACACCAGCA |
| | GCTTCTTCATCAGGG |
| | ACATCATCAAACCTG |
| | ACCCACCCAAGAACT |
| | TGCAGCTGAAGCCAT |
| | TAAAGAATTCTAGGC |
| | AGGTGGAGGTCAGCT |
| | GGGAGTACCCTGACA |
| | CCTGGAGTACTCCAC |
| | ATTCCTACTTCTCCC |
| | TGACATTCTGTGTTC |
| | AaGTCCAGGGCAAGA |
| | GCAAGAGAGAAAAGA |
| | AAGATAGAGTCTTCA |
| | CTGACAAGACCTCAG |
| | CCACAGTCATCTGCC |
| | GCAAAAATGCCAGCA |
| | TTTCAGTGCGGGCCC |
| | AGGACCGCTACTATA |
| | GCTCATCTTGGTCAG |
| | AATGGGCATCTGTGC |
| | CCTGCAGTTGA |
| | (SEQ ID NO: 9) |
| Human IL-12B | ATGTGTCACCAGCAG |
| (p40) | TTGGTCATCTCTTGG |
| (with CpG | TTTTCCCTGGTTTTT |
| modification) | CTGGCATCTCCCCTT |
| | GTGGCCATATGGGAA |
| | CTGAAGAAAGATGTT |
| | TATGTGGTAGAATTG |
| | GATTGGTATCCTGAT |
| | GCCCCTGGAGAAATG |
| | GTGGTCCTCACCTGT |
| | GACACCCCTGAAGAA |
| | GATGGTATCACCTGG |
| | ACCTTGGACCAGAGC |
| | AGTGAGGTCTTAGGC |
| | TCTGGCAAAACCCTG |
| | ACCATCCAAGTCAAA |
| | GAGTTTGGAGATGCT |
| | GGCCAGTACACCTGT |
| | CACAAAGGAGGGGAG |
| | GTTCTAAGCCATTCC |
| | CTCCTGCTGCTTCAC |
| | AAAAAGGAAGATGGA |
| | ATTTGGTCCACTGAT |
| | ATTTTAAAGGACCAG |
| | AAAGAACCCAAAAAT |
| | AAGACCTTTCTAAGA |
| | TGTGAGGCCAAGAAT |
| | TATTCTGGAAGGTTC |
| | ACCTGCTGGTGGCTG |
| | ACTACAATCAGTACT |
| | GATTTGACATTCAGT |
| | GTCAAAAGCAGCAGA |
| | GGCTCTTCTGACCCC |
| | CAAGGGGTGACATGT |
| | GGAGCTGCTACACTC |
| | TCTGCAGAGAGAGTC |
| | AGAGGGGACAACAAG |
| | GAGTATGAGTACTCA |
| | GTGGAGTGCCAGGAG |
| | GACAGTGCCTGCCCA |
| | GCTGCTGAGGAGAGT |
| | CTGCCCATTGAGGTg |
| | ATGGTGGATGCAGTT |
| | CACAAGCTCAAGTAT |
| | GAAAACTACACCAGC |
| | AGCTTCTTCATCAGG |
| | GACATCATCAAACCT |
| | GACCCACCCAAGAAC |
| | TTGCAGCTGAAGCCA |
| | TTAAAGAATTCTAGG |
| | CAGGTGGAGGTCAGC |
| | TGGGAGTACCCTGAC |
| | ACCTGGAGTACTCCA |
| | CATTCCTACTTCTCC |
| | CTGACATTCTGTGTT |
| | CAaGTCCAGGGCAAG |
| | AGCAAGAGAGAAAAG |
| | AAAGATAGAGTCTTC |
| | ACTGACAAGACCTCA |
| | GCCACAGTCATCTGC |
| | AGGAAAAATGCCAGC |
| | ATTTCAGTGAGGGCC |
| | CAGGACAGaTACTAT |
| | AGCTCATCTTGGTCA |
| | GAATGGGCATCTGTG |
| | CCCTGCAGTTGA |
| | (SEQ ID NO: 10) |
| WT mouse IL-12A | atggtcagcgttcca |
| (p35)(no | acagcctcaccctcg |
| in-frame | gcatccagcagctcc |
| stop codon) | tctcagtgccggtcc |
| | agcatgtgtcaatca |
| | cgctacctcctcttt |
| | ttggccacccttgcc |
| | ctcctaaaccacctc |
| | agtttggccagggtc |
| | attccagtctctgga |
| | cctgccaggtgtctt |
| | agccagtcccgaaac |
| | ctgctgaagaccaca |
| | gatgacatggtgaag |
| | acggccagagaaaaa |
| | ctgaaacattattcc |
| | tgcactgctgaagac |
| | atcgatcatgaagac |
| | atcacacgggaccaa |
| | accagcacattgaag |
| | acctgtttaccactg |
| | gaactacacaagaac |
| | gagagttgcctggct |
| | actagagagacttct |
| | tccacaacaagaggg |
| | agctgcctgccccca |
| | cagaagacgtctttg |
| | atgatgaccctgtgc |
| | cttggtagcatctat |
| | gaggacttgaagatg |
| | taccagacagagttc |
| | caggccatcaacgca |
| | gcacttcagaatcac |
| | aaccatcagcagatc |
| | attctagacaagggc |
| | atgctggtggccatc |
| | gatgagctgatgcag |
| | tctctgaatcataat |
| | ggcgagactctgcgc |
| | cagaaacctcctgtg |
| | ggagaagcagaccct |
| | tacagagtgaaaatg |
| | aagctctgcatcctg |
| | cttcacgccttcagc |
| | acccgcgtcgtgacc |
| | atcaacagggtgatg |
| | ggctatctgagctcc |
| | gcc |
| | (SEQ ID NO: 90) |
| WT mouse IL-12B | atgtgtcctcagaag |
| (p40) | ctaaccatctcctgg |
| | tttgccatcgttttg |
| | ctggtgtctccactc |
| | atggccatgtgggag |
| | ctggagaaagacgtt |
| | tatgttgtagaggtg |
| | gactggactcccgat |
| | gcccctggagaaaca |
| | gtgaacctcacctgt |
| | gacacgcctgaagaa |
| | gatgacatcacctgg |
| | acctcagaccagaga |
| | catggagtcataggc |
| | tctggaaagaccctg |
| | accatcactgtcaaa |
| | gagtttctagatgct |
| | ggccagtacacctgc |
| | cacaaaggaggcgag |
| | actctgagccactca |
| | catctgctgctccac |
| | aagaaggaaaatgga |
| | atttggtccactgaa |
| | attttaaaaaatttc |
| | aaaaacaagactttc |
| | ctgaagtgtgaagca |
| | ccaaattactccgga |
| | cggttcacgtgctca |
| | tggctggtgcaaaga |
| | aacatggacttgaag |
| | ttcaacatcaagagc |
| | agtagcagttcccct |
| | gactctcgggcagtg |
| | acatgtggaatggcg |
| | tctctgtctgcagag |
| | aaggtcacactggac |
| | caaagggactatgag |
| | aagtattcagtgtcc |
| | tgccaggaggatgtc |
| | acctgcccaactgcc |
| | gaggagaccctgccc |
| | attgaactggcgttg |
| | gaagcacggcagcag |
| | aataaatatgagaac |
| | tacagcaccagcttc |
| | ttcatcagggacatc |
| | atcaaaccagacccg |
| | cccaagaacttgcag |
| | atgaagcctttgaag |
| | aactcacaggtggag |
| | gtcagctgggagtac |
| | cctgactcctggagc |
| | actccccattcctac |
| | ttctccctcaagttc |
| | tttgttcgaatccag |
| | cgcaagaaagaaaag |
| | atgaaggagacagag |
| | gaggggtgtaaccag |
| | aaaggagcgttcctc |
| | gtagagaagacatct |
| | accgaagtccaatgc |
| | aaaggcgggaatgtc |
| | tgcgtgcaagctcag |
| | gatcgctattacaat |
| | tcctcatgcagcaag |
| | tgggcatgtgttccc |
| | tgcagggtccgatcc |
| | tga |
| | (SEQ ID NO: 91) |
| Human IL-12B | ATGTGTCACCAGCAG |
| (p40) (“wild- | TTGGTCATCTCTTGG |
| type” no CpG | TTTTCCCTGGTTTTT |
| modification | CTGGCATCTCCCCTC |
| and no | GTGGCCATATGGGAA |
| in-frame | CTGAAGAAAGATGTT |
| stop | TATGTCGTAGAATTG |
| codon) | GATTGGTATCCGGAT |
| | GCCCCTGGAGAAATG |
| | GTGGTCCTCACCTGT |
| | GACACCCCTGAAGAA |
| | GATGGTATCACCTGG |
| | ACCTTGGACCAGAGC |
| | AGTGAGGTCTTAGGC |
| | TCTGGCAAAACCCTG |
| | ACCATCCAAGTCAAA |
| | GAGTTTGGAGATGCT |
| | GGCCAGTACACCTGT |
| | CACAAAGGAGGCGAG |
| | GTTCTAAGCCATTCG |
| | CTCCTGCTGCTTCAC |
| | AAAAAGGAAGATGGA |
| | ATTTGGTCCACTGAT |
| | ATTTTAAAGGACCAG |
| | AAAGAACCCAAAAAT |
| | AAGACCTTTCTAAGA |
| | TGCGAGGCCAAGAAT |
| | TATTCTGGACGTTTC |
| | ACCTGCTGGTGGCTG |
| | ACGACAATCAGTACT |
| | GATTTGACATTCAGT |
| | GTCAAAAGCAGCAGA |
| | GGCTCTTCTGACCCC |
| | CAAGGGGTGACGTGC |
| | GGAGCTGCTACACTC |
| | TCTGCAGAGAGAGTC |
| | AGAGGGGACAACAAG |
| | GAGTATGAGTACTCA |
| | GTGGAGTGCCAGGAG |
| | GACAGTGCCTGCCCA |
| | GCTGCTGAGGAGAGT |
| | CTGCCCATTGAGGTg |
| | ATGGTGGATGCCGTT |
| | CACAAGCTCAAGTAT |
| | GAAAACTACACCAGC |
| | AGCTTCTTCATCAGG |
| | GACATCATCAAACCT |
| | GACCCACCCAAGAAC |
| | TTGCAGCTGAAGCCA |
| | TTAAAGAATTCTCGG |
| | CAGGTGGAGGTCAGC |
| | TGGGAGTACCCTGAC |
| | ACCTGGAGTACTCCA |
| | CATTCCTACTTCTCC |
| | CTGACATTCTGCGTT |
| | CAaGTCCAGGGCAAG |
| | AGCAAGAGAGAAAAG |
| | AAAGATAGAGTCTTC |
| | ACGGACAAGACCTCA |
| | GCCACGGTCATCTGC |
| | CGCAAAAATGCCAGC |
| | ATTAGCGTGCGGGCC |
| | CAGGACCGCTACTAT |
| | AGCTCATCTTGGAGC |
| | GAATGGGCATCTGTG |
| | CCCTGCAGT |
| | (SEQ ID NO: 92) |

TABLE 3
IL-12 Amino Acid Sequences

| | IL-12 Amino Acid |
|---|---|
| IL-12 | Sequences (SEQ ID NO) |
| Mouse IL-12A | MVSVPTASPSASSSSSQCRS |
| (p35) | SMCQSRYLLFLATLALLNHL |
| NP_001152896 | SLARVIPVSGPARCLSQSRN |
| | LLKTTDDMVKTAREKLKHYS |
| | CTAEDIDHEDITRDQTSTLK |
| | TCLPLELHKNESCLATRETS |
| | STTRGSCLPPQKTSLMMTLC |
| | LGSTYEDLKMYQTEFQAINA |
| | ALQNHNHQQIILDKGMLVAI |
| | DELMQSLNHNGETLRQKPPV |
| | GEADPYRVKMKLCILLHAFS |
| | TRVVTINRVMGYLSSA |
| | (SEQ ID NO: 78) |
| Mouse IL-12B | MCPQKLTISWFAIVLLVSPL |
| (p40) | MAMWELEKDVYVVEVDWTPD |
| NP_001290173 | APGETVNLTCDTPEEDDITW |
| | TSDQRHGVIGSGKTLTITVK |
| | EFLDAGQYTCHKGGETLSHS |
| | HLLLHKKENGIWSTEILKNE |
| | KNKTFLKCEAPNYSGRFTCS |
| | WLVQRNMDLKENIKSSSSSP |
| | DSRAVTCGMASLSAEKVTLD |
| | QRDYEKYSVSCQEDVTCPTA |
| | EETLPIELALEARQQNKYEN |
| | YSTSFFIRDIIKPDPPKNLQ |
| | MKPLKNSQVEVSWEYPDSWS |
| | TPHSYFSLKFFVRIQRKKEK |
| | MKETEEGCNQKGAFLVEKTS |
| | TEVQCKGGNVCVQAQDRYYN |
| | SSCSKWACVPCRVRS |
| | (SEQ ID NO: 79) |
| Human IL-12A | MWPPGSASQPPPSPAAATGL |
| (p35) | HPAARPVSLQCRLSMCPARS |
| NP_000873 | LLLVATLVLLDHLSLARNLP |
| | VATPDPGMFPCLHHSQNLLR |
| | AVSNMLQKARQTLEFYPCTS |
| | EEIDHEDITKDKTSTVEACL |
| | PLELTKNESCLNSRETSFIT |
| | NGSCLASRKTSFMMALCLSS |
| | IYEDLKMYQVEEKTMNAKLL |
| | MDPKRINFLDQNMLAVIDEL |
| | MQALNENSETVPQKSSLEEP |
| | DFYKTKIKLCILLHAFRIRA |
| | VTIDRVMSYLNAS |
| | (SEQ ID NO: 80) |
| Human IL-12B | MCHQQLVISWFSLVFLASPL |
| (p40) | VAIWELKKDVYVVELDWYPD |
| NP_002178 | APGEMVVLTCDTPEEDGITW |
| | TLDQSSEVLGSGKTLTDQVK |
| | EFGDAGQYTCHKGGEVLSHS |
| | LLLLHKKEDGIWSTDILKDQ |
| | KEPKNKTFLRCEAKNYSGRF |
| | TCWWLTTISTDLTFSVKSSR |
| | GSSDPQGVTCGAATLSAERV |
| | RGDNKEYEYSVECQEDSACP |
| | AAEESLPIEVMVDAVHKLKY |
| | ENYTSSFFIRDIIKPDPPKN |
| | LQLKPLKNSRQVEVSWEYPD |
| | TWSTPHSYFSLTFCVQVQGK |
| | SKREKKDRVFTDKTSATVIC |
| | RKNASISVRAQDRYYSSSWS |
| | EWASVPCS |
| | (SEQ ID NO: 81) |

### IL-12

In some aspects, the cytokine is an IL-12 molecule, e.g., a full length, a functional fragment of IL-12, or a variant of IL-12, e.g., human IL-12. In some aspects, the IL-12 molecule is a wild-type, human IL12. In other embodiments, the IL-12 molecule is a variant of human IL-12, e.g., having one or more amino acid modifications. In some aspects, the composition (e.g., gene therapy composition) comprises a construct of the disclosure, which is a multicistronic (e.g., bicistronic) construct (e.g., comprising IL-12A and IL-12B subunits).

Interleukin-12 (IL-12) is a heterodimeric cytokine composed of p35 and p40 subunits which are encoded by 2 separate genes, IL-12A and IL-12B, respectively. IL-12 is involved in the differentiation of naive T cells into Th1 cells. It is known as a T cell-stimulating factor, which can stimulate the growth and function of T cells. It stimulates the production of IFN-γ and TNF-α from T cells and natural killer (NK) cells, and reduces IL-4 mediated suppression of IFN-γ. T cells that produce IL-12 have a coreceptor, CD30, which is associated with IL-12 activity.

IL-12 plays an important role in the activities of NK cells and T lymphocytes. IL-12 mediates enhancement of the cytotoxic activity of NK cells and CD8+ cytotoxic T lymphocytes. There also seems to be a link between IL-2 and the signal transduction of IL-12 in NK cells. IL-2 stimulates the expression of two IL-12 receptors, IL-12R-pi and IL-12R-P2, maintaining the expression of a critical protein involved in IL-12 signaling in NK cells. Enhanced functional response is demonstrated by IFN-γ production and killing of target cells.

IL-12 also has anti-angiogenic activity, which means it can block the formation of new blood vessels. It does this by increasing production of IFN-γ, which in turn increases the production of a chemokine called inducible protein-10 (IP-10 or CXCL10). IP-10 then mediates this anti-angiogenic effect. Because of its ability to induce immune responses and its anti-angiogenic activity, there has been an interest in testing IL-12 as a possible anti-cancer drug.

IL-12 binds to the IL-12 receptor, which is a heterodimeric receptor formed by IL-12R-β\hat{ι} and IL-12R-P2. IL-12R-P2 is considered to play a key role in IL-12 function, since it is found on activated T cells and is stimulated by cytokines that promote Th1 cells development and inhibited by those that promote Th2 cells development. Upon binding, IL-12R-P2 becomes tyrosine phosphorylated and provides binding sites for kinases, Tyk2 and Jak2. These are important in activating critical transcription factor proteins such as STAT4 that are implicated in IL-12 signaling in T cells and NK cells. IL-12 is a potent cytokine with the potential to reshape the anti-inflammatory environment in solid tumors. However, its clinical utility has been limited by severe toxicities both from soluble administration or from adoptively transferred T cells engineered to secrete IL-12. See e.g., U.S. Pat. No. 8,026,223B1; Int'l Publ. No. WO2019/010219; Liu, Y., et al., Cancer Gene Ther., 9:9-15 (2002); Heinzerling L, et al., Hum Gene Ther., 16(1):35-48 (2005).

In some aspects, provided herein is are polynucleotides, compositions, and viral vectors comprising an enhancer (e.g., a CMV enhancer), a promoter, for example, a constituative promoter (e.g., a CBA promoter or fragment thereof) operably linked to a first nucleic acid encoding a first IL-12 subunit or a functional fragment thereof (e.g., an IL-12 p35 subunit or an IL-12 p40 subunit), a translation modification sequence (e.g., a furin cleavage sequence followed by a 2A self-processing peptide (F2A)), a second nucleic acid encoding an IL-12 subunit or a functional fragment thereof (e.g., an IL-12 p35 subunit or an IL-12 p40 subunit), and a pA sequence (e.g., a human growth hormone (HGH) or a bovine growth hormone (BGH) pA sequence). In some aspects, the polynucleotides, compositions, and viral vectors further comprise an intron (e.g., a CAG intron). In some aspects, the polynucleotides, compositions, and viral vectors further comprise a Kozak sequence.

In some aspects, the first nucleic acid encodes a polypeptide having at least 85%, 90%, 95%, 96%, 97%, 98%, 99%, or 100% identity to the amino acid sequence of any of SEQ ID NO: 80 or 81, or any amino acid sequence in Table 3. In some aspects, the second nucleic acid encodes a polypeptide having at least 85%, 90%, 95%, 96%, 97%, 98%, 99%, or 100% identity to the amino acid sequence of any of SEQ ID NO: 80 or 81, or any amino acid sequence in Table 3. In some aspects, the first nucleic acid (e.g., a nucleic acid encoding an IL-12 p35 subunit) does not include an in-frame stop codon (e.g., TGA, TAG, and TAA).

In some aspects, the nucleic acid encoding the IL-12 p35 subunit comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77. In some aspects, the nucleic acid encoding the IL-12 p40 subunit comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92.

In some aspects, the polynucleotide of the disclosure comprises a promoter operably linked to an IL-12 p35 gene or functional fragment thereof (e.g., corresponding to SEQ ID NO: 77), a furin cleavage sequence and/or a 2A self-processing peptide sequence (including an F2A, E2A, P2A or T2A self-processing peptide), and an IL-12 p40 gene or a functional fragment thereof (e.g., corresponding to SEQ ID NO: 8); and a pA sequence (e.g., a growth hormone pA). In some aspects, the promoter comprises a CBA promoter or a fragment thereof; and optionally, a CMV enhancer is included. In some aspects, the promoter is a CBA promoter or fragment thereof. In some aspects, the polynucleotide further comprise an intron (e.g., a CAG intron). In some aspects, the polynucleotide further comprise a Kozak sequence

In some aspects, the second cytokine is IL-15, IL-2, or a functional fragment thereof. In some aspects, the second nucleic acid comprises an IL-15 gene. In some aspects, the second nucleic acid comprises a nucleic acid sequence having at least 85%, 90%, 95% 96%, 97%, 98%, 99%, or 100% identity to SEQ ID NOs: 11-13, 58, or any nucleic acid shown in Table 4, or any combination thereof. In some aspects, the second nucleic acid comprises a nucleic acid sequence having at least 85%, 90%, 95% 96%, 97%, 98%, 99%, or 100% identity to a reverse complement of SEQ ID NOs: 11-13, 58, or any nucleic acid shown in Table 4, or any combination thereof. In some aspects, the polynucleotide of the disclosure comprises one or more nucleic acids comprising a nucleic acid sequence having at least 85%, 90%, 95% 96%, 97%, 98%, 99%, or 100% identity to SEQ ID NOs: 11-13, 58, or any nucleic acid sequence shown in Table 4, or any combination thereof. In some aspects, the polynucleotide of the disclosure comprises one or more nucleic acids comprising a nucleic acid sequence having at least 85%, 90%, 95% 96%, 97%, 98%, 99%, or 100% identity to a reverse complement of SEQ ID NOs: 11-13, 58, or any nucleic acid sequence shown in Table 4, or any combination thereof.

TABLE 4
IL-15 Nucleic Acid Sequences

| | IL-15 Nucleic |
|---|---|
| | Acid Sequence |
| IL-15 | (SEQ ID NO) |
| IL-15 (“wild- | ATGAGAATTTCGAAACCACA |
| type” no CpG | TTTGAGAAGTATTTCCATCC |
| modification) | AGTGCTACTTGTGTTTACTT |
| | CTAAACAGTCATTTTCTAAC |
| | TGAAGCTGGCATTCATGTCT |
| | TCATTTTGGGCTGTTTCtct |
| | GCAGGGCTTCCTAAAACAGA |
| | AGCCAACTGGGTGAATGTAA |
| | TAAGTGATTTGAAAAAAATT |
| | GAAGATCTTATTCAATCTAT |
| | GCATATTGATGCTACTTTAT |
| | ATACGGAAAGTGATGTTCAC |
| | CCCAGTTGCAAAGTAACAGC |
| | AATGAAGTGCTTTCTCTTGG |
| | AGTTACAAGTTATTTCACTT |
| | GAGTCCGGAGATGCAAGTAT |
| | TCATGATACAGTAGAAAATC |
| | TGATCATCCTAGCAAACAAC |
| | AGTTTGTCTTCTAATGGGAA |
| | TGTAACAGAATCTGGATGCA |
| | AAGAATGTGAGGAACTGGAG |
| | GAAAAAAATATTAAAGAATT |
| | TTTGCAGAGTTTTGTACATA |
| | TTGTCCAAATGTTCATCAAC |
| | ACTTCTTGA |
| | (SEQ ID NO: 11) |
| IL-15 | ATGAGAATTAGCAAACCACA |
| (with CpG | TTTGAGAAGTATTTCCATCC |
| modification) | AGTGCTACTTGTGTTTACTT |
| | CTAAACAGTCATTTTCTAAC |
| | TGAAGCTGGCATTCATGTCT |
| | TCATTTTGGGCTGTTTCtct |
| | GCAGGGCTTCCTAAAACAGA |
| | AGCCAACTGGGTGAATGTAA |
| | TAAGTGATTTGAAAAAAATT |
| | GAAGATCTTATTCAATCTAT |
| | GCATATTGATGCTACTTTAT |
| | ATACTGAAAGTGATGTTCAC |
| | CCCAGTTGCAAAGTAACAGC |
| | AATGAAGTGCTTTCTCTTGG |
| | AGTTACAAGTTATTTCACTT |
| | GAGAGCGGAGATGCAAGTAT |
| | TCATGATACAGTAGAAAATC |
| | TGATCATCCTAGCAAACAAC |
| | AGTTTGTCTTCTAATGGGAA |
| | TGTAACAGAATCTGGATGCA |
| | AAGAATGTGAGGAACTGGAG |
| | GAAAAAAATATTAAAGAATT |
| | TTTGCAGAGTTTTGTACATA |
| | TTGTCCAAATGTTCATCAAC |
| | ACTTCTTGA |
| | (SEQ ID NO: 12) |
| IL-15 | ATGAGAATTAGCAAACCACA |
| (with CpG | TTTGAGAAGTATTTCCATCC |
| modification) | AGTGCTACTTGTGTTTACTT |
| | CTAAACAGTCATTTTCTAAC |
| | TGAAGCTGGCATTCATGTCT |
| | TCATTTTGGGCTGTTTCtct |
| | GCAGGGCTTCCTAAAACAGA |
| | AGCCAACTGGGTGAATGTAA |
| | TAAGTGATTTGAAAAAAATT |
| | GAAGATCTTATTCAATCTAT |
| | GCATATTGATGCTACTTTAT |
| | ATACTGAAAGTGATGTTCAC |
| | CCCAGTTGCAAAGTAACAGC |
| | AATGAAGTGCTTTCTCTTGG |
| | AGTTACAAGTTATTTCACTT |
| | GAGTCAGGAGATGCAAGTAT |
| | TCATGATACAGTAGAAAATC |
| | TGATCATCCTAGCAAACAAC |
| | AGTTTGTCTTCTAATGGGAA |
| | TGTAACAGAATCTGGATGCA |
| | AAGAATGTGAGGAACTGGAG |
| | GAAAAAAATATTAAAGAATT |
| | TTTGCAGAGTTTTGTACATA |
| | TTGTCCAAATGTTCATCAAC |
| | ACTTCTTGA |
| | (SEQ ID NO: 13) |
| IL-15(splice | ATGAGAATTTCGAAACCACA |
| variant | TTTGAGAAGTATTTCCATCC |
| modification) | AGTGCTACTTGTGTTTACTT |
| | CTAAACAGTCATTTTCTAAC |
| | TGAAGCTGGCATTCATGTCT |
| | TCATTTTGGGCTGTTTCTCT |
| | GCTGGGCTTCCTAAAACAGA |
| | AGCCAACTGGGTGAATGTAA |
| | TAAGTGATTTGAAAAAAATT |
| | GAAGATCTTATTCAATCTAT |
| | GCATATTGATGCTACTTTAT |
| | ATACGGAAAGTGATGTTCAC |
| | CCCAGTTGCAAAGTAACAGC |
| | AATGAAGTGCTTTCTCTTGG |
| | AGTTACAAGTTATTTCACTT |
| | GAGTCCGGAGATGCAAGTAT |
| | TCATGATACAGTAGAAAATC |
| | TGATCATCCTAGCAAACAAC |
| | AGTTTGTCTTCTAATGGGAA |
| | TGTAACAGAATCTGGATGCA |
| | AAGAATGTGAGGAACTGGAG |
| | GAAAAAAATATTAAAGAATT |
| | TTTGCAGAGTTTTGTACATA |
| | TTGTCCAAATGTTCATCAAC |
| | ACTTCTTGA |
| | (SEQ ID NO: 58) |

### IL-15

In some aspects, the cytokine is an IL-15 molecule, e.g., a full length, a functional fragment of IL-15, or a variant of IL-15, e.g., human IL-15. In some aspects, the IL-15 molecule is a wild-type, human IL-15. In other embodiments, the IL-15 molecule is a variant of human IL-15, e.g., having one or more amino acid modifications.

IL-15 is a four α-helix bundle cytokine displaying IL-2-like immunomodulatory functions, supporting the proliferation and differentiation of T cells, B cells, and NK cells. In addition, IL-15 has been reported to control differentiation of NK cells from bone marrow precursors, to stimulate NK antitumor cytolytic functions, and to act as a chemotactic stimulus for NK cells. The critical role of IL-15 in NK cell development and function was also evidenced in IFN-regulatory factor 1 (IRF-1) knockout mice, which lack IL-15 expression and display an NK-deficient phenotype. Thus, IL-15 has been regarded as a suitable candidate for cancer immunotherapy or gene therapy strategies. See e.g., Di Carlo et al., J Immunol, 165(6):3111-3118 (2000).

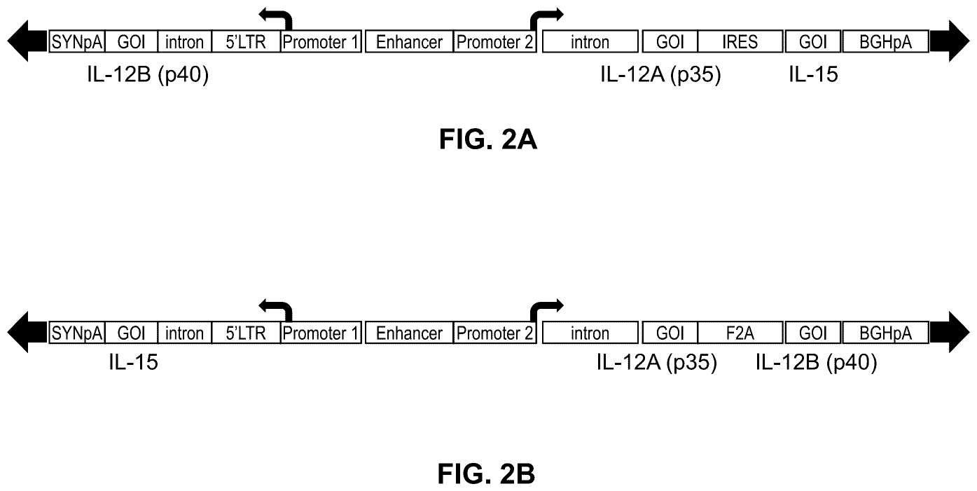

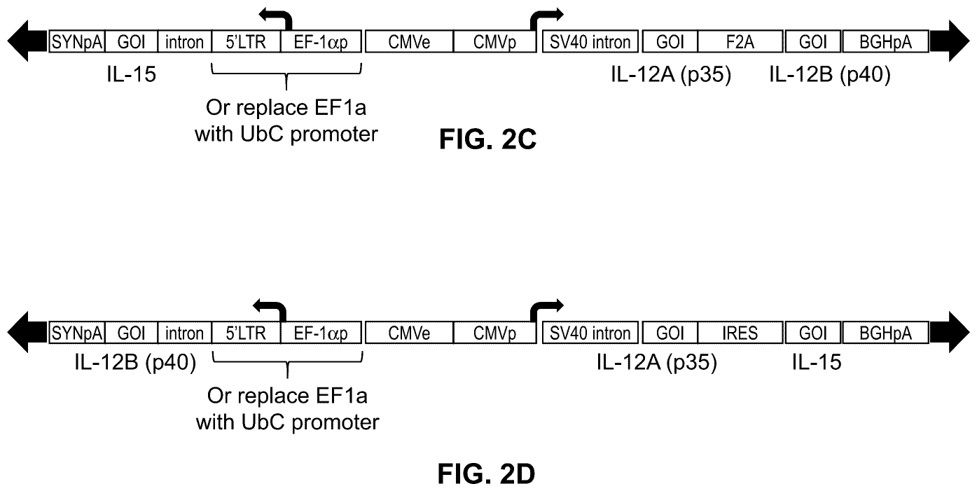

In some aspects, the first nucleic acid comprises a promoter operably linked to an IL-12 p35 gene or functional fragment thereof, an IRES sequence, and an IL-15 gene or a functional fragment thereof; and the second nucleic acid comprises a promoter operably linked to an IL-12 p40 gene or functional fragment thereof. (see FIG. 2A). In some aspects, the first nucleic acid comprises a promoter operably linked to a nucleic acid encoding an IL-12 p35 subunit or functional fragment thereof, an IRES sequence, and, optionally, an IL-15 gene or a functional fragment thereof; and the second nucleic acid comprises a promoter operably linked to a nucleic acid encoding an IL-12 p40 subunit or functional fragment thereof. In some aspects, the first nucleic acid comprises a promoter operably linked to a nucleic acid encoding an IL-12 p40 subunit or functional fragment thereof, an IRES sequence, and, optionally, an IL-15 gene or a functional fragment thereof; and the second nucleic acid comprises a promoter operably linked to a nucleic acid encoding an IL-12 p35 subunit or functional fragment thereof.

In some aspects, the first nucleic acid comprises a promoter operably linked to an IL-12 p35 gene or functional fragment thereof, a furin cleavage sequence and/or a 2A self-processing peptide (including an F2A, E2A, P2A or T2A self-processing peptide), and an IL-12 p40 gene or a functional fragment thereof; and the second nucleic acid comprises a promoter operably linked to an IL-15 gene or functional fragment thereof. (see FIG. 2B). In some aspects, the first nucleic acid comprises a promoter operably linked to a nucleic acid encoding an IL-12 p35 subunit or functional fragment thereof, a furin cleavage sequence and/or a 2A self-processing peptide (including an F2A, E2A, P2A or T2A self-processing peptide), and a nucleic acid encoding an IL-12 p40 subunit or a functional fragment thereof; and, optionally, a second nucleic acid comprises a promoter operably linked to an IL-15 gene or functional fragment thereof. In some aspects, the first nucleic acid comprises a promoter operably linked to a nucleic acid encoding an IL-12 p40 subunit or functional fragment thereof, a furin cleavage sequence and/or a 2A self-processing peptide (including an F2A, E2A, P2A or T2A self-processing peptide), and a nucleic acid encoding an IL-12 p35 subunit or a functional fragment thereof, and, optionally, a second nucleic acid comprises a promoter operably linked to an IL-15 gene or functional fragment thereof.

In some aspects, the first nucleic acid comprises a promoter operably linked to an IL-12 p35 gene or functional fragment thereof, an IRES sequence, and an IL-15 gene or a functional fragment thereof; and the second nucleic acid comprises a promoter operably linked to an IL-12 p40 gene or functional fragment thereof, wherein the construct comprises a nucleic acid sequence having at least 80%, 85%, 90%, 95% 96%, 97%, 98%, 99%, or 100% identity to any of SEQ ID NOs: 14-37 and 48-57 or any nucleic acid shown in Table 5.

In some aspects, the first nucleic acid comprises a promoter operably linked to an IL-12 p35 gene or functional fragment thereof, a furin cleavage sequence and/or a 2A self-processing peptide (including an F2A, E2A, P2A or T2A self-processing peptide), and an IL-12 p40 gene or a functional fragment thereof; and the second nucleic acid comprises a promoter operably linked to an IL-15 gene or functional fragment thereof, wherein the construct comprises a nucleic acid sequence having at least 80%, 85%, 90%, 95%, 96%, 97%, 98%, 99%, or 10000 identity to any of SEQ ID NOs: 14-37 and 48-57 or any nucleic acid shown in Table 5.

TABLE 5
IL-12 and/or IL-15 Exemplary Expression Cassette or
Construct Nucleic Acid Sequence

| | | IL-12 and/or IL-15 Expression |
|---|---|---|
| | | Cassette or Construct |
| 1 | Expression Cassette | Nucleic Acid Sequence (SEQ ID NO) |
| | Human EF-1a-wild-type IL- | (SEQ ID NO: 14) |
| | 12A/IL-12B/IL-15-IRES | |
| | (no CpG modification) | |
| 2 | Human EF-1a-wild-type IL- | (SEQ ID NO: 15) |
| | 12A/IL-12B/IL-15-F2A(no | |
| | CpG modification) | |
| 3 | Human EF-1a-modified IL- | (SEQ ID NO: 16) |
| | 12A/IL-12B/IL-15- | |
| | IRES(with CpG | |
| | modification) | |
| 4 | Human EF1a-modified IL- | (SEQ ID NO: 17) |
| | 12A/IL-12B/IL-15- | |
| | F2A(with CpG modification) | |
| 5 | Human EF1a-modified IL- | (SEQ ID NO: 18) |
| | 12A/IL-12B/IL-15-IRES | |
| | (with CpG modification) | |
| 6 | Human EF-1a-modified IL- | (SEQ ID NO: 19) |
| | 12A/IL-12B/IL-15- | |
| | F2A(with CpG modification) | |
| 7 | Human EF-1a-wild-type IL- | (SEQ ID NO: 20) |
| | 12A/IL-12B/IL-15-ODN- | |
| | IRES (no CpG modification) | |
| 8 | Human EF-1a-wild-type | (SEQ ID NO: 21) |
| | IL-12A/IL-12B/IL-15-ODN- | |
| | F2A (no CpG | |
| | modification) | |
| 9 | Human UbC-wild-type IL- | (SEQ ID NO: 22) |
| | 12A/IL-12B/IL-15-IRES | |
| | (no CpG modification) | |
| 10 | Human UbC-wild-type IL- | (SEQ ID NO: 23) |
| | 12A/IL-12B/IL-15-F2A(no | |
| | CpG modification) | |
| 11 | Human UbC-modified IL- | (SEQ ID NO: 24) |
| | 12A/IL-12B/IL-15- | |
| | IRES(with CpG | |
| | modification) | |
| 12 | Human UbC-modified IL- | (SEQ ID NO: 25) |
| | 12A/IL-12B/IL-15- | |
| | F2A(with CpG modification) | |
| 13 | Human UbC-modified IL- | (SEQ ID NO: 26) |
| | 12A/IL-12B/IL-15-IRES | |
| | (with CpG modification) | |
| 14 | Human UbC-modified IL- | (SEQ ID NO: 27) |
| | 12A/IL-12B/IL-15-F2A- | |
| | (with CpG modification) | |
| 15 | Human UbC-wild-type IL- | (SEQ ID NO: 28) |
| | 12A/IL-12B/IL-15-ODN-IRES | |
| | (no CpG modification) | |
| 16 | Human UbC-wild-type IL- | (SEQ ID NO: 29) |
| | 12A/IL-12B/IL-15-ODN-F2A | |
| | (no CpG modifiation) | |
| 17 | Human CMV-wild-type IL- | (SEQ ID NO: 30) |
| | 12A/IL-12B--F2A(no CpG | |
| | modification) | |
| 18 | Human CMV-wild-type IL-15 | (SEQ ID NO: 31) |
| | (no CpG modification) | |
| 19 | Human CAG-wild-type IL-12A/IL- | CTGCGCGCTCGCTCGCTCACTGAGGCCGCCCGGGCAAAGCCCG |
| | 12B-IRES (no CpG modification) | GGCGTCGGGCGACCTTTGGTCGCCCGGCCTCAGTGAGCGAGCG |
| | construct | AGCGCGCAGAGAGGGAGTGGCCAACTCCATCACTAGGGGTTCC |
| | | TTAGTATTAAACGCGTGTCGACATTGATTATTGACTAGTTATT |
| | | AATAGTAATCAATTACGGGGTCATTAGTTCATAGCCCATATAT |
| | | GGAGTTCCGCGTTACATAACTTACGGTAAATGGCCCGCCTGGC |
| | | TGACCGCCCAACGACCCCCGCCCATTGACGTCAATAATGACGT |
| | | ATGTTCCCATAGTAACGCCAATAGGGACTTTCCATTGACGTCA |
| | | ATGGGTGGACTATTTACGGTAAACTGCCCACTTGGCAGTACAT |
| | | CAAGTGTATCATATGCCAAGTACGCCCCCTATTGACGTCAATG |
| | | ACGGTAAATGGCCCGCCTGGCATTATGCCCAGTACATGACCTT |
| | | ATGGGACTTTCCTACTTGGCAGTACATCTACGTATTAGTCATC |
| | | GCTATTACCATGGGTCGAGGTGAGCCCCACGTTCTGCTTCACT |
| | | CTCCCCATCTCCCCCCCCTCCCCACCCCCAATTTTGTATTTAT |
| | | TTATTTTTTAATTATTTTGTGCAGCGATGGGGGCGGGGGGGGG |
| | | GGGGGCGCGCGCCAGGCGGGGCGGGGCGGGGCGAGGGGCGGGG |
| | | CGGGGCGAGGCGGAGAGGTGCGGCGGCAGCCAATCAGAGCGGC |
| | | GCGCTCCGAAAGTTTCCTTTTATGGCGAGGCGGCGGCGGCGGC |
| | | GGCCCTATAAAAAGCGAAGCGCGCGGCGGGCGGGAGTCGCTGC |
| | | GCGCTGCCTTCGCCCCGTGCCCCGCTCCGCCGCCGCCTCGCGC |
| | | CGCCCGCCCCGGCTCTGACTGACCGCGTTACTCCCACAGGTGA |
| | | GCGGGCGGGACGGCCCTTCTCCTCCGGGCTGTAATTAGCGCTT |
| | | GGTTTAATGACGGCTCGTTTCTTTTCTGTGGCTGCGTGAAAGC |
| | | CTTGAGGGGCTCCGGGAGGGCCCTTTGTGCGGGGGGAGCGGCT |
| | | CGGGGGGTGCGTGCGTGTGTGTGTGCGTGGGGAGCGCCGCGTG |
| | | CGGCTCCGCGCTGCCCGGCGGCTGTGAGCGCTGCGGGCGCGGC |
| | | GCGGGGCTTTGTGCGCTCCGCAGTGTGCGCGAGAGGAGCGCGG |
| | | CCGGGGGCGGTGCCACGCGGTGCGGGGGGGGCTGCGAGGGGAA |
| | | CAAAGGCTGCGTGCGGGGTGTGTGCGTGGGGGGGGTGAGCAGG |
| | | GGGTGTGGGCGCGGCGGTCGGGCTGTAACCCCCCCCTGCACCC |
| | | CCCTCCCCGAGTTGCTGAGCACGGCCCGGCTTCGGGTGCGGGG |
| | | CTCCGTGCGGGGCGTGGCGCGGGGCTCGCCGTGCCGGGCGGGG |
| | | GGTGGCGGCGGGTGGGGGTGCCGGGCGGGGCGGGGCCGCCTCG |
| | | GGCCGGGGAGGGCTCGGGGGAGGGGCGCGGCGGCCCCCGGAGC |
| | | GCCGGCGGCTGTCGAGGCGCGGCGAGCCGCAGCCATTGCCTTT |
| | | TATGGTAATCGTGCGAGAGGGCGCAGGGACTTCCTTTGTCCCA |
| | | AATCTGTGCGGAGCCGAAATCTGGGAGGCGCCGCCGCACCCCC |
| | | TCTAGCGGGCGCGGGGCGAAGCGGTGCGGCGCCGGCAGGAAGG |
| | | AAATGGGCGGGGAGGGCCTTCGTGCGTCGCCGCGCCGCCGTCC |
| | | CCTTCTCCCTCTCCAGCCTCGGGGCTGTCCGCGGGGGGACGGC |
| | | TGCCTTCGGGGGGGACGGGGCAGGGCGGGGTTCGGCTTCTGGC |
| | | GTGTGACCGGCGGTCTAGACTCTGCTAACCTTGTTCATGCCTT |
| | | CTTCTCTTTCCTACAGCTCCTGGGCAACGTGCTGGTTGTTGTG |
| | | CTGTCTCATCATTTTGGCAAAGAATTCAAGCTTCCAGCTAGCG |
| | | CCACCATGTGTCCAGCGCGCAGCCTCCTCCTTGTGGCTACCCT |
| | | GGTCCTCCTGGACCACCTCAGTTTGGCCAGAAACCTCCCCGTG |
| | | GCCACTCCAGACCCAGGAATGTTCCCATGCCTTCACCACTCCC |
| | | AAAACCTGCTGAGGGCCGTCAGCAACATGCTCCAGAAGGCCAG |
| | | ACAAACTCTAGAATTTTACCCTTGCACTTCTGAAGAGATTGAT |
| | | CATGAAGATATCACAAAAGATAAAACCAGCACAGTGGAGGCCT |
| | | GTTTACCATTGGAATTAACCAAGAATGAGAGTTGCCTAAATTC |
| | | CAGAGAGACCTCTTTCATAACTAATGGGAGTTGCCTGGCCTCC |
| | | AGAAAGACCTCTTTTATGATGGCCCTGTGCCTTAGTAGTATTT |
| | | ATGAAGACTTGAAGATGTACCAGGTGGAGTTCAAGACCATGAA |
| | | TGCAAAGCTTCTGATGGATCCTAAGAGGCAGATCTTTCTAGAT |
| | | CAAAACATGCTGGCAGTTATTGATGAGCTGATGCAGGCCCTGA |
| | | ATTTCAACAGTGAGACTGTGCCACAAAAATCCTCCCTTGAAGA |
| | | ACCGGATTTTTATAAAACTAAAATCAAGCTCTGCATACTTCTT |
| | | CATGCTTTCCGGATTCGGGCAGTGACTATTGATAGAGTGATGA |
| | | GCTATCTGAATGCTTCCTGAGCCCCTCTCCCTCCCCCCCCCCT |
| | | AACGTTACTGGCCGAAGCCGCTTGGAATAAGGCCGGTGTGCGT |
| | | TTGTCTATATGTTATTTTCCACCATATTGCCGTCTTTTGGCAA |
| | | TGTGAGGGCCCGGAAACCTGGCCCTGTCTTCTTGACGAGCATT |
| | | CCTAGGGGTCTTTCCCCTCTCGCCAAAGGAATGCAAGGTCTGT |
| | | TGAATGTCGTGAAGGAAGCAGTTCCTCTGGAAGCTTCTTGAAG |
| | | ACAAACAACGTCTGTAGCGACCCTTTGCAGGCAGCGGAACCCC |
| | | CCACCTGGCGACAGGTGCCTCTGCGGCCAAAAGCCACGTGTAT |
| | | AAGATACACCTGCAAAGGCGGCACAACCCCAGTGCCACGTTGT |
| | | GAGTTGGATAGTTGTGGAAAGAGTCAAATGGCTCTCCTCAAGC |
| | | GTATTCAACAAGGGGCTGAAGGATGCCCAGAAGGTACCCCATT |
| | | GTATGGGATCTGATCTGGGGCCTCGGTACACATGCTTTACATG |
| | | TGTTTAGTCGAGGTTAAAAAAACGTCTAGGCCCCCCGAACCAC |
| | | GGGGACGTGGTTTTCCTTTGAAAAACACGATGATAATATGGCC |
| | | ACAATGTGTCACCAGCAGTTGGTCATCTCTTGGTTTTCCCTGG |
| | | TTTTTCTGGCATCTCCCCTCGTGGCCATATGGGAACTGAAGAA |
| | | AGATGTTTATGTCGTAGAATTGGATTGGTATCCGGATGCCCCT |
| | | GGAGAAATGGTGGTCCTCACCTGTGACACCCCTGAAGAAGATG |
| | | GTATCACCTGGACCTTGGACCAGAGCAGTGAGGTCTTAGGCTC |
| | | TGGCAAAACCCTGACCATCCAAGTCAAAGAGTTTGGAGATGCT |
| | | GGCCAGTACACCTGTCACAAAGGAGGCGAGGTTCTAAGCCATT |
| | | CGCTCCTGCTGCTTCACAAAAAGGAAGATGGAATTTGGTCCAC |
| | | TGATATTTTAAAGGACCAGAAAGAACCCAAAAATAAGACCTTT |
| | | CTAAGATGCGAGGCCAAGAATTATTCTGGACGTTTCACCTGCT |
| | | GGTGGCTGACGACAATCAGTACTGATTTGACATTCAGTGTCAA |
| | | AAGCAGCAGAGGCTCTTCTGACCCCCAAGGGGTGACGTGCGGA |
| | | GCTGCTACACTCTCTGCAGAGAGAGTCAGAGGGGACAACAAGG |
| | | AGTATGAGTACTCAGTGGAGTGCCAGGAGGACAGTGCCTGCCC |
| | | AGCTGCTGAGGAGAGTCTGCCCATTGAGGTGATGGTGGATGCC |
| | | GTTCACAAGCTCAAGTATGAAAACTACACCAGCAGCTTCTTCA |
| | | TCAGGGACATCATCAAACCTGACCCACCCAAGAACTTGCAGCT |
| | | GAAGCCATTAAAGAATTCTCGGCAGGTGGAGGTCAGCTGGGAG |
| | | TACCCTGACACCTGGAGTACTCCACATTCCTACTTCTCCCTGA |
| | | CATTCTGCGTTCAAGTCCAGGGCAAGAGCAAGAGAGAAAAGAA |
| | | AGATAGAGTCTTCACGGACAAGACCTCAGCCACGGTCATCTGC |
| | | CGCAAAAATGCCAGCATTAGCGTGCGGGCCCAGGACCGCTACT |
| | | ATAGCTCATCTTGGAGCGAATGGGCATCTGTGCCCTGCAGTTG |
| | | ACAATTGCTGTGCCTTCTAGTTGCCAGCCATCTGTTGTTTGCC |
| | | CCTCCCCCGTGCCTTCCTTGACCCTGGAAGGTGCCACTCCCAC |
| | | TGTCCTTTCCTAATAAAATGAGGAAATTGCATCGCATTGTCTG |
| | | AGTAGGTGTCATTCTATTCTGGGGGGTGGGGTGGGGCAGGACA |
| | | GCAAGGGGGAGGATTGGGAAGACAATAGCAGGCATGCTGGGGA |
| | | TGCGGTGGGCTCTATGGAGCTCGAGAGGAACCCCTAGTGATGG |
| | | AGTTGGCCACTCCCTCTCTGCGCGCTCGCTCGCTCACTGAGGC |
| | | CGGGCGACCAAAGGTCGCCCGACGCCCGGGCTTTGCCCGGGCG |
| | | GCCTCAGTGAGCGAGCGAGCGCGCAGCTGCAGATCTG (SEQ |
| | | ID NO: 34) |
| 19 | Human CAG-wild-type IL-12A/IL- | GCTAGCGCCACCATGTGTCCAGCGCGCAGCCTCCTCCTTGTGG |
| | 12B-IRES (no CpG modification) | CTACCCTGGTCCTCCTGGACCACCTCAGTTTGGCCAGAAACCT |
| | expression sequence | CCCCGTGGCCACTCCAGACCCAGGAATGTTCCCATGCCTTCAC |
| | | CACTCCCAAAACCTGCTGAGGGCCGTCAGCAACATGCTCCAGA |
| | | AGGCCAGACAAACTCTAGAATTTTACCCTTGCACTTCTGAAGA |
| | | GATTGATCATGAAGATATCACAAAAGATAAAACCAGCACAGTG |
| | | GAGGCCTGTTTACCATTGGAATTAACCAAGAATGAGAGTTGCC |
| | | TAAATTCCAGAGAGACCTCTTTCATAACTAATGGGAGTTGCCT |
| | | GGCCTCCAGAAAGACCTCTTTTATGATGGCCCTGTGCCTTAGT |
| | | AGTATTTATGAAGACTTGAAGATGTACCAGGTGGAGTTCAAGA |
| | | CCATGAATGCAAAGCTTCTGATGGATCCTAAGAGGCAGATCTT |
| | | TCTAGATCAAAACATGCTGGCAGTTATTGATGAGCTGATGCAG |
| | | GCCCTGAATTTCAACAGTGAGACTGTGCCACAAAAATCCTCCC |
| | | TTGAAGAACCGGATTTTTATAAAACTAAAATCAAGCTCTGCAT |
| | | ACTTCTTCATGCTTTCCGGATTCGGGCAGTGACTATTGATAGA |
| | | GTGATGAGCTATCTGAATGCTTCCTGAGCCCCTCTCCCTCCCC |
| | | CCCCCCTAACGTTACTGGCCGAAGCCGCTTGGAATAAGGCCGG |
| | | TGTGCGTTTGTCTATATGTTATTTTCCACCATATTGCCGTCTT |
| | | TTGGCAATGTGAGGGCCCGGAAACCTGGCCCTGTCTTCTTGAC |
| | | GAGCATTCCTAGGGGTCTTTCCCCTCTCGCCAAAGGAATGCAA |
| | | GGTCTGTTGAATGTCGTGAAGGAAGCAGTTCCTCTGGAAGCTT |
| | | CTTGAAGACAAACAACGTCTGTAGCGACCCTTTGCAGGCAGCG |
| | | GAACCCCCCACCTGGCGACAGGTGCCTCTGCGGCCAAAAGCCA |
| | | CGTGTATAAGATACACCTGCAAAGGCGGCACAACCCCAGTGCC |
| | | ACGTTGTGAGTTGGATAGTTGTGGAAAGAGTCAAATGGCTCTC |
| | | CTCAAGCGTATTCAACAAGGGGCTGAAGGATGCCCAGAAGGTA |
| | | CCCCATTGTATGGGATCTGATCTGGGGCCTCGGTACACATGCT |
| | | TTACATGTGTTTAGTCGAGGTTAAAAAAACGTCTAGGCCCCCC |
| | | GAACCACGGGGACGTGGTTTTCCTTTGAAAAACACGATGATAA |
| | | TATGGCCACAATGTGTCACCAGCAGTTGGTCATCTCTTGGTTT |
| | | TCCCTGGTTTTTCTGGCATCTCCCCTCGTGGCCATATGGGAAC |
| | | TGAAGAAAGATGTTTATGTCGTAGAATTGGATTGGTATCCGGA |
| | | TGCCCCTGGAGAAATGGTGGTCCTCACCTGTGACACCCCTGAA |
| | | GAAGATGGTATCACCTGGACCTTGGACCAGAGCAGTGAGGTCT |
| | | TAGGCTCTGGCAAAACCCTGACCATCCAAGTCAAAGAGTTTGG |
| | | AGATGCTGGCCAGTACACCTGTCACAAAGGAGGCGAGGTTCTA |
| | | AGCCATTCGCTCCTGCTGCTTCACAAAAAGGAAGATGGAATTT |
| | | GGTCCACTGATATTTTAAAGGACCAGAAAGAACCCAAAAATAA |
| | | GACCTTTCTAAGATGCGAGGCCAAGAATTATTCTGGACGTTTC |
| | | ACCTGCTGGTGGCTGACGACAATCAGTACTGATTTGACATTCA |
| | | GTGTCAAAAGCAGCAGAGGCTCTTCTGACCCCCAAGGGGTGAC |
| | | GTGCGGAGCTGCTACACTCTCTGCAGAGAGAGTCAGAGGGGAC |
| | | AACAAGGAGTATGAGTACTCAGTGGAGTGCCAGGAGGACAGTG |
| | | CCTGCCCAGCTGCTGAGGAGAGTCTGCCCATTGAGGTGATGGT |
| | | GGATGCCGTTCACAAGCTCAAGTATGAAAACTACACCAGCAGC |
| | | TTCTTCATCAGGGACATCATCAAACCTGACCCACCCAAGAACT |
| | | TGCAGCTGAAGCCATTAAAGAATTCTCGGCAGGTGGAGGTCAG |
| | | CTGGGAGTACCCTGACACCTGGAGTACTCCACATTCCTACTTC |
| | | TCCCTGACATTCTGCGTTCAAGTCCAGGGCAAGAGCAAGAGAG |
| | | AAAAGAAAGATAGAGTCTTCACGGACAAGACCTCAGCCACGGT |
| | | CATCTGCCGCAAAAATGCCAGCATTAGCGTGCGGGCCCAGGAC |
| | | CGCTACTATAGCTCATCTTGGAGCGAATGGGCATCTGTGCCCT |
| | | GCAGTTGACAATTG (SEQ ID NO: 35) |
| 20 | Human CAG-wild-type IL-15 (no | (SEQ ID NO: 36) |
| | CpG modification) construct | |
| 20 | Human CAG-wild-type IL-15 (no | (SEQ ID NO: 37) |
| | CpG modification) expression | |
| | sequence | |
| 21 | Mouse EF1a-wild-type IL-15 (no | SEQ ID NOs: 38 and 39, respectively |
| | CpG modification) and modified IL- | |
| | 12B (with CpG modification) full | |
| | construct and expression | |
| | sequences | |
| 22 | Mouse EF1a-modified IL-12B/IL- | SEQ ID NOs: 40 and 41, respectively |
| | 15 (CpG modified) full construct | |
| | and expression sequences | |
| 23 | Mouse CAG-wild-type IL-12B- | SEQ ID NOs: 42 and 43, respectively |
| | IRES (not CpG modified) full | |
| | construct and expression | |
| | sequences | |
| 24 | Mouse CAG-wild-type IL-15 (not | SEQ ID NOs: 44 and 45, respectively |
| | CpG modified) full construct and | |
| | expression sequences | |
| 25 | Mouse CAG-wild-type IL-12B- | SEQ ID NOs: 46 and 47, respectively |
| | F2A (not CpG modified) expression | |
| | sequence | |
| 26 | Human CAG-modified IL- | CTGCGCGCTCGCTCGCTCACTGAGGCCGCCCGGGCAAAGCCCG |
| | 12A/IL-12B-F2A (CpG | GGCGTCGGGCGACCTTTGGTCGCCCGGCCTCAGTGAGCGAGCG |
| | modified) construct | AGCGCGCAGAGAGGGAGTGGCCAACTCCATCACTAGGGGTTCC |
| | | TTAGTATTAAACGCGTGTCGACATTGATTATTGACTAGTTATT |
| | | AATAGTAATCAATTACGGGGTCATTAGTTCATAGCCCATATAT |
| | | GGAGTTCCGCGTTACATAACTTACGGTAAATGGCCCGCCTGGC |
| | | TGACCGCCCAACGACCCCCGCCCATTGACGTCAATAATGACGT |
| | | ATGTTCCCATAGTAACGCCAATAGGGACTTTCCATTGACGTCA |
| | | ATGGGTGGACTATTTACGGTAAACTGCCCACTTGGCAGTACAT |
| | | CAAGTGTATCATATGCCAAGTACGCCCCCTATTGACGTCAATG |
| | | ACGGTAAATGGCCCGCCTGGCATTATGCCCAGTACATGACCTT |
| | | ATGGGACTTTCCTACTTGGCAGTACATCTACGTATTAGTCATC |
| | | GCTATTACCATGGGTCGAGGTGAGCCCCACGTTCTGCTTCACT |
| | | CTCCCCATCTCCCCCCCCTCCCCACCCCCAATTTTGTATTTAT |
| | | TTATTTTTTAATTATTTTGTGCAGCGATGGGGGCGGGGGGGGG |
| | | GGGGGCGCGCGCCAGGCGGGGCGGGGCGGGGCGAGGGGCGGGG |
| | | CGGGGCGAGGCGGAGAGGTGCGGCGGCAGCCAATCAGAGCGGC |
| | | GCGCTCCGAAAGTTTCCTTTTATGGCGAGGCGGCGGCGGCGGC |
| | | GGCCCTATAAAAAGCGAAGCGCGCGGCGGGCGGGAGTCGCTGC |
| | | GCGCTGCCTTCGCCCCGTGCCCCGCTCCGCCGCCGCCTCGCGC |
| | | CGCCCGCCCCGGCTCTGACTGACCGCGTTACTCCCACAGGTGA |
| | | GCGGGCGGGACGGCCCTTCTCCTCCGGGCTGTAATTAGCGCTT |
| | | GGTTTAATGACGGCTCGTTTCTTTTCTGTGGCTGCGTGAAAGC |
| | | CTTGAGGGGCTCCGGGAGGGCCCTTTGTGCGGGGGGAGCGGCT |
| | | CGGGGGGTGCGTGCGTGTGTGTGTGCGTGGGGAGCGCCGCGTG |
| | | CGGCTCCGCGCTGCCCGGCGGCTGTGAGCGCTGCGGGCGCGGC |
| | | GCGGGGCTTTGTGCGCTCCGCAGTGTGCGCGAGAGGAGCGCGG |
| | | CCGGGGGCGGTGCCACGCGGTGCGGGGGGGGCTGCGAGGGGAA |
| | | CAAAGGCTGCGTGCGGGGTGTGTGCGTGGGGGGGGTGAGCAGG |
| | | GGGTGTGGGCGCGGCGGTCGGGCTGTAACCCCCCCCTGCACCC |
| | | CCCTCCCCGAGTTGCTGAGCACGGCCCGGCTTCGGGTGCGGGG |
| | | CTCCGTGCGGGGCGTGGCGCGGGGCTCGCCGTGCCGGGCGGGG |
| | | GGTGGCGGCGGGTGGGGGTGCCGGGCGGGGCGGGGCCGCCTCG |
| | | GGCCGGGGAGGGCTCGGGGGAGGGGCGCGGCGGCCCCCGGAGC |
| | | GCCGGCGGCTGTCGAGGCGCGGCGAGCCGCAGCCATTGCCTTT |
| | | TATGGTAATCGTGCGAGAGGGCGCAGGGACTTCCTTTGTCCCA |
| | | AATCTGTGCGGAGCCGAAATCTGGGAGGCGCCGCCGCACCCCC |
| | | TCTAGCGGGCGCGGGGCGAAGCGGTGCGGCGCCGGCAGGAAGG |
| | | AAATGGGCGGGGAGGGCCTTCGTGCGTCGCCGCGCCGCCGTCC |
| | | CCTTCTCCCTCTCCAGCCTCGGGGCTGTCCGCGGGGGGACGGC |
| | | TGCCTTCGGGGGGGACGGGGCAGGGCGGGGTTCGGCTTCTGGC |
| | | GTGTGACCGGCGGTCTAGACTCTGCTAACCATGTTCATGCCTT |
| | | CTTCTCTTTCCTACAGCTCCTGGGCAACGTGCTGGTTGTTGTG |
| | | CTGTCTCATCATTTTGGCAAAGAATTCAAGCTTCCAGCTAGCG |
| | | CCACCATGTGTCCAGCTAGGAGCCTCCTCCTTGTGGCTACCCT |
| | | GGTCCTCCTGGACCACCTCAGTTTGGCCAGAAACCTCCCTGTG |
| | | GCCACTCCAGACCCAGGAATGTTCCCATGCCTTCACCACTCCC |
| | | AAAACCTGCTGAGGGCAGTCAGCAACATGCTCCAGAAGGCCAG |
| | | ACAAACTCTAGAATTTTACCCTTGCACTTCTGAAGAGATTGAT |
| | | CATGAAGATATCACAAAAGATAAAACCAGCACAGTGGAGGCCT |
| | | GTTTACCATTGGAATTAACCAAGAATGAGAGTTGCCTAAATTC |
| | | CAGAGAGACCTCTTTCATAACTAATGGGAGTTGCCTGGCCTCC |
| | | AGAAAGACCTCTTTTATGATGGCCCTGTGCCTTAGTAGTATTT |
| | | ATGAAGACTTGAAGATGTACCAGGTGGAGTTCAAGACCATGAA |
| | | TGCAAAGCTTCTGATGGATCCTAAGAGGCAGATCTTTCTAGAT |
| | | CAAAACATGCTGGCAGTTATTGATGAGCTGATGCAGGCCCTGA |
| | | ATTTCAACAGTGAGACTGTGCCACAAAAATCCTCCCTTGAAGA |
| | | ACCTGATTTTTATAAAACTAAAATCAAGCTCTGCATACTTCTT |
| | | CATGCTTTCCGGATTAGGGCAGTGACTATTGATAGAGTGATGA |
| | | GCTATCTGAATGCTTCCAGAAAGAGAAGGAGTGGCTCAGGAGC |
| | | CCCTGTGAAACAGACCCTGAACTTTGACCTCTTGAAGCTTGCT |
| | | GGGGATGTGGAGTCTAATCCTGGTCCAATGTGTCACCAGCAGT |
| | | TGGTCATCTCTTGGTTTTCCCTGGTTTTTCTGGCATCTCCCCT |
| | | TGTGGCCATATGGGAACTGAAGAAAGATGTTTATGTGGTAGAA |
| | | TTGGATTGGTATCCTGATGCCCCTGGAGAAATGGTGGTCCTCA |
| | | CCTGTGACACCCCTGAAGAAGATGGTATCACCTGGACCTTGGA |
| | | CCAGAGCAGTGAGGTCTTAGGCTCTGGCAAAACCCTGACCATC |
| | | CAAGTCAAAGAGTTTGGAGATGCTGGCCAGTACACCTGTCACA |
| | | AAGGAGGGGAGGTTCTAAGCCATTCCCTCCTGCTGCTTCACAA |
| | | AAAGGAAGATGGAATTTGGTCCACTGATATTTTAAAGGACCAG |
| | | AAAGAACCCAAAAATAAGACCTTTCTAAGATGTGAGGCCAAGA |
| | | ATTATTCTGGAAGGTTCACCTGCTGGTGGCTGACTACAATCAG |
| | | TACTGATTTGACATTCAGTGTCAAAAGCAGCAGAGGCTCTTCT |
| | | GACCCCCAAGGGGTGACATGTGGAGCTGCTACACTCTCTGCAG |
| | | AGAGAGTCAGAGGGGACAACAAGGAGTATGAGTACTCAGTGGA |
| | | GTGCCAGGAGGACAGTGCCTGCCCAGCTGCTGAGGAGAGTCTG |
| | | CCCATTGAGGTGATGGTGGATGCAGTTCACAAGCTCAAGTATG |
| | | AAAACTACACCAGCAGCTTCTTCATCAGGGACATCATCAAACC |
| | | TGACCCACCCAAGAACTTGCAGCTGAAGCCATTAAAGAATTCT |
| | | AGGCAGGTGGAGGTCAGCTGGGAGTACCCTGACACCTGGAGTA |
| | | CTCCACATTCCTACTTCTCCCTGACATTCTGTGTTCAAGTCCA |
| | | GGGCAAGAGCAAGAGAGAAAAGAAAGATAGAGTCTTCACTGAC |
| | | AAGACCTCAGCCACAGTCATCTGCAGGAAAAATGCCAGCATTT |
| | | CAGTGAGGGCCCAGGACAGATACTATAGCTCATCTTGGTCAGA |
| | | ATGGGCATCTGTGCCCTGCAGTTGACAATTGCTGTGCCTTCTA |
| | | GTTGCCAGCCATCTGTTGTTTGCCCCTCCCCCGTGCCTTCCTT |
| | | GACCCTGGAAGGTGCCACTCCCACTGTCCTTTCCTAATAAAAT |
| | | GAGGAAATTGCATCGCATTGTCTGAGTAGGTGTCATTCTATTC |
| | | TGGGGGGTGGGGTGGGGCAGGACAGCAAGGGGGAGGATTGGGA |
| | | AGACAATAGCAGGCATGCTGGGGATGCGGTGGGCTCTATGGAG |
| | | CTCGAGAGGAACCCCTAGTGATGGAGTTGGCCACTCCCTCTCT |
| | | GCGCGCTCGCTCGCTCACTGAGGCCGGGCGACCAAAGGTCGCC |
| | | CGACGCCCGGGCTTTGCCCGGGCGGCCTCAGTGAGCGAGCGAG |
| | | CGCGCAGCTGCAGATCTG (SEQ ID NO: 48) |
| 26 | Human CAG-modified IL-12A/IL- | GCTAGCGCCACCATGTGTCCAGCTAGGAGCCTCCTCCTTGTGG |
| | 12B-F2A (CpG modified) | CTACCCTGGTCCTCCTGGACCACCTCAGTTTGGCCAGAAACCT |
| | expression sequence | CCCTGTGGCCACTCCAGACCCAGGAATGTTCCCATGCCTTCAC |
| | | CACTCCCAAAACCTGCTGAGGGCAGTCAGCAACATGCTCCAGA |
| | | AGGCCAGACAAACTCTAGAATTTTACCCTTGCACTTCTGAAGA |
| | | GATTGATCATGAAGATATCACAAAAGATAAAACCAGCACAGTG |
| | | GAGGCCTGTTTACCATTGGAATTAACCAAGAATGAGAGTTGCC |
| | | TAAATTCCAGAGAGACCTCTTTCATAACTAATGGGAGTTGCCT |
| | | GGCCTCCAGAAAGACCTCTTTTATGATGGCCCTGTGCCTTAGT |
| | | AGTATTTATGAAGACTTGAAGATGTACCAGGTGGAGTTCAAGA |
| | | CCATGAATGCAAAGCTTCTGATGGATCCTAAGAGGCAGATCTT |
| | | TCTAGATCAAAACATGCTGGCAGTTATTGATGAGCTGATGCAG |
| | | GCCCTGAATTTCAACAGTGAGACTGTGCCACAAAAATCCTCCC |
| | | TTGAAGAACCTGATTTTTATAAAACTAAAATCAAGCTCTGCAT |
| | | ACTTCTTCATGCTTTCCGGATTAGGGCAGTGACTATTGATAGA |
| | | GTGATGAGCTATCTGAATGCTTCCAGAAAGAGAAGGAGTGGCT |
| | | CAGGAGCCCCTGTGAAACAGACCCTGAACTTTGACCTCTTGAA |
| | | GCTTGCTGGGGATGTGGAGTCTAATCCTGGTCCAATGTGTCAC |
| | | CAGCAGTTGGTCATCTCTTGGTTTTCCCTGGTTTTTCTGGCAT |
| | | CTCCCCTTGTGGCCATATGGGAACTGAAGAAAGATGTTTATGT |
| | | GGTAGAATTGGATTGGTATCCTGATGCCCCTGGAGAAATGGTG |
| | | GTCCTCACCTGTGACACCCCTGAAGAAGATGGTATCACCTGGA |
| | | CCTTGGACCAGAGCAGTGAGGTCTTAGGCTCTGGCAAAACCCT |
| | | GACCATCCAAGTCAAAGAGTTTGGAGATGCTGGCCAGTACACC |
| | | TGTCACAAAGGAGGGGAGGTTCTAAGCCATTCCCTCCTGCTGC |
| | | TTCACAAAAAGGAAGATGGAATTTGGTCCACTGATATTTTAAA |
| | | GGACCAGAAAGAACCCAAAAATAAGACCTTTCTAAGATGTGAG |
| | | GCCAAGAATTATTCTGGAAGGTTCACCTGCTGGTGGCTGACTA |
| | | CAATCAGTACTGATTTGACATTCAGTGTCAAAAGCAGCAGAGG |
| | | CTCTTCTGACCCCCAAGGGGTGACATGTGGAGCTGCTACACTC |
| | | TCTGCAGAGAGAGTCAGAGGGGACAACAAGGAGTATGAGTACT |
| | | CAGTGGAGTGCCAGGAGGACAGTGCCTGCCCAGCTGCTGAGGA |
| | | GAGTCTGCCCATTGAGGTGATGGTGGATGCAGTTCACAAGCTC |
| | | AAGTATGAAAACTACACCAGCAGCTTCTTCATCAGGGACATCA |
| | | TCAAACCTGACCCACCCAAGAACTTGCAGCTGAAGCCATTAAA |
| | | GAATTCTAGGCAGGTGGAGGTCAGCTGGGAGTACCCTGACACC |
| | | TGGAGTACTCCACATTCCTACTTCTCCCTGACATTCTGTGTTC |
| | | AAGTCCAGGGCAAGAGCAAGAGAGAAAAGAAAGATAGAGTCTT |
| | | CACTGACAAGACCTCAGCCACAGTCATCTGCAGGAAAAATGCC |
| | | AGCATTTCAGTGAGGGCCCAGGACAGATACTATAGCTCATCTT |
| | | GGTCAGAATGGGCATCTGTGCCCTGCAGTTGACAATTG (SEQ |
| | | ID NO: 49) |
| 27 | Human CAG-modified IL-12A/IL- | CTGCGCGCTCGCTCGCTCACTGAGGCCGCCCGGGCAAAGCCCG |
| | 12B-F2A (CpG modified) construct | GGCGTCGGGCGACCTTTGGTCGCCCGGCCTCAGTGAGCGAGCG |
| | | AGCGCGCAGAGAGGGAGTGGCCAACTCCATCACTAGGGGTTCC |
| | | TTAGTATTAAACGCGTGTCGACATTGATTATTGACTAGTTATT |
| | | AATAGTAATCAATTACGGGGTCATTAGTTCATAGCCCATATAT |
| | | GGAGTTCCGCGTTACATAACTTACGGTAAATGGCCCGCCTGGC |
| | | TGACCGCCCAACGACCCCCGCCCATTGACGTCAATAATGACGT |
| | | ATGTTCCCATAGTAACGCCAATAGGGACTTTCCATTGACGTCA |
| | | ATGGGTGGACTATTTACGGTAAACTGCCCACTTGGCAGTACAT |
| | | CAAGTGTATCATATGCCAAGTACGCCCCCTATTGACGTCAATG |
| | | ACGGTAAATGGCCCGCCTGGCATTATGCCCAGTACATGACCTT |
| | | ATGGGACTTTCCTACTTGGCAGTACATCTACGTATTAGTCATC |
| | | GCTATTACCATGGGTCGAGGTGAGCCCCACGTTCTGCTTCACT |
| | | CTCCCCATCTCCCCCCCCTCCCCACCCCCAATTTTGTATTTAT |
| | | TTATTTTTTAATTATTTTGTGCAGCGATGGGGGCGGGGGGGGG |
| | | GGGGGCGCGCGCCAGGCGGGGCGGGGCGGGGCGAGGGGCGGGG |
| | | CGGGGCGAGGCGGAGAGGTGCGGCGGCAGCCAATCAGAGCGGC |
| | | GCGCTCCGAAAGTTTCCTTTTATGGCGAGGCGGCGGCGGCGGC |
| | | GGCCCTATAAAAAGCGAAGCGCGCGGCGGGCGGGAGTCGCTGC |
| | | GCGCTGCCTTCGCCCCGTGCCCCGCTCCGCCGCCGCCTCGCGC |
| | | CGCCCGCCCCGGCTCTGACTGACCGCGTTACTCCCACAGGTGA |
| | | GCGGGCGGGACGGCCCTTCTCCTCCGGGCTGTAATTAGCGCTT |
| | | GGTTTAATGACGGCTCGTTTCTTTTCTGTGGCTGCGTGAAAGC |
| | | CTTGAGGGGCTCCGGGAGGGCCCTTTGTGCGGGGGGAGCGGCT |
| | | CGGGGGGTGCGTGCGTGTGTGTGTGCGTGGGGAGCGCCGCGTG |
| | | CGGCTCCGCGCTGCCCGGCGGCTGTGAGCGCTGCGGGCGCGGC |
| | | GCGGGGCTTTGTGCGCTCCGCAGTGTGCGCGAGAGGAGCGCGG |
| | | CCGGGGGCGGTGCCACGCGGTGCGGGGGGGGCTGCGAGGGGAA |
| | | CAAAGGCTGCGTGCGGGGTGTGTGCGTGGGGGGGGTGAGCAGG |
| | | GGGTGTGGGCGCGGCGGTCGGGCTGTAACCCCCCCCTGCACCC |
| | | CCCTCCCCGAGTTGCTGAGCACGGCCCGGCTTCGGGTGCGGGG |
| | | CTCCGTGCGGGGCGTGGCGCGGGGCTCGCCGTGCCGGGCGGGG |
| | | GGTGGCGGCGGGTGGGGGTGCCGGGCGGGGCGGGGCCGCCTCG |
| | | GGCCGGGGAGGGCTCGGGGGAGGGGCGCGGCGGCCCCCGGAGC |
| | | GCCGGCGGCTGTCGAGGCGCGGCGAGCCGCAGCCATTGCCTTT |
| | | TATGGTAATCGTGCGAGAGGGCGCAGGGACTTCCTTTGTCCCA |
| | | AATCTGTGCGGAGCCGAAATCTGGGAGGCGCCGCCGCACCCCC |
| | | TCTAGCGGGCGCGGGGCGAAGCGGTGCGGCGCCGGCAGGAAGG |
| | | AAATGGGCGGGGAGGGCCTTCGTGCGTCGCCGCGCCGCCGTCC |
| | | CCTTCTCCCTCTCCAGCCTCGGGGCTGTCCGCGGGGGGACGGC |
| | | TGCCTTCGGGGGGGACGGGGCAGGGCGGGGTTCGGCTTCTGGC |
| | | GTGTGACCGGCGGTCTAGACTCTGCTAACCATGTTCATGCCTT |
| | | CTTCTCTTTCCTACAGCTCCTGGGCAACGTGCTGGTTGTTGTG |
| | | CTGTCTCATCATTTTGGCAAAGAATTCAAGCTTCCAGCTAGCG |
| | | CCACCATGTGTCCAGCGCGCAGCCTCCTCCTTGTGGCTACCCT |
| | | GGTCCTCCTGGACCACCTCAGTTTGGCCAGAAACCTCCCTGTG |
| | | GCCACTCCAGACCCAGGAATGTTCCCATGCCTTCACCACTCCC |
| | | AAAACCTGCTGAGGGCTGTCAGCAACATGCTCCAGAAGGCCAG |
| | | ACAAACTCTAGAATTTTACCCTTGCACTTCTGAAGAGATTGAT |
| | | CATGAAGATATCACAAAAGATAAAACCAGCACAGTGGAGGCCT |
| | | GTTTACCATTGGAATTAACCAAGAATGAGAGTTGCCTAAATTC |
| | | CAGAGAGACCTCTTTCATAACTAATGGGAGTTGCCTGGCCTCC |
| | | AGAAAGACCTCTTTTATGATGGCCCTGTGCCTTAGTAGTATTT |
| | | ATGAAGACTTGAAGATGTACCAGGTGGAGTTCAAGACCATGAA |
| | | TGCAAAGCTTCTGATGGATCCTAAGAGGCAGATCTTTCTAGAT |
| | | CAAAACATGCTGGCAGTTATTGATGAGCTGATGCAGGCCCTGA |
| | | ATTTCAACAGTGAGACTGTGCCACAAAAATCCTCCCTTGAAGA |
| | | ACCTGATTTTTATAAAACTAAAATCAAGCTCTGCATACTTCTT |
| | | CATGCTTTCCGGATTCGCGCAGTGACTATTGATAGAGTGATGA |
| | | GCTATCTGAATGCTTCCAGAAAGAGAAGGAGTGGCTCAGGAGC |
| | | CCCTGTGAAACAGACCCTGAACTTTGACCTCTTGAAGCTTGCT |
| | | GGGGATGTGGAGTCTAATCCTGGTCCAATGTGTCACCAGCAGT |
| | | TGGTCATCTCTTGGTTTTCCCTGGTTTTTCTGGCATCTCCCCT |
| | | TGTGGCCATATGGGAACTGAAGAAAGATGTTTATGTGGTAGAA |
| | | TTGGATTGGTATCCTGATGCCCCTGGAGAAATGGTGGTCCTCA |
| | | CCTGTGACACCCCTGAAGAAGATGGTATCACCTGGACCTTGGA |
| | | CCAGAGCAGTGAGGTCTTAGGCTCTGGCAAAACCCTGACCATC |
| | | CAAGTCAAAGAGTTTGGAGATGCTGGCCAGTACACCTGTCACA |
| | | AAGGAGGGGAGGTTCTAAGCCATTCCCTCCTGCTGCTTCACAA |
| | | AAAGGAAGATGGAATTTGGTCCACTGATATTTTAAAGGACCAG |
| | | AAAGAACCCAAAAATAAGACCTTTCTAAGATGTGAGGCCAAGA |
| | | ATTATTCTGGAAGGTTCACCTGCTGGTGGCTGACTACAATCAG |
| | | TACTGATTTGACATTCAGTGTCAAAAGCAGCAGAGGCTCTTCT |
| | | GACCCCCAAGGGGTGACATGCGGAGCTGCTACACTCTCTGCAG |
| | | AGAGAGTCAGAGGGGACAACAAGGAGTATGAGTACTCAGTGGA |
| | | GTGCCAGGAGGACAGTGCCTGCCCAGCTGCTGAGGAGAGTCTG |
| | | CCCATTGAGGTGATGGTGGATGCGGTTCACAAGCTCAAGTATG |
| | | AAAACTACACCAGCAGCTTCTTCATCAGGGACATCATCAAACC |
| | | TGACCCACCCAAGAACTTGCAGCTGAAGCCATTAAAGAATTCT |
| | | AGGCAGGTGGAGGTCAGCTGGGAGTACCCTGACACCTGGAGTA |
| | | CTCCACATTCCTACTTCTCCCTGACATTCTGTGTTCAAGTCCA |
| | | GGGCAAGAGCAAGAGAGAAAAGAAAGATAGAGTCTTCACTGAC |
| | | AAGACCTCAGCCACAGTCATCTGCCGCAAAAATGCCAGCATTT |
| | | CAGTGCGGGCCCAGGACCGCTACTATAGCTCATCTTGGTCAGA |
| | | ATGGGCATCTGTGCCCTGCAGTTGACAATTGCTGTGCCTTCTA |
| | | GTTGCCAGCCATCTGTTGTTTGCCCCTCCCCCGTGCCTTCCTT |
| | | GACCCTGGAAGGTGCCACTCCCACTGTCCTTTCCTAATAAAAT |
| | | GAGGAAATTGCATCGCATTGTCTGAGTAGGTGTCATTCTATTC |
| | | TGGGGGGTGGGGTGGGGCAGGACAGCAAGGGGGAGGATTGGGA |
| | | AGACAATAGCAGGCATGCTGGGGATGCGGTGGGCTCTATGGAG |
| | | CTCGAGAGGAACCCCTAGTGATGGAGTTGGCCACTCCCTCTCT |
| | | GCGCGCTCGCTCGCTCACTGAGGCCGGGCGACCAAAGGTCGCC |
| | | CGACGCCCGGGCTTTGCCCGGGCGGCCTCAGTGAGCGAGCGAG |
| | | CGCGCAGCTGCAGATCTG (SEQ ID NO:50) |
| 27 | Human CAG-modified IL-12A/IL- | GCTAGCGCCACCATGTGTCCAGCGCGCAGCCTCCTCCTTGTGG |
| | 12B-F2A (CpG modified) | CTACCCTGGTCCTCCTGGACCACCTCAGTTTGGCCAGAAACCT |
| | expression sequence | CCCTGTGGCCACTCCAGACCCAGGAATGTTCCCATGCCTTCAC |
| | | CACTCCCAAAACCTGCTGAGGGCTGTCAGCAACATGCTCCAGA |
| | | AGGCCAGACAAACTCTAGAATTTTACCCTTGCACTTCTGAAGA |
| | | GATTGATCATGAAGATATCACAAAAGATAAAACCAGCACAGTG |
| | | GAGGCCTGTTTACCATTGGAATTAACCAAGAATGAGAGTTGCC |
| | | TAAATTCCAGAGAGACCTCTTTCATAACTAATGGGAGTTGCCT |
| | | GGCCTCCAGAAAGACCTCTTTTATGATGGCCCTGTGCCTTAGT |
| | | AGTATTTATGAAGACTTGAAGATGTACCAGGTGGAGTTCAAGA |
| | | CCATGAATGCAAAGCTTCTGATGGATCCTAAGAGGCAGATCTT |
| | | TCTAGATCAAAACATGCTGGCAGTTATTGATGAGCTGATGCAG |
| | | GCCCTGAATTTCAACAGTGAGACTGTGCCACAAAAATCCTCCC |
| | | TTGAAGAACCTGATTTTTATAAAACTAAAATCAAGCTCTGCAT |
| | | ACTTCTTCATGCTTTCCGGATTCGCGCAGTGACTATTGATAGA |
| | | GTGATGAGCTATCTGAATGCTTCCAGAAAGAGAAGGAGTGGCT |
| | | CAGGAGCCCCTGTGAAACAGACCCTGAACTTTGACCTCTTGAA |
| | | GCTTGCTGGGGATGTGGAGTCTAATCCTGGTCCAATGTGTCAC |
| | | CAGCAGTTGGTCATCTCTTGGTTTTCCCTGGTTTTTCTGGCAT |
| | | CTCCCCTTGTGGCCATATGGGAACTGAAGAAAGATGTTTATGT |
| | | GGTAGAATTGGATTGGTATCCTGATGCCCCTGGAGAAATGGTG |
| | | GTCCTCACCTGTGACACCCCTGAAGAAGATGGTATCACCTGGA |
| | | CCTTGGACCAGAGCAGTGAGGTCTTAGGCTCTGGCAAAACCCT |
| | | GACCATCCAAGTCAAAGAGTTTGGAGATGCTGGCCAGTACACC |
| | | TGTCACAAAGGAGGGGAGGTTCTAAGCCATTCCCTCCTGCTGC |
| | | TTCACAAAAAGGAAGATGGAATTTGGTCCACTGATATTTTAAA |
| | | GGACCAGAAAGAACCCAAAAATAAGACCTTTCTAAGATGTGAG |
| | | GCCAAGAATTATTCTGGAAGGTTCACCTGCTGGTGGCTGACTA |
| | | CAATCAGTACTGATTTGACATTCAGTGTCAAAAGCAGCAGAGG |
| | | CTCTTCTGACCCCCAAGGGGTGACATGCGGAGCTGCTACACTC |
| | | TCTGCAGAGAGAGTCAGAGGGGACAACAAGGAGTATGAGTACT |
| | | CAGTGGAGTGCCAGGAGGACAGTGCCTGCCCAGCTGCTGAGGA |
| | | GAGTCTGCCCATTGAGGTGATGGTGGATGCGGTTCACAAGCTC |
| | | AAGTATGAAAACTACACCAGCAGCTTCTTCATCAGGGACATCA |
| | | TCAAACCTGACCCACCCAAGAACTTGCAGCTGAAGCCATTAAA |
| | | GAATTCTAGGCAGGTGGAGGTCAGCTGGGAGTACCCTGACACC |
| | | TGGAGTACTCCACATTCCTACTTCTCCCTGACATTCTGTGTTC |
| | | AAGTCCAGGGCAAGAGCAAGAGAGAAAAGAAAGATAGAGTCTT |
| | | CACTGACAAGACCTCAGCCACAGTCATCTGCCGCAAAAATGCC |
| | | AGCATTTCAGTGCGGGCCCAGGACCGCTACTATAGCTCATCTT |
| | | GGTCAGAATGGGCATCTGTGCCCTGCAGTTGACAATTG (SEQ |
| | | ID NO: 51) |
| 28 | Human CAG-modified IL-12A/IL- | CTGCGCGCTCGCTCGCTCACTGAGGCCGCCCGGGCAAAGCCCG |
| | 12B-IRES (CpG modified) | GGCGTCGGGCGACCTTTGGTCGCCCGGCCTCAGTGAGCGAGCG |
| | construct | AGCGCGCAGAGAGGGAGTGGCCAACTCCATCACTAGGGGTTCC |
| | | TTAGTATTAAACGCGTGTCGACATTGATTATTGACTAGTTATT |
| | | AATAGTAATCAATTACGGGGTCATTAGTTCATAGCCCATATAT |
| | | GGAGTTCCGCGTTACATAACTTACGGTAAATGGCCCGCCTGGC |
| | | TGACCGCCCAACGACCCCCGCCCATTGACGTCAATAATGACGT |
| | | ATGTTCCCATAGTAACGCCAATAGGGACTTTCCATTGACGTCA |
| | | ATGGGTGGACTATTTACGGTAAACTGCCCACTTGGCAGTACAT |
| | | CAAGTGTATCATATGCCAAGTACGCCCCCTATTGACGTCAATG |
| | | ACGGTAAATGGCCCGCCTGGCATTATGCCCAGTACATGACCTT |
| | | ATGGGACTTTCCTACTTGGCAGTACATCTACGTATTAGTCATC |
| | | GCTATTACCATGGGTCGAGGTGAGCCCCACGTTCTGCTTCACT |
| | | CTCCCCATCTCCCCCCCCTCCCCACCCCCAATTTTGTATTTAT |
| | | TTATTTTTTAATTATTTTGTGCAGCGATGGGGGCGGGGGGGGG |
| | | GGGGGCGCGCGCCAGGCGGGGCGGGGCGGGGCGAGGGGCGGGG |
| | | CGGGGCGAGGCGGAGAGGTGCGGCGGCAGCCAATCAGAGCGGC |
| | | GCGCTCCGAAAGTTTCCTTTTATGGCGAGGCGGCGGCGGCGGC |
| | | GGCCCTATAAAAAGCGAAGCGCGCGGCGGGCGGGAGTCGCTGC |
| | | GCGCTGCCTTCGCCCCGTGCCCCGCTCCGCCGCCGCCTCGCGC |
| | | CGCCCGCCCCGGCTCTGACTGACCGCGTTACTCCCACAGGTGA |
| | | GCGGGCGGGACGGCCCTTCTCCTCCGGGCTGTAATTAGCGCTT |
| | | GGTTTAATGACGGCTCGTTTCTTTTCTGTGGCTGCGTGAAAGC |
| | | CTTGAGGGGCTCCGGGAGGGCCCTTTGTGCGGGGGGAGCGGCT |
| | | CGGGGGGTGCGTGCGTGTGTGTGTGCGTGGGGAGCGCCGCGTG |
| | | CGGCTCCGCGCTGCCCGGCGGCTGTGAGCGCTGCGGGCGCGGC |
| | | GCGGGGCTTTGTGCGCTCCGCAGTGTGCGCGAGAGGAGCGCGG |
| | | CCGGGGGCGGTGCCACGCGGTGCGGGGGGGGCTGCGAGGGGAA |
| | | CAAAGGCTGCGTGCGGGGTGTGTGCGTGGGGGGGGTGAGCAGG |
| | | GGGTGTGGGCGCGGCGGTCGGGCTGTAACCCCCCCCTGCACCC |
| | | CCCTCCCCGAGTTGCTGAGCACGGCCCGGCTTCGGGTGCGGGG |
| | | CTCCGTGCGGGGCGTGGCGCGGGGCTCGCCGTGCCGGGCGGGG |
| | | GGTGGCGGCGGGTGGGGGTGCCGGGCGGGGCGGGGCCGCCTCG |
| | | GGCCGGGGAGGGCTCGGGGGAGGGGCGCGGCGGCCCCCGGAGC |
| | | GCCGGCGGCTGTCGAGGCGCGGCGAGCCGCAGCCATTGCCTTT |
| | | TATGGTAATCGTGCGAGAGGGCGCAGGGACTTCCTTTGTCCCA |
| | | AATCTGTGCGGAGCCGAAATCTGGGAGGCGCCGCCGCACCCCC |
| | | TCTAGCGGGCGCGGGGCGAAGCGGTGCGGCGCCGGCAGGAAGG |
| | | AAATGGGCGGGGAGGGCCTTCGTGCGTCGCCGCGCCGCCGTCC |
| | | CCTTCTCCCTCTCCAGCCTCGGGGCTGTCCGCGGGGGGACGGC |
| | | TGCCTTCGGGGGGGACGGGGCAGGGCGGGGTTCGGCTTCTGGC |
| | | GTGTGACCGGCGGTCTAGACTCTGCTAACCATGTTCATGCCTT |
| | | CTTCTCTTTCCTACAGCTCCTGGGCAACGTGCTGGTTGTTGTG |
| | | CTGTCTCATCATTTTGGCAAAGAATTCAAGCTTCCAGCTAGCG |
| | | CCACCATGTGTCCAGCTAGGAGCCTCCTCCTTGTGGCTACCCT |
| | | GGTCCTCCTGGACCACCTCAGTTTGGCCAGAAACCTCCCTGTG |
| | | GCCACTCCAGACCCAGGAATGTTCCCATGCCTTCACCACTCCC |
| | | AAAACCTGCTGAGGGCAGTCAGCAACATGCTCCAGAAGGCCAG |
| | | ACAAACTCTAGAATTTTACCCTTGCACTTCTGAAGAGATTGAT |
| | | CATGAAGATATCACAAAAGATAAAACCAGCACAGTGGAGGCCT |
| | | GTTTACCATTGGAATTAACCAAGAATGAGAGTTGCCTAAATTC |
| | | CAGAGAGACCTCTTTCATAACTAATGGGAGTTGCCTGGCCTCC |
| | | AGAAAGACCTCTTTTATGATGGCCCTGTGCCTTAGTAGTATTT |
| | | ATGAAGACTTGAAGATGTACCAGGTGGAGTTCAAGACCATGAA |
| | | TGCAAAGCTTCTGATGGATCCTAAGAGGCAGATCTTTCTAGAT |
| | | CAAAACATGCTGGCAGTTATTGATGAGCTGATGCAGGCCCTGA |
| | | ATTTCAACAGTGAGACTGTGCCACAAAAATCCTCCCTTGAAGA |
| | | ACCTGATTTTTATAAAACTAAAATCAAGCTCTGCATACTTCTT |
| | | CATGCTTTCCGGATTAGGGCAGTGACTATTGATAGAGTGATGA |
| | | GCTATCTGAATGCTTCCTGAGCCCCTCTCCCTCCCCCCCCCCT |
| | | AACGTTACTGGCCGAAGCCGCTTGGAATAAGGCCGGTGTGCGT |
| | | TTGTCTATATGTTATTTTCCACCATATTGCCGTCTTTTGGCAA |
| | | TGTGAGGGCCCGGAAACCTGGCCCTGTCTTCTTGACGAGCATT |
| | | CCTAGGGGTCTTTCCCCTCTCGCCAAAGGAATGCAAGGTCTGT |
| | | TGAATGTCGTGAAGGAAGCAGTTCCTCTGGAAGCTTCTTGAAG |
| | | ACAAACAACGTCTGTAGCGACCCTTTGCAGGCAGCGGAACCCC |
| | | CCACCTGGCGACAGGTGCCTCTGCGGCCAAAAGCCACGTGTAT |
| | | AAGATACACCTGCAAAGGCGGCACAACCCCAGTGCCACGTTGT |
| | | GAGTTGGATAGTTGTGGAAAGAGTCAAATGGCTCTCCTCAAGC |
| | | GTATTCAACAAGGGGCTGAAGGATGCCCAGAAGGTACCCCATT |
| | | GTATGGGATCTGATCTGGGGCCTCGGTACACATGCTTTACATG |
| | | TGTTTAGTCGAGGTTAAAAAAACGTCTAGGCCCCCCGAACCAC |
| | | GGGGACGTGGTTTTCCTTTGAAAAACACGATGATAATATGGCC |
| | | ACAATGTGTCACCAGCAGTTGGTCATCTCTTGGTTTTCCCTGG |
| | | TTTTTCTGGCATCTCCCCTTGTGGCCATATGGGAACTGAAGAA |
| | | AGATGTTTATGTGGTAGAATTGGATTGGTATCCTGATGCCCCT |
| | | GGAGAAATGGTGGTCCTCACCTGTGACACCCCTGAAGAAGATG |
| | | GTATCACCTGGACCTTGGACCAGAGCAGTGAGGTCTTAGGCTC |
| | | TGGCAAAACCCTGACCATCCAAGTCAAAGAGTTTGGAGATGCT |
| | | GGCCAGTACACCTGTCACAAAGGAGGGGAGGTTCTAAGCCATT |
| | | CCCTCCTGCTGCTTCACAAAAAGGAAGATGGAATTTGGTCCAC |
| | | TGATATTTTAAAGGACCAGAAAGAACCCAAAAATAAGACCTTT |
| | | CTAAGATGTGAGGCCAAGAATTATTCTGGAAGGTTCACCTGCT |
| | | GGTGGCTGACTACAATCAGTACTGATTTGACATTCAGTGTCAA |
| | | AAGCAGCAGAGGCTCTTCTGACCCCCAAGGGGTGACATGTGGA |
| | | GCTGCTACACTCTCTGCAGAGAGAGTCAGAGGGGACAACAAGG |
| | | AGTATGAGTACTCAGTGGAGTGCCAGGAGGACAGTGCCTGCCC |
| | | AGCTGCTGAGGAGAGTCTGCCCATTGAGGTGATGGTGGATGCA |
| | | GTTCACAAGCTCAAGTATGAAAACTACACCAGCAGCTTCTTCA |
| | | TCAGGGACATCATCAAACCTGACCCACCCAAGAACTTGCAGCT |
| | | GAAGCCATTAAAGAATTCTAGGCAGGTGGAGGTCAGCTGGGAG |
| | | TACCCTGACACCTGGAGTACTCCACATTCCTACTTCTCCCTGA |
| | | CATTCTGTGTTCAAGTCCAGGGCAAGAGCAAGAGAGAAAAGAA |
| | | AGATAGAGTCTTCACTGACAAGACCTCAGCCACAGTCATCTGC |
| | | AGGAAAAATGCCAGCATTTCAGTGAGGGCCCAGGACAGATACT |
| | | ATAGCTCATCTTGGTCAGAATGGGCATCTGTGCCCTGCAGTTG |
| | | ACAATTGCTGTGCCTTCTAGTTGCCAGCCATCTGTTGTTTGCC |
| | | CCTCCCCCGTGCCTTCCTTGACCCTGGAAGGTGCCACTCCCAC |
| | | TGTCCTTTCCTAATAAAATGAGGAAATTGCATCGCATTGTCTG |
| | | AGTAGGTGTCATTCTATTCTGGGGGGTGGGGTGGGGCAGGACA |
| | | GCAAGGGGGAGGATTGGGAAGACAATAGCAGGCATGCTGGGGA |
| | | TGCGGTGGGCTCTATGGAGCTCGAGAGGAACCCCTAGTGATGG |
| | | AGTTGGCCACTCCCTCTCTGCGCGCTCGCTCGCTCACTGAGGC |
| | | CGGGCGACCAAAGGTCGCCCGACGCCCGGGCTTTGCCCGGGCG |
| | | GCCTCAGTGAGCGAGCGAGCGCGCAGCTGCAGATCTG (SEQ |
| | | ID NO: 52) |
| 28 | Human CAG-modified IL-12A/IL- | GCTAGCGCCACCATGTGTCCAGCTAGGAGCCTCCTCCTTGTGG |
| | 12B-IRES (CpG modified) | CTACCCTGGTCCTCCTGGACCACCTCAGTTTGGCCAGAAACCT |
| | expression sequence | CCCTGTGGCCACTCCAGACCCAGGAATGTTCCCATGCCTTCAC |
| | | CACTCCCAAAACCTGCTGAGGGCAGTCAGCAACATGCTCCAGA |
| | | AGGCCAGACAAACTCTAGAATTTTACCCTTGCACTTCTGAAGA |
| | | GATTGATCATGAAGATATCACAAAAGATAAAACCAGCACAGTG |
| | | GAGGCCTGTTTACCATTGGAATTAACCAAGAATGAGAGTTGCC |
| | | TAAATTCCAGAGAGACCTCTTTCATAACTAATGGGAGTTGCCT |
| | | GGCCTCCAGAAAGACCTCTTTTATGATGGCCCTGTGCCTTAGT |
| | | AGTATTTATGAAGACTTGAAGATGTACCAGGTGGAGTTCAAGA |
| | | CCATGAATGCAAAGCTTCTGATGGATCCTAAGAGGCAGATCTT |
| | | TCTAGATCAAAACATGCTGGCAGTTATTGATGAGCTGATGCAG |
| | | GCCCTGAATTTCAACAGTGAGACTGTGCCACAAAAATCCTCCC |
| | | TTGAAGAACCTGATTTTTATAAAACTAAAATCAAGCTCTGCAT |
| | | ACTTCTTCATGCTTTCCGGATTAGGGCAGTGACTATTGATAGA |
| | | GTGATGAGCTATCTGAATGCTTCCTGAGCCCCTCTCCCTCCCC |
| | | CCCCCCTAACGTTACTGGCCGAAGCCGCTTGGAATAAGGCCGG |
| | | TGTGCGTTTGTCTATATGTTATTTTCCACCATATTGCCGTCTT |
| | | TTGGCAATGTGAGGGCCCGGAAACCTGGCCCTGTCTTCTTGAC |
| | | GAGCATTCCTAGGGGTCTTTCCCCTCTCGCCAAAGGAATGCAA |
| | | GGTCTGTTGAATGTCGTGAAGGAAGCAGTTCCTCTGGAAGCTT |
| | | CTTGAAGACAAACAACGTCTGTAGCGACCCTTTGCAGGCAGCG |
| | | GAACCCCCCACCTGGCGACAGGTGCCTCTGCGGCCAAAAGCCA |
| | | CGTGTATAAGATACACCTGCAAAGGCGGCACAACCCCAGTGCC |
| | | ACGTTGTGAGTTGGATAGTTGTGGAAAGAGTCAAATGGCTCTC |
| | | CTCAAGCGTATTCAACAAGGGGCTGAAGGATGCCCAGAAGGTA |
| | | CCCCATTGTATGGGATCTGATCTGGGGCCTCGGTACACATGCT |
| | | TTACATGTGTTTAGTCGAGGTTAAAAAAACGTCTAGGCCCCCC |
| | | GAACCACGGGGACGTGGTTTTCCTTTGAAAAACACGATGATAA |
| | | TATGGCCACAATGTGTCACCAGCAGTTGGTCATCTCTTGGTTT |
| | | TCCCTGGTTTTTCTGGCATCTCCCCTTGTGGCCATATGGGAAC |
| | | TGAAGAAAGATGTTTATGTGGTAGAATTGGATTGGTATCCTGA |
| | | TGCCCCTGGAGAAATGGTGGTCCTCACCTGTGACACCCCTGAA |
| | | GAAGATGGTATCACCTGGACCTTGGACCAGAGCAGTGAGGTCT |
| | | TAGGCTCTGGCAAAACCCTGACCATCCAAGTCAAAGAGTTTGG |
| | | AGATGCTGGCCAGTACACCTGTCACAAAGGAGGGGAGGTTCTA |
| | | AGCCATTCCCTCCTGCTGCTTCACAAAAAGGAAGATGGAATTT |
| | | GGTCCACTGATATTTTAAAGGACCAGAAAGAACCCAAAAATAA |
| | | GACCTTTCTAAGATGTGAGGCCAAGAATTATTCTGGAAGGTTC |
| | | ACCTGCTGGTGGCTGACTACAATCAGTACTGATTTGACATTCA |
| | | GTGTCAAAAGCAGCAGAGGCTCTTCTGACCCCCAAGGGGTGAC |
| | | ATGTGGAGCTGCTACACTCTCTGCAGAGAGAGTCAGAGGGGAC |
| | | AACAAGGAGTATGAGTACTCAGTGGAGTGCCAGGAGGACAGTG |
| | | CCTGCCCAGCTGCTGAGGAGAGTCTGCCCATTGAGGTGATGGT |
| | | GGATGCAGTTCACAAGCTCAAGTATGAAAACTACACCAGCAGC |
| | | TTCTTCATCAGGGACATCATCAAACCTGACCCACCCAAGAACT |
| | | TGCAGCTGAAGCCATTAAAGAATTCTAGGCAGGTGGAGGTCAG |
| | | CTGGGAGTACCCTGACACCTGGAGTACTCCACATTCCTACTTC |
| | | TCCCTGACATTCTGTGTTCAAGTCCAGGGCAAGAGCAAGAGAG |
| | | AAAAGAAAGATAGAGTCTTCACTGACAAGACCTCAGCCACAGT |
| | | CATCTGCAGGAAAAATGCCAGCATTTCAGTGAGGGCCCAGGAC |
| | | AGATACTATAGCTCATCTTGGTCAGAATGGGCATCTGTGCCCT |
| | | GCAGTTGACAATTG (SEQ ID NO: 53) |
| 29 | Human CAG-modified IL-12A/IL- | CTGCGCGCTCGCTCGCTCACTGAGGCCGCCCGGGCAAAGCCCG |
| | 12B-IRES (CpG modified) | GGCGTCGGGCGACCTTTGGTCGCCCGGCCTCAGTGAGCGAGCG |
| | construct | AGCGCGCAGAGAGGGAGTGGCCAACTCCATCACTAGGGGTTCC |
| | | TTAGTATTAAACGCGTGTCGACATTGATTATTGACTAGTTATT |
| | | AATAGTAATCAATTACGGGGTCATTAGTTCATAGCCCATATAT |
| | | GGAGTTCCGCGTTACATAACTTACGGTAAATGGCCCGCCTGGC |
| | | TGACCGCCCAACGACCCCCGCCCATTGACGTCAATAATGACGT |
| | | ATGTTCCCATAGTAACGCCAATAGGGACTTTCCATTGACGTCA |
| | | ATGGGTGGACTATTTACGGTAAACTGCCCACTTGGCAGTACAT |
| | | CAAGTGTATCATATGCCAAGTACGCCCCCTATTGACGTCAATG |
| | | ACGGTAAATGGCCCGCCTGGCATTATGCCCAGTACATGACCTT |
| | | ATGGGACTTTCCTACTTGGCAGTACATCTACGTATTAGTCATC |
| | | GCTATTACCATGGGTCGAGGTGAGCCCCACGTTCTGCTTCACT |
| | | CTCCCCATCTCCCCCCCCTCCCCACCCCCAATTTTGTATTTAT |
| | | TTATTTTTTAATTATTTTGTGCAGCGATGGGGGCGGGGGGGGG |
| | | GGGGGCGCGCGCCAGGCGGGGCGGGGCGGGGCGAGGGGCGGGG |
| | | CGGGGCGAGGCGGAGAGGTGCGGCGGCAGCCAATCAGAGCGGC |
| | | GCGCTCCGAAAGTTTCCTTTTATGGCGAGGCGGCGGCGGCGGC |
| | | GGCCCTATAAAAAGCGAAGCGCGCGGCGGGCGGGAGTCGCTGC |
| | | GCGCTGCCTTCGCCCCGTGCCCCGCTCCGCCGCCGCCTCGCGC |
| | | CGCCCGCCCCGGCTCTGACTGACCGCGTTACTCCCACAGGTGA |
| | | GCGGGCGGGACGGCCCTTCTCCTCCGGGCTGTAATTAGCGCTT |
| | | GGTTTAATGACGGCTCGTTTCTTTTCTGTGGCTGCGTGAAAGC |
| | | CTTGAGGGGCTCCGGGAGGGCCCTTTGTGCGGGGGGAGCGGCT |
| | | CGGGGGGTGCGTGCGTGTGTGTGTGCGTGGGGAGCGCCGCGTG |
| | | CGGCTCCGCGCTGCCCGGCGGCTGTGAGCGCTGCGGGCGCGGC |
| | | GCGGGGCTTTGTGCGCTCCGCAGTGTGCGCGAGAGGAGCGCGG |
| | | CCGGGGGCGGTGCCACGCGGTGCGGGGGGGGCTGCGAGGGGAA |
| | | CAAAGGCTGCGTGCGGGGTGTGTGCGTGGGGGGGGTGAGCAGG |
| | | GGGTGTGGGCGCGGCGGTCGGGCTGTAACCCCCCCCTGCACCC |
| | | CCCTCCCCGAGTTGCTGAGCACGGCCCGGCTTCGGGTGCGGGG |
| | | CTCCGTGCGGGGCGTGGCGCGGGGCTCGCCGTGCCGGGCGGGG |
| | | GGTGGCGGCGGGTGGGGGTGCCGGGCGGGGCGGGGCCGCCTCG |
| | | GGCCGGGGAGGGCTCGGGGGAGGGGCGCGGCGGCCCCCGGAGC |
| | | GCCGGCGGCTGTCGAGGCGCGGCGAGCCGCAGCCATTGCCTTT |
| | | TATGGTAATCGTGCGAGAGGGCGCAGGGACTTCCTTTGTCCCA |
| | | AATCTGTGCGGAGCCGAAATCTGGGAGGCGCCGCCGCACCCCC |
| | | TCTAGCGGGCGCGGGGCGAAGCGGTGCGGCGCCGGCAGGAAGG |
| | | AAATGGGCGGGGAGGGCCTTCGTGCGTCGCCGCGCCGCCGTCC |
| | | CCTTCTCCCTCTCCAGCCTCGGGGCTGTCCGCGGGGGGACGGC |
| | | TGCCTTCGGGGGGGACGGGGCAGGGCGGGGTTCGGCTTCTGGC |
| | | GTGTGACCGGCGGTCTAGACTCTGCTAACCATGTTCATGCCTT |
| | | CTTCTCTTTCCTACAGCTCCTGGGCAACGTGCTGGTTGTTGTG |
| | | CTGTCTCATCATTTTGGCAAAGAATTCAAGCTTCCAGCTAGCG |
| | | CCACCATGTGTCCAGCGCGCAGCCTCCTCCTTGTGGCTACCCT |
| | | GGTCCTCCTGGACCACCTCAGTTTGGCCAGAAACCTCCCTGTG |
| | | GCCACTCCAGACCCAGGAATGTTCCCATGCCTTCACCACTCCC |
| | | AAAACCTGCTGAGGGCTGTCAGCAACATGCTCCAGAAGGCCAG |
| | | ACAAACTCTAGAATTTTACCCTTGCACTTCTGAAGAGATTGAT |
| | | CATGAAGATATCACAAAAGATAAAACCAGCACAGTGGAGGCCT |
| | | GTTTACCATTGGAATTAACCAAGAATGAGAGTTGCCTAAATTC |
| | | CAGAGAGACCTCTTTCATAACTAATGGGAGTTGCCTGGCCTCC |
| | | AGAAAGACCTCTTTTATGATGGCCCTGTGCCTTAGTAGTATTT |
| | | ATGAAGACTTGAAGATGTACCAGGTGGAGTTCAAGACCATGAA |
| | | TGCAAAGCTTCTGATGGATCCTAAGAGGCAGATCTTTCTAGAT |
| | | CAAAACATGCTGGCAGTTATTGATGAGCTGATGCAGGCCCTGA |
| | | ATTTCAACAGTGAGACTGTGCCACAAAAATCCTCCCTTGAAGA |
| | | ACCTGATTTTTATAAAACTAAAATCAAGCTCTGCATACTTCTT |
| | | CATGCTTTCCGGATTCGCGCAGTGACTATTGATAGAGTGATGA |
| | | GCTATCTGAATGCTTCCTGAGCCCCTCTCCCTCCCCCCCCCCT |
| | | AACGTTACTGGCCGAAGCCGCTTGGAATAAGGCCGGTGTGCGT |
| | | TTGTCTATATGTTATTTTCCACCATATTGCCGTCTTTTGGCAA |
| | | TGTGAGGGCCCGGAAACCTGGCCCTGTCTTCTTGACGAGCATT |
| | | CCTAGGGGTCTTTCCCCTCTCGCCAAAGGAATGCAAGGTCTGT |
| | | TGAATGTCGTGAAGGAAGCAGTTCCTCTGGAAGCTTCTTGAAG |
| | | ACAAACAACGTCTGTAGCGACCCTTTGCAGGCAGCGGAACCCC |
| | | CCACCTGGCGACAGGTGCCTCTGCGGCCAAAAGCCACGTGTAT |
| | | AAGATACACCTGCAAAGGCGGCACAACCCCAGTGCCACGTTGT |
| | | GAGTTGGATAGTTGTGGAAAGAGTCAAATGGCTCTCCTCAAGC |
| | | GTATTCAACAAGGGGCTGAAGGATGCCCAGAAGGTACCCCATT |
| | | GTATGGGATCTGATCTGGGGCCTCGGTACACATGCTTTACATG |
| | | TGTTTAGTCGAGGTTAAAAAAACGTCTAGGCCCCCCGAACCAC |
| | | GGGGACGTGGTTTTCCTTTGAAAAACACGATGATAATATGGCC |
| | | ACAATGTGTCACCAGCAGTTGGTCATCTCTTGGTTTTCCCTGG |
| | | TTTTTCTGGCATCTCCCCTTGTGGCCATATGGGAACTGAAGAA |
| | | AGATGTTTATGTGGTAGAATTGGATTGGTATCCTGATGCCCCT |
| | | GGAGAAATGGTGGTCCTCACCTGTGACACCCCTGAAGAAGATG |
| | | GTATCACCTGGACCTTGGACCAGAGCAGTGAGGTCTTAGGCTC |
| | | TGGCAAAACCCTGACCATCCAAGTCAAAGAGTTTGGAGATGCT |
| | | GGCCAGTACACCTGTCACAAAGGAGGGGAGGTTCTAAGCCATT |
| | | CCCTCCTGCTGCTTCACAAAAAGGAAGATGGAATTTGGTCCAC |
| | | TGATATTTTAAAGGACCAGAAAGAACCCAAAAATAAGACCTTT |
| | | CTAAGATGTGAGGCCAAGAATTATTCTGGAAGGTTCACCTGCT |
| | | GGTGGCTGACTACAATCAGTACTGATTTGACATTCAGTGTCAA |
| | | AAGCAGCAGAGGCTCTTCTGACCCCCAAGGGGTGACATGCGGA |
| | | GCTGCTACACTCTCTGCAGAGAGAGTCAGAGGGGACAACAAGG |
| | | AGTATGAGTACTCAGTGGAGTGCCAGGAGGACAGTGCCTGCCC |
| | | AGCTGCTGAGGAGAGTCTGCCCATTGAGGTGATGGTGGATGCG |
| | | GTTCACAAGCTCAAGTATGAAAACTACACCAGCAGCTTCTTCA |
| | | TCAGGGACATCATCAAACCTGACCCACCCAAGAACTTGCAGCT |
| | | GAAGCCATTAAAGAATTCTAGGCAGGTGGAGGTCAGCTGGGAG |
| | | TACCCTGACACCTGGAGTACTCCACATTCCTACTTCTCCCTGA |
| | | CATTCTGTGTTCAAGTCCAGGGCAAGAGCAAGAGAGAAAAGAA |
| | | AGATAGAGTCTTCACTGACAAGACCTCAGCCACAGTCATCTGC |
| | | CGCAAAAATGCCAGCATTTCAGTGCGGGCCCAGGACCGCTACT |
| | | ATAGCTCATCTTGGTCAGAATGGGCATCTGTGCCCTGCAGTTG |
| | | ACAATTGCTGTGCCTTCTAGTTGCCAGCCATCTGTTGTTTGCC |
| | | CCTCCCCCGTGCCTTCCTTGACCCTGGAAGGTGCCACTCCCAC |
| | | TGTCCTTTCCTAATAAAATGAGGAAATTGCATCGCATTGTCTG |
| | | AGTAGGTGTCATTCTATTCTGGGGGGTGGGGTGGGGCAGGACA |
| | | GCAAGGGGGAGGATTGGGAAGACAATAGCAGGCATGCTGGGGA |
| | | TGCGGTGGGCTCTATGGAGCTCGAGAGGAACCCCTAGTGATGG |
| | | AGTTGGCCACTCCCTCTCTGCGCGCTCGCTCGCTCACTGAGGC |
| | | CGGGCGACCAAAGGTCGCCCGACGCCCGGGCTTTGCCCGGGCG |
| | | GCCTCAGTGAGCGAGCGAGCGCGCAGCTGCAGATCTG (SEQ |
| | | ID NO: 54) |
| 29 | Human CAG-modified IL-12A/IL- | GCTAGCGCCACCATGTGTCCAGCGCGCAGCCTCCTCCTTGTGG |
| | 12B-IRES (CpG modified) | CTACCCTGGTCCTCCTGGACCACCTCAGTTTGGCCAGAAACCT |
| | expression sequence | CCCTGTGGCCACTCCAGACCCAGGAATGTTCCCATGCCTTCAC |
| | | CACTCCCAAAACCTGCTGAGGGCTGTCAGCAACATGCTCCAGA |
| | | AGGCCAGACAAACTCTAGAATTTTACCCTTGCACTTCTGAAGA |
| | | GATTGATCATGAAGATATCACAAAAGATAAAACCAGCACAGTG |
| | | GAGGCCTGTTTACCATTGGAATTAACCAAGAATGAGAGTTGCC |
| | | TAAATTCCAGAGAGACCTCTTTCATAACTAATGGGAGTTGCCT |
| | | GGCCTCCAGAAAGACCTCTTTTATGATGGCCCTGTGCCTTAGT |
| | | AGTATTTATGAAGACTTGAAGATGTACCAGGTGGAGTTCAAGA |
| | | CCATGAATGCAAAGCTTCTGATGGATCCTAAGAGGCAGATCTT |
| | | TCTAGATCAAAACATGCTGGCAGTTATTGATGAGCTGATGCAG |
| | | GCCCTGAATTTCAACAGTGAGACTGTGCCACAAAAATCCTCCC |
| | | TTGAAGAACCTGATTTTTATAAAACTAAAATCAAGCTCTGCAT |
| | | ACTTCTTCATGCTTTCCGGATTCGCGCAGTGACTATTGATAGA |
| | | GTGATGAGCTATCTGAATGCTTCCTGAGCCCCTCTCCCTCCCC |
| | | CCCCCCTAACGTTACTGGCCGAAGCCGCTTGGAATAAGGCCGG |
| | | TGTGCGTTTGTCTATATGTTATTTTCCACCATATTGCCGTCTT |
| | | TTGGCAATGTGAGGGCCCGGAAACCTGGCCCTGTCTTCTTGAC |
| | | GAGCATTCCTAGGGGTCTTTCCCCTCTCGCCAAAGGAATGCAA |
| | | GGTCTGTTGAATGTCGTGAAGGAAGCAGTTCCTCTGGAAGCTT |
| | | CTTGAAGACAAACAACGTCTGTAGCGACCCTTTGCAGGCAGCG |
| | | GAACCCCCCACCTGGCGACAGGTGCCTCTGCGGCCAAAAGCCA |
| | | CGTGTATAAGATACACCTGCAAAGGCGGCACAACCCCAGTGCC |
| | | ACGTTGTGAGTTGGATAGTTGTGGAAAGAGTCAAATGGCTCTC |
| | | CTCAAGCGTATTCAACAAGGGGCTGAAGGATGCCCAGAAGGTA |
| | | CCCCATTGTATGGGATCTGATCTGGGGCCTCGGTACACATGCT |
| | | TTACATGTGTTTAGTCGAGGTTAAAAAAACGTCTAGGCCCCCC |
| | | GAACCACGGGGACGTGGTTTTCCTTTGAAAAACACGATGATAA |
| | | TATGGCCACAATGTGTCACCAGCAGTTGGTCATCTCTTGGTTT |
| | | TCCCTGGTTTTTCTGGCATCTCCCCTTGTGGCCATATGGGAAC |
| | | TGAAGAAAGATGTTTATGTGGTAGAATTGGATTGGTATCCTGA |
| | | TGCCCCTGGAGAAATGGTGGTCCTCACCTGTGACACCCCTGAA |
| | | GAAGATGGTATCACCTGGACCTTGGACCAGAGCAGTGAGGTCT |
| | | TAGGCTCTGGCAAAACCCTGACCATCCAAGTCAAAGAGTTTGG |
| | | AGATGCTGGCCAGTACACCTGTCACAAAGGAGGGGAGGTTCTA |
| | | AGCCATTCCCTCCTGCTGCTTCACAAAAAGGAAGATGGAATTT |
| | | GGTCCACTGATATTTTAAAGGACCAGAAAGAACCCAAAAATAA |
| | | GACCTTTCTAAGATGTGAGGCCAAGAATTATTCTGGAAGGTTC |
| | | ACCTGCTGGTGGCTGACTACAATCAGTACTGATTTGACATTCA |
| | | GTGTCAAAAGCAGCAGAGGCTCTTCTGACCCCCAAGGGGTGAC |
| | | ATGCGGAGCTGCTACACTCTCTGCAGAGAGAGTCAGAGGGGAC |
| | | AACAAGGAGTATGAGTACTCAGTGGAGTGCCAGGAGGACAGTG |
| | | CCTGCCCAGCTGCTGAGGAGAGTCTGCCCATTGAGGTGATGGT |
| | | GGATGCGGTTCACAAGCTCAAGTATGAAAACTACACCAGCAGC |
| | | TTCTTCATCAGGGACATCATCAAACCTGACCCACCCAAGAACT |
| | | TGCAGCTGAAGCCATTAAAGAATTCTAGGCAGGTGGAGGTCAG |
| | | CTGGGAGTACCCTGACACCTGGAGTACTCCACATTCCTACTTC |
| | | TCCCTGACATTCTGTGTTCAAGTCCAGGGCAAGAGCAAGAGAG |
| | | AAAAGAAAGATAGAGTCTTCACTGACAAGACCTCAGCCACAGT |
| | | CATCTGCCGCAAAAATGCCAGCATTTCAGTGCGGGCCCAGGAC |
| | | CGCTACTATAGCTCATCTTGGTCAGAATGGGCATCTGTGCCCT |
| | | GCAGTTGACAATTG (SEQ ID NO: 55) |
| 30 | Human CAG-wild-type IL-12A/IL- | CTGCGCGCTCGCTCGCTCACTGAGGCCGCCCGGGCAAAGCCCG |
| | 12B-F2A (CpG modified) construct | GGCGTCGGGCGACCTTTGGTCGCCCGGCCTCAGTGAGCGAGCG |
| | | AGCGCGCAGAGAGGGAGTGGCCAACTCCATCACTAGGGGTTCC |
| | | TTAGTATTAAACGCGTGTCGACATTGATTATTGACTAGTTATT |
| | | AATAGTAATCAATTACGGGGTCATTAGTTCATAGCCCATATAT |
| | | GGAGTTCCGCGTTACATAACTTACGGTAAATGGCCCGCCTGGC |
| | | TGACCGCCCAACGACCCCCGCCCATTGACGTCAATAATGACGT |
| | | ATGTTCCCATAGTAACGCCAATAGGGACTTTCCATTGACGTCA |
| | | ATGGGTGGACTATTTACGGTAAACTGCCCACTTGGCAGTACAT |
| | | CAAGTGTATCATATGCCAAGTACGCCCCCTATTGACGTCAATG |
| | | ACGGTAAATGGCCCGCCTGGCATTATGCCCAGTACATGACCTT |
| | | ATGGGACTTTCCTACTTGGCAGTACATCTACGTATTAGTCATC |
| | | GCTATTACCATGGGTCGAGGTGAGCCCCACGTTCTGCTTCACT |
| | | CTCCCCATCTCCCCCCCCTCCCCACCCCCAATTTTGTATTTAT |
| | | TTATTTTTTAATTATTTTGTGCAGCGATGGGGGCGGGGGGGGG |
| | | GGGGGCGCGCGCCAGGCGGGGCGGGGCGGGGCGAGGGGCGGGG |
| | | CGGGGCGAGGCGGAGAGGTGCGGCGGCAGCCAATCAGAGCGGC |
| | | GCGCTCCGAAAGTTTCCTTTTATGGCGAGGCGGCGGCGGCGGC |
| | | GGCCCTATAAAAAGCGAAGCGCGCGGCGGGCGGGAGTCGCTGC |
| | | GCGCTGCCTTCGCCCCGTGCCCCGCTCCGCCGCCGCCTCGCGC |
| | | CGCCCGCCCCGGCTCTGACTGACCGCGTTACTCCCACAGGTGA |
| | | GCGGGCGGGACGGCCCTTCTCCTCCGGGCTGTAATTAGCGCTT |
| | | GGTTTAATGACGGCTCGTTTCTTTTCTGTGGCTGCGTGAAAGC |
| | | CTTGAGGGGCTCCGGGAGGGCCCTTTGTGCGGGGGGAGCGGCT |
| | | CGGGGGGTGCGTGCGTGTGTGTGTGCGTGGGGAGCGCCGCGTG |
| | | CGGCTCCGCGCTGCCCGGCGGCTGTGAGCGCTGCGGGCGCGGC |
| | | GCGGGGCTTTGTGCGCTCCGCAGTGTGCGCGAGAGGAGCGCGG |
| | | CCGGGGGCGGTGCCACGCGGTGCGGGGGGGGCTGCGAGGGGAA |
| | | CAAAGGCTGCGTGCGGGGTGTGTGCGTGGGGGGGGTGAGCAGG |
| | | GGGTGTGGGCGCGGCGGTCGGGCTGTAACCCCCCCCTGCACCC |
| | | CCCTCCCCGAGTTGCTGAGCACGGCCCGGCTTCGGGTGCGGGG |
| | | CTCCGTGCGGGGCGTGGCGCGGGGCTCGCCGTGCCGGGCGGGG |
| | | GGTGGCGGCGGGTGGGGGTGCCGGGCGGGGCGGGGCCGCCTCG |
| | | GGCCGGGGAGGGCTCGGGGGAGGGGCGCGGCGGCCCCCGGAGC |
| | | GCCGGCGGCTGTCGAGGCGCGGCGAGCCGCAGCCATTGCCTTT |
| | | TATGGTAATCGTGCGAGAGGGCGCAGGGACTTCCTTTGTCCCA |
| | | AATCTGTGCGGAGCCGAAATCTGGGAGGCGCCGCCGCACCCCC |
| | | TCTAGCGGGCGCGGGGCGAAGCGGTGCGGCGCCGGCAGGAAGG |
| | | AAATGGGCGGGGAGGGCCTTCGTGCGTCGCCGCGCCGCCGTCC |
| | | CCTTCTCCCTCTCCAGCCTCGGGGCTGTCCGCGGGGGGACGGC |
| | | TGCCTTCGGGGGGGACGGGGCAGGGCGGGGTTCGGCTTCTGGC |
| | | GTGTGACCGGCGGTCTAGACTCTGCTAACCATGTTCATGCCTT |
| | | CTTCTCTTTCCTACAGCTCCTGGGCAACGTGCTGGTTGTTGTG |
| | | CTGTCTCATCATTTTGGCAAAGAATTCAAGCTTCCAGCTAGCG |
| | | CCACCATGTGTCCAGCGCGCAGCCTCCTCCTTGTGGCTACCCT |
| | | GGTCCTCCTGGACCACCTCAGTTTGGCCAGAAACCTCCCCGTG |
| | | GCCACTCCAGACCCAGGAATGTTCCCATGCCTTCACCACTCCC |
| | | AAAACCTGCTGAGGGCCGTCAGCAACATGCTCCAGAAGGCCAG |
| | | ACAAACTCTAGAATTTTACCCTTGCACTTCTGAAGAGATTGAT |
| | | CATGAAGATATCACAAAAGATAAAACCAGCACAGTGGAGGCCT |
| | | GTTTACCATTGGAATTAACCAAGAATGAGAGTTGCCTAAATTC |
| | | CAGAGAGACCTCTTTCATAACTAATGGGAGTTGCCTGGCCTCC |
| | | AGAAAGACCTCTTTTATGATGGCCCTGTGCCTTAGTAGTATTT |
| | | ATGAAGACTTGAAGATGTACCAGGTGGAGTTCAAGACCATGAA |
| | | TGCAAAGCTTCTGATGGATCCTAAGAGGCAGATCTTTCTAGAT |
| | | CAAAACATGCTGGCAGTTATTGATGAGCTGATGCAGGCCCTGA |
| | | ATTTCAACAGTGAGACTGTGCCACAAAAATCCTCCCTTGAAGA |
| | | ACCGGATTTTTATAAAACTAAAATCAAGCTCTGCATACTTCTT |
| | | CATGCTTTCCGGATTCGGGCAGTGACTATTGATAGAGTGATGA |
| | | GCTATCTGAATGCTTCCAGAAAGAGAAGGAGTGGCTCAGGAGC |
| | | CCCTGTGAAACAGACCCTGAACTTTGACCTCTTGAAGCTTGCT |
| | | GGGGATGTGGAGTCTAATCCTGGTCCAATGTGTCACCAGCAGT |
| | | TGGTCATCTCTTGGTTTTCCCTGGTTTTTCTGGCATCTCCCCT |
| | | CGTGGCCATATGGGAACTGAAGAAAGATGTTTATGTCGTAGAA |
| | | TTGGATTGGTATCCGGATGCCCCTGGAGAAATGGTGGTCCTCA |
| | | CCTGTGACACCCCTGAAGAAGATGGTATCACCTGGACCTTGGA |
| | | CCAGAGCAGTGAGGTCTTAGGCTCTGGCAAAACCCTGACCATC |
| | | CAAGTCAAAGAGTTTGGAGATGCTGGCCAGTACACCTGTCACA |
| | | AAGGAGGCGAGGTTCTAAGCCATTCGCTCCTGCTGCTTCACAA |
| | | AAAGGAAGATGGAATTTGGTCCACTGATATTTTAAAGGACCAG |
| | | AAAGAACCCAAAAATAAGACCTTTCTAAGATGCGAGGCCAAGA |
| | | ATTATTCTGGACGTTTCACCTGCTGGTGGCTGACGACAATCAG |
| | | TACTGATTTGACATTCAGTGTCAAAAGCAGCAGAGGCTCTTCT |
| | | GACCCCCAAGGGGTGACGTGCGGAGCTGCTACACTCTCTGCAG |
| | | AGAGAGTCAGAGGGGACAACAAGGAGTATGAGTACTCAGTGGA |
| | | GTGCCAGGAGGACAGTGCCTGCCCAGCTGCTGAGGAGAGTCTG |
| | | CCCATTGAGGTGATGGTGGATGCCGTTCACAAGCTCAAGTATG |
| | | AAAACTACACCAGCAGCTTCTTCATCAGGGACATCATCAAACC |
| | | TGACCCACCCAAGAACTTGCAGCTGAAGCCATTAAAGAATTCT |
| | | CGGCAGGTGGAGGTCAGCTGGGAGTACCCTGACACCTGGAGTA |
| | | CTCCACATTCCTACTTCTCCCTGACATTCTGCGTTCAAGTCCA |
| | | GGGCAAGAGCAAGAGAGAAAAGAAAGATAGAGTCTTCACGGAC |
| | | AAGACCTCAGCCACGGTCATCTGCCGCAAAAATGCCAGCATTA |
| | | GCGTGCGGGCCCAGGACCGCTACTATAGCTCATCTTGGAGCGA |
| | | ATGGGCATCTGTGCCCTGCAGTTGACAATTGCTGTGCCTTCTA |
| | | GTTGCCAGCCATCTGTTGTTTGCCCCTCCCCCGTGCCTTCCTT |
| | | GACCCTGGAAGGTGCCACTCCCACTGTCCTTTCCTAATAAAAT |
| | | GAGGAAATTGCATCGCATTGTCTGAGTAGGTGTCATTCTATTC |
| | | TGGGGGGTGGGGTGGGGCAGGACAGCAAGGGGGAGGATTGGGA |
| | | AGACAATAGCAGGCATGCTGGGGATGCGGTGGGCTCTATGGAG |
| | | CTCGAGAGGAACCCCTAGTGATGGAGTTGGCCACTCCCTCTCT |
| | | GCGCGCTCGCTCGCTCACTGAGGCCGGGCGACCAAAGGTCGCC |
| | | CGACGCCCGGGCTTTGCCCGGGCGGCCTCAGTGAGCGAGCGAG |
| | | CGCGCAGCTGCAGATCTG (SEQ ID NO: 56) |
| 31 | Human CAG-wild-type IL-12A/IL- | GCTAGCGCCACCATGTGTCCAGCGCGCAGCCTCCTCCTTGTGG |
| | 12B-F2A (CpG modified) | CTACCCTGGTCCTCCTGGACCACCTCAGTTTGGCCAGAAACCT |
| | expression sequence | CCCCGTGGCCACTCCAGACCCAGGAATGTTCCCATGCCTTCAC |
| | | CACTCCCAAAACCTGCTGAGGGCCGTCAGCAACATGCTCCAGA |
| | | AGGCCAGACAAACTCTAGAATTTTACCCTTGCACTTCTGAAGA |
| | | GATTGATCATGAAGATATCACAAAAGATAAAACCAGCACAGTG |
| | | GAGGCCTGTTTACCATTGGAATTAACCAAGAATGAGAGTTGCC |
| | | TAAATTCCAGAGAGACCTCTTTCATAACTAATGGGAGTTGCCT |
| | | GGCCTCCAGAAAGACCTCTTTTATGATGGCCCTGTGCCTTAGT |
| | | AGTATTTATGAAGACTTGAAGATGTACCAGGTGGAGTTCAAGA |
| | | CCATGAATGCAAAGCTTCTGATGGATCCTAAGAGGCAGATCTT |
| | | TCTAGATCAAAACATGCTGGCAGTTATTGATGAGCTGATGCAG |
| | | GCCCTGAATTTCAACAGTGAGACTGTGCCACAAAAATCCTCCC |
| | | TTGAAGAACCGGATTTTTATAAAACTAAAATCAAGCTCTGCAT |
| | | ACTTCTTCATGCTTTCCGGATTCGGGCAGTGACTATTGATAGA |
| | | GTGATGAGCTATCTGAATGCTTCCAGAAAGAGAAGGAGTGGCT |
| | | CAGGAGCCCCTGTGAAACAGACCCTGAACTTTGACCTCTTGAA |
| | | GCTTGCTGGGGATGTGGAGTCTAATCCTGGTCCAATGTGTCAC |
| | | CAGCAGTTGGTCATCTCTTGGTTTTCCCTGGTTTTTCTGGCAT |
| | | CTCCCCTCGTGGCCATATGGGAACTGAAGAAAGATGTTTATGT |
| | | CGTAGAATTGGATTGGTATCCGGATGCCCCTGGAGAAATGGTG |
| | | GTCCTCACCTGTGACACCCCTGAAGAAGATGGTATCACCTGGA |
| | | CCTTGGACCAGAGCAGTGAGGTCTTAGGCTCTGGCAAAACCCT |
| | | GACCATCCAAGTCAAAGAGTTTGGAGATGCTGGCCAGTACACC |
| | | TGTCACAAAGGAGGCGAGGTTCTAAGCCATTCGCTCCTGCTGC |
| | | TTCACAAAAAGGAAGATGGAATTTGGTCCACTGATATTTTAAA |
| | | GGACCAGAAAGAACCCAAAAATAAGACCTTTCTAAGATGCGAG |
| | | GCCAAGAATTATTCTGGACGTTTCACCTGCTGGTGGCTGACGA |
| | | CAATCAGTACTGATTTGACATTCAGTGTCAAAAGCAGCAGAGG |
| | | CTCTTCTGACCCCCAAGGGGTGACGTGCGGAGCTGCTACACTC |
| | | TCTGCAGAGAGAGTCAGAGGGGACAACAAGGAGTATGAGTACT |
| | | CAGTGGAGTGCCAGGAGGACAGTGCCTGCCCAGCTGCTGAGGA |
| | | GAGTCTGCCCATTGAGGTGATGGTGGATGCCGTTCACAAGCTC |
| | | AAGTATGAAAACTACACCAGCAGCTTCTTCATCAGGGACATCA |
| | | TCAAACCTGACCCACCCAAGAACTTGCAGCTGAAGCCATTAAA |
| | | GAATTCTCGGCAGGTGGAGGTCAGCTGGGAGTACCCTGACACC |
| | | TGGAGTACTCCACATTCCTACTTCTCCCTGACATTCTGCGTTC |
| | | AAGTCCAGGGCAAGAGCAAGAGAGAAAAGAAAGATAGAGTCTT |
| | | CACGGACAAGACCTCAGCCACGGTCATCTGCCGCAAAAATGCC |
| | | AGCATTAGCGTGCGGGCCCAGGACCGCTACTATAGCTCATCTT |
| | | GGAGCGAATGGGCATCTGTGCCCTGCAGTTGACAATTG (SEQ |
| | | ID NO: 57) |
| 32 | CMV enhancer-CBA promoter-WT | ctgcgcgctcgctcgctcactgaggccgcccgggcaaagcccg |
| | human IL-12A (no in-frame stop | ggcgtcgggcgacctttggtcgcccggcctcagtgagcgagcg |
| | codon)-F2A-WT human IL-12B | agcgcgcagagagggagtggccaactccatcactaggggttcc |
| | (with stop codon)-HGH polyA | ttagtattaaacgcgtgtcgacattgattattgactagttatt |
| | | aatagtaatcaattacggggtcattagttcatagcccatatat |
| | | ggagttccgcgttacataacttacggtaaatggcccgcctggc |
| | | tgaccgcccaacgacccccgcccattgacgtcaataatgacgt |
| | | atgttcccatagtaacgccaatagggactttccattgacgtca |
| | | atgggtggactatttacggtaaactgcccacttggcagtacat |
| | | caagtgtatcatatgccaagtacgccccctattgacgtcaatg |
| | | acggtaaatggcccgcctggcattatgcccagtacatgacctt |
| | | atgggactttcctacttggcagtacatctacgtattagtcatc |
| | | gctattaccatgggtcgaggtgagccccacgttctgcttcact |
| | | ctccccatctcccccccctccccacccccaattttgtatttat |
| | | ttattttttaattattttgtgcagcgatgggggcggggggggg |
| | | gggggcgcgcgccaggcggggcggggcggggcgaggggcgggg |
| | | cggggcgaggcggagaggtgcggcggcagccaatcagagcggc |
| | | gcgctccgaaagtttccttttatggcgaggcggcggcggcggc |
| | | ggccctataaaaagcgaagcgcgcggcgggcgggagtcgctgc |
| | | gcgctgccttcgccccgtgccccgctccgccgccgcctcgcgc |
| | | cgcccgccccggctctgactgaccgcgttactcccacaggtga |
| | | gcgggcgggacggcccttctcctccgggctgtaattagcgctt |
| | | ggtttaatgacggctcgtttcttttctgtggctgcgtgaaagc |
| | | cttgaggggctccgggagggccctttgtgcggggggagcggct |
| | | cggggggtgcgtgcgtgtgtgtgtgcgtggggagcgccgcgtg |
| | | cggctccgcgctgcccggcggctgtgagcgctgcgggcgcggc |
| | | gcggggctttgtgcgctccgcagtgtgcgcgagaggagcgcgg |
| | | ccgggggcggtgccacgcggtgcggggggggctgcgaggggaa |
| | | caaaggctgcgtgcggggtgtgtgcgtggggggggtgagcagg |
| | | gggtgtgggcgcggcggtcgggctgtaacccccccctgcaccc |
| | | ccctccccgagttgctgagcacggcccggcttcgggtgcgggg |
| | | ctccgtgcggggcgtggcgcggggctcgccgtgccgggcgggg |
| | | ggtggcggcgggtgggggtgccgggcggggcggggccgcctcg |
| | | ggccggggagggctcgggggaggggcgcggcggcccccggagc |
| | | gccggcggctgtcgaggcgcggcgagccgcagccattgccttt |
| | | tatggtaatcgtgcgagagggcgcagggacttcctttgtccca |
| | | aatctgtgcggagccgaaatctgggaggcgccgccgcaccccc |
| | | tctagcgggcgcggggcgaagcggtgcggcgccggcaggaagg |
| | | aaatgggcggggagggccttcgtgcgtcgccgcgccgccgtcc |
| | | ccttctccctctccagcctcggggctgtccgcggggggacggc |
| | | tgccttcgggggggacggggcagggcggggttcggcttctggc |
| | | gtgtgaccggcggtctagactctgctaaccatgttcatgcctt |
| | | cttctctttcctacagctcctgggcaacgtgctggttgttgtg |
| | | ctgtctcatcattttggcaaagaattcaagcttccagctagcg |
| | | ccaccatgtgtccagcgcgcagcctcctccttgtggctaccct |
| | | ggtcctcctggaccacctcagtttggccagaaacctccccgtg |
| | | gccactccagacccaggaatgttcccatgccttcaccactccc |
| | | aaaacctgctgagggccgtcagcaacatgctccagaaggccag |
| | | acaaactctagaattttacccttgcacttctgaagagattgat |
| | | catgaagatatcacaaaagataaaaccagcacagtggaggcct |
| | | gtttaccattggaattaaccaagaatgagagttgcctaaattc |
| | | cagagagacctctttcataactaatgggagttgcctggcctcc |
| | | agaaagacctcttttatgatggccctgtgccttagtagtattt |
| | | atgaagacttgaagatgtaccaggtggagttcaagaccatgaa |
| | | tgcaaagcttctgatggatcctaagaggcagatctttctagat |
| | | caaaacatgctggcagttattgatgagctgatgcaggccctga |
| | | atttcaacagtgagactgtgccacaaaaatcctcccttgaaga |
| | | accggatttttataaaactaaaatcaagctctgcatacttctt |
| | | catgctttccggattcgggcagtgactattgatagagtgatga |
| | | gctatctgaatgcttccagaaagagaaggagtggctcaggagc |
| | | ccctgtgaaacagaccctgaactttgacctcttgaagcttgct |
| | | ggggatgtggagtctaatcctggtccaatgtgtcaccagcagt |
| | | tggtcatctcttggttttccctggtttttctggcatctcccct |
| | | cgtggccatatgggaactgaagaaagatgtttatgtcgtagaa |
| | | ttggattggtatccggatgcccctggagaaatggtggtcctca |
| | | cctgtgacacccctgaagaagatggtatcacctggaccttgga |
| | | ccagagcagtgaggtcttaggctctggcaaaaccctgaccatc |
| | | caagtcaaagagtttggagatgctggccagtacacctgtcaca |
| | | aaggaggcgaggttctaagccattcgctcctgctgcttcacaa |
| | | aaaggaagatggaatttggtccactgatattttaaaggaccag |
| | | aaagaacccaaaaataagacctttctaagatgcgaggccaaga |
| | | attattctggacgtttcacctgctggtggctgacgacaatcag |
| | | tactgatttgacattcagtgtcaaaagcagcagaggctcttct |
| | | gacccccaaggggtgacgtgcggagctgctacactctctgcag |
| | | agagagtcagaggggacaacaaggagtatgagtactcagtgga |
| | | gtgccaggaggacagtgcctgcccagctgctgaggagagtctg |
| | | cccattgaggtgatggtggatgccgttcacaagctcaagtatg |
| | | aaaactacaccagcagcttcttcatcagggacatcatcaaacc |
| | | tgacccacccaagaacttgcagctgaagccattaaagaattct |
| | | cggcaggtggaggtcagctgggagtaccctgacacctggagta |
| | | ctccacattcctacttctccctgacattctgcgttcaagtcca |
| | | gggcaagagcaagagagaaaagaaagatagagtcttcacggac |
| | | aagacctcagccacggtcatctgccgcaaaaatgccagcatta |
| | | gcgtgcgggcccaggaccgctactatagctcatcttggagcga |
| | | atgggcatctgtgccctgcagttgacaattggatctacgggtg |
| | | gcatccctgtgacccctccccagtgcctctcctggccctggaa |
| | | gttgccactccagtgcccaccagccttgtcctaataaaattaa |
| | | gttgcatcattttgtctgactaggtgtccttctataatattat |
| | | ggggtggaggggggtggtatggagcaaggggcaagttgggaag |
| | | acaacctgtagggcctgcggggtctattgggaaccaagctgga |
| | | gtgcagtggcacaatcttggctcactgcaatctccgcctcctg |
| | | ggttcaagcgattctcctgcctcagcctcccgagttgttggga |
| | | ttccaggcatgcatgaccaggctcagctaatttttgttttttt |
| | | ggtagagacggggtttcaccatattggccaggctggtctccaa |
| | | ctcctaatctcaggtgatctacccaccttggcctcccaaattg |
| | | ctgggattacaggcgtgaaccactgctcccttccctgtccttc |
| | | tggagctcgagaggaacccctagtgatggagttggccactccc |
| | | tctctgcgcgctcgctcgctcactgaggccgggcgaccaaagg |
| | | tcgcccgacgcccgggctttgcccgggcggcctcagtgagcga |
| | | gcgagcgcgcagctgcagatctg |
| | | (SEQ ID NO: 82) |
| 33 | CMV enhancer-CBA promoter-WT | ctgcgcgctcgctcgctcactgaggccgcccgggcaaagcccg |
| | mouse IL-12A (no in-frame stop | ggcgtcgggcgacctttggtcgcccggcctcagtgagcgagcg |
| | codon)-F2A-WT mouse IL-12B | agcgcgcagagagggagtggccaactccatcactaggggttcc |
| | (with stop codon)-HGH polyA | ttagtattaaacgcgtgtcgacattgattattgactagttatt |
| | | aatagtaatcaattacggggtcattagttcatagcccatatat |
| | | ggagttccgcgttacataacttacggtaaatggcccgcctggc |
| | | tgaccgcccaacgacccccgcccattgacgtcaataatgacgt |
| | | atgttcccatagtaacgccaatagggactttccattgacgtca |
| | | atgggtggactatttacggtaaactgcccacttggcagtacat |
| | | caagtgtatcatatgccaagtacgccccctattgacgtcaatg |
| | | acggtaaatggcccgcctggcattatgcccagtacatgacctt |
| | | atgggactttcctacttggcagtacatctacgtattagtcatc |
| | | gctattaccatgggtcgaggtgagccccacgttctgcttcact |
| | | ctccccatctcccccccctccccacccccaattttgtatttat |
| | | ttattttttaattattttgtgcagcgatgggggcggggggggg |
| | | gggggcgcgcgccaggcggggcggggcggggcgaggggcgggg |
| | | cggggcgaggcggagaggtgcggcggcagccaatcagagcggc |
| | | gcgctccgaaagtttccttttatggcgaggcggcggcggcggc |
| | | ggccctataaaaagcgaagcgcgcggcgggcgggagtcgctgc |
| | | gcgctgccttcgccccgtgccccgctccgccgccgcctcgcgc |
| | | cgcccgccccggctctgactgaccgcgttactcccacaggtga |
| | | gcgggcgggacggcccttctcctccgggctgtaattagcgctt |
| | | ggtttaatgacggctcgtttcttttctgtggctgcgtgaaagc |
| | | cttgaggggctccgggagggccctttgtgcggggggagcggct |
| | | cggggggtgcgtgcgtgtgtgtgtgcgtggggagcgccgcgtg |
| | | cggctccgcgctgcccggcggctgtgagcgctgcgggcgcggc |
| | | gcggggctttgtgcgctccgcagtgtgcgcgagaggagcgcgg |
| | | ccgggggcggtgccacgcggtgcggggggggctgcgaggggaa |
| | | caaaggctgcgtgcggggtgtgtgcgtggggggggtgagcagg |
| | | gggtgtgggcgcggcggtcgggctgtaacccccccctgcaccc |
| | | ccctccccgagttgctgagcacggcccggcttcgggtgcgggg |
| | | ctccgtgcggggcgtggcgcggggctcgccgtgccgggcgggg |
| | | ggtggcggcgggtgggggtgccgggcggggcggggccgcctcg |
| | | ggccggggagggctcgggggaggggcgcggcggcccccggagc |
| | | gccggcggctgtcgaggcgcggcgagccgcagccattgccttt |
| | | tatggtaatcgtgcgagagggcgcagggacttcctttgtccca |
| | | aatctgtgcggagccgaaatctgggaggcgccgccgcaccccc |
| | | tctagcgggcgcggggcgaagcggtgcggcgccggcaggaagg |
| | | aaatgggcggggagggccttcgtgcgtcgccgcgccgccgtcc |
| | | ccttctccctctccagcctcggggctgtccgcggggggacggc |
| | | tgccttcgggggggacggggcagggcggggttcggcttctggc |
| | | gtgtgaccggcggtctagactctgctaaccatgttcatgcctt |
| | | cttctctttcctacagctcctgggcaacgtgctggttgttgtg |
| | | ctgtctcatcattttggcaaagaattcaagcttccagctagcg |
| | | ccaccatggtcagcgttccaacagcctcaccctcggcatccag |
| | | cagctcctctcagtgccggtccagcatgtgtcaatcacgctac |
| | | ctcctctttttggccacccttgccctcctaaaccacctcagtt |
| | | tggccagggtcattccagtctctggacctgccaggtgtcttag |
| | | ccagtcccgaaacctgctgaagaccacagatgacatggtgaag |
| | | acggccagagaaaaactgaaacattattcctgcactgctgaag |
| | | acatcgatcatgaagacatcacacgggaccaaaccagcacatt |
| | | gaagacctgtttaccactggaactacacaagaacgagagttgc |
| | | ctggctactagagagacttcttccacaacaagagggagctgcc |
| | | tgcccccacagaagacgtctttgatgatgaccctgtgccttgg |
| | | tagcatctatgaggacttgaagatgtaccagacagagttccag |
| | | gccatcaacgcagcacttcagaatcacaaccatcagcagatca |
| | | ttctagacaagggcatgctggtggccatcgatgagctgatgca |
| | | gtctctgaatcataatggcgagactctgcgccagaaacctcct |
| | | gtgggagaagcagacccttacagagtgaaaatgaagctctgca |
| | | tcctgcttcacgccttcagcacccgcgtcgtgaccatcaacag |
| | | ggtgatgggctatctgagctccgccagaaagagaaggagtggc |
| | | tcaggagcccctgtgaaacagaccctgaactttgacctcttga |
| | | agcttgctggggatgtggagtctaatcctggtccaatgtgtcc |
| | | tcagaagctaaccatctcctggtttgccatcgttttgctggtg |
| | | tctccactcatggccatgtgggagctggagaaagacgtttatg |
| | | ttgtagaggtggactggactcccgatgcccctggagaaacagt |
| | | gaacctcacctgtgacacgcctgaagaagatgacatcacctgg |
| | | acctcagaccagagacatggagtcataggctctggaaagaccc |
| | | tgaccatcactgtcaaagagtttctagatgctggccagtacac |
| | | ctgccacaaaggaggcgagactctgagccactcacatctgctg |
| | | ctccacaagaaggaaaatggaatttggtccactgaaattttaa |
| | | aaaatttcaaaaacaagactttcctgaagtgtgaagcaccaaa |
| | | ttactccggacggttcacgtgctcatggctggtgcaaagaaac |
| | | atggacttgaagttcaacatcaagagcagtagcagttcccctg |
| | | actctcgggcagtgacatgtggaatggcgtctctgtctgcaga |
| | | gaaggtcacactggaccaaagggactatgagaagtattcagtg |
| | | tcctgccaggaggatgtcacctgcccaactgccgaggagaccc |
| | | tgcccattgaactggcgttggaagcacggcagcagaataaata |
| | | tgagaactacagcaccagcttcttcatcagggacatcatcaaa |
| | | ccagacccgcccaagaacttgcagatgaagcctttgaagaact |
| | | cacaggtggaggtcagctgggagtaccctgactcctggagcac |
| | | tccccattcctacttctccctcaagttctttgttcgaatccag |
| | | cgcaagaaagaaaagatgaaggagacagaggaggggtgtaacc |
| | | agaaaggagcgttcctcgtagagaagacatctaccgaagtcca |
| | | atgcaaaggcgggaatgtctgcgtgcaagctcaggatcgctat |
| | | tacaattcctcatgcagcaagtgggcatgtgttccctgcaggg |
| | | tccgatcctgacaattggatctacgggtggcatccctgtgacc |
| | | cctccccagtgcctctcctggccctggaagttgccactccagt |
| | | gcccaccagccttgtcctaataaaattaagttgcatcattttg |
| | | tctgactaggtgtccttctataatattatggggtggagggggg |
| | | tggtatggagcaaggggcaagttgggaagacaacctgtagggc |
| | | ctgcggggtctattgggaaccaagctggagtgcagtggcacaa |
| | | tcttggctcactgcaatctccgcctcctgggttcaagcgattc |
| | | tcctgcctcagcctcccgagttgttgggattccaggcatgcat |
| | | gaccaggctcagctaatttttgtttttttggtagagacggggt |
| | | ttcaccatattggccaggctggtctccaactcctaatctcagg |
| | | tgatctacccaccttggcctcccaaattgctgggattacaggc |
| | | gtgaaccactgctcccttccctgtccttctggagctcgagagg |
| | | aacccctagtgatggagttggccactccctctctgcgcgctcg |
| | | ctcgctcactgaggccgggcgaccaaaggtcgcccgacgcccg |
| | | ggctttgcccgggcggcctcagtgagcgagcgagcgcgcagct |
| | | gcagatctg (SEQ ID NO: 83) |

TABLE 6
Human IL-12A (p35) and IL-12B (p40) Exemplary Construct
Nucleic Acid Sequences

| SEQ ID NOs | Name | Nucleic Acid Sequence |
|---|---|---|
| SEQ ID NO: 84 | AAV2 5′ ITR | ctgcgcgctcgctcgctcactgaggccgcccgggcaaagcccgggcgtcgg |
| | | gcgacctttggtcgcccggcctcagtgagcgagcgagcgcgcagagaggga |
| | | gtggccaactccatcactaggggttcct |
| SEQ ID NO: 68 | CMV Enhancer | gacattgattattgactagttattaatagtaatcaattacggggtcattagttcatag |
| | | cccatatatggagttccgcgttacataacttacggtaaatggcccgcctggctga |
| | | ccgcccaacgacccccgcccattgacgtcaataatgacgtatgttcccatagta |
| | | acgccaatagggactttccattgacgtcaatgggtggactatttacggtaaactg |
| | | cccacttggcagtacatcaagtgtatcatatgccaagtacgccccctattgacgt |
| | | caatgacggtaaatggcccgcctggcattatgcccagtacatgaccttatggga |
| | | ctttcctacttggcagtacatctacgtattagtcatcgctattaccatg |
| SEQ ID NO: 85 | Chicken Beta | tcgaggtgagccccacgttctgatcactctccccatctcccccccctccccacc |
| | Actin (CBA) | cccaattttgtatttatttattttttaattattttgtgcagcgatgggggcggggggg |
| | Promoter | gggggggcgcgcgccaggcggggcggggcggggcgaggggcggggcg |
| | | gggcgaggcggagaggtgcggcggcagccaatcagagcggcgcgctccg |
| | | aaagtttccttttatggcgaggcggcggcggcggcggccctataaaaagcgaa |
| | | gcgcgcggcgggcg |
| SEQ ID NO: 86 | CAG Intron | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctcgttt |
| | | cttttctgtggctgcgtgaaagccttgaggggctccgggagggccctttgtgcg |
| | | gggggagcggctcggggggtgcgtgcgtgtgtgtgtgcgtggggagcgccg |
| | | cgtgcggctccgcgctgcccggcggctgtgagcgctgcgggcgcggcgcg |
| | | gggctttgtgcgctccgcagtgtgcgcgagaggagcgcggccgggggcggt |
| | | gccacgcggtgcggggggggctgcgaggggaacaaaggctgcgtgcggg |
| | | gtgtgtgcgtggggggggtgagcagggggtgtgggcgcggcggtcgggctg |
| | | taacccccccctgcacccccctccccgagttgctgagcacggcccggcttcgg |
| | | gtgcggggctccgtgcggggcgtggcgcggggctcgccgtgccgggcggg |
| | | gggtggcggcgggtgggggtgccgggcggggcggggccgcctcgggccg |
| | | gggagggctcgggggaggggcgcggcggcccccggagcgccggcggct |
| | | gtcgaggcgcggcgagccgcagccattgccttttatggtaatcgtgcgagagg |
| | | gcgcagggacttcctttgtcccaaatctgtgcggagccgaaatctgggaggcg |
| | | ccgccgcaccccctctagcgggcgcggggcgaagcggtgcggcgccggca |
| | | ggaaggaaatgggcggggagggccttcgtgcgtcgccgcgccgccgtcccc |
| | | ttctccctctccagcctcggggctgtccgcggggggacggctgccttcggggg |
| | | ggacggggcagggcggggttcggcttctggcgtgtgaccggcggtctagact |
| | | ctgctaaccatgttcatgccttatctattcctacagctcctgggcaacgtgctggt |
| | | tgttgtgctgtctcatcattttggcaaa |
| SEQ ID NO: 87 | Kozak | gccac |
| SEQ ID NO: 77 | IL-12A (p35) | ATGTGTCCAGCGCGCAGCCTCCTCCTTGTGGCTACCCTGGTCCTCC |
| | (“wild-type” no | TGGACCACCTCAGTTTGGCCAGAAACCTCCCCGTGGCCACTCCAGA |
| | CpG | CCCAGGAATGTTCCCATGCCTTCACCACTCCCAAAACCTGCTGAGG |
| | modification | GCCGTCAGCAACATGCTCCAGAAGGCCAGACAAACTCTAGAATTTT |
| | and no in-frame | ACCCTTGCACTTCTGAAGAGATTGATCATGAAGATATCACAAAAGA |
| | stop codon) | TAAAACCAGCACAGTGGAGGCCTGTTTACCATTGGAATTAACCAAG |
| | | AATGAGAGTTGCCTAAATTCCAGAGAGACCTCTTTCATAACTAATG |
| | | GGAGTTGCCTGGCCTCCAGAAAGACCTCTTTTATGATGGCCCTGTG |
| | | CCTTAGTAGTATTTATGAAGACTTGAAGATGTACCAGGTGGAGTTC |
| | | AAGACCATGAATGCAAAGCTTCTGATGGATCCTAAGAGGCAGATCT |
| | | TTCTAGATCAAAACATGCTGGCAGTTATTGATGAGCTGATGCAGGC |
| | | CCTGAATTTCAACAGTGAGACTGTGCCACAAAAATCCTCCCTTGAA |
| | | GAACCGGATTTTTATAAAACTAAAATCAAGCTCTGCATACTTCTTC |
| | | ATGCTTTCcggATTCGGGCAGTGACTATTGATAGAGTGATGAGCTA |
| | | TCTGAATGCTTCC |
| SEQ ID NO: 60 | Combined | agaaagagaaggagtggctcagga |
| | Furin Cleavage | |
| | Site and Linker | |
| SEQ ID NO: 61 | 2A site | gcccctgtgaaacagaccctgaactttgacctcttgaagatgctggggatgtgg |
| | | agtctaatcctggtcca |
| SEQ ID NO: 8 | IL-12B (p40) | ATGTGTCACCAGCAGTTGGTCATCTCTTGGTTTTCCCTGGTTTTTC |
| | (“wild-type” no | TGGCATCTCCCCTCGTGGCCATATGGGAACTGAAGAAAGATGTTTA |
| | CpG | TGTCGTAGAATTGGATTGGTATCCGGATGCCCCTGGAGAAATGGTG |
| | modification | GTCCTCACCTGTGACACCCCTGAAGAAGATGGTATCACCTGGACCT |
| | and with stop | TGGACCAGAGCAGTGAGGTCTTAGGCTCTGGCAAAACCCTGACCAT |
| | codon) | CCAAGTCAAAGAGTTTGGAGATGCTGGCCAGTACACCTGTCACAAA |
| | | GGAGGCGAGGTTCTAAGCCATTCGCTCCTGCTGCTTCACAAAAAGG |
| | | AAGATGGAATTTGGTCCACTGATATTTTAAAGGACCAGAAAGAACC |
| | | CAAAAATAAGACCTTTCTAAGATGCGAGGCCAAGAATTATTCTGGA |
| | | CGTTTCACCTGCTGGTGGCTGACGACAATCAGTACTGATTTGACAT |
| | | TCAGTGTCAAAAGCAGCAGAGGCTCTTCTGACCCCCAAGGGGTGAC |
| | | GTGCGGAGCTGCTACACTCTCTGCAGAGAGAGTCAGAGGGGACAAC |
| | | AAGGAGTATGAGTACTCAGTGGAGTGCCAGGAGGACAGTGCCTGCC |
| | | CAGCTGCTGAGGAGAGTCTGCCCATTGAGGTgATGGTGGATGCCGT |
| | | TCACAAGCTCAAGTATGAAAACTACACCAGCAGCTTCTTCATCAGG |
| | | GACATCATCAAACCTGACCCACCCAAGAACTTGCAGCTGAAGCCAT |
| | | TAAAGAATTCTCGGCAGGTGGAGGTCAGCTGGGAGTACCCTGACAC |
| | | CTGGAGTACTCCACATTCCTACTTCTCCCTGACATTCTGCGTTCAa |
| | | GTCCAGGGCAAGAGCAAGAGAGAAAAGAAAGATAGAGTCTTCACGG |
| | | ACAAGACCTCAGCCACGGTCATCTGCCGCAAAAATGCCAGCATTAG |
| | | CGTGCGGGCCCAGGACCGCTACTATAGCTCATCTTGGAGCGAATGG |
| | | GCATCTGTGCCCTGCAGTTGA |
| SEQ ID NO: 88 | Human growth | gatctacgggtggcatccctgtgacccctccccagtgcctctcctggccctgga |
| | hormone | agttgccactccagtgcccaccagccttgtcctaataaaattaagttgcatcatttt |
| | (HGH) poly(A) | gtctgactaggtgtccttctataatattatggggtggaggggggtggtatggagc |
| | | aaggggcaagttgggaagacaacctgtagggcctgcggggtctattgggaac |
| | | caagctggagtgcagtggcacaatcttggctcactgcaatctccgcctcctggg |
| | | ttcaagcgattctcctgcctcagcctcccgagttgttgggattccaggcatgcatg |
| | | accaggctcagctaatttttgtttttttggtagagacggggtttcaccatattggcc |
| | | aggctggtctccaactcctaatctcaggtgatctacccaccttggcctcccaaatt |
| | | gctgggattacaggcgtgaaccactgctcccttccctgtccttctg |
| SEQ ID NO: 89 | AAV2 3′ ITR | aggaacccctagtgatggagttggccactccctctctgcgcgctcgctcgctca |
| | | ctgaggccgggcgaccaaaggtcgcccgacgcccgggctttgcccgggcgg |
| | | cctcagtgagcgagcgagcgcgcagctgcagatctg |

In some aspects of the disclosure are directed to a polynucleotide comprising a nucleic acid sequence having at least 80%, 85%, 90%, 95% 96%, 97%, 98%, 99%, or 100% identity to any of SEQ ID NOs: 5-37 and 48-57, any nucleic acid shown in Table 2, Table 4, or Table 5, or any combinations thereof.

In some aspects of the disclosure are directed to a polynucleotide comprising a nucleic acid sequence having at least 80%, 85%, 90%, 95% 96%, 97%, 98%, 99%, or 100% identity to any of SEQ ID NOs: 8, 60, 61, 68, 71, 77, 82, and 84-89, any nucleic acid shown in Table 6, or any combinations thereof. In some aspects of the disclosure are directed to a polynucleotide comprising a nucleic acid sequence having at least 80%, 85%, 90%, 95% 96%, 97%, 98%, 99%, or 100% identity to SEQ ID NOs: 82.

In some aspects of the disclosure are directed to a polynucleotide encoding an amino acid sequence having at least 80%, 85%, 90%, 95% 96%, 97%, 98%, 99%, or 100% identity to SEQ ID NOs: 78, 79, or a combinations thereof.

In some aspects of the disclosure are directed to a polynucleotide encoding an amino acid sequence having at least 80%, 85%, 90%, 95% 96%, 97%, 98%, 99%, or 100% identity to SEQ ID NOs: 80, 81, or a combinations thereof.

In some aspects, the polynucleotide comprises: (i) a first nucleic acid encoding a first cytokine subunit or functional fragment thereof; and (ii) a second nucleic acid encoding a second cytokine subunit or functional fragment thereof. In some aspects, the polynucleotide comprises a nucleic acid encoding an IL-12 p35 subunit, wherein the nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77. In some aspects, the polynucleotide comprises a nucleic acid encoding the IL-12 p40 subunit, wherein the nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92. In some aspects, the polynucleotide comprises (i) a nucleic acid encoding an IL-12 p35 subunit, wherein the nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77; and (ii) a nucleic acid encoding the IL-12 p40 subunit, wherein the nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, SEQ ID NO: 92.

In some aspects, the immunomodulatory protein is a soluble natural killer (NK) cell, B cell, T cell, neutrophil, or macrophage ligand. In some aspects, the first immunomodulatory protein or functional fragment thereof or the second immunomodulatory protein or functional fragment thereof is selected from the group consisting of soluble CD40 ligand (CD40L), CD19 ligand (CD19L), CD48 ligand (CD48L), CD20 ligand (CD20L), any functional fragment thereof, and any combination thereof.

Also provided herein are polynucleotides encoding the immunomodulatory proteins or functional fragments thereof described herein that are modified, e.g., by codon/RNA modification, replacement with heterologous signal sequences, and elimination of mRNA instability elements. Methods to generate modified nucleic acids encoding an immunomodulatory protein or functional fragment thereof described herein for recombinant expression by introducing codon changes (e.g., a codon change that encodes the same amino acid due to the degeneracy of the genetic code) and/or eliminating inhibitory regions in the mRNA can be carried out by adapting the methods described in, e.g., U.S. Pat. Nos. 5,965,726; 6,174,666; 6,291,664; 6,414,132; and 6,794,498, accordingly, each of which is incorporated herein by reference in its entirety.

A polynucleotide encoding an immunomodulatory protein or functional fragment thereof can be generated from nucleic acid from a suitable source using methods well known in the art (e.g., PCR and other molecular cloning methods).

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components. In some aspects, the construct of the disclosure is a multicistronic (e.g., bicistronic) construct (e.g., comprising nucleic acids encoding cytokine (e.g., IL-12) subunits). In some aspects, the multicistronic (e.g., bicistronic) construct further comprises an F2A or IRES element. In some aspects, the multicistronic construct comprises an F2A, E2A, P2A or T2A element. In some aspects, the multicistronic construct comprises an F2A element.

In some aspects, the viral vector comprises a polynucleotide comprising coding regions for two or more immunomodulatory proteins of interest or functional fragments thereof. The two or more immunomodulatory proteins of interest or functional fragments thereof can be the same or different from each other. When it is desired to include coding regions for two or more immunomodulatory proteins of interest or functional fragments thereof, two or more individual polypeptide chains, or two or more subunits of a immunomodulatory protein of interest or functional fragment thereof in one viral vector, each additional coding region beyond the first is preferably linked to an element that facilitates co-expression of the proteins in host cells, such as an internal ribosomal entry sequence (IRES) element (See e.g., U.S. Pat. No. 4,937,190), or a 2A element. In some aspects, IRES or 2A elements (e.g., F2A, E2A, P2A or T2A) can be used when a single vector comprises sequences encoding each subunit of a multi-subunit protein.

The viral vector can include coding regions for two or more immunomodulatory proteins of interest or functional fragments thereof. For example, the viral vector can include the coding region for a first immunomodulatory protein of interest or functional fragment thereof and the coding region for a second immunomodulatory protein of interest or functional fragments thereof. The first immunomodulatory protein of interest or functional fragment thereof and the second immunomodulatory protein of interest or functional fragment thereof can be the same or different. In some aspects, the viral vector can include the coding region(s) for a third or a fourth immunomodulatory protein of interest or functional fragments thereof. The third and the fourth immunomodulatory protein of interest or functional fragments thereof can be the same or different. The total length of the two or more immunomodulatory proteins of interest or functional fragments thereof encoded by one viral vector can vary. For example, the total length of the two or more proteins can be at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer.

In some aspects, provided herein is a polynucleotide comprising a nucleic acid encoding a cytokine or functional fragment thereof, wherein the cytokine is selected from the group consisting of interleukin (IL)-12, tumor necrosis factor alpha (TNF-α), a type I interferon (INF), a type II IFN, IL-2, IL-15, IL-21, IL-23, IL-27, IL-18, IL-1, IL-3, IL-4, IL-5, IL-6, IL-7, IL-8, IL-9, IL-10, IL17, IL33, TL1A, CD40L, granulocyte-macrophage colony-stimulating factor (GM-CSF), any functional fragment thereof, and any combination thereof.

In some aspects, the cytokine is IL-12, IL-18, IL-21, or a functional fragment thereof. In some aspects, the polynucleotide comprises: (i) a first nucleic acid encoding a first IL-12 subunit or functional fragment thereof; and (ii) a second nucleic acid encoding a second IL-12 subunit or functional fragment thereof. In some aspects, the nucleic acid comprises a nucleic acid encoding an IL-12 p35 subunit and/or a nucleic acide encoding an IL-12 p40 subunit. In some aspects, the nucleic acid comprises a nucleic acid sequence having at least 85%, 90%, 95% 96%, 97%, 98%, 99%, or 100% identity any of SEQ ID NO: 5-10 or any nucleic acid sequence in Table 2, or any combination thereof. In some aspects, the polynucleotide comprises (i) a nucleic acid encoding an IL-12 p35 subunit, wherein the nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77; and/or (ii) a nucleic acid encoding the IL-12 p40 subunit, wherein the nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, SEQ ID NO: 92.

The Kozak consensus sequence, Kozak consensus or Kozak sequence, is known as a sequence which occurs on eukaryotic mRNA and has the consensus (gcc)gccRccAUGG, where R is a purine (adenine or guanine) three bases upstream of the start codon (AUG), which is followed by another “G.” In some aspects, the vector comprises a nucleotide sequence having at least about 85%, at least about 90%, at least about 95% sequence identity, or more to the Kozak consensus sequence. In some aspects, the vector comprises a Kozak consensus sequence. In some aspects, the vector includes a Kozak consensus sequence after the polynucleotide encoding one or more immunomodulatory proteins of interest of functional fragments thereof is inserted into the vector, e.g., at the restrict site downstream of the promoter. For example, the vector can include a nucleotide sequence of GCCGCCATG (SEQ ID NO: 2), where the ATG is the start codon of the protein of interest. In some aspects, the vector comprises a nucleotide sequence of GCGGCCGCCATG (SEQ ID NO: 3), where the ATG is the start codon of the protein of interest.

The immunomodulatory protein of interest can be isolated and purified, if desired, in accordance with conventional methods known to those skilled in the art. For example, a lysate can be prepared of the expression host cells and the lysate can be purified using HPLC, hydrophobic interaction chromatography (HIC), anion exchange chromatography, cation exchange chromatography, size exclusion chromatography, ultrafiltration, gel electrophoresis, affinity chromatography, and/or other purification techniques.

### III. Constructs

Some aspects of the disclosure are directed to a nucleic acid construct or an expression construct (e.g., comprising an expression cassette) having a eukaryotic promoter operably linked to a DNA of interest that encodes an immunomodulatory protein, one or more subunits thereof, or functional fragment thereof. In some aspects, the constructs containing the DNA sequence (or the corresponding RNA sequence) which can be used in accordance with the disclosure can be any eukaryotic expression construct containing the DNA or the RNA sequence of interest. For example, a plasmid or viral construct (e.g. an AAV vector) can be cleaved to provide linear DNA having ligatable termini. These termini are bound to exogenous DNA having complementary, like ligatable termini to provide a biologically functional recombinant DNA molecule having an intact replicon and a desired phenotypic property. In some aspects, the construct is capable of replication in both eukaryotic and prokaryotic hosts, which constructs can comprise backbones known in the art and are commercially available.

In some aspects, the exogenous (i.e., donor) DNA used in the disclosure is obtained from suitable cells, and the constructs can be prepared using techniques well known in the art. Likewise, techniques for obtaining expression of exogenous DNA or RNA sequences in a genetically altered host cell are known in the art (see e.g., Kormal et al., Proc. Natl. Acad. Sci. USA, 84:2150-2154 (1987); Sambrook et al. Molecular Cloning: a Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

In some aspects, the expression construct of the disclosure is a multicistronic (e.g., bicistronic) construct (e.g., comprising nucleic acids encoding cytokine (e.g., IL-12) subunits). In some aspects, the multicistronic (e.g., bicistronic) construct further comprises an F2A or IRES element. In some aspects, the multicistronic construct comprises an F2A element.

In some aspects, the disclosure is directed to an expression construct (e.g., comprising an expression cassette) comprising or defined by a formula selected from:

X-I1-P1-E-P2-I2-X′-T-Y; (a)

Y-I1-P1-E-P2-I2-X-T-X′; (b)

pA-X-I1-L-P1-E-P2-I2-X-T-Y-pA; (c)

pA-Y-I1-L-P1-E-P2-I2-X-T-X′-pA; (d)

E-P1-I1-X-T-X′-pA; or (e)

P1-X-T-X′-pA. (f)

wherein X and X′ encode subunits of a first immunomodulatory molecule; I1 and I2 is an intron; P1 and P2 is a promoter; E is an enhancer; T is a translation modification sequence; Y encodes a second immunomodulatory molecule; L is a long-terminal repeat; and pA is a polyA sequence. In some aspects, X can be a nucleic acid encoding an IL-12 p35 subunit or a functional fragment thereof or a nucleic acid encoding an IL-12 p40 subunit or a functional fragment thereof. In some aspects, X′ can be a nucleic acid encoding an IL-12 p35 subunit or a functional fragment thereof or a nucleic acid encoding an IL-12 p40 subunit or a functional fragment thereof.

In some aspects, E is a CMV enhancer, P1 is CBA promoter, I1 or I2 is a CAG intron, X is a nucleic acid encoding an IL-12 p35 subunit or a functional fragment thereof, T is a furin cleavage sequence followed by the 2A self-processing peptide sequence (F2A), X′ is a nucleic acid encoding an IL-12 p40 subunit or a functional fragment thereof, and pA is a a human growth hormone (HGH) pA.

In some aspects, provided herein is construct comprising a polynucleotide comprising a CMV enhancer, a CBA promoter operably linked to a first nucleic acid encoding a human IL-12 p35 subunit or a functional fragment thereof, a furin cleavage sequence followed by a 2A self-processing peptide (F2A), a second nucleic acid encoding a human IL-12 p40 subunit or a functional fragment thereof, and a human growth hormone (HGH) pA sequence. In some aspects, the construct further comprises an intron sequence (e.g., a CAG intron) and a Kozak sequence.

In some aspects, the nucleic acid encoding an IL-12 p35 subunit does not include an in-frame stop codon (e.g., TGA, TAG, and TAA).

In some aspects, the construct contains a promoter to facilitate expression of the DNA of interest (e.g., a first or second immunomodulatory molecule, one or more subunits thereof, or a functional fragment thereof) within a tumor. In some aspects, the promoter is a strong, eukaryotic promoter such as a promoter from cytomegalovirus (CMV), mouse mammary tumor virus (MMTV), Rous sarcoma virus (RSV), or adenovirus. Exemplary promoters include, but are not limited to the promoter from the immediate early gene of human CMV (Boshart et al., Cell 41:521-530 (1985) and the promoter from the long terminal repeat (LTR) of RSV (Gorman et al., Proc. Natl. Acad. Sci. USA 79:6777-6781 (1982)). In some aspects, the promoter is a CBA promoter, a CMV promoter, an EF-1a (Elongation Factor 1a) promoter, aRSV (Rous Sarcoma Virus) promoter, an Ubiquitin (UbC) promoter, or any combination thereof.

Alternatively, the promoter used can be a tissue-specific promoter. In some aspects, the promoter can target expression to a tumor or limit expression to the tumor (e.g., to the tumor being treated by a method of the disclosure).

In some aspects, the construct (e.g., expression construct) can comprise two or more promoters, e.g., a first promoter for the first nucleic acid encoding a first immunomodulatory protein, subunit thereof, or functional fragment thereof and a second promoter for the second nucleic acid sequence encoding a second immunomodulatory protein, subunit thereof, or functional fragment thereof. In some aspects, the two or more promoters are the same. In some aspects the two or more promoters are different. In some aspects, the promoter comprises a CBA promoter, a CMV promoter, an EF-1a (Elongation Factor 1a) promoter, aRSV (Rous Sarcoma Virus) promoter, an Ubiquitin (UbC) promoter, or any combination thereof.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 74.

In some aspects, the construct (e.g., expression construct) can comprise a translation modification sequence. In some aspects, the translation modification sequence is selected from an internal ribosomal entry site (IRES) sequence, a 2A self-processing peptide (e.g., a F2A peptide, a P2A peptide, an E2A peptide, or a T2A peptide), a furin cleavage sequence, or any combination thereof. In some aspects, the constructs of the disclosure can include an internal ribosomal entry site (IRES) sequence, a F2A peptide, a P2A peptide, an E2A peptide, a T2A peptide, a furin cleavage sequence, or a combination thereof. In some aspects, the IRES comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 59. In some aspects, the furin cleavage site comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 60. In some aspects, the 2a site comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 61.

In some aspects, the constructs of the disclosure (e.g., expression constructs) can further comprise an enhancer sequence, an intron sequence, a long terminal repeat (LTR) sequence, or any combination thereof. In some aspects, the enhancer sequence is a CMV enhancer sequence. In some aspects, the CMV enhancer comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 68. In some aspects, the intron is an SV40 intron. In some aspects, the intron is a chimera of the human betaglobin intron and the immunoglobulin heavy chain intron. In some aspects, the intron is a CAG intron. In some aspects, the intron has a nucleotide sequence with at least 85%, 86%, 87%, 88%, 89%, 90%, 91%, 92%, 93%, 94%, 95%, 96%, 97%, 98%, 99%, or 100% identical to any of SEQ ID NOs: 66, 71, 76, and 86.

In some aspects, the constructs of the disclosure (e.g., expression constructs) can further comprise a poly(A) (pA) sequence. In some aspects, the pA sequence is a synthetic pA (SYNpA) sequence, a BGHpA sequence, a HGHpA, or a combination thereof. In some aspects, the SYNpA comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 72. In some aspects, the BGHpA comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 73. In some aspects, the HGHpA comprises a nucleotide sequence with at least 85%, 86%, 87%, 88%, 89%, 90%, 91%, 92%, 93%, 94%, 95%, 96%, 97%, 98%, 99%, or 100% identical to SEQ ID NO: 88.

In some aspects, the constructs of the disclosure (e.g., expression constructs) can include the first nucleic acid comprising a promoter operably linked to an IL-12 p35 gene or functional fragment thereof, an IRES sequence, and an IL-15 gene or a functional fragment thereof; and the second nucleic acid comprising a promoter operably linked to an IL-12 p40 gene or functional fragment thereof. In some aspects, the constructs of the disclosure can include the first nucleic acid comprising a promoter operably linked to an IL-12 p35 gene or functional fragment thereof, a furin cleavage sequence and/or a 2A self-processing peptide (e.g., an F2A, E2A, P2A or T2A self-processing peptide), and an IL-12 p40 gene or a functional fragment thereof, and the second nucleic acid comprising a promoter operably linked to an IL-15 gene or functional fragment thereof.

In some aspects, the constructs of the disclosure (e.g., expression constructs) can include the first nucleic acid comprising a promoter operably linked to a nucleic acid encoding an IL-12 p35 subunit or functional fragment thereof, an IRES sequence; and the second nucleic acid comprising a promoter operably linked to a nucleic acid encoding an IL-12 p40 subunit or functional fragment thereof. In some aspects, the constructs of the disclosure (e.g., expression constructs) can include the first nucleic acid comprising a promoter operably linked to a nucleic acid encoding an IL-12 p40 subunit or functional fragment thereof, an IRES sequence; and the second nucleic acid comprising a promoter operably linked to a nucleic acid encoding an IL-12 p35 subunit or functional fragment thereof. In some aspects, the constructs of the disclosure can include the first nucleic acid comprising a promoter operably linked to a nucleic acid encoding an IL-12 p35 subunit or functional fragment thereof, a furin cleavage sequence and/or a 2A self-processing peptide (e.g., an F2A, E2A, P2A or T2A self-processing peptide), and a second nucleic acid encoding an IL-12 p40 subunit or a functional fragment thereof. In some aspects, the constructs of the disclosure can include the first nucleic acid comprising a promoter operably linked to a nucleic acid encoding an IL-12 p40 subunit or functional fragment thereof, a furin cleavage sequence and/or a 2A self-processing peptide, and a second nucleic acid encoding an IL-12 p35 subunit or a functional fragment thereof.

In some aspects, first nucleic acid (e.g., the nucleic acid encoding an IL-12 p35 subunit) does not include an in-frame stop codon (e.g., TGA, TAG, and TAA).

In some aspects, the first nucleic acid further comprises an intron sequence, a pA sequence, an enhancer sequence, or any combination thereof. In some aspects, the first nucleic acid further comprises an intron sequence and a human growth hormone (HGH) pA sequence. In some aspects, the first nucleic acid further comprises an intron sequence and a bovine growth hormone (BGH) pA sequence.

In some aspects, the first nucleic acid further comprises an intron sequence (e.g, a CAG intron). In some aspects, the second nucleic acid further comprises an intron sequence, a pA sequence, an enhancer sequence, or any combination thereof. In some aspects, the second nucleic acid further comprises an SV40 intron sequence and a synthetic (SYN) pA sequence.

In some aspects, the promoter of the first nucleic acid comprises a CBA promoter, a CMV promoter, a EF-1a (Elongation Factor 1a) promoter, aRSV (Rous Sarcoma Virus) promoter, an Ubiquitin (UbC) promoter, or any combination thereof. In some aspects, the promoter of the second nucleic acid comprises a CBA promoter, a CMV promoter, a EF-1a (Elongation Factor 1a) promoter, aRSV (Rous Sarcoma Virus) promoter, an Ubiquitin (UbC) promoter, a CMV enhancer fused to a EF1α promoter, CMV promoter fused to an SV40 intron, a CMV promoter fused to a CMV enhancer, or any combination thereof. In some aspects, the one or more promoters has a nucleic acid sequence comprising at least 85%, 86%, 87%, 88%, 89%, 90%, 91%, 92%, 93%, 94%, 95%, 96%, 97%, 98%, 99%, or 100% identical to any one of SEQ ID NOs: 67-71 or 85.

In some aspects, the constructs of the disclosure (e.g., expression constructs) can also comprise a microRNA (miR) binding site. In some aspects, the microRNA binding site is included to improve safety and allow tumor-specific expression. In some aspects, the microRNA binding site can act as conditional switches to control expression based on the expression of its complementary microRNA that is selected because it is absent or expressed at low level in tumors compared to expression in normal healthy tissues. In some aspects, inclusion of a microRNA binding site can restrict expression of immunomodulatory proteins disclosed herein to tumor cells while sparing normal tissues. In some aspects, the microRNA target binding sites are selected from a binding site for miR-1-3p, miR-122-5p, miR-124-3p, miR-126-3p, miR-128-3p, miR-137, miR-143-3p, miR-204-5p, miR-217, miR-219a-5p, or any combination thereof. In some aspects, the miRNA binding site is a miR-142 binding site. In some aspects, In some aspects, the miRNA binding site comprises four miR-142 binding sites separated by spacers. In some aspects, the miR-142 binding site has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 64. In some aspects, the 4× miR-142 binding site has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 65.

In some aspects, provided herein is a construct comprising a polynucleotide comprising a CMV enhancer (e.g., SEQ ID NO: 68), a CBA promoter (e.g., SEQ ID NO: 85) operably linked to a first nucleic acid encoding a human IL-12 p35 subunit or a functional fragment thereof (e.g., SEQ ID NO: 77), a furin cleavage sequence followed by a 2A self-processing peptide (F2A), a second nucleic acid encoding a human IL-12 p40 subunit or a functional fragment thereof (e.g., SEQ ID NO: 8), and a human growth hormone (HGH) pA sequence (e.g., SEQ ID NO: 88). In some aspects, the construct further comprises an intron sequence (e.g., a CAG intron, e.g., SEQ ID NO: 86) and a Kozak sequence. In some aspects, the construct comprises a CMV enhancer, a CBA promoter, and a CAG intron (e.g., SEQ ID NO: 71).

In some aspects, the constructs of the disclosure (e.g., expression constructs) can also include other components such as a marker (e.g., an antibiotic resistance gene (such as an ampicillin resistance gene) or β-galactosidase) to aid in selection of cells containing and/or expressing the construct, an origin of replication for stable replication of the construct in a bacterial cell (preferably, a high copy number origin of replication), a nuclear localization signal, or other elements which facilitate production of the DNA construct, the protein encoded thereby, or both.

For eukaryotic expression, the construct (e.g., expression construct) can comprise at a minimum a eukaryotic promoter operably linked to a DNA of interest, which is in turn operably linked to a polyadenylation sequence. The polyadenylation signal sequence can be selected from any of a variety of polyadenylation signal sequences known in the art. In some aspects, the polyadenylation signal sequence is the SV40 early polyadenylation signal sequence. The construct can also include one or more introns, which can increase levels of expression of the DNA of interest, particularly where the DNA of interest is a cDNA (e.g., contains no introns of the naturally-occurring sequence). Any of a variety of introns known in the art can be used (e.g., the human β-globin intron, which is inserted in the construct at a position 5′ to the DNA of interest).

The DNA of interest can be inserted into a construct so that the immunomodulatory protein, one or more subunits thereof, or functional fragment thereof is expressed as a fusion protein (e.g., a fusion protein having β-galactosidase or a portion thereof at the N-terminus and the therapeutic protein at the C-terminal portion). Production of a fusion protein can facilitate identification of transformed cells expressing the protein (e.g., by enzyme-linked immunosorbent assay (ELISA) using an antibody which binds to the fusion protein).

In some aspects, the nucleic acids, the constructs, polynucleotides, and/or expression cassettes disclosed herein comprise sequences with modified CpG content; and/or reduced or eliminated toll-like receptor 9 (TLR9) stimulatory motifs.

The vectors for delivery of the DNA of interest can be either viral or non-viral, or can be composed of naked DNA admixed with an adjuvant such as viral particles (e.g., AAV particle) or cationic lipids or liposomes. An “adjuvant” is a substance that does not by itself produce the desired effect, but acts to enhance or otherwise improve the action of the active compound. The precise vector and vector formulation used will depend upon several factors such as the tumor targeted for gene transfer.

In certain aspects, a composition comprising a delivery vector, e.g., a viral vector, comprising a nucleic acid construct or an expression construct comprising a first nucleic acid encoding a first immunomodulatory protein, subunit thereof, or functional fragment thereof and/or a second nucleic acid encoding a second immunomodulatory protein, subunit thereof, or functional fragment thereof is suitable for intratumoral delivery.

In certain aspects, a composition comprising a delivery vector, e.g., a viral vector, comprising a nucleic acid construct or an expression construct comprising a first nucleic acid encoding a first immunomodulatory protein, subunit thereof, or functional fragment thereof and/or a second nucleic acid encoding a second immunomodulatory protein, subunit thereof, or functional fragment thereof is suitable for intravenous delivery.

In certain aspects, a composition comprising a delivery vector, e.g., a viral vector, comprising a nucleic acid construct or an expression construct comprising a first nucleic acid encoding a first immunomodulatory protein, subunit thereof, or functional fragment thereof and/or a second nucleic acid encoding a second immunomodulatory protein, subunit thereof or functional fragment thereof is suitable for a delivery to a lymphoid organ (e.g., a spleen, a lymph node, or a bone marrow).

In some aspects, the constructs disclosed herein comprise one or more of the elements listed in Table 7.

TABLE 7
Nucleic Acid Sequences of Elements in
Constructs to Generate a Cytokine

| SEQ ID | | |
|---|---|---|
| NOs | Name | Nucleic Acid Sequence |
| SEQ ID | IRES | GCCCCTCTCCCTCCCCCCCC |
| NO: 59 | | CCTAACGTTACTGGCCGAAG |
| | | CCGCTTGGAATAAGGCCGGT |
| | | GTGCGTTTGTCTATATGTTA |
| | | TTTTCCACCATATTGCCGTC |
| | | TTTTGGCAATGTGAGGGCCC |
| | | GGAAACCTGGCCCTGTCTTC |
| | | TTGACGAGCATTCCTAGGGG |
| | | TCTTTCCCCTCTCGCCAAAG |
| | | GAATGCAAGGTCTGTTGAAT |
| | | GTCGTGAAGGAAGCAGTTCC |
| | | TCTGGAAGCTTCTTGAAGAC |
| | | AAACAACGTCTGTAGCGACC |
| | | CTTTGCAGGCAGCGGAACCC |
| | | CCCACCTGGCGACAGGTGCC |
| | | TCTGCGGCCAAAAGCCACGT |
| | | GTATAAGATACACCTGCAAA |
| | | GGCGGCACAACCCCAGTGCC |
| | | ACGTTGTGAGTTGGATAGTT |
| | | GTGGAAAGAGTCAAATGGCT |
| | | CTCCTCAAGCGTATTCAACA |
| | | AGGGGCTGAAGGATGCCCAG |
| | | AAGGTACCCCATTGTATGGG |
| | | ATCTGATCTGGGGCCTCGGT |
| | | ACACATGCTTTACATGTGTT |
| | | TAGTCGAGGTTAAAAAAACG |
| | | TCTAGGCCCCCCGAACCACG |
| | | GGGACGTGGTTTTCCTTTGA |
| | | AAAACACGATGATAATATGG |
| | | CCACA |
| SEQ ID | Furin cleaveage | AGAAAGAGAAGGAGTGGCTC |
| NO: 60 | site | AGGA |
| SEQ ID | 2A site | GCCCCTGTGAAACAGACCCT |
| NO: 61 | | GAACTTTGACCTCTTGAAGC |
| | | TTGCTGGGGATGTGGAGTCT |
| | | AATCCTGGTCCA |
| SEQ ID | IL-2 leader | ATGTACAGGATGCAACTCCT |
| NO: 62 | | GTCTTGCATTGCACTAAGTC |
| | | TTGCACTTGTCACAAACTCG |
| SEQ ID | IL-10 leader | ATGCACAGCTCAGCACTGCT |
| NO: 63 | | CTGTTGCCTGGTCCTCCTGA |
| | | CTGGGGTGAGGGCC |
| SEQ ID | miR-142 | TCCATAAAGTAGGAAACACT |
| NO: 64 | binding | ACA |
| | site | |
| SEQ ID | 4x miR-142 | TCCATAAAGTAGGAAACACT |
| NO: 65 | binding site | ACActatTCCATAAAGTAGG |
| | | AAACACTACAtcacTCCATA |
| | | AAGTAGGAAACACTACAagt |
| | | cTCCATAAAGTAGGAAACAC |
| | | TACA |
| SEQ ID | SV40 intron | GTAAGTTTAGTCTTTTTGTC |
| NO: 66 | | TTTTATTTCAGGTCCCGGAT |
| | | CCGGTGGTGGTGCAAATCAA |
| | | AGAACTGCTCCTCAGTGGAT |
| | | GTTGCCTTTACTTCTAG |
| SEQ ID | EF-1α | GCTCCGGTGCCCGTCAGTGG |
| NO: 67 | | GCAGAGCGCACATCGCCCAC |
| | | AGTCCCCGAGAAGTTGGGGG |
| | | GAGGGGTCGGCAATTGAACC |
| | | GGTGCCTAGAGAAGGTGGCG |
| | | CGGGGTAAACTGGGAAAGTG |
| | | ATGTCGTGTACTGGCTCCGC |
| | | CTTTTTCCCGAGGGTGGGGG |
| | | AGAACCGTATATAAGTGCAG |
| | | TAGTCGCCGTGAACGTTCTT |
| | | TTTCGCAACGGGTTTGCCGC |
| | | CAGAACACAG |
| SEQ ID | CMV enhancer | GACATTGATTATTGACTAGT |
| NO: 68 | | TATTAATAGTAATCAATTAC |
| | | GGGGTCATTAGTTCATAGCC |
| | | CATATATGGAGTTCCGCGTT |
| | | ACATAACTTACGGTAAATGG |
| | | CCCGCCTGGCTGACCGCCCA |
| | | ACGACCCCCGCCCATTGACG |
| | | TCAATAATGACGTATGTTCC |
| | | CATAGTAACGCCAATAGGGA |
| | | CTTTCCATTGACGTCAATGG |
| | | GTGGACTATTTACGGTAAAC |
| | | TGCCCACTTGGCAGTACATC |
| | | AAGTGTATCATATGCCAAGT |
| | | ACGCCCCCTATTGACGTCAA |
| | | TGACGGTAAATGGCCCGCCT |
| | | GGCATTATGCCCAGTACATG |
| | | ACCTTATGGGACTTTCCTAC |
| | | TTGGCAGTACATCTACGTAT |
| | | TAGTCATCGCTATTACCATG |
| SEQ ID | CMVp | GTGATGCGGTTTTGGCAGTA |
| NO: 69 | | CATCAATGGGCGTGGATAGC |
| | | GGTTTGACTCACGGGGATTT |
| | | CCAAGTCTCCACCCCATTGA |
| | | CGTCAATGGGAGTTTGTTTT |
| | | GGCACCAAAATCAACGGGAC |
| | | TTTCCAAAATGTCGTAACAA |
| | | CTCCGCCCCATTGACGCAAA |
| | | TGGGCGGTAGGCGTGTACGG |
| | | TGGGAGGTCTATATAAGCAG |
| | | AGCTCGTTTAGTGAACCG |
| SEQ ID | CMV ehancer | GACATTGATTATTGACTAGT |
| NO: 70 | and promoter | TATTAATAGTAATCAATTAC |
| | (CMVe/p) | GGGGTCATTAGTTCATAGCC |
| | | CATATATGGAGTTCCGCGTT |
| | | ACATAACTTACGGTAAATGG |
| | | CCCGCCTGGCTGACCGCCCA |
| | | ACGACCCCCGCCCATTGACG |
| | | TCAATAATGACGTATGTTCC |
| | | CATAGTAACGCCAATAGGGA |
| | | CTTTCCATTGACGTCAATGG |
| | | GTGGACTATTTACGGTAAAC |
| | | TGCCCACTTGGCAGTACATC |
| | | AAGTGTATCATATGCCAAGT |
| | | ACGCCCCCTATTGACGTCAA |
| | | TGACGGTAAATGGCCCGCCT |
| | | GGCATTATGCCCAGTACATG |
| | | ACCTTATGGGACTTTCCTAC |
| | | TTGGCAGTACATCTACGTAT |
| | | TAGTCATCGCTATTACCATG |
| | | GTGATGCGGTTTTGGCAGTA |
| | | CATCAATGGGCGTGGATAGC |
| | | GGTTTGACTCACGGGGATTT |
| | | CCAAGTCTCCACCCCATTGA |
| | | CGTCAATGGGAGTTTGTTTT |
| | | GGCACCAAAATCAACGGGAC |
| | | TTTCCAAAATGTCGTAACAA |
| | | CTCCGCCCCATTGACGCAAA |
| | | TGGGCGGTAGGCGTGTACGG |
| | | TGGGAGGTCTATATAAGCAG |
| | | AGCTCGTTTAGTGAACCG |
| SEQ ID | CAG Promoter | GTCGACATTGATTATTGACT |
| NO: 71 | | AGTTATTAATAGTAATCAAT |
| | | TACGGGGTCATTAGTTCATA |
| | | GCCCATATATGGAGTTCCGC |
| | | GTTACATAACTTACGGTAAA |
| | | TGGCCCGCCTGGCTGACCGC |
| | | CCAACGACCCCCGCCCATTG |
| | | ACGTCAATAATGACGTATGT |
| | | TCCCATAGTAACGCCAATAG |
| | | GGACTTTCCATTGACGTCAA |
| | | TGGGTGGACTATTTACGGTA |
| | | AACTGCCCACTTGGCAGTAC |
| | | ATCAAGTGTATCATATGCCA |
| | | AGTACGCCCCCTATTGACGT |
| | | CAATGACGGTAAATGGCCCG |
| | | CCTGGCATTATGCCCAGTAC |
| | | ATGACCTTATGGGACTTTCC |
| | | TACTTGGCAGTACATCTACG |
| | | TATTAGTCATCGCTATTACC |
| | | ATGGGTCGAGGTGAGCCCCA |
| | | CGTTCTGCTTCACTCTCCCC |
| | | ATCTCCCCCCCCTCCCCACC |
| | | CCCAATTTTGTATTTATTTA |
| | | TTTTTTAATTATTTTGTGCA |
| | | GCGATGGGGGCGGGGGGGGG |
| | | GGGGGCGCGCGCCAGGCGGG |
| | | GCGGGGCGGGGCGAGGGGCG |
| | | GGGCGGGGCGAGGCGGAGAG |
| | | GTGCGGCGGCAGCCAATCAG |
| | | AGCGGCGCGCTCCGAAAGTT |
| | | TCCTTTTATGGCGAGGCGGC |
| | | GGCGGCGGCGGCCCTATAAA |
| | | AAGCGAAGCGCGCGGCGGGC |
| | | GGGAGTCGCTGCGCGCTGCC |
| | | TTCGCCCCGTGCCCCGCTCC |
| | | GCCGCCGCCTCGCGCCGCCC |
| | | GCCCCGGCTCTGACTGACCG |
| | | CGTTACTCCCACAGGTGAGC |
| | | GGGCGGGACGGCCCTTCTCC |
| | | TCCGGGCTGTAATTAGCGCT |
| | | TGGTTTAATGACGGCTCGTT |
| | | TCTTTTCTGTGGCTGCGTGA |
| | | AAGCCTTGAGGGGCTCCGGG |
| | | AGGGCCCTTTGTGCGGGGGG |
| | | AGCGGCTCGGGGGGTGCGTG |
| | | CGTGTGTGTGTGCGTGGGGA |
| | | GCGCCGCGTGCGGCTCCGCG |
| | | CTGCCCGGCGGCTGTGAGCG |
| | | CTGCGGGCGCGGCGCGGGGC |
| | | TTTGTGCGCTCCGCAGTGTG |
| | | CGCGAGAGGAGCGCGGCCGG |
| | | GGGCGGTGCCACGCGGTGCG |
| | | GGGGGGGCTGCGAGGGGAAC |
| | | AAAGGCTGCGTGCGGGGTGT |
| | | GTGCGTGGGGGGGGTGAGCA |
| | | GGGGGTGTGGGCGCGGCGGT |
| | | CGGGCTGTAACCCCCCCCTG |
| | | CACCCCCCTCCCCGAGTTGC |
| | | TGAGCACGGCCCGGCTTCGG |
| | | GTGCGGGGCTCCGTGCGGGG |
| | | CGTGGCGCGGGGCTCGCCGT |
| | | GCCGGGCGGGGGGTGGCGGC |
| | | GGGTGGGGGTGCCGGGCGGG |
| | | GCGGGGCCGCCTCGGGCCGG |
| | | GGAGGGCTCGGGGGAGGGGC |
| | | GCGGCGGCCCCCGGAGCGCC |
| | | GGCGGCTGTCGAGGCGCGGC |
| | | GAGCCGCAGCCATTGCCTTT |
| | | TATGGTAATCGTGCGAGAGG |
| | | GCGCAGGGACTTCCTTTGTC |
| | | CCAAATCTGTGCGGAGCCGA |
| | | AATCTGGGAGGCGCCGCCGC |
| | | ACCCCCTCTAGCGGGCGCGG |
| | | GGCGAAGCGGTGCGGCGCCG |
| | | GCAGGAAGGAAATGGGCGGG |
| | | GAGGGCCTTCGTGCGTCGCC |
| | | GCGCCGCCGTCCCCTTCTCC |
| | | CTCTCCAGCCTCGGGGCTGT |
| | | CCGCGGGGGGACGGCTGCCT |
| | | TCGGGGGGGACGGGGCAGGG |
| | | CGGGGTTCGGCTTCTGGCGT |
| | | GTGACCGGCGGTCTAGACTC |
| | | TGCTAACCATGTTCATGCCT |
| | | TCTTCTCTTTCCTACAGCTC |
| | | CTGGGCAACGTGCTGGTTGT |
| | | TGTGCTGTCTCATCATTTTG |
| | | GCAAA |
| SEQ ID | Synthetic | AATAAAAGATCTTTATTTTC |
| NO: 72 | poly(A) | ATTAGATCTGTGTGTTGGTT |
| | | TTTTGTGTG |
| SEQ ID | BGH poly(A) | CTGTGCCTTCTAGTTGCCAG |
| NO: 73 | | CCATCTGTTGTTTGCCCCTC |
| | | CCCCGTGCCTTCCTTGACCC |
| | | TGGAAGGTGCCACTCCCACT |
| | | GTCCTTTCCTAATAAAATGA |
| | | GGAAATTGCATCGCATTGTC |
| | | TGAGTAGGTGTCATTCTATT |
| | | CTGGGGGGTGGGGTGGGGCA |
| | | GGACAGCAAGGGGGAGGATT |
| | | GGGAAGACAATAGCAGGCAT |
| | | GCTGGGGATGCGGTGGGCTC |
| | | TATG |
| SEQ ID | Pause element | AACATACGCTCTCCATCAAA |
| NO: 74 | | ACAAAACGAAACAAAACAAA |
| | | CTAGCAAAATAGGCTGTCCC |
| | | CAGTGCAAGTGCAGGTGCCA |
| | | GAACATTTCTCT |
| SEQ ID | 5′ LTR | GGGGCTCGCATCTCTCCTTC |
| NO: 75 | | ACGCGCCCGCCGCCCTACCT |
| | | GAGGCCGCCATCCACGCCGG |
| | | TTGAGTCGCGTTCTGCCGCC |
| | | TCCCGCCTGTGGTGCCTCCT |
| | | GAACTGCGTCCGCCGTCTAG |
| | | GTAAGTTTAAAGCTCAGGTC |
| | | GAGACCGGGCCTTTGTCCGG |
| | | CGCTCCCTTGGAGCCTACCT |
| | | AGACTCAGCCGGCTCTCCAC |
| | | GCTTTGCCTGACCCTGCTTG |
| | | CTCAACTCTACGTCTTTGTT |
| | | TCGTTTTCTGTTCTGCGCCG |
| | | TTACAGATC |
| SEQ ID | Intron (chimera | GTAAGTATCAAGGTTACAAG |
| NO: 76 | of Human | ACAGGTTTAAGGAGACCAAT |
| | betaglobin | AGAAACTGGGCTTGTCGAGA |
| | intron | CAGAGAAGACTCTTGCGTTT |
| | and | CTGATAGGCACCTATTGGTC |
| | immunoglobulin | TTACTGACATCCACTTTGCC |
| | heavy chain | TTTCTCTCCACAG |
| | intron) | |

### III.A. Delivery Vectors

In some aspects, the delivery vector is a viral vector, a non-viral vectors, a plasmid, a lipid, a protein particle, a bacterial vector, a lysosome, a virus-like particle, a polymeric particle, an exosome, or a vault particle.

In certain aspects, a composition comprising a delivery vector (e.g., a viral vector, a non-viral vectors, a plasmid, a lipid, a protein particle, a bacterial vector, a lysosome, a virus-like particle, a polymeric particle, an exosome, or a vault particle) comprising a first nucleic acid encoding or comprising a first immunomodulatory protein, one or or more subunits thereof, or a functional fragment thereof and/or a second nucleic acid encoding or comprising a second immunomodulatory protein, one or more subunits thereof, or a functional fragment thereof is suitable for delivery to a tumor.

### III.A.1. Non-Viral Vectors

In some aspects, the DNA of interest can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, the non-viral vector is a bacterial vector. See e.g., Baban et al., Bioeng Bugs., 1(6):385-394 (2010).

In some aspects, the DNA of interest can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N′,N′-dimethylethylene diamine).

Lipids which can be used in accordance with the disclosure include, but are not limited to, DOPE (Dioleoyl phosphatidylethanolamine), cholesterol, and CUDMEDA (N-(5-cholestrum-3-ol 3 urethanyl)-N′,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). The concentration of the liposome solutions range from about 2.5% to 15% volume:volume, preferably about 6% to 12% volume:volume. Further exemplary methods and compositions for formulation of nucleic acid (e.g., DNA, including DNA or RNA not contained within a viral particle) for delivery according to the method of the disclosure are described in U.S. Pat. Nos. 5,892,071; 5,744,625; 5,925,623; 5,527,928; 5,824,812; 5,869,715.

In some aspects, protein particles can be used in accordance with the disclosure for polymer-based gene delivery. See e.g., Putnam et al., PNAS 98 (3): 1200-1205 (2001).

In some aspects, the DNA of interest can be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof.

In certain aspects, a composition comprising a non-viral delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for intratumoral delivery.

In certain aspects, a composition comprising a non-viral delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for intravenous delivery.

In certain aspects, a composition comprising a non-viral delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for a delivery to a lymphoid organ (e.g., a spleen, a lymph node, or a bone marrow).

### III.A.2. Viral Vectors

In general, viral vectors used in accordance with the disclosure are composed of a viral particle derived from a naturally-occurring virus which has been genetically altered to render the virus replication-defective and to express a recombinant gene of interest in accordance with the disclosure. Once the virus delivers its genetic material to a cell, it does not generate additional infectious virus but does introduce exogenous recombinant genes into the cell, preferably into the genome of the cell.

In some aspects, the viral vector is a retrovirus, an adenovirus, an adeno-associated virus (AAV), a herpes simplex virus (HSV), a cytomegalovirus (CMV), a vaccinia or a poliovirus vectors. In some aspects, retroviral vectors are less preferred since retroviruses require replicating cells and secretory glands are composed of mostly slowly replicating and/or terminally differentiated cells. In some aspects, adenovirus and AAV are preferred viral vectors since this virus efficiently infects slowly replicating and/or terminally differentiated cells. In some aspects, the delivery vector (e.g., viral vector) is selected from the group consisting of an adeno-associated viral (AAV) vector, an adenoviral vector, a lentiviral vector, or a retroviral vector.

Where a replication-deficient virus is used as the viral vector, the production of infective virus particles containing either DNA or RNA corresponding to the DNA of interest can be produced by introducing the viral construct into a recombinant cell line which provides the missing components essential for viral replication. In some aspects, transformation of the recombinant cell line with the recombinant viral vector will not result in production of replication-competent viruses, e.g., by homologous recombination of the viral sequences of the recombinant cell line into the introduced viral vector. Methods for production of replication-deficient viral particles containing a nucleic acid of interest are well known in the art and are described in, e.g., Rosenfeld et al., Science 252:431-434 (1991) and Rosenfeld et al., Cell 68:143-155 (1992) (adenovirus); U.S. Pat. No. 5,139,941 (adeno-associated virus); U.S. Pat. No. 4,861,719 (retrovirus); and U.S. Pat. No. 5,356,806 (vaccinia virus).

Certain aspects of the disclosure are directed to a viral delivery vector comprising a polynucleotide comprising a first nucleic acid encoding an IL-12 subunit (e.g., IL-12 p35) or a functional fragment thereof and/or a second nucleic acid encoding an IL-12 subunit (e.g., IL-12 p40) or functional fragment thereof disclosed herein.

Certain aspects of the disclosure are directed to a viral delivery vector comprising a polynucleotide comprising a CMV enhancer (e.g., SEQ ID NO: 68), a CBA promoter (e.g., SEQ ID NO: 71) operably linked to a first nucleic acid encoding a human IL-12 p35 subunit or a functional fragment thereof (e.g., SEQ ID NO: 77), a furin cleavage sequence followed by a 2A self-processing peptide (F2A), a second nucleic acid encoding a human IL-12 p40 subunit or a functional fragment thereof (e.g., SEQ ID NO: 8), and a human growth hormone (HGH) pA sequence (e.g., SEQ ID NO: 88). In some aspects, the construct further comprises an intron sequence (e.g., a CAG intron, e.g., SEQ ID NO: 86) and a Kozak sequence. In some aspects, the construct comprises a CMV enhancer, a CBA promoter, and a CAG intron (e.g., SEQ ID NO: 71).

In certain aspects, a composition comprising a viral delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for intratumoral delivery.

In certain aspects, a composition comprising a viral delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for intravenous delivery.

In certain aspects, a composition comprising a viral delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for a delivery to a lymphoid organ (e.g., a spleen, a lymph node, or a bone marrow).

In some aspects, the viral vector is suitable for delivery to a solid tumor (e.g., a sarcoma, a carcinomas, or a lymphoma) by direct injection. In some aspects, the solid tumor is a surface lesion. In some aspects, the solid tumor is a visceral tumor.

In some aspects of the disclosure, the viral vector disclosed herein can be administered in combination with a checkpoint inhibitor.

### IV. Adeno-Associated Virus (AAV)-Mediated Gene Therapy

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing the HNA, a suitable host cell line is transfected with an AAV vector containing the HNA, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the HNA enters and is expressed in the patient's cells. Because the patient's cells lack the rep and cap genes, as well as the accessory function genes, the rAAV virion cannot further replicate and package its genomes. Moreover, without a source of rep and cap genes, wt AAV virions cannot be formed in the patient's cells. See e.g., U.S. Appl. Publ. No. 2003/0147853.

In some aspects, AAV vectors of the present disclosure can comprise or be derived from any natural or recombinant AAV serotype. According to the present disclosure, the AAV serotype can be, but is not limited to, AAV1, AAV2, AAV3, AAV4, AAV5, AAV6, AAV7, AAV8, AAVrh8, AAV9, AAV10, AAVrh10, AAV11, and AAV12. In some aspects, the AAV serotype is AAV2.

Certain aspects of the disclosure are directed to a composition comprising an AAV delivery vector comprising a polynucleotide comprising a first nucleic acid encoding an IL-12 subunit (e.g., IL-12 p35) or a functional fragment thereof and/or a second nucleic acid encoding an IL-12 subunit (e.g., IL-12 p40) or functional fragment thereof disclosed herein.

Certain aspects of the disclosure are directed to a composition comprising an AAV delivery vector comprising a polynucleotide comprising a CMV enhancer (e.g., SEQ ID NO: 68), a CBA promoter (e.g., SEQ ID NO: 85) operably linked to a first nucleic acid encoding a human IL-12 p35 subunit or a functional fragment thereof (e.g., SEQ ID NO: 77), a furin cleavage sequence followed by a 2A self-processing peptide (F2A), a second nucleic acid encoding a human IL-12 p40 subunit or a functional fragment thereof (e.g., SEQ ID NO: 8), and a human growth hormone (HGH) pA sequence (e.g., SEQ ID NO: 88). In some aspects, the polynucleotide further comprises an intron sequence (e.g., a CAG intron, e.g., SEQ ID NO: 86) and a Kozak sequence. In some aspects, the polynucleotide comprises a CMV enhancer, a CBA promoter, and a CAG intron (e.g., SEQ ID NO: 71).

In certain aspects, a composition comprising an AAV delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for intratumoral delivery.

In certain aspects, a composition comprising an AAV delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for intravenous delivery.

In certain aspects, a composition comprising an AAV delivery vector comprising a first nucleic acid encoding or comprising a first immunomodulatory protein or functional fragment thereof and/or a second nucleic acid encoding or comprising a second immunomodulatory protein or functional fragment thereof disclosed herein is suitable for a delivery to a lymphoid organ (e.g., a spleen, a lymph node, or a bone marrow).

In some aspects, a composition comprising an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intratumoral delivery.

In some aspects, a composition comprising an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intravenous delivery.

In some aspects, a composition comprising an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for a delivery to a lymphoid organ (e.g., a spleen, a lymph node, or a bone marrow).

In some aspects, the viral vector and the checkpoint inhibitor are delivered in combination (e.g., simultaneously or sequentially). In some aspects, the viral vector described herein can be administered prior to, at the same time, or after the administration of a checkpoint inhibitor. In some aspects, the polynucleotide comprises: (i) a first nucleic acid encoding a first cytokine subunit (e.g., an IL-12 p35 subunit or an IL-12 p40 subunit) or functional fragment thereof; and (ii) a second nucleic acid encoding a second cytokine subunit (e.g., an IL-12 p35 subunit or an IL-12 p40 subunit) or functional fragment thereof. In some aspects, the nucleic acid encoding an IL-12 p35 subunit does not include an in-frame stop codon (e.g., TGA, TAG, and TAA).

In some aspects, the AAV delivery vector is suitable for delivery to a solid tumor (e.g., a sarcoma, a carcinoma, or a lymphoma) by direct injection. In some aspects, the solid tumor is a surface lesion. In some aspects, the solid tumor is a visceral tumor.

### IV.A. AAV Vector Components

### IV.A.1. Inverted Terminal Repeats (ITRs)

The AAV vectors of the present disclosure comprise a viral genome with at least one ITR region and a payload region, e.g., a polynucleotide encoding one or more immunomodulatory protein (e.g., IL-12 or one or more subunits thereof) or functional fragment thereof. In some aspects the AAV vector comprises two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into AAV vectors of the disclosure can be comprised of naturally occurring polynucleotide sequences or recombinantly derived polynucleotide sequences.

The ITRs can be derived from the same serotype as the capsid, selected from any of the serotypes listed herein, or a derivative thereof. The ITR can be of a different serotype from the capsid. In some aspects, the AAV vector has more than one ITR. In a non-limiting example, the AAV vector has a viral genome comprising two ITRs. In some aspects, the ITRs are of the same serotype as one another. In some aspects, the ITRs are of different serotypes. Non-limiting examples include zero, one or both of the ITRs having the same serotype as the capsid. In some aspects both ITRs of the AAV vector are AAV2 ITRs.

Independently, each ITR can be about 75 to about 175 nucleotides in length. An ITR can be about 100-105 nucleotides in length, about 106-110 nucleotides in length, about 111-115 nucleotides in length, about 116-120 nucleotides in length, about 121-125 nucleotides in length, about 126-130 nucleotides in length, about 131-135 nucleotides in length, about 136-140 nucleotides in length, about 141-145 nucleotides in length or about 146-150 nucleotides in length. In some aspects, the ITRs are about 140-142 nucleotides in length. Non-limiting examples of ITR length are about 102, about 140, about 141, about 142, about 145 nucleotides in length, and those having at least 95% identity thereto.

In some aspects, the AAV vector comprises at least one inverted terminal repeat having a length such as, but not limited to, about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, or about 170-175 nucleotides.

In some aspects, the length of a first and/or a second ITR regions for the AAV vector can be about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, and about 170-175 nucleotides.

In some aspects, the AAV vector comprises a nucleic acid encoding an immunomodulatory protein (e.g., IL-12 or one or more subunits thereof) or functional fragment thereof which can be located near the 5′ end of the flip ITR in the vector. In some aspects, the AAV vector comprises a nucleic acid encoding an immunomodulatory protein (e.g., IL-12 or one or more subunits thereof) or functional fragment thereof disclosed herein which can be located near the 3′ end of the flip ITR in the vector. In some aspects, the AAV vector comprises a nucleic acid sequence encoding an immunomodulatory protein (e.g., IL-12 or one or more subunits thereof) or functional fragment thereof disclosed herein which can be located near the 5′ end of the flop ITR in the vector. In some aspects, the AAV vector comprises a nucleic acid sequence encoding an immunomodulatory protein (e.g., IL-12 or one or more subunits thereof) or functional fragment thereof disclosed herein which can be located near the 3′ end of the flop ITR in the vector. In some aspects, the AAV vector comprises a nucleic acid sequence encoding an immunomodulatory protein (e.g., IL-12 or one or more subunits thereof) or functional fragment thereof disclosed herein which can be located between the 5′ end of the flip ITR and the 3′ end of the flop ITR in the vector. In some aspects, the AAV vector comprises a nucleic acid sequence encoding an immunomodulatory protein (e.g., IL-12 or one or more subunits thereof) or functional fragment thereof disclosed herein which can be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in the vector.

In some aspects, the AAV vector comprises a nucleic acid sequence encoding an immunomodulatory protein (e.g., IL-12 or one or more subunits thereof) or functional fragment thereof disclosed herein which can be located within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more than about 30 nucleotides downstream or upstream from the 5′ or 3′ end of an ITR (e.g., Flip or Flop ITR) in the vector.

As another non-limiting example, the AAV vector comprises a nucleic acid sequence encoding an immunomodulatory protein (e.g., IL-12 or one or more subunits thereof) or functional fragment thereof disclosed herein which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream or upstream from the 5′ or 3′ end of an ITR (e.g., Flip or Flop ITR) in the vector.

In some aspects, the AAV vector comprises a nucleic acid sequence encoding an immunomodulatory protein (e.g., IL-12 or one or more subunits thereof) or functional fragment thereof disclosed herein which can be located within the first about 1%, about 2%, about 3%, about 4%, about 5%, about 6%, about 7%, about 8%, about 9%, about 10%, about 15%, about 20%, about 25% or more than about 25% of the nucleotides upstream from the 5′ or 3′ end of an ITR (e.g., Flip or Flop ITR) in the vector.

As another non-limiting example, the AAV vector comprises a nucleic acid sequence encoding an immunomodulatory protein (e.g., IL-12 or one or more subunits thereof) or functional fragment thereof disclosed herein which can be located with the first about 1-5%, about 1-10%, about 1-15%, about 1-20%, about 1-25%, about 5-10%, about 5-15%, about 5-20%, about 5-25%, about 10-15%, about 10-20%, about 10-25%, about 15-20%, about 15-25%, or about 20-25% downstream from the 5′ or 3′ end of an ITR (e.g., Flip or Flop ITR) in the vector.

Certain aspects of the disclosure are directed to an AAV vector comprising a polynucleotide comprising polynucleotide comprising a first nucleic acid encoding an IL-12 subunit (e.g., IL-12 p35) or a functional fragment thereof and/or a second nucleic acid encoding an IL-12 subunit (e.g., IL-12 p40) or functional fragment thereof disclosed herein, wherein the polynucleotide further comprises ITR sequences (e.g., AAV2 ITRs).

Certain aspects of the disclosure are directed to a viral delivery vector comprising a polynucleotide comprising a CMV enhancer (e.g., SEQ ID NO: 68), a CBA promoter (e.g., SEQ ID NO: 85) operably linked to a first nucleic acid encoding a human IL-12 p35 subunit or a functional fragment thereof (e.g., SEQ ID NO: 77), a furin cleavage sequence followed by a 2A self-processing peptide (F2A), a second nucleic acid encoding a human IL-12 p40 subunit or a functional fragment thereof (e.g., SEQ ID NO: 8), and a human growth hormone (HGH) pA sequence (e.g., SEQ ID NO: 88). In some aspects, the polynucleotide further comprises an intron sequence (e.g., a CAG intron, e.g., SEQ ID NO: 86), a Kozak sequence, and ITR sequences (e.g., AAV2 ITRs). In some aspects, the polynucleotide comprises a CMV enhancer, a CBA promoter, and a CAG intron (e.g., SEQ ID NO: 71).

### IV.A.2. Promoters

In some aspects, the payload region of the AAV vector comprises at least one element to enhance the nucleic acid specificity and/or expression. Non-limiting examples of elements to enhance the nucleic acid specificity and expression include, e.g., promoters, endogenous miRNAs, post-transcriptional regulatory elements (PREs), polyadenylation (Poly A) signal sequences and upstream enhancers (USEs), CMV enhancers, and introns.

Expression of nucleic acid of the present disclosure after delivery to or integration in the genomic DNA of a target cell can require a specific promoter, including but not limited to, a promoter that is species specific, inducible, tissue-specific, or cell cycle-specific (Parr et al., Nat. Med. 3: 1145-9 (1997); the contents of which are herein incorporated by reference in their entirety).

In some aspects, the promoter is deemed to be efficient when it drives expression of the immunomodulatory protein (e.g., IL-12 or one or more subunits thereof) or functional fragment thereof disclosed herein carried in the payload region of the AAV vector. In some aspects, the promoter is a promoter deemed to be efficient when it drives expression of the immunomodulatory protein (e.g., IL-12 or one or more subunits thereof) or functional fragment thereof of the present disclosure in the cell being targeted (e.g., a tumor cell).

Promoters can be naturally occurring or non-naturally occurring. Non-limiting examples of promoters include viral promoters and mammalian promoters. In some aspects, the promoters can be human promoters. In some aspects, the promoter can be truncated. Promoters which drive or promote expression in most tissues include, but are not limited to, human elongation factor 1a-subunit (EF 1a), cytomegalovirus (CMV) immediate-early enhancer and/or promoter, chicken β-actin (CBA) and its derivative CAG, β glucuronidase (GUSB), or ubiquitin C (UBC). In some aspects, the promoter is a CBA promoter, a CMV promoter, a EF-1a (Elongation Factor 1a) promoter, aRSV (Rous Sarcoma Virus) promoter, an Ubiquitin (UbC) promoter, or any combination thereof. In some aspects, tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety). Non-limiting examples of tissue-specific expression elements for neurons include neuron-specific enolase (NSE), platelet-derived growth factor (PDGF), platelet-derived growth factor B-chain (PDGF-0), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene ηβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter. In some aspects, the promoter is a CAG promoter.

In some aspects, the promoter can be less than 1 kb. In some aspects, the promoter can have a length between about 15-20, about 10-50, about 20-30, about 30-40, about 40-50, about 50-60, about 50-100, about 60-70, about 70-80, about 80-90, about 90-100, about 100-110, about 100-150, about 110-120, about 120-130, about 130-140, about 140-150, about 150-160, about 150-200, about 160-170, about 170-180, about 180-190, about 190-200, about 200-210, about 200-250, about 210-220, about 220-230, about 230-240, about 240-250, about 250-260, about 250-300, about 260-270, about 270-280, about 280-290, about 290-300, about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides.

In some aspects, the promoter can be a combination of two or more components of the same or different starting or parental promoters such as, but not limited to, CMV and CBA. In some aspects, the promoter comprises a CBA promoter, a CMV promoter, a EF-1a (Elongation Factor 1a) promoter, aRSV (Rous Sarcoma Virus) promoter, an Ubiquitin (UbC) promoter, or any combination thereof. In some aspects, one of the two or more promoters is a CMV enhancer fused to a EF1α promoter. In some aspects, one of the two or more promoters is a CMV promoter fused to an SV40 intron. In some aspects, one of the two or more promoters is a CMV enhancer fused to a CMV promoter. In some aspects, one of the two or more promoters has a nucleic acid sequence comprising at least 85%, 86%, 87%, 88%, 89%, 90%, 91%, 92%, 93%, 94%, 95%, 96%, 97%, 98%, 99%, or 100% identical to any one of SEQ ID NOs: 67-71 or 85.

In some aspects, each component in the promoter can have a length between about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides. In some aspects, the promoter is a combination of a 382 nucleotide CMV-enhancer sequence and a 260 nucleotide CBA-promoter sequence.

In some aspects, the promoter combination comprises a CMV enhancer, a CBA promoter, and a CAG intron (e.g., SEQ ID NO: 71).

In some aspects, the AAV vector comprises a ubiquitous promoter. Non-limiting examples of ubiquitous promoters include, e.g., CMV, CBA (including derivatives CAG, CBh, etc.), EF-1a, PGK, UBC, GUSB (hGBp), and UCOE (promoter of HNRPA2B1-CBX3).

In some aspects, the promoter is not cell specific. In some aspects, the promoter is a ubiquitin c (UBC) promoter. The UBC promoter can have a size of 300-350 nucleotides. In some aspects, the UBC promoter is 332 nucleotides. In some aspects, the promoter is a β-glucuronidase (GUSB) promoter. The GUSB promoter can have a size of 350-400 nucleotides. In some aspects, the GUSB promoter is 378 nucleotides. In some aspects, the promoter is a neurofilament light (NFL) promoter. The NFL promoter can have a size of 600-700 nucleotides. In some aspects, the NFL promoter is 650 nucleotides. In some aspects, the construct can be AAV-promoter-CMV/globin intron-modulatory polynucleotide-RBG, where the AAV can be self-complementary and the AAV can be the DJ serotype.

In some aspects, the AAV vector comprises a Pol III promoter. In some aspects, the AAV vector comprises a PI promoter. In some aspects, the AAV vector comprises a FXN promoter. In some aspects, the promoter is a phosphogly cerate kinase 1 (PGK) promoter. In some aspects, the promoter is a chicken β-actin (CBA) promoter. In some aspects, the promoter is a CAG promoter which is a construct comprising the cytomegalovirus (CMV) enhancer fused to the chicken beta-actin (CBA) promoter with a chimeric intron. In some aspects, the promoter is a cytomegalovirus (CMV) promoter. In some aspects, the AAV vector comprises a H1 promoter. In some aspects, the AAV vector comprises a U6 promoter. In some aspects, the AAV vector comprises a SP6 promoter.

In some aspects, the promoter is a liver or a skeletal muscle promoter. Non-limiting examples of liver promoters include human a-1-antitrypsin (hAAT) and thyroxine binding globulin (TBG). Non-limiting examples of skeletal muscle promoters include Desmin, MCK or synthetic C5-12. In some aspects, the promoter is an RNA pol III promoter. In some aspects, the RNA pol III promoter is U6. In some aspects, the RNA pol III promoter is H1. In some aspects, the AAV vector comprises two promoters. In some aspects, the promoters are an EF1a promoter and a CMV promoter.

In some aspects, the AAV vector comprises an enhancer element, a promoter and/or a 5′UTR intron. The enhancer element, also referred to herein as an “enhancer,” can be, but is not limited to, a CMV enhancer, the promoter can be, but is not limited to, a CMV, CBA, UBC, GUSB, NSE, Synapsin, MeCP2, EF 1α, and GFAP promoter and the 5′UTR/intron can be, but is not limited to, SV40, a chimera of a betaglobin intron and a immunoglobulin heavy chain intron, and CBA-MVM (Minute virus of mice). In some aspects, the enhancer, promoter and/or intron used in combination can be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter, (9) GFAP promoter, (10) H1 promoter; (11) U6 promoter; (12) 12) CMV enhancer, EF1α promoter; (13) CMV promoter, SV40 intron; (14) CMV enhancer, CMV promoter; or (15) chimera of human betaglobin intron and immunoglobulin heavy chain intron, CMV enhancer, EF1α promoter, CMV promoter, SV40 intron. In some aspects, the AAV vector comprises a CMV early enhancer/chicken β actin (CAG) promoter. In some aspects, the AAV vector comprises an engineered promoter. In some aspects the AAV vector comprises a promoter from a naturally expressed protein.

In some aspects, the construct can comprise two or more promoters, e.g., a promoter for the nucleic acid encoding a heavy chain and a promoter for the nucleic acid sequence encoding a light chain. In some aspects, the two or more promoters are the same. In some aspects, the construct can comprise a promoter for the nucleic acid encoding a first cytokine subunit (e.g., an IL-12 p35 subunit or an IL-12 p40 subunit) and a promoter for the nucleic acid sequence encoding a second cytokine subunit (e.g., an IL-12 p40 subunit or IL-12 p35 subunit). In some aspects, the two or more promoters are the same. In some aspects, the two or more promoters are different.

### IV.A.3. Untranslated Regions (UTRs)

By definition, wild-type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs can be engineered into UTRs to enhance transcribed product stability and production. In some aspects, a 5′ UTR from mRNA normally expressed in the liver (e.g., albumin, serum amyloid A, Apolipoprotein A/B/E, transferrin, alpha fetoprotein, erythropoietin, or Factor VIII) can be used in AAV vector of the disclosure to enhance expression, e.g., in brain tissue, and specifically in neuronal cells.

Wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG (SEQ ID NO: 4), where R is a purine (adenine or guanine) three bases upstream of the start codon (ATG), which is followed by another ‘G. In some aspects, the 5′UTR in a AAV vector of the present disclosure includes a Kozak sequence. In some aspects, the 5′UTR in a AAV vector of the present disclosure does not include a Kozak sequence.

Wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety). Class I AREs, such as, but not limited to, c-Myc and MyoD, contain several dispersed copies of an AUUUA (SEQ ID NO: 32) motif within U-rich regions. Class II AREs, such as, but not limited to, GM-CSF and TNF-α, possess two or more overlapping UUAUUUA(U/A)(U/A) (SEQ ID NO: 33) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some aspects, the 3′ UTR of an AAV vector of the present disclosure can include an oligo(dT) sequence for addition of a poly-A tail. In some aspects, an AAV vector of the present disclosure can include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some aspects, an AAV vector of the present disclosure can be engineered to include, alter or remove at least one miRNA binding site, sequence or seed region.

Any UTR from any gene known in the art can be incorporated into an AAV vector of the present disclosure. These UTRs, or portions thereof, can be placed in the same orientation as in the gene from which they were selected or they can be altered in orientation or location. In some aspects, the UTR used in an AAV vector of the present disclosure can be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR can be altered relative to a wild-type or native UTR by the change in orientation or location as taught above or can be altered by the inclusion of additional nucleotides, deletion of nucleotides, swapping or transposition of nucleotides. In some aspects, an AAV vector of the present disclosure comprises at least one artificial UTRs which is not a variant of a wild-type UTR. In some aspects, an AAV vector of the present disclosure comprises UTRs which have been selected from a family of transcripts whose proteins share a common function, structure, feature or property.

### IV.A.4. Polyadenylation Sequence

In some aspects, the AAV vectors of the present disclosure comprise at least one polyadenylation sequence. The AAV vectors of the present disclosure can comprise a polyadenylation sequence between the 3′ end of the payload coding sequence and the 5′ end of the 3′ITR.

In some aspects, the polyadenylation sequence or “polyA sequence” can range from absent to about 500 nucleotides in length.

In some aspects, the polyadenylation sequence is about 50-100, about 50-150, about 50-160, about 50-200, about 60-100, about 60-150, about 60-160, about 60-200, about 70-100, about 70-150, about 70-160, about 70-200, about 80-100, about 80-150, about 80-160, about 80-200, about 90-100, about 90-150, about 90-160, or about 90-200 nucleotides in length.

In some aspects, the AAV vector comprises a nucleic acid sequence encoding an immunomodulatory protein (e.g., IL-12 or one or more subunits thereof) or functional fragment thereof disclosed herein which can be located upstream of the polyadenylation sequence in the vector. In some aspects, the AAV vector comprises a nucleic acid sequence encoding an immunomodulatory protein or functional fragment thereof disclosed herein which can be located downstream of a promoter such as, but not limited to, CMV, U6, CAG, CBA or a CBA promoter with a SV40 intron, MVM intron, or a human betaglobin intron in the vector.

In some aspects, the AAV vector of the present disclosure comprises a nucleic acid sequence encoding an immunomodulatory protein (e.g., IL-12 or one or more subunits thereof) or functional fragment thereof disclosed herein which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

In some aspects, the AAV vector comprises a rabbit globin polyadenylation (poly A) signal sequence. In some aspects, the AAV vector comprises a human growth hormone polyadenylation (poly A) signal sequence. In some aspects, the AAV vector comprises a human growth hormone polyadenylation (poly A) (HGHpA) signal sequence. In some aspects, HGHpA has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 88. In some aspects, the AAV vector comprises a bovine growth hormone polyadenylation (poly A) (BGHpA) signal sequence. In some aspects, BGHpA has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 73. In some aspects, the AAV vector comprises a synthetic (SYN) polyadenylation (poly A) (SYNpA) signal sequence. In some aspects, SYNpA has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 72.

### IV.A.5. Introns

In some aspects, the payload region of an AAV vector of the present disclosure comprises at least one element to enhance the expression such as one or more introns or portions thereof. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps). In some aspects, the chimeric intron can be a chimera of a human betaglobin intron and a human immunoglobin heavy chain intron. In some aspects, the intron can have a nucleotide sequence with at least 85%, 86%, 87%, 88%, 89%, 90%, 91%, 92%, 93%, 94%, 95%, 96%, 97%, 98%, 99%, or 100% identical to any of SEQ ID NOs: 66 and 76.

In some aspects, the intron or intron portion can be between about 100 and about 500 nucleotides in length. In some aspects, the intron can have a length between about 80-100, about 80-120, about 80-140, about 80-160, about 80-180, about 80-200, about 80-250, about 80-300, about 80-350, about 80-400, about 80-450, about 80-500, about 200-300, about 200-400, about 200-500, about 300-400, about 300-500, or about 400-500 nucleotides.

In some aspects, the AAV vector can comprise a promoter such as, but not limited to, CMV, CBA, or U6. In some aspects, the promoter for an AAV vector of the present disclosure is a CMV promoter. In some aspects, the promoter for an AAV vector of the present disclosure is a CBA promoter. In some aspects, the AAV vector can comprise a CBA promoter, a CMV promoter, a EF-1a (Elongation Factor 1a) promoter, aRSV (Rous Sarcoma Virus) promoter, an Ubiquitin (UbC) promoter, or any combination thereof. As another non-limiting example, the promoter for an AAV vector of the present disclosure is a U6 promoter. In some aspects, the AAV vector can comprise a CMV and a U6 promoter. In some aspects, the AAV vector can comprise a H1 promoter. In some aspects, the AAV vector can comprise a CBA promoter. In some aspects, the AAV vector can comprise a chimeric intron. In some aspects, the AAV vector of the present disclosure is a CBA promoter, a CMV promoter, a EF-1a (Elongation Factor 1a) promoter, aRSV (Rous Sarcoma Virus) promoter, an Ubiquitin (UbC) promoter, a CMV enhancer fused to a EF1α promoter, a CMV promoter fused to an SV40 intron, a CMV enhancer fused to a CMV promoter, or any combination thereof. In some aspects, the promoter comprises a nucleotide sequence with at least 85%, 86%, 87%, 88%, 89%, 90%, 91%, 92%, 93%, 94%, 95%, 96%, 97%, 98%, 99%, or 100% identical to any one of SEQ ID NOs: 67-71 or 85.

In some aspects, the encoded immunomodulatory protein (e.g., IL-12 or one or more subunits thereof) or functional fragment thereof disclosed herein can be located downstream of a promoter in an expression vector such as, but not limited to, CMV, U6, HI, CBA, CAG, or a CBA promoter with an intron such as SV40 or others known in the art. Further, the encoded immunomodulatory protein or functional fragment thereof disclosed herein can also be located upstream of the polyadenylation sequence in an expression vector. In some aspects, the encoded immunomodulatory protein (e.g., IL-12 or one or more subunits thereof) or functional fragment thereof disclosed herein can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

### IV.A.6. Filler Sequences

In some aspects, the AAV vector comprises one or more filler sequences (also referred to as “stuffer sequences”). In some aspects, the AAV vector comprises one or more filler sequences in order to have the length of the AAV vector be the optimal size for packaging. In some aspects, the AAV vector comprises at least one filler sequence in order to have the length of the AAV vector be about 2.0-2.5 kb, e.g., about 2.3 kb. In some aspects, the AAV vector comprises at least one filler sequence in order to have the length of the AAV vector be about 4.6 kb. In some aspects, the vector backbone comprises a filler sequence.

In some aspects, the AAV vector comprises one or more filler sequences in order to reduce the likelihood that a hairpin structure of the vector genome (e.g., a modulatory polynucleotide described herein, e.g., IL-12 or one or more subunits thereof) can be read as an inverted terminal repeat (ITR) during expression and/or packaging. In some aspects, the AAV vector comprises at least one filler sequence in order to have the length of the AAV vector be about 2.0-2.5 kb, e.g., about 2.3 kb. In some aspects, the AAV vector comprises at least one filler sequence in order to have the length of the AAV vector be about 4.6 kb.

In some aspects, the AAV vector is a single stranded (ss) AAV vector and comprises one or more filler sequences which have a length about between 0.1 kb and about 3.8 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, about 3.3 kb, about 3.4 kb, about 3.5 kb, about 3.6 kb, about 3.7 kb, or about 3.8 kb.

In some aspects, the AAV vector is a self-complementary (sc) AAV vector and comprises one or more filler sequences which have a length about between about 0.1 kb and about 1.5 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, or about 1.5 kb.

In some aspects, the AAV vector comprises any portion of a filler sequence. The vector can comprise, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of a filler sequence.

In some aspects, the AAV vector is a single stranded (ss) AAV vector and comprises one or more filler sequences in order to have the length of the AAV vector be about 4.6 kb. In some aspects, the AAV vector comprises at least one filler sequence and the filler sequence is located 3′ to the 5′ ITR sequence. In some aspects, the AAV vector comprises at least one filler sequence and the filler sequence is located 5′ to a promoter sequence. In some aspects, the AAV vector comprises at least one filler sequence and the filler sequence is located 3′ to the polyadenylation signal sequence. In some aspects, the AAV vector comprises at least one filler sequence and the filler sequence is located 5′ to the 3′ ITR sequence. In some aspects, the AAV vector comprises at least one filler sequence, and the filler sequence is located between two intron sequences. In some aspects, the AAV vector comprises at least one filler sequence, and the filler sequence is located within an intron sequence. In some aspects, the AAV vector comprises two filler sequences, and the first filler sequence is located 3′ to the 5′ ITR sequence and the second filler sequence is located 3′ to the polyadenylation signal sequence. In some aspects, the AAV vector comprises two filler sequences, and the first filler sequence is located 5′ to a promoter sequence and the second filler sequence is located 3′ to the polyadenylation signal sequence. In some aspects, the AAV vector comprises two filler sequences, and the first filler sequence is located 3′ to the 5′ ITR sequence and the second filler sequence is located 5′ to the 5′ ITR sequence.

In some aspects, the AAV vector is a self-complementary (sc) AAV vector and comprises one or more filler sequences in order to have the length of the AAV vector be about 2.3 kb. In some aspects, the AAV vector comprises at least one filler sequence and the filler sequence is located 3′ to the 5′ ITR sequence. In some aspects, the AAV vector comprises at least one filler sequence and the filler sequence is located 5′ to a promoter sequence. In some aspects, the AAV vector comprises at least one filler sequence and the filler sequence is located 3′ to the polyadenylation signal sequence. In some aspects, the AAV vector comprises at least one filler sequence and the filler sequence is located 5′ to the 3′ ITR sequence.

In some aspects, the AAV vector comprises at least one filler sequence, and the filler sequence is located between two intron sequences. In some aspects, the AAV vector comprises at least one filler sequence, and the filler sequence is located within an intron sequence. In some aspects, the AAV vector comprises two filler sequences, and the first filler sequence is located 3′ to the 5′ ITR sequence and the second filler sequence is located 3′ to the polyadenylation signal sequence. In some aspects, the AAV vector comprises two filler sequences, and the first filler sequence is located 5′ to a promoter sequence and the second filler sequence is located 3′ to the polyadenylation signal sequence. In some aspects, the AAV vector comprises two filler sequences, and the first filler sequence is located 3′ to the 5′ ITR sequence and the second filler sequence is located 5′ to the 5′ ITR sequence.

In some aspects, the AAV vector can comprise one or more filler sequences between one of more regions of the AAV vector. In some aspects, the filler region can be located before a region such as, but not limited to, a payload region, an ITR, a promoter region, an intron region, an enhancer region, and/or a polyadenylation signal sequence region. In some aspects, the filler region can be located after a region such as, but not limited to, a payload region, an ITR, a promoter region, an intron region, an enhancer region, and/or a polyadenylation signal sequence region. In some aspects, the filler region can be located before and after a region such as, but not limited to, a payload region, an ITR, a promoter region, an intron region, an enhancer region, and/or a polyadenylation signal sequence region.

In some aspects, the AAV vector can comprise one or more filler sequences which bifurcates at least one region of the AAV vector. The bifurcated region of the AAV vector can comprise about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the of the region to the 5′ of the filler sequence region.

In some aspects, the filler sequence can bifurcate at least one region so that about 10% of the region is located 5′ to the filler sequence and about 90% of the region is located 3′ to the filler sequence. In some aspects, the filler sequence can bifurcate at least one region so that about 20% of the region is located 5′ to the filler sequence and about 80% of the region is located 3′ to the filler sequence. In some aspects, the filler sequence can bifurcate at least one region so that about 30% of the region is located 5′ to the filler sequence and about 70% of the region is located 3′ to the filler sequence. In some aspects, the filler sequence can bifurcate at least one region so that about 40% of the region is located 5′ to the filler sequence and about 60% of the region is located 3′ to the filler sequence. In some aspects, the filler sequence can bifurcate at least one region so that about 50% of the region is located 5′ to the filler sequence and about 50% of the region is located 3′ to the filler sequence. In some aspects, the filler sequence can bifurcate at least one region so that about 60% of the region is located 5′ to the filler sequence and about 40% of the region is located 3′ to the filler sequence. In some aspects, the filler sequence can bifurcate at least one region so that about 70% of the region is located 5′ to the filler sequence and about 30% of the region is located 3′ to the filler sequence. In some aspects, the filler sequence can bifurcate at least one region so that about 80% of the region is located 5′ to the filler sequence and about 20% of the region is located 3′ to the filler sequence. In some aspects, the filler sequence can bifurcate at least one region so that about 90% of the region is located 5′ to the filler sequence and about 10% of the region is located 3′ to the filler sequence.

In some aspects, the AAV vector comprises a filler sequence after the 5′ ITR. In some aspects, the AAV vector comprises a filler sequence after the promoter region. In some aspects, the AAV vector comprises a filler sequence after the payload region. In some aspects, the AAV vector comprises a filler sequence after the intron region. In some aspects, the AAV vector comprises a filler sequence after the enhancer region. In some aspects, the AAV vector comprises a filler sequence after the polyadenylation signal sequence region. In some aspects, the AAV vector comprises a filler sequence before the promoter region. In some aspects, the AAV vector comprises a filler sequence before the payload region. In some aspects, the AAV vector comprises a filler sequence before the intron region.

In some aspects, the AAV vector comprises a filler sequence before the enhancer region. In some aspects, the AAV vector comprises a filler sequence before the polyadenylation signal sequence region. In some aspects, the AAV vector comprises a filler sequence before the 3′ ITR. In some aspects, a filler sequence can be located between two regions, such as, but not limited to, the 5′ ITR and the promoter region. In some aspects, a filler sequence can be located between two regions, such as, but not limited to, the 5′ ITR and the payload region.

In some aspects, a filler sequence can be located between two regions, such as, but not limited to, the 5′ ITR and the intron region. In some aspects, a filler sequence can be located between two regions, such as, but not limited to, the 5′ ITR and the enhancer region. In some aspects, a filler sequence can be located between two regions, such as, but not limited to, the 5′ ITR and the polyadenylation signal sequence region. In some aspects, a filler sequence can be located between two regions, such as, but not limited to, the promoter region and the payload region.

In some aspects, a filler sequence can be located between two regions, such as, but not limited to, the promoter region and the intron region. In some aspects, a filler sequence can be located between two regions, such as, but not limited to, the promoter region and the enhancer region. In some aspects, a filler sequence can be located between two regions, such as, but not limited to, the promoter region and the polyadenylation signal sequence region. In some aspects, a filler sequence can be located between two regions, such as, but not limited to, the promoter region and the 3′ ITR.

In some aspects, a filler sequence can be located between two regions, such as, but not limited to, the payload region and the intron region. In some aspects, a filler sequence can be located between two regions, such as, but not limited to, the payload region and the enhancer region. In some aspects, a filler sequence can be located between two regions, such as, but not limited to, the payload region and the polyadenylation signal sequence region. In some aspects, a filler sequence can be located between two regions, such as, but not limited to, the payload region and the 3′ ITR.

In some aspects, a filler sequence can be located between two regions, such as, but not limited to, the intron region and the enhancer region. In some aspects, a filler sequence can be located between two regions, such as, but not limited to, the intron region and the polyadenylation signal sequence region. In some aspects, a filler sequence can be located between two regions, such as, but not limited to, the intron region and the 3′ ITR. In some aspects, a filler sequence can be located between two regions, such as, but not limited to, the enhancer region and the polyadenylation signal sequence region. In some aspects, a filler sequence can be located between two regions, such as, but not limited to, the enhancer region and the 3′ ITR. In some aspects, a filler sequence can be located between two regions, such as, but not limited to, the polyadenylation signal sequence region and the 3′ ITR.

In some aspects, an AAV vector can comprise two filler sequences. The two filler sequences can be located between two regions as described herein.

### IV.A. 7. Method for Producing Recombinant AAVs

The present disclosure provides also methods for the generation of AAV particles, by viral genome replication in a viral replication cell comprising contacting the viral replication cell with an AAV polynucleotide or AAV genome (e.g., an AAV vector of the present disclosure). In the context of the present disclosure, the AAV vectors disclosed herein, e.g., AAV vectors comprising at least one polynucleotide encoding an immunomodulatory protein (e.g., IL-12 or one or more subunits thereof) or functional fragment thereof disclosed herein, are considered AAV payload construct vectors.

In some aspects, an AAV particle is produced by a method comprising the steps of:
(1) co-transfecting competent bacterial cells with a bacmid vector and either a viral construct vector and/or AAV payload construct vector,
(2) isolating the resultant viral construct expression vector and AAV payload construct expression vector and separately transfecting viral replication cells,
(3) isolating and purifying resultant payload and viral construct particles comprising viral construct expression vector or AAV payload construct expression vector,
(4) co-infecting a viral replication cell with both the AAV payload and viral construct particles comprising viral construct expression vector or AAV payload construct expression vector, and
(5) harvesting and purifying the viral particle comprising a parvoviral genome.

In one aspect, the present disclosure provides a method for producing an AAV particle comprising the steps of (1) simultaneously co-transfecting mammalian cells, such as, but not limited to HEK293 cells, with a payload region (e.g., polynucleotide encoding an immunomodulatory protein or functional fragment thereof of the present disclosure), a construct expressing rep and cap genes and a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO.

W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide sequence encoding a payload such as an immunomodulatory protein or functional fragment thereof disclosed herein.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection (MOI), see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65(2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production.

In some cases, bioreactors comprise stirred tank reactors.

### IV.A.8. Cell Lysis

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in U.S. Patent Application 20090275107.

### IV.A.9. AAV Purification

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

### V. Methods of Treatment and Use

Some aspects of the present disclosure are directed to a method of delivering an immunomodulatory protein (e.g., two or more immunomodulatory proteins (e.g., cytokines), one or more subunits thereof, or functional fragments thereof) to a tumor, comprising administering a polynucleotide, an AAV capsid (e.g., AAV particle), or a composition (e.g., gene therapy composition) of the present disclosure to a subject in need thereof. In some aspects, the methods of the disclosure comprise delivery of a multicistronic (e.g., bicistronic) construct (e.g., comprising nucleic acids encoding cytokine (e.g., IL-12) subunits). In some aspects, the methods of the disclosure further comprise administering a checkpoint inhibitor (e.g., a PD-1 inhibitor).

In some aspects, the method of delivering two or more immunomodulatory proteins (e.g., cytokines), one or more subunits thereof, or functional fragments thereof comprises administering to a tumor by direct injection of a composition comprising one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof.

In some aspects, the method of delivering an immunomodulatory protein (e.g., a cytokine or one or more subunits thereof, or functional fragments thereof) comprises administering to a tumor by direct injection of a composition comprising a delivery vector (e.g., a viral vector) for delivery of a (i) a first nucleic acid encoding a first cytokine subunit (e.g., an IL-12 p35 subunit or an IL-12 p40 subunit), or functional fragment thereof and (ii) a second nucleic acid encoding a second cytokine subunit (e.g., an IL-12 p40 subunit or an IL-12 p35 subunit), or functional fragment thereof. In some aspects, the first nucleic acid (e.g., nucleic acid encoding an IL-12 p35 subunit) does not include an in-frame stop codon (e.g., TGA, TAG, and TAA).

In some aspects, the nucleic acid encoding an IL-12 p35 subunit comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77. In some aspects, the nucleic acid encoding the IL-12 p40 subunit comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92. In some aspects, the delivery vector (e.g., AAV vector) comprises a polynucleotide comprising (i) a nucleic acid encoding an IL-12 p35 subunit, wherein the nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77; and (ii) a nucleic acid encoding the IL-12 p40 subunit, wherein the nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92.

Certain aspects of the disclosure are directed to a combination therapy comprising: (a) a first composition comprising an adeno-associated virus (AAV) vector comprising a polynucleotide comprising (i) a promoter, (ii) a first nucleic acid encoding a first Interleukin-12 (IL-12) subunit or functional fragment thereof, (iii) a translation modification sequence, and (iv) a second nucleic acid encoding a second IL-12 subunit or functional fragment thereof, and (b) a second composition comprising a checkpoint inhibitor.

In some aspects, the AAV vector is an AAV serotype 2 (AAV2) vector.

In some aspects, the translation modification sequence comprises a furin cleavage sequence, a 2A self-processing peptide sequence, an internal ribosomal entry site (IRES) sequence, or any combination thereof. In some aspects, the translation modification sequence comprises a furin cleavage sequence and a 2A self-processing peptide sequence (F2A).

In some aspects, the promoter comprises a CAG promoter, a CBA promoter, a CMV promoter, an EF-1a (Elongation Factor 1a) promoter, a RSV (Rous Sarcoma Virus) promoter, an Ubiquitin (UbC) promoter, or any combination thereof.

In some aspects, the polynucleotide further comprises a poly(A) (pA) sequence.

In some aspects, the polynucleotide further comprises two inverted terminal repeat (ITR) sequences.

In some aspects, the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

In some aspects, the checkpoint inhibitor comprises a programmed cell death protein 1 (PD-1) inhibitor (e.g., an anti-PD-1 antibody). In some aspects, the anti-PD-1 antibody is pembrolizumab or nivolumab.

In some aspects, the first Interleukin-12 (IL-12) subunit comprises an amino acid sequence having at least 85%, 90%, 95%, 96%, 97%, 98%, 99%, or 100% identity to SEQ ID NO: 80 and the second IL-12 subunit comprises an amino acid sequence having at least 85%, 90%, 95%, 96%, 97%, 98%, 99%, or 100% identity to SEQ ID NO: 81.

In some aspects, the first nucleic acid comprises a nucleic acid sequence having at least 85%, 90%, 95%, 96%, 97%, 98%, 99%, or 100% identity to SEQ ID NO: 5, SEQ ID NO: 6, SEQ ID NO: 7, or SEQ ID NO: 77, and the second nucleic acid comprises a nucleic acid sequence having at least 85%, 90%, 95%, 96%, 97%, 98%, 99%, or 100% identity to SEQ ID NO: 8, SEQ ID NO: 9, SEQ ID NO: 10, or SEQ ID NO: 92.

In some aspects, the polynucleotide comprises a nucleic acid sequence having at least 85%, 90%, 95%, 96%, 97%, 98%, 99%, or 100% identity to SEQ ID NO: 82.

In some aspects, the first composition is suitable for an intratumoral delivery and the second composition is suitable for an intravenous delivery.

Certain aspects of the disclosure are directed to methods of treating a subject suffering from a tumor comprising administering a combination therapy disclosed herein to the subject. In some aspects, the first composition is administered intratumorally. In some aspects, the first composition is administered to multiple sites of the tumor. In some aspects, the first composition is administered prior to, at the same time, or after the administration of the second composition.

In some aspects, the tumor is derived from a cancer selected from the group consisting of a skin cancer; a breast cancer; a brain cancer; a bone cancer; a head and neck cancer; a salivary gland cancer; a gynecologic cancer; a urologic cancer; a gastrointestinal cancer; an ocular cancer; a thoracic cancer; a blood cancer; a cancer of the endocrine system; a sarcoma of soft tissue; a neoplasm of the central nervous system; and any combination thereof.

In some aspects, the tumor is derived from a cancer selected from the group consisting of hepatocellular carcinoma, colon carcinoma, and melanoma.

Certain aspects of the disclosure are directed to a method of use comprising administering a composition comprising a delivery vector (e.g., a viral vector) for delivery of a (i) a first nucleic acid encoding a first cytokine subunit (e.g., an IL-12 p35 subunit or an IL-12 p40 subunit), or functional fragment thereof and (ii) a second nucleic acid encoding a second cytokine subunit (e.g., an IL-12 p40 subunit or an IL-12 p35 subunit), or functional fragment thereof. In some aspects, the nucleic acid encoding an IL-12 p35 subunit comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77. In some aspects, the nucleic acid encoding the IL-12 p40 subunit comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92.

Certain aspects of the disclosure are directed to a method of use comprising administering a polynucleotide comprising a polynucleotide comprising (i) a nucleic acid encoding an IL-12 p35 subunit, wherein the nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77; and/or (ii) a nucleic acid encoding the IL-12 p40 subunit, wherein the nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92.

Certain aspects of the disclosure are directed to a method of use comprising administering a composition comprising (a) a polynucleotide comprising a CMV enhancer, a CBA promoter or fragment thereof operably linked to a first nucleic acid encoding an IL-12 p35 subunit or a functional fragment thereof, a furin cleavage sequence followed by a 2A self-processing peptide (F2A), a second nucleic acid encoding an IL-12 p40 subunit or a functional fragment thereof, and a human growth hormone (HGH) pA sequence; and (b) a viral vector (e.g., an AAV capsid). In some aspects, the polynucleotide further comprises an intron (e.g., a CAG intron), a Kozak sequence and/or ITR sequences.

Certain aspects of the disclosure are directed to a composition, a polynucleotide, AAV capsid, or the expression construct of the disclosure for use in therapy or prophylaxis.

Certain aspects of the disclosure are directed to the composition, the polynucleotide, AAV capsid or the expression construct for use in gene therapy.

Certain aspects of the disclosure are directed to the composition, the polynucleotide, AAV capsid or the expression construct for use in cancer therapy.

Certain aspects of the disclosure are directed to the composition, the polynucleotide, AAV capsid or the expression construct for use in a method as defined in the present disclosure.

Use of the composition, the polynucleotide, AAV capsid, or the expression construct of the present disclosure, for the manufacture of a medicament for use in therapy or prophylaxis. In certain aspects, the therapy or prophylaxis comprises a method as defined in the disclosure.

Certain aspects of the disclosure are directed to a plurality of compostions for use in therapy or prophylaxis.

Certain aspects of the disclosure are directed to a plurality of compostions for use in gene therapy.

Certain aspects of the disclosure are directed to a plurality of compostions for use in cancer therapy.

In some aspects, the plurality of compostions comprise a composition comprising an AAV delivery vector comprising a polynucleotide comprising a nucleic acid encoding a cytokine, one or more subunits thereof, or functional fragment thereof (e.g., IL-12, or one or more subunits thereof); and (b) a checkpoint inhibitor (e.g., a PD-1 inhibitor).

The compositions, the polynucleotides, AAV capsids, and the expression constructs of the present disclosure can be tested in proven animal models and the resulting data will provide the human version of the compositions, the polynucleotides, AAV capsids, and the expression constructs for use in human subjects.

In some aspects, each delivery vector (e.g., viral vector) comprises a polynucleotide comprising the first nucleic acid (e.g., encoding a first cytokine subunit) and the second nucleic acid (e.g., encoding a second cytokine subunit). In some aspects, each delivery vector (e.g., viral vector) comprises either the first nucleic acid or the second nucleic acid, wherein two or more delivery vectors are delivered in combination (e.g., simultaneously or sequentially).

In some aspects, the method of delivering two or more immunomodulatory proteins (e.g., cytokines) or functional fragments thereof comprises administering to a tumor by direct injection of a composition comprising: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector).

In some aspects, the method of delivering two or more immunomodulatory proteins (e.g., cytokines) or functional fragments thereof comprises administering to a tumor by direct injection of a composition comprising a polynucleotide comprising: (a) a first nucleic acid encoding a first cytokine, one or more subunits thereof, or functional fragment thereof, and (b) a second nucleic acid encoding a second cytokine, one or more subunits thereof, or functional fragment thereof.

In some aspects, the method of delivering an immunomodulatory protein (e.g., cytokine) or functional fragments thereof comprises administering to a tumor by direct injection of a composition comprising a polynucleotide comprising: (a) a first nucleic acid encoding a first cytokine subunit, or functional fragment thereof; and (b) a second nucleic acid encoding a second cytokine subunit, or functional fragment thereof.

In some aspects, the method of delivering two or more immunomodulatory proteins (e.g., cytokines) or functional fragments thereof comprises administering to a tumor by direct injection of a delivery vector (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof.

In some aspects, the method of delivering a cytokine or functional fragment thereof comprises administering to a tumor by direct injection of a delivery vector (e.g., a viral vector, a non-viral vectors, a plasmid, a lipid, protein particle, a bacterial vector, a lysosome, a virus-like particle, a polymeric particle, an exosome, or a vault particle) comprising an expression construct comprising a promoter which is operably linked to a first nucleic acid encoding a first cytokine subunit (e.g., an IL-12 subunit), or functional fragment thereof and a translation modification sequence (e.g., an F2A) linked to a second nucleic acid encoding a second cytokine subunit (e.g., an IL-12 subunit), or functional fragment thereof. In some aspects, the translation modification sequence is selected from a 2A self-processing peptide (e.g., a F2A peptide, a P2A peptide, an E2A peptide, or a T2A peptide), a furin cleavage sequence, an internal ribosomal entry site (IRES) sequence, or any combination thereof. In some aspects, the expressing construct can further comprise a polyA.

Some aspects of the present disclosure are directed to a method of expressing one or more immunomodulatory proteins (e.g., cytokines), one or more subunits thereof, or functional fragments thereof in a subject in need thereof, comprising administering a polynucleotide, an AAV capsid, or a composition (e.g., gene therapy composition) of the present disclosure to a subject in need thereof.

In some aspects, the method of expressing two or more immunomodulatory proteins (e.g., cytokines) or functional fragments thereof in a subject in need thereof comprises administering to a tumor by direct injection of a composition comprising one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof. In some aspects, each delivery vector (e.g., viral vector) comprises a polynucleotide comprising the first nucleic acid and the second nucleic acid. In some aspects, each delivery vector (e.g., viral vector) comprises either the first nucleic acid or the second nucleic acid, wherein two or more delivery vectors are delivered in combination (e.g., simultaneously or sequentially).

In some aspects, the method of expressing one or more immunomodulatory proteins (e.g., cytokines) or functional fragments thereof in a subject in need thereof comprises administering to a tumor by direct injection of a composition comprising one or more delivery vectors (e.g., viral vectors) comprising a promoter which is operably linked to a first nucleic acid encoding a first cytokine subunit (e.g., an IL-12 subunit), or functional fragment thereof and a translation modification sequence (e.g., an F2A) linked to a second nucleic acid encoding a second cytokine subunit (e.g., an IL-12 subunit), or functional fragment thereof.

In some aspects, the method of expressing two or more immunomodulatory proteins (e.g., cytokines) or functional fragments thereof in a subject in need thereof comprises administering to a tumor by direct injection of a composition comprising: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector).

In some aspects, the method of expressing one or more immunomodulatory proteins (e.g., cytokines) or functional fragments thereof in a subject in need thereof comprises administering to a tumor by direct injection of a composition comprising an expression construct comprising: a promoter which is operably linked to a first nucleic acid encoding a first cytokine subunit (e.g., an IL-12 subunit), or functional fragment thereof and a translation modification sequence (e.g., an F2A) linked to a second nucleic acid encoding a second cytokine subunit (e.g., an IL-12 subunit), or functional fragment thereof.

In some aspects, the method of expressing two or more immunomodulatory proteins (e.g., cytokines) or functional fragments thereof in a subject in need thereof comprises administering an effective amount of the composition (e.g., gene therapy composition), the polynucleotide, or the AAV capsid of the present disclosure to the subject. In some aspects, the method of expressing two or more immunomodulatory proteins (e.g., cytokines) or functional fragments thereof comprises intratumorally administering a delivery vector (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof.

In some aspects, the method of expressing one or more immunomodulatory proteins (e.g., cytokines) or functional fragments thereof in a subject in need thereof comprises administering an effective amount of the composition (e.g., gene therapy composition), the polynucleotide, or the AAV capsid of the present disclosure to the subject. In some aspects, the method of expressing one or more immunomodulatory proteins (e.g., cytokines) or functional fragments thereof comprises intratumorally administering a delivery vector (e.g., a viral vector) comprising: a promoter which is operably linked to a first nucleic acid encoding a first cytokine subunit (e.g., an IL-12 subunit), or functional fragment thereof and a translation modification sequence (e.g., an F2A) linked to a second nucleic acid encoding a second cytokine subunit (e.g., an IL-12 subunit), or functional fragment thereof.

In some aspects, the method of expressing two or more immunomodulatory proteins (e.g., cytokines) or functional fragments thereof comprises intravenously administering a delivery vector (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof.

Some aspects of the present disclosure are directed to a method of treating or reducing symptoms in a subject in need thereof, comprising administering a polynucleotide, an AAV capsid, or a composition (e.g., gene therapy composition) of the present disclosure to a subject suffering from a tumor.

In some aspects, the method of treating or reducing symptoms in a subject suffering from a tumor comprises administering to a tumor by direct injection of a composition comprising one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof. In some aspects, each delivery vector (e.g., viral vector) comprises a polynucleotide comprising the first nucleic acid and the second nucleic acid. In some aspects, each delivery vector (e.g., viral vector) comprises either the first nucleic acid or the second nucleic acid, wherein two or more delivery vectors are delivered in combination (e.g., simultaneously or sequentially). In some aspects, a delivery vector (e.g., viral vector) for delivery of (i) a first nucleic acid encoding a first immunomodulatory protein, one or more subunits thereof, or functional fragment thereof and/or (ii) a second nucleic acid encoding a second immunomodulatory protein, one or more subunits thereof, or functional fragment thereof is administered in combination (e.g., simultaneously or sequentially) with a checkpoint inhibitor.

In some aspects, the method of treating or reducing symptoms in a subject suffering from a tumor comprises administering to a tumor by direct injection of a composition comprising one or more delivery vectors (e.g., viral vectors) comprising: a promoter which is operably linked to a first nucleic acid encoding a first cytokine subunit (e.g., an IL-12 subunit), or functional fragment thereof and a translation modification sequence (e.g., an F2A) linked to a second nucleic acid encoding a second cytokine subunit (e.g., an IL-12 subunit), or functional fragment thereof. In some aspects, the delivery vector (e.g., viral vector) is delivered in combination (e.g., simultaneously or sequentially) with a checkpoint inhibitor (e.g., a PD-1 inhibitor).

In some aspects, two or more delivery vectors described herein are delivered in combination (e.g., simultaneously or sequentially) and further in combination with a checkpoint inhibitor.

In some aspects, the method of treating or reducing symptoms in a subject suffering from a tumor comprises administering to a tumor by direct injection of a composition comprising: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector).

In some aspects, the method of treating or reducing symptoms in a subject suffering from a tumor comprises administering to a tumor by direct injection of a composition comprising: (a) a polynucleotide comprising (i) a first nucleic acid encoding an IL-12 p35 subunit or functional fragment thereof and (ii) a second nucleic acid encoding an IL-12 p40 subunit or functional fragment thereof, and (b) a delivery vector (e.g., an AAV vector). In some aspects, the delivery vector (e.g., viral vector) is delivered in combination (e.g., simultaneously or sequentially) with a checkpoint inhibitor (e.g., a PD-1 inhibitor).

In some aspects, provided herein is a method of reducing the size of a tumor in a subject in need thereof comprising administering to the subject a composition comprising (a) a polynucleotide comprising (i) a first nucleic acid encoding an IL-12 p35 subunit or functional fragment thereof and (ii) a second nucleic acid encoding an IL-12 p40 subunit or functional fragment thereof, and (b) (b) a delivery vector (e.g., an AAV vector). In some aspects, the delivery vector (e.g., viral vector) is delivered in combination (e.g., simultaneously or sequentially) with a checkpoint inhibitor (e.g., a PD-1 inhibitor).

In some aspects, the method of treating or reducing symptoms in a subject in need thereof comprises administering the gene therapy, the polynucleotide, or the AAV capsid of the present disclosure to the subject.

In some aspects, the methods disclosed herein comprise re-administering a gene therapy, a polynucleotide, a AAV capsid, or a composition of the present disclosure to the subject.

In some aspects, the subject suffers from a tumor. In some aspects, the tumor is derived from a cancer selected from the group consisting of skin cancer (e.g., metastatic melanoma, cutaneous malignant melanoma, cutaneous squamous cell carcinoma, basal cell carcinoma); breast cancer (e.g., invasive breast cancer, triple-negative breast cancer, inflammatory breast cancer); brain cancer (e.g., glioblastoma multiforme, medulloblastoma, pituitary carcinoma, brain stem gliomas, astrocytomas, oligodendrogliomas, hemangiopericytomas, germ cell tumors, pineal tumors); bone cancer (e.g., chordomas, chondrosarcomas, osteosarcomas, Ewing sarcomas, fibrosarcomas, adamantiomas, giant cell tumors); head and neck cancer (e.g., head and neck squamous cell carcinoma; salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer); gynecologic cancer (e.g., ovarian cancer, uterine cancer, fallopian tube cancer, endometrial cancer, cervical cancer, vaginal cancer, vulvar cancer); urologic cancer (e.g., renal cancer, urethral cancer, urothelial cancer, bladder cancer, cancer of the kidney or ureter, cancer of the renal pelvis, testicular cancer, penile cancer, cancer of the adrenal gland); gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer); ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors); thoracic cancer (e.g., non-small cell lung cancer, small cell lung cancer, mesothelioma, thymic malignancies, tracheal tumors, some esophageal cancer), leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)), lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma), other blood cancer (e.g., multiple myeloma), cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination of said cancers. In some aspects, the tumor is derived from a cancer selected from the group consisting of hepatocellular carcinoma, colon carcinoma, melanoma, and any combination thereof.

In some aspects, the subject suffers from a cancer. In some aspects, the subject suffers from a cancer selected from the group consisting of skin cancer (e.g., metastatic melanoma, cutaneous malignant melanoma, cutaneous squamous cell carcinoma, basal cell carcinoma); breast cancer (e.g., invasive breast cancer, triple-negative breast cancer, inflammatory breast cancer); brain cancer (e.g., glioblastoma multiforme, medulloblastoma, pituitary carcinoma, brain stem gliomas, astrocytomas, oligodendrogliomas, hemangiopericytomas, germ cell tumors, pineal tumors); bone cancer (e.g., chordomas, chondrosarcomas, osteosarcomas, Ewing sarcomas, fibrosarcomas, adamantiomas, giant cell tumors); head and neck cancer (e.g., head and neck squamous cell carcinoma; salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer); gynecologic cancer (e.g., ovarian cancer, uterine cancer, fallopian tube cancer, endometrial cancer, cervical cancer, vaginal cancer, vulvar cancer); urologic cancer (e.g., renal cancer, urethral cancer, urothelial cancer, bladder cancer, cancer of the kidney or ureter, cancer of the renal pelvis, testicular cancer, penile cancer, cancer of the adrenal gland); gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer); ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors); thoracic cancer (e.g., non-small cell lung cancer, small cell lung cancer, mesothelioma, thymic malignancies, tracheal tumors, some esophageal cancer), leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)), lymphoma (e.g., Hodgkin's lymphoma, non-Hodgkin's lymphoma (NHL), Waldenström macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma), other blood cancer (e.g., multiple myeloma), cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination of said cancers. In some aspects, the subject suffers from a cancer selected from the group consisting of a skin cancer, a gastrointestinal cancer, breast cancer, brain cancer, or any combination thereof.

In some aspects, cancer is selected from a group consisting of melanoma, colorectal cancer, breast cancer, glioblastoma multiforme, and any combination thereof.

In some aspects, the subject suffers from melanoma (e.g., uveal melanoma). See e.g., Sheppard et al., PLoS 8(4): e62753 (2013); Yang J. et al., Ther Adv Med Oncol. 2018; 10, published 2018 Feb. 21, 2018; Pelster et al., Journal of Clinical Oncology 37(15):9522-9522 (2019). In some aspects, the subject suffers from metastatic melanoma.

In some aspects, the subject suffers from colorectal cancer. See e.g., Tu S P et al., Gastroenterology, 128:361-375 (2005); Xue Z. et al., Oncology Reports., 25:1039-1046 (2011).

In some aspects, the subject suffers from breast cancer. See e.g., Lu L. et al., Oncol Rep., 28(4):1332-1338 (2012).

In some aspects, the subject suffers from glioblastoma multiforme (GMB). See e.g., Maguire C A, et al., J Neurooncol. 96(3):337-347 (2010).

In some aspects, the methods of the present disclosure can be used to reduce the tumor size of a tumor derived from, for example, skin cancer (e.g., metastatic melanoma, cutaneous malignant melanoma, cutaneous squamous cell carcinoma, basal cell carcinoma); breast cancer (e.g., invasive breast cancer, triple-negative breast cancer, inflammatory breast cancer); brain cancer (e.g., glioblastoma multiforme, medulloblastoma, pituitary carcinoma, brain stem gliomas, astrocytomas, oligodendrogliomas, hemangiopericytomas, germ cell tumors, pineal tumors); bone cancer (e.g., chordomas, chondrosarcomas, osteosarcomas, Ewing sarcomas, fibrosarcomas, adamantiomas, giant cell tumors); head and neck cancer (e.g., head and neck squamous cell carcinoma; salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer); gynecologic cancer (e.g., ovarian cancer, uterine cancer, fallopian tube cancer, endometrial cancer, cervical cancer, vaginal cancer, vulvar cancer); urologic cancer (e.g., renal cancer, urethral cancer, urothelial cancer, bladder cancer, cancer of the kidney or ureter, cancer of the renal pelvis, testicular cancer, penile cancer, cancer of the adrenal gland); gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer); ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors); thoracic cancer (e.g., non-small cell lung cancer, small cell lung cancer, mesothelioma, thymic malignancies, tracheal tumors, some esophageal cancer), leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)), lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma), other blood cancer (e.g., multiple myeloma), cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and combinations of said cancers. In some aspects, the solid tumor is a surface lesion. In some aspects, the solid tumor is a visceral tumor.

In some aspects, the subject suffers from metastatic cancer. In some aspects, the subject suffers from advance or metastatic melanoma, cutaneous squamous cell carcinoma, colorectal cancer, prostate cancer, breast cancer or esophageal cancer.

In some aspects, the method of treating or reducing symptoms in a subject in need thereof comprises administering a gene therapy, a polynucleotide, an AAV capsid, or a composition of the present disclosure to the subject in combination with a checkpoint inhibitor.

In some aspects, the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

In some aspects, the checkpoint inhibitor is a PD-1 inhibitor. In some aspects, the checkpoint inhibitor is a PD-L1 inhibitor. In some aspects, the PD-1 inhibitor is an anti-PD-1 antibody. In some aspects, the checkpoint inhibitor comprises pembrolizumab, nivolumab, cemiplimab, toripalimab, spartalizumab, dostarlimab-gxly, tislelizumab, balstilimab, BI-754091, zimberelimab, INCMGA00012, camrelizumab, or any combination thereof.

In some aspects, the checkpoint inhibitor is a PD-L1 inhibitor. In some aspects, the PD-L1 inhibitor is an anti-PD-L1 antibody. In some aspects, the checkpoint inhibitor comprises durvalumab, atezolizumab, avelumabor, or any combination thereof.

In some aspects, the checkpoint inhibitor is a CTLA-4 inhibitor. In some aspects, the checkpoint inhibitor comprises ipilimumab, tremelimumab (formerly ticilimumab), zalifrelimab, quavonlimab, BA3071, YH001, or any combination thereof.

In some aspects, the gene therapy, the polynucleotide, or the AAV capsid of the present disclosure can be administered prior to, at the same time, or after the administration of a checkpoint inhibitor.

In some aspects, the methods disclosed herein can be practiced through the administration of the composition (e.g., gene therapy composition) comprising the AAV vector, the AAV vector, the AAV capsid, a cell comprising an AAV vector of the present disclosure, a cell comprising the AAV capsid of the present disclosure, a cell comprising a polynucleotide encoding an immunomodulatory protein (e.g., a second cytokine) or functional fragment thereof of the present disclosure integrated into its genomic DNA, or a pharmaceutical compositions comprising any of the above. Thus, methods disclosed herein reciting the administration of an AAV vector of the present disclosure can be also practiced by administering any of these compositions.

Based on the methods disclosed herein, the composition (e.g., gene therapy composition) comprising an AAV vector, an AAV vector, or an AAV capsid of the present disclosure for use in therapy, or for use as a medicament, or for use in treating a disease or disorder (e.g., a tumor or a cancer) in a subject in need thereof is contemplated. In some aspects, the gene therapy constructs comprising the coding sequences for the immunomodulatory proteins (e.g., cytokines), one or more subunits thereof, or functional fragments thereof, as disclosed herein, can provide durable passive protection.

In some aspects, the composition (e.g., gene therapy composition), the polynucleotide, or AAV capsid disclosed herein is administered intratumorally, e.g., by direct injection to the tumor.

In some aspects, the composition (e.g., gene therapy composition), the polynucleotide, or AAV capsid disclosed herein is administered intravenously.

In some aspects, the composition (e.g., gene therapy composition), the polynucleotide, or AAV capsid disclosed herein is administered intrathecally.

In some aspects, the composition (e.g., gene therapy composition), the polynucleotide, or AAV capsid disclosed herein is administered intrahepatically.

When administered to a subject with a cancer, in certain aspects, the composition (e.g., gene therapy composition), the polynucleotide, or AAV capsid of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, the composition (e.g., gene therapy composition), the polynucleotide, or AAV capsid of the present disclosure is administered in an amount and for a time sufficient to convert a “cold tumor” into a “hot tumor”, i.e., said administering results in the infiltration of leukocytes (such as T-cells) into the tumor microenvironment. The term, “distal tumor” or “distant tumor” as used herein refers to a tumor that has spread from the original (or primary) tumor to distant organs or distant tissues, e.g., lymph nodes. In some aspects, the composition (e.g., gene therapy composition), the polynucleotide, or AAV capsid of the present disclosure treats a tumor after the metastatic spread. In some aspects, the composition (e.g., gene therapy composition), the polynucleotide, or AAV capsid disclosed herein is administered intrasplenically. In some aspects, the composition (e.g., gene therapy composition), the polynucleotide, or AAV capsid disclosed herein is administered intrathecally. In some aspects, the composition (e.g., gene therapy composition), the polynucleotide, or AAV capsid disclosed herein is administered intrahepatically. In some aspects, the composition (e.g., gene therapy composition), the polynucleotide, or AAV capsid disclosed herein is administered via intraosseous infusion. In some aspects, the composition (e.g., gene therapy composition), the polynucleotide, or AAV capsid disclosed herein is administered intradermally. In some aspects, the composition (e.g., gene therapy composition), the polynucleotide, or AAV capsid disclosed herein is administered via intraparenchymal route. In some aspects, the composition (e.g., gene therapy composition), the polynucleotide, or AAV capsid disclosed herein is administered to salivary gland. In some aspects, the composition (e.g., gene therapy composition), the polynucleotide, or AAV capsid disclosed herein is administered into a lymph node (e.g., via direct injection). In some aspects, the lymph node is a metastatic lymph node.

In some aspects, anti-tumor efficacy in a subject can be assessed for overall response rate (using RECIST v1.1 (or similar) (see e.g., Eisenhauer et al., Eur J Cancer, 45:228-247 (2009))) for a gene therapy, a polynucleotide, an AAV capsid, or a composition disclosed herein, which can be administered either as a monotherapy or a combination therapy with a checkpoint inhibitor in a subject in need thereof. In some aspects, the subject has progressed on an anti-PD-1 antibody therapy.

In some aspects, the best overall objective response rate (ORR) is improved against the known rates for checkpoint inhibition as a monotherapy in a similar subject population. The ORR is equal to the sum of the complete response (CR) plus the partial response (PR).

In some aspects, useful biomarkers can include the PD-L1 expression levels (e.g., as assessed by immunohistochemistry (IHC)), and tumor-infiltrating lymphocytes (TIL) profile (e.g., as assessed by CD8+ T cell density in tumor tissue), as described in e.g., Buisseret L., et al., Oncoimmunology. 6(1): e1257452 (2017). In some aspects, changes in PD-L1 expression can be assessed pre- and post-treatment.

### VI. Pharmaceutical Compositions

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for intratumoral administration.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intratumoral administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for intravenous administration.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intravenous administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for intrasplenic administration.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intrasplenic administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for intrathecal administration.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intrathecal administration.

In some aspects, a pharmaceutical composition disclosed herein comprises a delivery vector of the present disclosure (e.g., a viral vector, a non-viral vectors, a plasmid, a lipid, protein particle, a bacterial vector, or a lysosome) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for intrahepatic administration.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intrahepatic administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for intraosseous administration.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intraosseous administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a promoter operably linked to a nucleic acid sequence that encodes an immunomodulatory protein (e.g., a cytokine), one or more subunits thereof, or functional fragment thereof as disclosed herein and a pharmaceutically-acceptable excipient or carrier in a form suitable for administration into a lymph node.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for administration into a lymph node.

In some aspects, a pharmaceutical composition disclosed herein comprises an AAV delivery vector comprising a polynucleotide comprising a nucleic acid encoding a cytokine, one or more subunits thereof, or functional fragment thereof (e.g., IL-12, or one or more subunits thereof); and, optionally, (b) a checkpoint inhibitor (e.g., a PD-1 inhibitor) is suitable for intraperitoneal administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intratumoral administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intratumoral administration. In some aspects, each delivery vector (e.g., viral vector) comprises a polynucleotide comprising the first nucleic acid and the second nucleic acid.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intravenous administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intravenous administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intrasplenic administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intrasplenic administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intrathecal administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intrathecal administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intrahepatic administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intrahepatic administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intraosseous administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intraosseous administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intradermal administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intradermal administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for intraparenchymal administration. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intraparenchymal administration.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for administration via delivery to salivary gland. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for administration via delivery to salivary gland.

In some aspects, the pharmaceutical composition comprises one or more delivery vectors (e.g., viral vectors) for delivery of a combination of (i) a first nucleic acid encoding a first immunomodulatory protein, one or more subunits thereof, or functional fragment thereof and (ii) a second nucleic acid encoding a second immunomodulatory protein, one or more subunits thereof, or functional fragment thereof, and a pharmaceutically-acceptable excipient or carrier in a form suitable for administration into a lymph node. In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable foradministration into a lymph node.

In some aspects, the pharmaceutical composition comprises one or more delivery vectors (e.g., viral vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and a pharmaceutically-acceptable excipient or carrier in a form suitable for intraperitoneal administration.

In some aspects, each delivery vector (e.g., viral vector) comprises either the first nucleic acid or the second nucleic acid, wherein two or more delivery vectors are delivered in combination (e.g., simultaneously or sequentially).

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector), and a pharmaceutically-acceptable excipient or carrier in a form suitable for intratumoral administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intratumoral administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector), and a pharmaceutically-acceptable excipient or carrier in a form suitable for intravenous administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intravenous administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector), and a pharmaceutically-acceptable excipient or carrier in a form suitable for intrasplenic administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intrasplenic administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector), and a pharmaceutically-acceptable excipient or carrier in a form suitable for intrathecal administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intrathecal administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector), and a pharmaceutically-acceptable excipient or carrier in a form suitable for intrahepatic administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intrahepatic administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector), and a pharmaceutically-acceptable excipient or carrier in a form suitable for intraosseous administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intraosseous administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector), and a pharmaceutically-acceptable excipient or carrier in a form suitable for intradermal administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; and (b) a delivery vector (e.g., viral vector), and a pharmaceutically-acceptable excipient or carrier in a form suitable for intradermal administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector), and a pharmaceutically-acceptable excipient or carrier in a form suitable for intraparenchymal administration. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and (b) a delivery vector (e.g., viral vector), and a pharmaceutically-acceptable excipient or carrier in a form suitable for intraparenchymal administration.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector), and a pharmaceutically-acceptable excipient or carrier in a form suitable for administration via delivery to salivary gland. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and (b) a delivery vector (e.g., viral vector), and a pharmaceutically-acceptable excipient or carrier in a form suitable for administration via delivery to salivary gland.

In some aspects, the pharmaceutical composition comprises: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof, and (b) a delivery vector (e.g., viral vector), and a pharmaceutically-acceptable excipient or carrier in a form suitable for administration into a lymph node. In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and (b) a delivery vector (e.g., viral vector), and a pharmaceutically-acceptable excipient or carrier in a form suitable for administration into a lymph node.

In some aspects, the pharmaceutical composition comprises: (a) a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof, and (b) a delivery vector (e.g., viral vector), and a pharmaceutically-acceptable excipient or carrier in a form suitable for intraperitoneal administration.

In some aspects, the pharmaceutical composition comprises a polynucleotide comprising: (a) a first nucleic acid encoding a first cytokine, one or more subunits thereof, or functional fragment thereof, and (b) a second nucleic acid encoding a second cytokine, one or more subunits thereof, or functional fragment thereof, and a pharmaceutically-acceptable excipient or carrier in a form suitable for intratumoral administration.

In some aspects, the pharmaceutical composition comprises a polynucleotide comprising: (a) a first nucleic acid encoding a first cytokine, one or more subunits thereof, or functional fragment thereof, and (b) a second nucleic acid encoding a second cytokine, one or more subunits thereof, or functional fragment thereof, and a pharmaceutically-acceptable excipient or carrier in a form suitable for intravenous administration. In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intratumoral administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intravenous administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intrasplenic administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intrathecal administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intrahepatic administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intraosseous administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intradermal administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for intraparenchymal administration.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for administration via delivery to salivary gland.

In some aspects, a pharmaceutical composition disclosed herein comprises a delivery vector of the present disclosure (e.g., a viral vector, a non-viral vectors, a plasmid, a lipid, protein particle, a bacterial vector, a lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof and a pharmaceutically-acceptable excipient or carrier in a form suitable for administration into a lymph node.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intratumoral administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intravenous administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intrasplenic administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intrathecal administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intrahepatic administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intraosseous administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intradermal administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intraparenchymal administration.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for administration via delivery to salivary gland.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for administration into a lymph node.

In some aspects, the pharmaceutical composition comprises a polynucleotide comprising: (i) a promoter, (ii) a first nucleic acid encoding a first cytokine subunit or functional fragment thereof, (iii) a translation modification sequence, and (iv) a second nucleic acid encoding a second cytokine subunit or functional fragment thereof, and a pharmaceutically-acceptable excipient or carrier in a form suitable for intraperitoneal administration.

In some aspects, the first cytokine subunit and the second cytokine subunit are IL-12 subunits. In some aspects, the first nucleic acid or the second nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77. In some aspects, the first nucleic acid or the second nucleic acid comprises a sequence at least 85%, at least 90%, at least 96%, at least 97%, at least 98%, at least 99%, or 100% identical to SEQ ID NO: 8, SEQ ID NO: 9, or SEQ ID NO: 10.

Pharmaceutically acceptable excipients or carriers are determined in part by the particular composition being administered, as well as by the particular method used to administer the composition.

Accordingly, there is a wide variety of suitable formulations of pharmaceutical compositions comprising a delivery vector of the present disclosure (e.g., an AAV vector) or a plurality thereof (see, e.g., Remington's Pharmaceutical Sciences, Mack Publishing Co., Easton, Pa. 18th ed. (1990)) and/or one or more shRNA disclosed herein. The pharmaceutical compositions are generally formulated sterile and in full compliance with all Good Manufacturing Practice (GMP) regulations of the U.S. Food and Drug Administration. In some aspects, the pharmaceutical composition comprises more than one AAV vector of the present disclosure, wherein each vector comprises at least one polynucleotide encoding at least one disclosed herein.

In some aspects, a pharmaceutical composition comprises (i) one or more delivery vectors disclosed herein (e.g., AAV vectors or AAV capsids), and (ii) one or more therapeutic agents for the treatment of a disorder. In some aspects, the one or more delivery vectors disclosed herein (e.g., AAV vectors or AAV capsids) and the one or more therapeutic agents for a disease or disorder (e.g., a cancer, a tumor) are co-administered as separate pharmaceutical compositions.

In some aspects, a pharmaceutical composition comprising one or more delivery vectors disclosed herein (e.g., AAV vectors or AAV capsids) is administered prior to the administration of a pharmaceutical composition comprising one or more therapeutic agents for the treatment of a disease or disorder (e.g., a cancer, a tumor). In some aspects, a pharmaceutical composition comprising one or more delivery vectors disclosed herein (e.g., AAV vectors or AAV capsids) is administered after the administration of a pharmaceutical composition comprising one or more therapeutic agents for the treatment of a disease or disorder (e.g., a cancer, a tumor). In some aspects, a pharmaceutical composition comprising one or more delivery vectors disclosed herein (e.g., AAV vectors or AAV capsids) is administered concurrently with a pharmaceutical composition comprising one or more therapeutic agents for the treatment of a disease or disorder (e.g., a cancer, a tumor).

Also provided herein are pharmaceutical compositions comprising delivery vectors disclosed herein (e.g., AAV vectors or AAV capsids) having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of vectors, e.g., AAV vectors described herein. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

Acceptable carriers, excipients, or stabilizers are nontoxic to recipients (e.g., animals or humans) at the dosages and concentrations employed.

Examples of carriers or diluents include, but are not limited to, water, saline, Ringer's solutions, dextrose solution, and 5% human serum albumin. Except insofar as any conventional media or compound is incompatible with the delivery vectors disclosed herein (e.g., AAV vectors or AAV capsids), use thereof in the compositions is contemplated. In some aspects, a pharmaceutical composition is formulated to be compatible with its intended route of administration. The delivery vectors disclosed herein (e.g., AAV vectors or AAV capsids) can be administered by intravenous, parenteral, topical, intravenous, oral, subcutaneous, intra-arterial, intradermal, transdermal, rectal, intracranial, intraperitoneal, intranasal, intratumoral, intramuscular route or as inhalants. In some aspects, the pharmaceutical composition comprising the delivery vectors disclosed herein (e.g., AAV vectors or AAV capsids) is administered intratumorally, e.g. by direct injection. In some aspects, the pharmaceutical composition comprising the delivery vectors disclosed herein (e.g., AAV vectors or AAV capsids) is administered intravenously. In some aspects, the pharmaceutical composition comprising the delivery vectors disclosed herein (e.g., AAV vectors or AAV capsids) is administered intrasplenically. In some aspects, the pharmaceutical composition comprising the delivery vectors disclosed herein (e.g., AAV vectors or AAV capsids) is administered intrathecally. In some aspects, the pharmaceutical composition comprising the delivery vectors disclosed herein (e.g., AAV vectors or AAV capsids) is administered intrahepatically. In some aspects, the pharmaceutical composition comprising the delivery vectors disclosed herein (e.g., AAV vectors or AAV capsids) is administered via intraosseous infusion. In some aspects, the pharmaceutical composition comprising the delivery vectors disclosed herein (e.g., AAV vectors or AAV capsids) is administered intradermally. In some aspects, the pharmaceutical composition comprising the delivery vectors disclosed herein (e.g., AAV vectors or AAV capsids) is administered via intraparenchymal route.

In some aspects, the pharmaceutical composition comprising the delivery vectors disclosed herein (e.g., AAV vectors or AAV capsids) is administered via delivery to salivary gland. In some aspects, the pharmaceutical composition comprising the delivery vectors disclosed herein (e.g., AAV vectors or AAV capsids) is administered via direct injection into a lymph node. In some aspects, the lymph node is a metastatic lymph node.

The delivery vectors disclosed herein (e.g., AAV vectors or AAV capsids) can optionally be administered in combination with other therapeutic agents that are at least partly effective in treating the disease, disorder or condition for which the delivery vectors disclosed herein (e.g., AAV vectors or AAV capsids) are intended. In some aspects, the vectors disclosed herein are administered in combination with a PD-1 inhibitor (e.g., an anti-PD-1 antibody).

The delivery vectors disclosed herein (e.g., AAV vectors or AAV capsids) can be formulated using one or more excipients to (1) increase stability; (2) increase cell transfection or transduction; (3) permit the sustained or delayed release; or (4) alter the biodistribution (e.g., target the AAV vector to specific tissues or cell types).

### VII. Administration

The compositions (e.g., gene therapy compositions) and delivery vectors disclosed herein (e.g., AAV vectors or AAV capsids) can be administered by any route which results in a therapeutically effective outcome. In some aspects, the methods disclosed herein can comprise re-administering a gene therapy, a polynucleotide, a AAV capsid, or a composition of the present disclosure to the subject.

In some aspects, the administration (or delivery) is intratumorally, intravenously, intrasplenically, intrathecally, intrahepatically, via intraosseous infusion, intradermal, intraparenchymal, via delivery to salivary gland, or via delivery into a lymph node.

In some aspects, the delivery can be intratumoral, e.g., by direct injection to the tumor. In some aspects, the compositions (e.g., gene therapy compositions) and delivery vectors disclosed herein (e.g., AAV vectors or AAV capsids) is administered as a single dose. In some aspects, the single dose can comprise one or more injections. In some aspects, the single dose includes multiple injections into different regions of the tumor.

In some aspects, the delivery can be intravenous.

In some aspects, the delivery can be intrasplenic. See e.g., Xiao et al., Molecular Therapy: Methods & Clinical Development, 1(4):323-329 (2000); Ahmed et al., “Gene transfer in the liver using recombinant adeno-associated virus.” Current Protocols in Microbiology vol. Chapter 14 (2013).

In some aspects, the delivery can be intrathecal delivery.

In some aspects, the delivery can be intrahepatic delivery.

In some aspects, the delivery can be intraosseous. See e.g., Lee et al., Molecular Therapy: Methods & Clinical Development, 15:101-111 (2019).

In some aspects, the delivery can be intradermal.

In some aspects, the delivery can be intraparenchymal.

In some aspects, the delivery can be to salivary gland.

In some aspects, the delivery can be directly into a lymph node. In some aspects, the lymph node is a metastatic lymph node. See e.g., Sato et al., PLoS One., 2015; 10(4): e0123619; published online Apr. 21, 2015.

In some aspects, a vector (e.g., either a viral vector (e.g., an AAV vector) or non-viral vector (including naked DNA)) comprising a nucleic acid is administered to a tumor. Because the vector is presented to the cells from “outside” the body, the immunological and inflammatory reactions that are commonly observed as a result of the administration of transforming formulations and their adjuvants into blood and interstitial fluid may be avoided.

The amount of nucleic acid to transform a sufficient number of tumor cells and provide for expression of therapeutic levels of the protein can be assessed, e.g., using an animal model (e.g., a rodent (mouse or rat) or other mammalian animal model) to assess factors such as the efficiency of transformation, the levels of protein expression achieved, the susceptibility of the targeted secretory gland cells to transformation, and the amounts of vector and/or nucleic acid required to transform tumor cells.

The precise amount of vector and/or nucleic acid administered will vary greatly according to a number of factors including the susceptibility of the target cells to transformation, the size and weight of the subject, the levels of protein expression desired, and the condition to be treated.

The methods of the disclosure can be used to accomplish delivery of the immunomodulatory proteins or functional fragments thereof to the bloodstream on either a long-term basis (e.g., by repeated administration of the construct) or on a short-term basis (e.g., for several hours or a few days). In this regard, the disclosure takes advantage of the normal turnover of the cells that are transformed by the introduced construct in order to provide a means for controlling dosage of the polypeptide to the bloodstream.

In some aspects, a delivery vector of the present disclosure (e.g., a viral vector, a non-viral vectors, a plasmid, a lipid, protein particle, a bacterial vector, protein particle, a bacterial vector, a lysosome, a virus-like particle, a polymeric particle, an exosome, or a vault particle) comprising a promoter operably linked to a nucleic acid sequence that encodes an immunomodulatory protein or functional fragment thereof is administered by direct injection to a tumor.

In some aspects, a delivery vector of the present disclosure (e.g., a viral vector, a non-viral vectors, a plasmid, a lipid, protein particle, a bacterial vector, protein particle, a bacterial vector, or lysosome, a virus-like particle, a polymeric particle, an exosome, or a vault particle) comprising a first promoter which is operably linked to a first nucleic acid encoding a first immunomodulatory protein (e.g., a first cytokine), one or more subunits thereof, or functional fragment thereof and a second promoter which is operably linked to a second nucleic acid encoding a second immunomodulatory protein (e.g., a second cytokine), one or more subunits thereof, or functional fragment thereof is administered by direct injection to a tumor.

In some aspects, a delivery vector of the present disclosure (e.g., a viral vector) comprises (i) a promoter, (ii) a first nucleic acid encoding a first cytokine subunit (e.g., an IL-12 p35 subunit) or functional fragment thereof, (iii) a translation modification sequence, and (iv) a second nucleic acid encoding a second cytokine subunit (e.g., an IL-12 p40 subunit) or functional fragment thereof is administered by direct injection to a tumor.

The compositions (e.g., gene therapy compositions) and delivery vectors disclosed herein (e.g., AAV vectors or AAV capsids) can be administered in any suitable form, either as a liquid solution or suspension, as a solid form suitable for liquid solution or suspension in a liquid solution.

In some aspects, the composition (e.g., gene therapy composition) or AAV capsid disclosed herein is administered to a human. In further aspects, the composition (e.g., gene therapy composition) or AAV capsid disclosed herein is administered to an elderly human (i.e., human≥65 years of age). In other aspects, the composition (e.g., gene therapy composition) or AAV capsid disclosed herein is administered to a human who is immunocompromised or immunodeficient. In some aspects, the human who is immunocompromised or immunodeficient has been diagnosed, is undergoing treatment, or has recovered from a B-cell immunodeficiencies, T-cell immunodeficiencies, phagocyte disorders, complement defects, chronic and/or serious disease, immunomodulatory medications, and other. B-cell immunodeficiencies include, for example, Brutons' agammaglobulinemia, common variable immunodeficiency, hyper-IgM syndrome and selective IgA deficiency. In some aspects, the T-cell immunodeficiencies include, for example, DiGeorge syndrome, Bare lymphocyte syndrome, and Omenn's syndrome. Phagocyte disorders include, for example, chronic granulomatous disease, Leukocyte adhesion deficiency, Chediak-Higashi syndrome, and Cyclic neutropenia Kostman diseases. Complement defects include C1, C2, or C4 deficiency—defects in clearing immunocomplexes, C3 or C5 deficiency—block in alternative and classical pathways, and C6, C7, C8, or C9 deficiency—defect in MAC assembly and function. Chronic and/or serious disease include, for example, HIV/AIDS, Cancer (e.g., leukemia, lymphoma), Chronic inflammatory diseases treated w/high-level immunosuppressive meds, and Metabolic diseases (e.g., diabetes, obesity, uremia). Immunomodulatory medications include, for example, Cytotoxic agents/chemotherapy, High-dose corticosteroids, and Disease-modifying anti-rheumatic drugs (DMARDs). Additional immunodeficiencies include, for example, malnutrition, ageing, surgery (e.g., splenectomy), and Inherited defects other than PIDs (e.g. Down Syndrome; Cystic Fibrosis, Sickle Cell Disease).

In some aspects, provided herein is a combination therapy comprising a) the composition (e.g., gene therapy composition) disclosed herein, the polynucleotide disclosed herein, or the AAV capsid disclosed herein, and b) a checkpoint inhibitor.

In some aspects, the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

In some aspects, the checkpoint inhibitor is a PD-1 inhibitor. In some aspects, the checkpoint inhibitor is a PD-L1 inhibitor. In some aspects, the PD-1 inhibitor is an anti-PD-1 antibody. In some aspects, the checkpoint inhibitor comprises pembrolizumab, nivolumab, cemiplimab, toripalimab, spartalizumab, dostarlimab-gxly, tislelizumab, balstilimab, BI-754091, zimberelimab, INCMGA00012, camrelizumab, or any combination thereof.

In some aspects, the checkpoint inhibitor is a PD-L1 inhibitor. In some aspects, the checkpoint inhibitor comprises durvalumab, atezolizumab, avelumabor, or any combination thereof.

In some aspects, the checkpoint inhibitor is a CTLA-4 inhibitor. In some aspects, the checkpoint inhibitor comprises ipilimumab, tremelimumab (formerly ticilimumab), zalifrelimab, quavonlimab, BA3071, YH001, or any combination thereof.

In some aspects, the composition (e.g., gene therapy composition) disclosed herein, the polynucleotide disclosed herein, or the AAV capsid disclosed herein is administered prior to, at the same time, or after the administration of the checkpoint inhibitor.

In some aspects, the composition (e.g., gene therapy composition) disclosed herein is administered prior to, at the same time, or after the administration of the checkpoint inhibitor.

In some aspects, the polynucleotide disclosed herein is administered prior to, at the same time, or after the administration of the checkpoint inhibitor.

In some aspects, the AAV capsid disclosed herein is administered prior to, at the same time, or after the administration of the checkpoint inhibitor.

In some aspects, the composition (e.g., gene therapy composition) disclosed herein is administered in the same composition as the checkpoint inhibitor.

In some aspects, the polynucleotide disclosed herein is administered in the same composition as of the checkpoint inhibitor.

In some aspects, the AAV capsid disclosed herein is administered in the same composition as the checkpoint inhibitor.

In some aspects, the composition (e.g., gene therapy composition) disclosed herein is administered in a different composition than the checkpoint inhibitor.

In some aspects, the polynucleotide disclosed herein is administered in a different composition than the checkpoint inhibitor.

In some aspects, the AAV capsid disclosed herein is administered in a different composition than the checkpoint inhibitor.

In some aspects, the checkpoint inhibitor is administered intravenously (e.g., intravenous infusion). In some aspects, the checkpoint inhibitor is administered as a single dose. In some aspects, the checkpoint inhibitor is administered in multiple doses.

In some aspects, the checkpoint inhibitor (e.g., nivolumab) is administered intravenously at a dose of about 240 mg every 2 weeks or about 480 mg every 4 weeks.

In some aspects, the checkpoint inhibitor (e.g., pembrolizumab) is administered intravenously at a dose of 200 mg every 3 weeks or 400 mg every 6 weeks. In some the checkpoint inhibitor (e.g., pembrolizumab) is administered intravenously at a dose of 2 mg/kg (up to 200 mg) every 3 weeks.

In some aspects, the subject to which the compositions (e.g., gene therapy compositions), polynucleotides, expression constructs, or AAV capsids (e.g., AAV particles) of the disclosure are administered is a subject that suffers from a solid tumor cancer. In some aspects, the solid tumor is a sarcoma, a carcinoma, or a lymphoma. In some aspects, the solid tumor is a surface lesion. In some aspects, the solid tumor is a visceral tumor.

In some aspects, the subject suffers from a cancer selected from the group consisting of skin cancer, gastrointestinal cancer, breast cancer, brain cancer, bone cancer, thoracic cancer, head and neck cancer, gynecologic cancer, urologic cancer, ocular cancer, and any combination thereof.

In some aspects, cancer is selected from a group consisting of:
skin cancer (e.g., metastatic melanoma, cutaneous malignant melanoma, cutaneous squamous cell carcinoma, basal cell carcinoma);
breast cancer (e.g., invasive breast cancer, triple-negative breast cancer, inflammatory breast cancer);
brain cancer (e.g., glioblastoma multiforme, medulloblastoma, pituitary carcinoma, brain stem gliomas, astrocytomas, oligodendrogliomas, hemangiopericytomas, germ cell tumors, pineal tumors);
bone cancer (e.g., chordomas, chondrosarcomas, osteosarcomas, Ewing sarcomas, fibrosarcomas, adamantiomas, giant cell tumors);
head and neck cancer (e.g., head and neck squamous cell carcinoma (HNSCC); salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer);
gynecologic cancer (e.g., ovarian cancer, uterine cancer, fallopian tube cancer, endometrial cancer, cervical cancer, vaginal cancer, vulvar cancer);
urologic cancer (e.g., renal cancer, urethral cancer, urothelial cancer, bladder cancer, cancer of the kidney or ureter, cancer of the renal pelvis, testicular cancer, penile cancer, cancer of the adrenal gland);
gastrointestinal cancer (e.g., cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer);
ocular cancer (e.g. uveal melanoma, retinoblastoma, Merkel cell carcinoma, intraocular malignant melanoma, mucosa-associated lymphoid tissue lymphoma, orbital lymphoma, orbital sarcoma, lacrimal gland tumors);
thoracic cancer (e.g., non-small cell lung cancer, small cell lung cancer, mesothelioma, thymic malignancies, tracheal tumors, some esophageal cancer),
leukemia (e.g., chronic or acute leukemia, acute myeloid leukemia (AML), chronic myeloid leukemia (CML), acute lymphoblastic leukemia (ALL) (including non-T cell ALL), chronic lymphocytic leukemia (CLL)),
lymphoma (e.g., Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma),
other blood cancer (e.g., multiple myeloma)
cancer of the endocrine system, sarcoma of soft tissue, neoplasm of the central nervous system (e.g., CNS; atypical teratoid/rhaboid tumor, spinal axis tumor), tumor angiogenesis, Kaposi's sarcoma, epidermoid cancer, environmentally induced cancers including those induced by asbestos, other B cell malignancies, neuroblastoma (including olfactory neuroblastoma), rhabdomyosarcoma, and any combination thereof.

In some aspects, the solid tumor is a surface lesion. In some aspects, the solid tumor is a visceral tumor.

### VIII. Kits

The present disclosure also provides kits, or products of manufacture, comprising (i) the delivery vector of the present disclosure, or a pharmaceutical composition of the present disclosure, and (ii) optionally instructions for use (e.g., a package insert with instructions to perform any of the methods described herein).

In some aspects, the kit or product of manufacture comprises (i) comprising the delivery vectors of the present disclosure (e.g., an AAV vector comprising a polynucleotide encoding immunomodulatory protein (e.g., a cytokine) or functional fragment thereof disclosed herein), or a pharmaceutical composition of the present disclosure, (ii) optionally, an additional therapeutic agent, and (iii) optionally, instructions for use (e.g., a package insert with instructions to perform any of the methods described herein are also contemplated).

In some aspects, the kit or product of manufacture comprises (i) comprising an AAV vector comprising a polynucleotide comprising a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; or a pharmaceutical composition of the present disclosure, (ii) optionally, an additional therapeutic agent (e.g., a checkpoint inhibitor (e.g., a PD-1 inhibitor)), and (iii) optionally, instructions for use (e.g., a package insert with instructions to perform any of the methods described herein are also contemplated).

In some aspects, the kit or product of manufacture comprises (i) a polynucleotide, an AAV capsid, or a composition (e.g., gene therapy composition) of the present disclosure, (ii) optionally, an additional therapeutic agent, and (iii) optionally, instructions for use (e.g., a package insert with instructions to perform any of the methods described herein are also contemplated)

In some aspects, the kit or product of manufacture comprises (i) a composition comprising one or more delivery vectors (e.g., viral vectors) for delivery of a combination of (a) a first nucleic acid encoding a first immunomodulatory protein, one or more subunits thereof, or functional fragment thereof and (b) a second nucleic acid encoding a second immunomodulatory protein, one or more subunits thereof, or functional fragment thereof; (ii) optionally, an additional therapeutic agent, and (iii) optionally, instructions for use (e.g., a package insert with instructions to perform any of the methods described herein are also contemplated). In some aspects, the kit or product of manufacture comprises (i) a composition comprising one or more delivery vectors (e.g., AAV vectors) for delivery of a combination of a first nucleic acid encoding a first cytokine subunit (e.g., an IL-12 subunit), or functional fragment thereof and a second nucleic acid encoding a second cytokine subunit (e.g., an IL-12 subunit), or functional fragment thereof; or a pharmaceutical composition of the present disclosure, (ii) optionally, an additional therapeutic agent (e.g., a checkpoint inhibitor (e.g., a PD-1 inhibitor)), and (iii) optionally, instructions for use (e.g., a package insert with instructions to perform any of the methods described herein are also contemplated).

In some aspects, each delivery vector (e.g., viral vector) comprises a polynucleotide comprising the first nucleic acid and the second nucleic acid. In some aspects, each delivery vector (e.g., viral vector) comprises either the first nucleic acid or the second nucleic acid, wherein two or more delivery vectors are delivered in combination (e.g., simultaneously or sequentially).

In some aspects, the kit or product of manufacture comprises (i) a composition comprising: (a) a polynucleotide comprising a first nucleic acid encoding a first immunomodulatory protein, one or more subunits thereof, or functional fragment thereof and a second nucleic acid encoding a second immunomodulatory protein, one or more subunits thereof, or functional fragment thereof; and (b) a delivery vector (e.g., viral vector); (ii) optionally, an additional therapeutic agent, and (iii) optionally, instructions for use (e.g., a package insert with instructions to perform any of the methods described herein are also contemplated).

In some aspects, the kit or product of manufacture comprises (i) a composition comprising: (a) a polynucleotide comprising (i) a promoter, (ii) a first nucleic acid encoding a first cytokine subunit (e.g., an IL-12 subunit) or functional fragment thereof, (iii) a translation modification sequence, and (iv) a second nucleic acid encoding a second cytokine subunit (e.g., an IL-12 subunit) or functional fragment thereof, and (b) a delivery vector (e.g., AAV vector); (ii) optionally, an additional therapeutic agent (e.g., a checkpoint inhibitor (e.g., a PD-1 inhibitor)), and (iii) optionally, instructions for use (e.g., a package insert with instructions to perform any of the methods described herein are also contemplated).

In some aspects, the kit or product of manufacture comprises (i) a delivery vectors of the present disclosure (e.g., an AAV vector comprising a polynucleotide comprising a first nucleic acid encoding a first immunomodulatory protein (e.g., a first cytokine), one or more subunits thereof, or functional fragment thereof and/or a second nucleic acid encoding a second immunomodulatory protein (e.g., a second cytokine), one or more subunits thereof, or functional fragment thereof disclosed herein), or a pharmaceutical composition of the present disclosure, (ii) optionally, an additional therapeutic agent, and (iii) optionally, instructions for use (e.g., a package insert with instructions to perform any of the methods described herein are also contemplated).

In some aspects, the kit or product of manufacture comprises (i) a delivery vectors of the present disclosure (e.g., an AAV vector comprising a polynucleotide comprising (i) a promoter, (ii) a first nucleic acid encoding a first cytokine subunit (e.g., an IL-12 subunit) or functional fragment thereof, (iii) a translation modification sequence, and (iv) a second nucleic acid encoding a second cytokine subunit (e.g., an IL-12 subunit) or functional fragment thereof), or a pharmaceutical composition of the present disclosure, (ii) optionally, an additional therapeutic agent (e.g., a checkpoint inhibitor (e.g., a PD-1 inhibitor)), and (iii) optionally, instructions for use (e.g., a package insert with instructions to perform any of the methods described herein are also contemplated).

In some aspects, the components of a kit or product of manufacture disclosed herein are in one or more containers. In some aspects, the kit or product of manufacture comprises (i) an AAV vector comprising a polynucleotide encoding immunomodulatory protein, one or more subunits thereof, or functional fragment thereof disclosed herein, and (ii) a brochure with instructions to insert the polynucleotide in the AAV vector.

In some aspects, the kit or product of manufacture comprises (i) an AAV vector comprising a polynucleotide encoding a cytokine subunit (e.g., an IL-12 subunit), and (ii) a brochure with instructions to insert the polynucleotide in the AAV vector.

In some aspects, the components of a kit or product of manufacture disclosed herein are in one or more containers. In some aspects, the kit or product of manufacture comprises (i) an AAV vector comprising a polynucleotide comprising a first nucleic acid encoding a first immunomodulatory protein (e.g., a first cytokine), one or more subunits thereof, or functional fragment thereof and a second nucleic acid encoding a second immunomodulatory protein (e.g., a second cytokine), one or more subunits thereof, or functional fragment thereof disclosed herein, and (ii) a brochure with instructions to insert the polynucleotide in the AAV vector.

In some aspects, the kit or product of manufacture comprises (i) an AAV vector comprising a polynucleotide comprising a first nucleic acid encoding a first cytokine subunit (e.g., an IL-12 subunit) or functional fragment thereof, and a second nucleic acid encoding a second cytokine subunit (e.g., an IL-12 subunit) or functional fragment thereof, and (ii) a brochure with instructions to insert the polynucleotide in the AAV vector.

In some aspects, a kit or product of manufacture of the present disclosure comprises at least one delivery vector (e.g., AAV vector or AAV capsid). In some aspects, a kit or product of manufacture of the present disclosure comprises at least one polynucleotide encoding at least one immunomodulatory protein (e.g., a cytokine), one or more subunits thereof, or functional fragment thereof disclosed herein. In some aspects, a kit or product of manufacture of the present disclosure comprises a polynucleotide encoding two or more immunomodulatory proteins (e.g., cytokines), one or more subunits thereof, or functional fragments thereof disclosed herein.

One skilled in the art will readily recognize that vectors, polynucleotides, and pharmaceutical compositions of the present disclosure, or combinations thereof, can be readily incorporated into one of the established kit formats which are well known in the art.

Certain aspects (E) of the disclosure include:

E1. A combination therapy comprising: (a) a first composition comprising an adeno-associated virus (AAV) vector comprising a polynucleotide comprising (i) a promoter, (ii) a first nucleic acid encoding a first Interleukin-12 (IL-12) subunit or functional fragment thereof, (iii) a translation modification sequence, and (iv) a second nucleic acid encoding a second IL-12 subunit or functional fragment thereof, and (b) a second composition comprising a checkpoint inhibitor.

E2. The combination therapy of E1, wherein the AAV vector is an AAV serotype 2 (AAV2) vector.

E3. The combination therapy of E1 or E2, wherein the translation modification sequence comprises a furin cleavage sequence, a 2A self-processing peptide sequence, an internal ribosomal entry site (IRES) sequence, or any combination thereof.

E4. The combination therapy of E3, wherein the translation modification sequence comprises a furin cleavage sequence and a 2A self-processing peptide sequence (F2A).

E5. The combination therapy of any one of E1-E5, wherein the promoter comprises a CAG promoter, a CBA promoter, a CMV promoter, an EF-1a (Elongation Factor 1a) promoter, a RSV (Rous Sarcoma Virus) promoter, an Ubiquitin (UbC) promoter, or any combination thereof.

E6. The combination therapy of any one of E1-E, wherein the polynucleotide further comprises a poly(A) (pA) sequence.

E7. The combination therapy of any one of E1-E6, wherein the polynucleotide further comprises two inverted terminal repeat (ITR) sequences.

E8. The combination therapy of any one of E1-E7, wherein the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

E9. The combination therapy of any one of E1-E8, wherein the checkpoint inhibitor comprises a programmed cell death protein 1 (PD-1) inhibitor.

E10. The combination therapy of E9, wherein the PD-1 inhibitor is an anti-PD-1 antibody.

E11. The combination therapy of any one of E1-E10, wherein the first IL-12 subunit comprises an amino acid sequence having at least 85%, 90%, 95%, 96%, 97%, 98%, 99%, or 100% identity to SEQ ID NO: 80 and the second IL-12 subunit comprises an amino acid sequence having at least 85%, 90%, 95%, 96%, 97%, 98%, 99%, or 100% identity to SEQ ID NO: 81.

E12. The combination therapy of any one of E1-E11, wherein the first nucleic acid comprises a nucleic acid sequence having at least 85%, 90%, 95%, 96%, 97%, 98%, 99%, or 100% identity to SEQ ID NO: 5, SEQ ID NO: 6, SEQ ID NO: 7, or SEQ ID NO: 77, and the second nucleic acid comprises a nucleic acid sequence having at least 85%, 90%, 95%, 96%, 97%, 98%, 99%, or 100% identity to SEQ ID NO: 8, SEQ ID NO: 9, SEQ ID NO: 10, or SEQ ID NO: 92.

E13. The combination therapy of any one of E1-E12, wherein the polynucleotide comprises a nucleic acid sequence having at least 85%, 90%, 95%, 96%, 97%, 98%, 99%, or 100% identity to SEQ ID NO: 82.

E14. The combination therapy of any one of E1-E13, wherein the first composition is suitable for an intratumoral delivery and the second composition is suitable for an intravenous delivery.

E15. A method of treating a subject suffering from a tumor comprising administering the combination therapy of any one of E1-E14 to the subject.

E16. The method of E15, wherein the first composition is administered intratumorally.

E17. The method of E16, wherein the first composition is administered to multiple sites of the tumor.

E18. The method of any one of E15-E17, wherein the first composition is administered prior to, at the same time, or after the administration of the second composition.

E19. The method of any one of E15-E18, wherein tumor is derived from a cancer selected from the group consisting of a skin cancer; a breast cancer; a brain cancer; a bone cancer; a head and neck cancer; a salivary gland cancer; a gynecologic cancer; a urologic cancer; a gastrointestinal cancer; an ocular cancer; a thoracic cancer; a blood cancer; a cancer of the endocrine system; a sarcoma of soft tissue; a neoplasm of the central nervous system; and any combination thereof.

E20. The method of any one of E15-E19, wherein tumor is derived from a cancer selected from the group consisting of a hepatocellular carcinoma, a colon carcinoma, and a melanoma.

E21. A composition comprising: (a) a polynucleotide comprising (i) a promoter, (ii) a first nucleic acid encoding a first cytokine subunit or functional fragment thereof, (iii) a translation modification sequence, and (iv) a second nucleic acid encoding a second cytokine subunit or functional fragment thereof; and (b) a delivery vector.

E22. The composition of E21, wherein the first cytokine subunit and the second cytokine subunit are IL-12 subunits.

E23. The composition of E22, wherein the first nucleic acid or the second nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77.

E24. The composition of E22 or E23, wherein first nucleic acid or the second nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92.

E25. A composition comprising: (a) a polynucleotide comprising a first nucleic acid encoding a first immunomodulatory protein, one or more subunits thereof, or functional fragment thereof and/or a second nucleic acid encoding a second immunomodulatory protein, one or more subunits thereof, or functional fragment thereof; and (b) a delivery vector.

E26. The composition of E25, wherein the first immunomodulatory protein, one or more subunits thereof, or functional fragment thereof and/or the second immunomodulatory protein, one or more subunits thereof, or functional fragment thereof is a cytokine or functional fragment thereof.

E27. The composition of E26, wherein the cytokine is selected from the group consisting of tumor necrosis factor alpha (TNF-α), a type I interferon (INF), a type II IFN, interleukin (IL)-2, IL-12, IL-15, IL-21, IL-23, IL-27, IL-18, IL-1, IL-3, IL-4, IL-5, IL-6, IL-7, IL-8, IL-9, IL-10, IL17, IL33, TL1A, CD40L, granulocyte-macrophage colony-stimulating factor (GM-CSF), any functional fragment thereof, and any combination thereof.

E28. The composition of any one of E25-E27, wherein the first immunomodulatory and the second immunomodulatory proteins are cytokines.

E29. The composition of any one of E25-28, wherein the first immunomodulatory protein is IL-12, one or more subunits thereof, or a functional fragment thereof

E30. The composition of any one of E25-29, wherein the first nucleic acid comprises a nucleic acid encoding an IL-12 p35 subunit and/or a nucleic acid encoding IL-12 p40 subunit.

E31. The composition of any one of E25-E30, wherein the first nucleic acid comprises a nucleic acid sequence having at least 85%, 90%, 95% 96%, 97%, 98%, 99%, or 100% identity to any of SEQ ID NO: 5-10, 77, or any nucleic acid sequence in Table 2, or any combination thereof.

E32. The composition of any one of E25-E31, wherein the second immunomodulatory protein comprises IL-15 or a functional fragment thereof.

E33. The composition of any one of E25-E32, wherein the second nucleic acid comprises an IL-15 gene.

E34. The composition of any one of E25-E33, wherein the second nucleic acid comprises a nucleic acid sequence having at least 85%, 90%, 95% 96%, 97%, 98%, 99%, or 100% identity to any of SEQ ID NO: 11-13 or any nucleic acid sequence in Table 4, or any combination thereof.

E35. The composition of any one of E25-E27 or E29-E34, wherein the first immunomodulatory protein, one or more subunits thereof, or functional fragment thereof or the second immunomodulatory protein, one or more subunits thereof, or functional fragment thereof is selected from the group consisting of soluble CD40 ligand (CD40L), CD19 ligand (CD19L), CD48 ligand (CD48L), CD20 ligand (CD20L), any functional fragment thereof, and any combination thereof.

E36. The composition of any one of E21-E35, wherein the composition is gene therapy composition.

E37. A polynucleotide comprising: (a) a first nucleic acid encoding a first cytokine, one or more subunits thereof, or functional fragment thereof; and/or (b) a second nucleic acid encoding a second cytokine, one or more subunits thereof, or functional fragment thereof.

E38. The polynucleotide of E37, wherein the first cytokine and the second cytokine are selected from the group consisting of tumor necrosis factor alpha (TNF-α), a type I interferon (INF), a type II IFN, interleukin (IL)-2, IL-12, IL-15, IL-21, IL-23, IL-27, IL-18, , IL-1, IL-3, IL-4, IL-5, IL-6, IL-7, IL-8, IL-9, IL-10, IL17, IL33, TL1A, CD40L, granulocyte-macrophage colony-stimulating factor (GM-CSF), any functional fragment thereof, and any combination thereof.

E39. The polynucleotide of E37 or E38, wherein the first cytokine is IL-12, IL-18, IL-21, or a functional fragment thereof.

E40. The polynucleotide of any one of E37-E39, wherein the first nucleic acid comprises a nucleic acid encoding an IL-12 p35 subunit and/or a nucleic acid encoding an IL-12 p40 subunit.

E41. The polynucleotide of any one of E37-E40, wherein the first nucleic acid comprises a nucleic acid sequence having at least 85%, 90%, 95% 96%, 97%, 98%, 99%, or 100% identity to any of SEQ ID NO: 5-10, 77, or any nucleic acid sequence in Table 2, or any combination thereof.

E42. The polynucleotide of any one of E37-E41, wherein the second cytokine is IL-15, IL-2, or a functional fragment thereof.

E43. The polynucleotide of any one of E37-E42, wherein the second nucleic acid comprises an IL-15 gene.

E44. The polynucleotide of any one of E37-E43, wherein the second nucleic acid comprises a nucleic acid sequence having at least 85%, 90%, 95% 96%, 97%, 98%, 99%, or 100% identity to any of SEQ ID NO: 11-13, 58, or any nucleic acid sequence in Table 4, or any combination thereof.

E45. A polynucleotide comprising (i) a promoter, (ii) a first nucleic acid encoding a first cytokine subunit or functional fragment thereof, (iii) a translation modification sequence, and (iv) a second nucleic acid encoding a second cytokine subunit or functional fragment thereof.

E46. The polynucleotide of E45, wherein the first cytokine subunit and the second cytokine subunit are IL-12 subunits.

E47. The polynucleotide of E45 or E46, wherein the first nucleic acid or the second nucleic acid comprises a sequence at least 85%, at least 90%, at least 96%, at least 97%, at least 98%, at least 99%, or 100% identical to SEQ ID NO: 5, SEQ ID NO: 6, SEQ ID NO: 7, or SEQ ID NO: 77.

E48. The polynucleotide of E45 or E46, wherein first nucleic acid or the second nucleic acid comprises a sequence at least 85%, at least 90%, at least 96%, at least 97%, at least 98%, at least 99%, or 100% identical to SEQ ID NO: 8, SEQ ID NO: 9, SEQ ID NO: 10, or SEQ ID NO: 92.

E49. The composition of any one of E21-E36 or the polynucleotide of any of E37-E48, wherein the polynucleotide further comprises a promoter.

E50. The composition or polynucleotide of E49, wherein the promoter comprises a first promoter which is operably linked to the first nucleic acid.

E51. The composition or polynucleotide of E49 or E50, wherein the promoter comprises a second promoter which is operably linked to the second nucleic acid.

E52. The composition or polynucleotide of any one of E48-E50, wherein the promoter is a constitutively active promoter, a cell-type specific promoter, or an inducible promoter.

E53. The composition or polynucleotide of any one of E48-E52, wherein the promoter comprises a CAG promoter, a CBA promoter, a CMV promoter, an EF-1a (Elongation Factor 1a) promoter, aRSV (Rous Sarcoma Virus) promoter, an Ubiquitin (UbC) promoter, or any combination thereof.

E54. The composition of any one of E21-E36 or E49-E53 or the polynucleotide of any of E37-E53, wherein the polynucleotide further comprises a translation modification sequence selected from the group consisting of a furin cleavage sequence, a 2A self-processing peptide sequence, an internal ribosomal entry site (IRES) sequence, or any combination thereof.

E55. The composition of any one of E21-E36 or E49-E54 or the polynucleotide of any of E37-E54, wherein the polynucleotide further comprises an enhancer sequence, an intron sequence, a microRNA binding site, or any combination thereof.

E56. The composition of any one of E21-E36 or E49-E55 or the polynucleotide of any of E37-E55, wherein the polynucleotide further comprises a poly(A) (pA) sequence.

E57. The composition any one of E21-E36 or E49-E56 or the polynucleotide of any of Es 37-56, wherein the first nucleic acid comprises a promoter operably linked to a nucleic acid encoding an IL-12 p35 subunit or functional fragment thereof, an IRES sequence, and an IL-15 gene or a functional fragment thereof, and the second nucleic acid comprises a promoter operably linked to a nucleic acid encoding an IL-12 p40 subunit or functional fragment thereof.

E58. The composition any one of E21-E36 or E49-E56 or the polynucleotide of any of E37-E56, wherein the first nucleic acid comprises a promoter operably linked to a nucleic acid encoding an IL-12 p35 subunit or functional fragment thereof, a furin cleavage sequence and/or a 2A self-processing peptide sequence, and a nucleic acid encoding an IL-12 p40 subunit or a functional fragment thereof, and the second nucleic acid comprises a promoter operably linked to an IL-15 gene or functional fragment thereof

E59. The composition or polynucleotide of E57 or E58, wherein the first nucleic acid further comprises an intron sequence, a pA sequence, an enhancer sequence, or any combination thereof.

E60. The composition or polynucleotide of E59, wherein the first nucleic acid further comprises an intron sequence and a human growth hormone (hGH) pA sequence or a bovine growth hormone (bGH) pA sequence.

E61. The composition or polynucleotide of any one of Es 57-60, wherein the second nucleic acid further comprises an intron sequence, a pA sequence, an enhancer sequence, or any combination thereof.

E62. The composition or polynucleotide of E61, wherein the second nucleic acid further comprises an SV40 intron sequence and a synthetic (SYN) pA sequence.

E63. The composition any one of E21-E36 or E49-E62 or the polynucleotide of any of Es 37-62, wherein the polynucleotide comprises a CMV enhancer sequence.

E64. The composition any one of E21-E36 or E49-E63 or the polynucleotide of any of Es 37-63, wherein the promoter of the first nucleic acid comprises a CAG promoter, a CBA promoter, a CMV promoter, an EF-1a (Elongation Factor 1a) promoter, aRSV (Rous Sarcoma Virus) promoter, or an Ubiquitin (UbC) promoter.

E65. The composition any one of E21-E36 or E49-E64 or the polynucleotide of any of Es 37-64, wherein the promoter of the second nucleic acid comprises a CAG promoter, a CBA promoter, a CMV promoter, an EF-1a (Elongation Factor 1a) promoter, aRSV (Rous Sarcoma Virus) promoter, or an Ubiquitin (UbC) promoter.

E66. The composition any one of E21-E36 or E49-E65 or the polynucleotide of any of Es 37-65, wherein the polynucleotide further comprises two inverted terminal repeats (ITRs).

E67. A composition comprising the polynucleotide of any one of E37-E66 or the expression construct of any of E107-E116, and a delivery vector.

E68. The composition of any one of E21-E36 or E49-E67, wherein the composition is suitable for intratumoral, intravenous, intraperitoneal, intrasplenic, intrathecal, intrahepatic, intraosseous, intradermal, or intraparenchymal administration, or delivery to salivary gland or into a lymph node.

E69. The composition of any one of E21-E36 or E49-E68, wherein the delivery vector is selected from the group consisting of a viral vector, a plasmid, a lipid, a protein particle, a bacterial vector, a lysosome, a virus-like particle, a polymeric particle, an exosome, and a vault particle.

E70. The composition of any one of E21-E36 or E49-E69, wherein the delivery vector is selected from the group consisting of an adeno-associated viral (AAV) vector, an adenoviral vector, a lentiviral vector, or a retroviral vector.

E71. The composition of any one of E21-E36 or E49-E70, wherein the delivery vector is a recombinant AAV (rAAV) vector comprising an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12.

E72. The composition of E71, wherein the rAAV vector is an AAV2 serotype.

E73. The composition of E71 or E72, wherein the rAAV is modified relative to the wild-type serotype.

E74. A composition comprising (a) a polynucleotide comprising a CAG promoter operably linked to a first nucleic acid encoding an IL-12 p35 subunit or a functional fragment thereof, a furin cleavage sequence followed by a 2A self-processing peptide (F2A) sequence, a second nucleic acid encoding an IL-12 p40 subunit or a functional fragment thereof, and a growth hormone pA sequence; and (b) an AAV vector.

E75. An adeno-associated virus (AAV) capsid comprising a polynucleotide of any one of Es 37-66, wherein the AAV capsid is suitable for intratumoral delivery.

E76. The AAV capsid of E75, wherein the AAV capsid serotype is selected from the group consisting of AAV1, AAV2, AAV3, AAV4, AAV5, AAV6, AAV7, AAV8, AAVrh8, AAV9, AAV10, AAVrh10, AAV11, and AAV12.

E77. The AAV capsid of E76, wherein the AAV serotype is AAV2.

E78. The AAV capsid of E76 or E77, wherein the AAV is modified relative to the wild-type serotype.

E79. A method of expressing one or more immunomodulatory proteins or functional fragments thereof in a subject in need thereof comprising administering an effective amount of the composition of any of E21-E36 or E49-E74, the polynucleotide of any of E37-E66, or the AAV capsid of any of E75-E78 to the subject.

E80. The method of E79, wherein the administration is intratumoral, intravenous, intrasplenic, intrathecal, intrahepatic, intraosseous, intradermal, intraparenchymal, via delivery to salivary gland, or into a lymph node.

E81. A method of delivering one or more immunomodulatory proteins or functional fragments thereof to a tumor comprising administering the composition of any of E21-E36 or E49-E64, the polynucleotide of E37-E66, or the AAV capsid of any of E75-E78 by a direct injection to the tumor.

E82. A method of treating or reducing symptoms in a subject suffering from a tumor, comprising administering the composition of any of E21-E36 or E49-E64, the polynucleotide of E37-E66, or the AAV capsid of any of E75-E78 to the subject, wherein the administration is intratumoral, intravenous, intrasplenic, intrathecal, intrahepatic, intraosseous, intradermal, intraparenchymal, via delivery to salivary gland, or into a lymph node.

E83. A method of reducing the size of a tumor in a subject in need thereof comprising administering the composition of any of E21-E36 or E49-E64, the polynucleotide of E37-E66, or the AAV capsid of any of E75-E78 to the subject, wherein the administration is intratumoral, intravenous, intrasplenic, intrathecal, intrahepatic, intraosseous, intradermal, intraparenchymal, via delivery to salivary gland, or into a lymph node.

E84. A method of treating or reducing symptoms in a subject suffering from a tumor, comprising administering to the subject a composition comprising (a) a polynucleotide comprising (i) a first nucleic acid encoding an IL-12 p35 subunit or functional fragment thereof and (ii) a second nucleic acid encoding an IL-12 p40 subunit or functional fragment thereof, and (b) an AAV vector, and optionally further comprising administering a checkpoint inhibitor.

E85. A method of reducing the size of a tumor in a subject in need thereof comprising administering to the subject a composition comprising (a) a polynucleotide comprising (i) a first nucleic acid encoding an IL-12 p35 subunit or functional fragment thereof and (ii) a second nucleic acid encoding an IL-12 p40 subunit or functional fragment thereof, and (b) an AAV vector, and optionally further comprising administering a checkpoint inhibitor.

E86. The method of E84 or E85, wherein the administration of the composition is intratumoral, intravenous, intraperitoneal, intrasplenic, intrathecal, intrahepatic, intraosseous, intradermal, intraparenchymal, via delivery to salivary gland, or into a lymph node.

E87. The method of any of E81-E86, wherein the tumor is derived from a cancer selected from the group consisting of hepatocellular carcinoma, colon carcinoma, melanoma, and any combination thereof.

E88. The method of any of E79-E86, wherein the subject suffers from a cancer selected from the group consisting of hepatocellular carcinoma, colon carcinoma, melanoma, and any combination thereof.

E89. The method of any of E81-E86, wherein the tumor is derived from a cancer selected from the group consisting of a skin cancer; a breast cancer; a brain cancer; a bone cancer; a head and neck cancer; a salivary gland cancer; a gynecologic cancer; a urologic cancer; a gastrointestinal cancer; an ocular cancer; a thoracic cancer; a blood cancer; a lymphoma, a cancer of the endocrine system; a sarcoma of soft tissue; a neoplasm of the central nervous system; and any combination thereof.

E90. The method of any of E15-E20 or E79-E89, wherein the subject suffers from a cancer selected from the group consisting of metastatic melanoma, cutaneous malignant melanoma, cutaneous squamous cell carcinoma, basal cell carcinoma, invasive breast cancer, triple-negative breast cancer, inflammatory breast cancer, glioblastoma multiforme, medulloblastoma, pituitary carcinoma, brain stem gliomas, astrocytomas, oligodendrogliomas, hemangiopericytomas, germ cell tumors, pineal tumors, chordomas, chondrosarcomas, osteosarcomas, Ewing sarcomas, fibrosarcomas, adamantiomas, giant cell tumors, head and neck squamous cell carcinoma (HNSCC), salivary gland cancer, oropharyngeal cancer, hypopharyngeal cancer, laryngeal cancer, lip and oral cavity cancer, nasopharyngeal cancer, thyroid cancer, cancer of the parathyroid gland, paranasal sinus and nasal cavity cancer, ovarian cancer, uterine cancer, fallopian tube cancer, endometrial cancer, cervical cancer, vaginal cancer, vulvar cancer, renal cancer, urethral cancer, urothelial cancer, bladder cancer, cancer of the kidney or ureter, cancer of the renal pelvis, testicular cancer, penile cancer, cancer of the adrenal gland, cancer of the anal region, bile duct cancer, colon cancer, cancer of the small intestine, esophageal cancer, gallbladder cancer, gastrointestinal stromal tumors, liver cancer, pancreatic cancer, rectal cancer, stomach/gastric cancer, uveal melanoma, retinoblastoma, Merkel cell carcinoma, intraocular malignant melanoma, mucosa-associated lymphoid tissue lymphoma, orbital lymphoma, orbital sarcoma, lacrimal gland tumors, non-small cell lung cancer, small cell lung cancer, mesothelioma, thymic malignancies, tracheal tumors, some esophageal cancer, chronic or acute leukemia, acute myeloid leukemia (AML), chronic myeloid leukemia (CML), acute lymphoblastic leukemia (ALL) (including non-T cell ALL), chronic lymphocytic leukemia (CLL), Hodgkin's lymphoma, non-Hodgkin's lymphoma (NHL), Waldenstrom macroglobulinaemia, primary mediastinal large B cell lymphoma (PMBC), diffuse large B cell lymphoma (DLBCL), follicular lymphoma (FL), transformed follicular lymphoma, splenic marginal zone lymphoma (SMZL), cutaneous T-cell lymphoma (CTCL), lymphocytic lymphoma, primary CNS lymphoma, multiple myeloma, CNS; atypical teratoid/rhaboid tumor, spinal axis tumor, tumor angiogenesis, Kaposi's sarcoma, epidermoid cancer, environmentally induced cancers including those induced by asbestos, other B cell malignancies, neuroblastoma, olfactory neuroblastoma, rhabdomyosarcoma, and any combination thereof.

E91. The method of any one of E81-E90, wherein the first nucleic acid and the second nucleic acid are administered simultaneously or sequentially.

E92. The method of E91, wherein the simultaneous administration comprises administering the composition of any of E21-E36 or E49-E64, the polynucleotide of E37-E66, or the AAV capsid of any of E75-E78, wherein the first nucleic acid and the second nucleic acid are in the same delivery vector or AAV capsid.

E93. The method of E91, wherein the simultaneous or sequential administration comprises administering the composition of any of E21-E36 or E49-E64, the polynucleotide of E37-E66, or the AAV capsid of any of E75-E78, wherein the first nucleic acid and the second nucleic acid are in different delivery vectors or AAV capsids.

E94. The method of any of E81-E93, wherein the composition of any of E21-E36 or E49-E64, the polynucleotide of E37-E66, or the AAV capsid of any of E75-E78 is administered in combination with a checkpoint inhibitor.

E95. The method of E94, wherein the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

E96. The method of E94 or E95, wherein the checkpoint inhibitor is a PD-1 inhibitor.

E97. The method of E96, wherein the PD-1 inhibitor is an anti-PD-1 antibody.

E98. The method of any one of E1-E8, E94 or E95, wherein the checkpoint inhibitor is a PD-L1 inhibitor.

E99. The method of any one of E1-E8, E94 or E95, wherein the checkpoint inhibitor is a CTLA-4 inhibitor.

E100. The method of any one of E1-E8 or E94-E99, wherein the checkpoint inhibitor comprises pembrolizumab, nivolumab, cemiplimab, toripalimab, spartalizumab, dostarlimab-gxly, tislelizumab, balstilimab, BI-754091, zimberelimab, INCMGA00012, camrelizumab, or any combination thereof.

E101. The method of any of Es 94-100, wherein the composition of any of E21-E36 or E49-E64, the polynucleotide of any one of E37-E66, or the AAV capsid of any one of E75-E78 is administered prior to, at the same time, or after the administration of the checkpoint inhibitor.

E102. The method of any one of E68, E82, E83, or E86, wherein the lymph node in a metastatic lymph node.

E103. The method of any one of E15-E20 and E81-E102, wherein the tumor is a metastatic tumor.

E104. The method of any one of E15-E20 and E81-E102, wherein the administration comprises direct injection of the first composition or the composition comprising the polynucleotide to the tumor.

E105. A polynucleotide comprising a nucleic acid sequence having at least 80%, 85%, 90%, 95% 96%, 97%, 98%, 99%, or 100% identity to any of SEQ ID NOs: 5-37, 48-57, 77, 82, or 92, any nucleic acid shown in Table 2, Table 4, or Table 5, or any combinations thereof.

E106. A polynucleotide comprising a nucleic acid sequence having at least 80%, 85%, 90%, 95% 96%, 97%, 98%, 99%, or 100% identity to SEQ ID NO: 82.

E107. An expression construct comprising a formula selected from:

X-I1-P1-E-P2-I2-X′-T-Y; (a)

Y-I1-P1-E-P2-I2-X-T-X′; (b)

pA-X-I1-L-P1-E-P2-I2-X-T-Y-pA; (c)

pA-Y-I-L-P1-E-P2-I2-X-T-X′-pA; (d)

P1-X-T-X′-pA; or (e)

E-P1-I1-X-T-X′-pA; (f)
wherein X and X′ encode subunits of a first immunomodulatory molecule; I1 and 12 each is an intron; P and P2 each is a promoter; E is an enhancer; T is a translation modification sequence; Y encodes a second immunomodulatory molecule; L is a long-terminal repeat; and pA is a polyA sequence.

E108. The expression construct of E107, wherein P1 or P2 comprises a CAG promoter, a CBA promoter, a CMV promoter, an EF-1a (Elongation Factor 1a) promoter, aRSV (Rous Sarcoma Virus) promoter, an Ubiquitin (UbC) promoter, or any combination thereof.

E109. The expression construct of E107 or E108, wherein T is an internal ribosomal entry site (TRES) sequence, a furin cleavage sequence, a 2A self-processing peptide sequence, or any combination thereof.

E110. The expression construct of any one of E107-E109 further comprising a microRNA binding site.

E111. The expression construct of any one of E107-E110, wherein X is a nucleic acid encoding an IL-12 subunit or a functional fragment thereof; X′ is a nucleic acid encoding an IL-12 subunit or functional fragment thereof, and Y is IL-15 gene or a functional fragment thereof.

E112. The expression construct of any one of E107-E110, wherein X is a nucleic acid encoding an IL-12 subunit or a functional fragment thereof and X′ is a nucleic acid encoding an IL-12 subunit or functional fragment thereof.

E113. The expression construct of E111 or E112, wherein the nucleic acid encoding an IL-12 subunit is a nucleic acid encoding an IL-12 p35 subunit or a nucleic acid encoding an IL-12 p40 subunit.

E114. The expression construct of any one of E107-E113, wherein the pA comprises a human growth hormone (hGH) pA sequence, a bovine growth hormone (BGH) pA sequence, or a synthetic (SYN) pA sequence.

E115. The expression construct of any one of E107-E114, wherein I1, 12, or both comprises a SV40 intron sequence or a CAG intron sequence.

E116. The expression construct of any one of E107-E115, wherein E comprises a CMV enhancer sequence.

E117. A method for inducing tumor regression in a subject comprising administering to a subject in need thereof (a) the composition of any of E21-E36 or E49-E64, the polynucleotide of any one of E37-E66 or E105-E106, the expression construct of any one of E107-E116, or the AAV capsid of any one of E75-E78 and (b) checkpoint inhibitor.

E118. The method of E117, wherein the checkpoint inhibitor is a PD-1 inhibitor.

E119. The method of E118, wherein the PD-1 inhibitor is an anti-PD-1 antibody.

E120. The combination therapy of E10 or the method of E97 or E119, wherein the anti-PD-1 antibody is pembrolizumab or nivolumab.

E121. The method of any of E117-E120, wherein the tumor is derived from a cancer selected from the group consisting of a hepatocellular carcinoma, a colon carcinoma, or a melanoma.

E122. The method of any one of E81-E102, E94-E104, or E117-E121, wherein the composition of any of E21-E36 or E49-E64, the polynucleotide of any one of E37-E66 or 105-106, the expression construct of any one of E107-E116, or the AAV capsid of any one of E75-E78 is administered by a direct injection to a tumor.

E123. The method of any one of E81, E104 or E122, wherein the direct injection comprises two or more injections into the tumor.

E124. The method of any one of E15-E20, E84, E85, E94-E104, or E117-E123, wherein the checkpoint inhibitor is administered by intravenous infusion.

E125. The combination therapy of any one of E1-E14, the composition of any one of E21-E36 or E49-E64, the polynucleotide of any one of E37-E66 or E105-106, the AAV capsid of any one of E75-E78 or the expression construct of any one of E107-E116, for use in therapy or prophylaxis.

E126. The combination therapy, composition, polynucleotide, AAV capsid or expression construct of E125 for use in gene therapy.

E127. The combination therapy, composition, polynucleotide, AAV capsid or expression construct of E125 or E127 for use in cancer therapy.

E128. The combination therapy, composition, polynucleotide, AAV capsid or expression construct of any one of E125 to E127 for use in a method as defined in any one of E15-E20, E79-E104, or E117-E124.

E129. A pharmaceutical composition comprising the composition of any one of E21-E36 or E49-E64, the polynucleotide of any one of E37-E66 or E105-E106, the AAV capsid of any one of E75-E78 or the expression construct of any one of E107-E116.

E130. The pharmaceutical composition of E129 further comprising a pharmaceutically acceptable excipient.

E131. Use of the combination therapy of any one of E1-E14, the composition of any one of E21-E36 or E49-E64, the polynucleotide of any one of E37-E66 or E105-E106, the AAV capsid of any one of E75-E78 or the expression construct of any one of E107-E116, for the manufacture of a medicament for use in therapy or prophylaxis.

E132. The use of E131 wherein the therapy or prophylaxis comprises a method as defined in any one of E15-E20, E79-E104, or E117-E124.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

### EXAMPLES

### Example 1: Assessing Biodistribution and Kinetics of Expression of AAV-2 Luciferase in Multiple Cancer Cell Line Models

Transduction of an AAV2-luciferase vector in five different syngeneic mouse cancer models (MC38, B16F10-p436, CloudmanS91, CT26-p928, and A20-p165) was tested. The aim of the study was to identify tumor types that were efficiently transduced by an AAV vector. Transduction efficiency and transgene expression levels and kinetics were evaluated both in vitro and in vivo. Transduction was evaluated over 21 days to determine kinetics after intratumoral injection. Luciferase expression was evaluated on multiple days per week for three weeks via whole body bioluminescence imaging. To assess biodistribution or escape of the AAV2 from the injection site, liver and spleen were taken for evaluation by immunohistochemistry and PCR. Ratios supporting maximal tumor transduction and transgene expression while limiting systemic exposure were assessed. Correlations between in vitro and in vivo transduction were also evaluated. Results showed that AAV2 was able to be successfully administered to all tumor models.

### Methods

### Vector Details

AAV2/2 Firefly luciferase (AAV2-luc) was purchased at a titer of 1.0×10¹³ GC/mL from Vector Biolabs (Malvern PA). The rAAV genome was comprised of AAV2 inverted terminal repeats (ITRs), a CAG promoter, transgene encoding for firefly luciferase, and a bovine growth hormone (bGH) polyadenylation signal.

### Murine Tumor Lines

The A20 lymphoma, CT26 colon carcinoma and Cloudman S91 were obtained from the American Type Culture Collection (ATCC). MC38 colorectal adenocarcinoma cells were obtained from the National Cancer Institute (NCI). The B16F10 murine colon carcinoma cell line was obtain from University of North Carolina (UNC) Lineberger Comprehensive Cancer Center. All cells were maintained in cell culture medium per vendor specifications.

### In Vitro Assay

Replicating cells were transfected with AAV2-luc at MOI's ranging from 2000 to 40500 vp/cell. Luciferase expression (determined by bioluminescence—IVIS spectrum CT, Perkin Elmer) was determined 2 and 5 days after transfection.

### In Vivo Assay

Established A20 lymphoma, CloudmanS91 melanoma, B16F10 melanoma, CT26 colorectal, and MC38 colorectal tumors grown subcutaneously in syngeneic BALB/c, C57B1/6, or DBA/2. Mice were directly injected subcutaneously in the right flank with 1×10¹¹ vp in 0.01 ml., 0.025 ml, or 0.05 ml of vehicle. Tumors were longitudinally imaged for bioluminescence (IVIS spectrum CT, Perkin Elmer) following injection of luciferin at regular intervals from day 5 post-transfection. The studies were terminated either at day 21 or when tumor volumes exceeded the maximum permitted volume (typically 1500-2000 mm³). At the end of the study, mice were euthanized and tumor, liver and spleen samples collected for further analysis.

### Results

### In Vitro Transfection Leads to Active Luciferase Expression

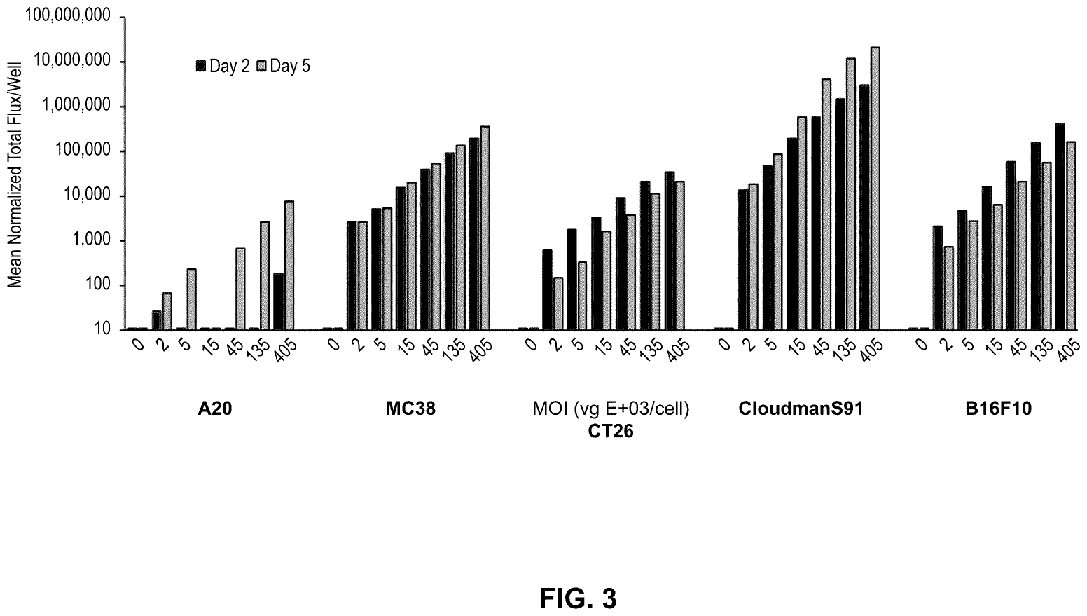

In B16F10, CloudmanS91, CT26 and MC38 luciferase expression was detected 2 days after transfection. (FIG. 3). In contrast, luciferase expression in A20 was not readily apparent until day 5. (FIG. 3). Based on luciferase expression at highest MOI, the rank order was CloudmanS91>B16F10=MC38>CT26>A20 in terms of photon intensity. (FIG. 3). The level of luciferase expression was MOI-dependent. Luciferase expression was detected in CloudmanS91 on day 2 at an MOI of 2000 whereas luciferase expression in A20 was only observed at markedly higher MOIs (>135000) and then not until day 5. (FIG. 3).

Intratumoral Injection of Murine Syngeneic Tumors with AAV2-Luc Results in Active Luciferase Expression

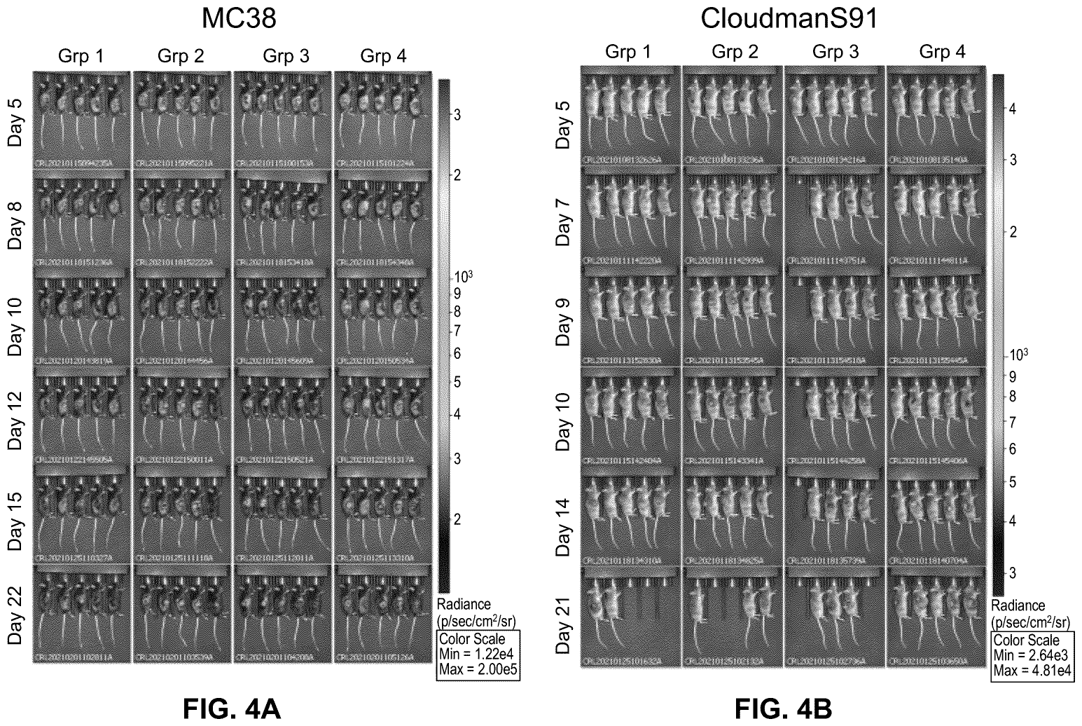

Transfection of syngeneic murine tumors with AAV2-Luc led to luciferase expression (determined by bioluminescence) in all five syngeneic murine tumor lines tested. The time course for B16F10 and CloudmanS91 are shown in FIG. 4A and FIG. 4B. Bioluminescence was detected in a majority of transfected tumors at the first time point (day 4, CloudmanS91; day 5, B16F10) and was still detectable in tumors at the last evaluable time point (day 21, CloudmanS91; day 12, B16F10) indicating sustained luciferase expression. (FIGS. 4A and 4B). Grp 1 was given control saline injection; Grp 2 was given a 10 uL injection of AAV2-luc representing 10% of the tumor volume; Grp 3 was given a 25 uL injection of AAV2-luc representing 25% of the tumor volume; Grp 4 was given a 50 uL injection of AAV2-luc representing 50% of the tumor volume.

### Pattern of Bioluminescence

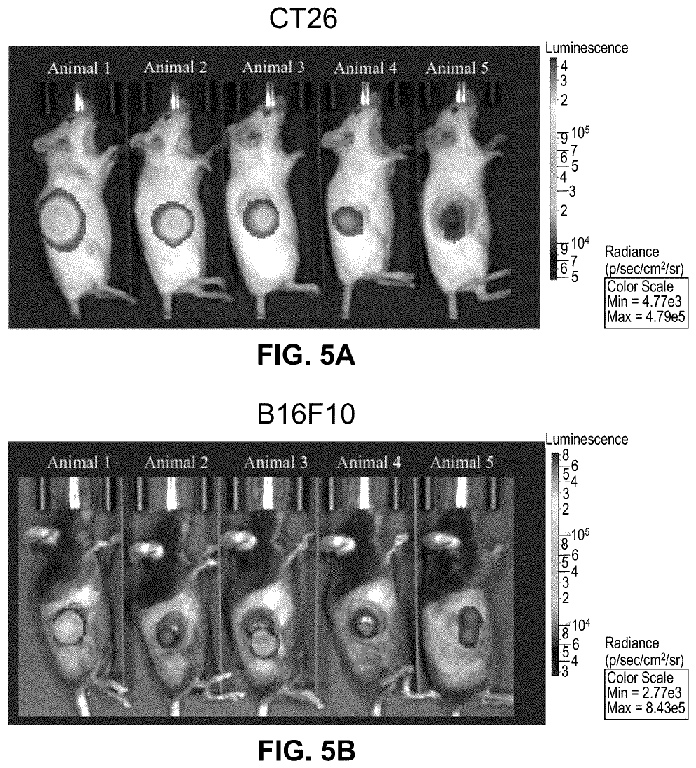

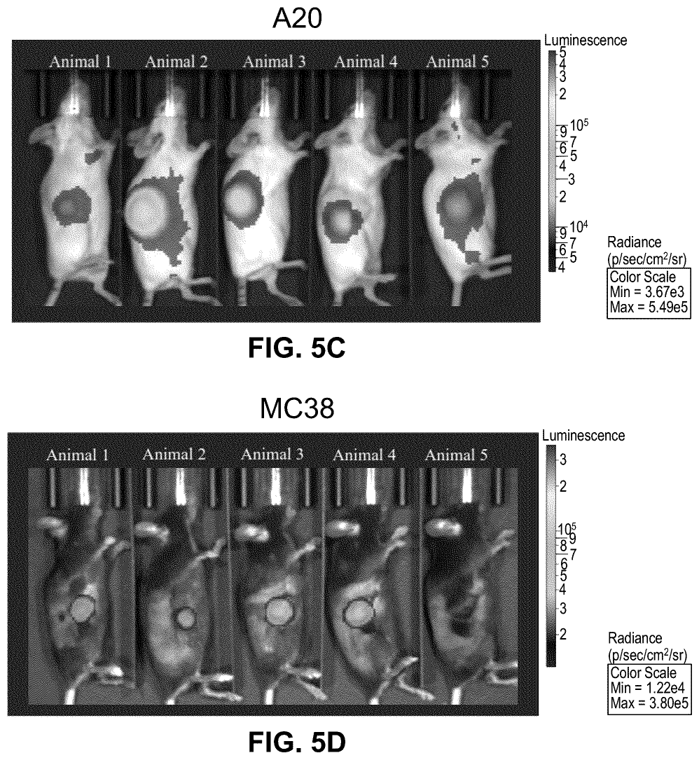

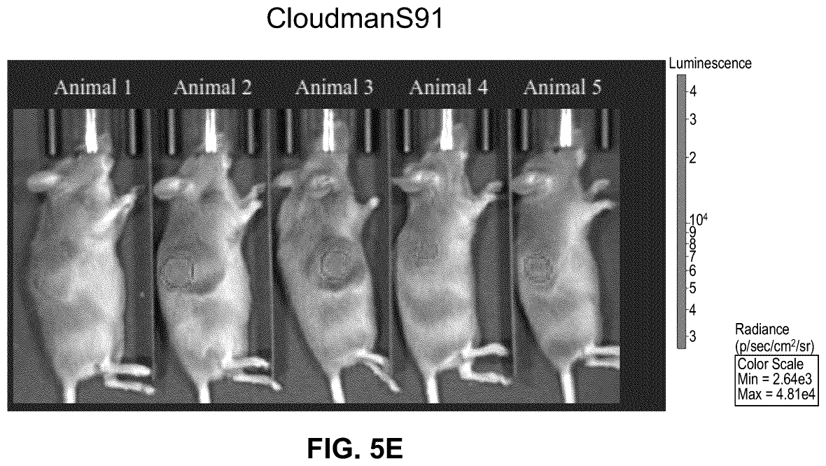

Bioluminescence was generally co-localized with the tumor mass/injection site for CT26 (FIG. 5A), B16F10 (FIG. 5B), MC38 (FIG. 5D) and CloudmanS91 (FIG. 5E). However, some minor bioluminescence signal was observed distal to the primary tumor mass (FIG. 5C, animal #'s 1 & 5) at the highest dose only (˜50% tumor volume) in the A20 lymphoma model. Representative images are shown for each tumor line at the 50 ul dose.

### Lack of Correlation Between In Vitro and In Vivo Luc Expression

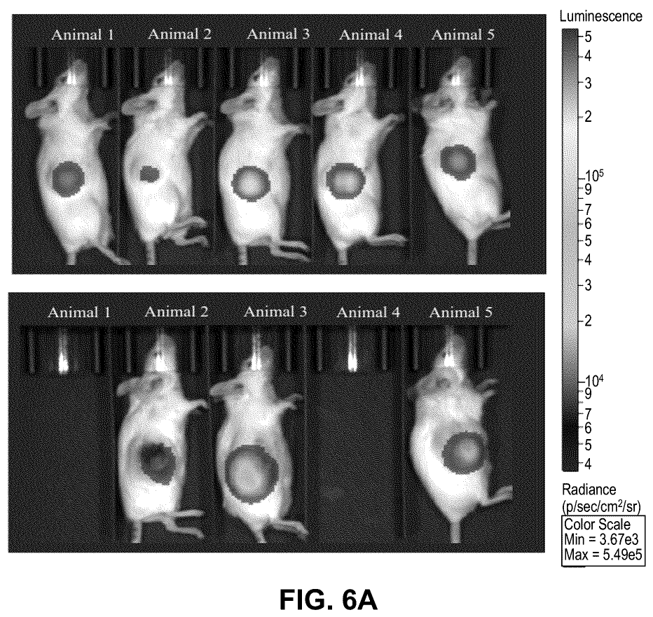

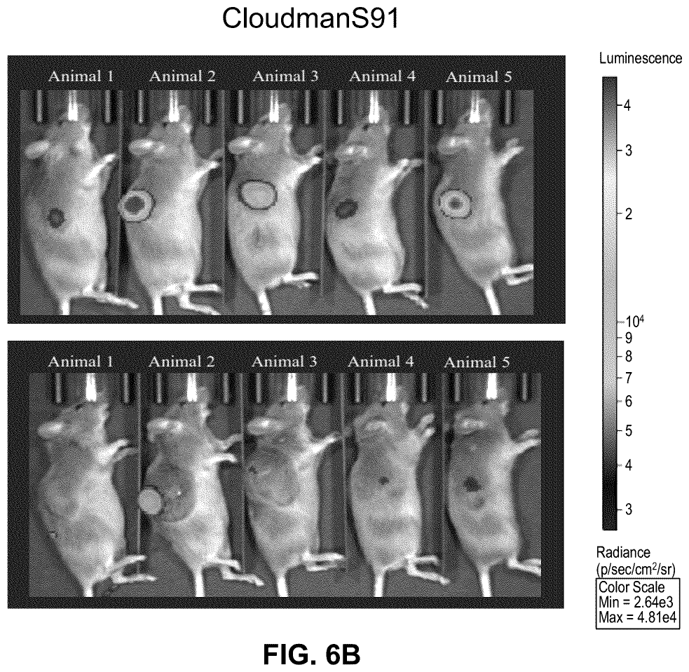

In contrast to the in vitro studies where AAV2-luc transfection of the A20 line appeared less efficient than the remaining four (4) cell lines (FIG. 3), the in vivo appearance of bioluminescence (indicative of luciferase expression) in A20 tumors was similar to the other tumor lines. (FIGS. 6A and 6B). Furthermore, bioluminescence was still detectable on day 14. (FIGS. 6A and 6B).

### Example 2: In Vivo Evaluation of AAV2-IL12 and Anti-PD-1 Antibody Therapy in H22 Hepatocellular Carcinoma Mouse Models

The anti-tumor efficacy of the AAV2-IL2 (AAV2-CAG-mIL12a-F2A-mIL12b-hgh PA) treatment alone and in combination with an anti-PD-1 antibody (RMP1-14) therapy was evaluated in the H22 Hepatocellular Carcinoma (HCC) model in female BALB/c mice. The study was designed as shown in Table 8 for vehicle, AAV2-IL12, and RMP1-14.

TABLE 8
Study Design for H22

| | |
|---|---|
| Model(s) | H22 |
| Strain(s) | BALB/c |
| Number of mice on study | 32 |
| Number of groups | 4 |
| N per group | 8 |
| Inoculation | SubQ-Hind Right Flank |
| Dosing regimen | Single (AAV)/BIW × 3 (PD-1) |
| Dosing route | Intratumoral (IT)-(AAV)/ |
| | Intraperitoneal (IP) |
| | (RMP1-14) |
| Dosing duration | 3 weeks (anti PD-1 antibody) |
| BW and TV measurement | 2×/week |
| frequency | |
| Randomization range | 80-120 mm³ |
| Dietary supplements | Body Weight Loss of >10%, |
| | all in group |
| Dosing holiday (PD-1) | Body Weight Loss of >20%, |
| | individual mouse |
| Tissue collections | Blood (cheek) Weeks: −1, 2, 4 |
| | Tumor (IHC) |
| Maximal tumor volume (MTV) | 3000 mm³ |

### 2.1 Tumor Cell Preparation.

Cryogenic vials containing tumor cells were thawed and cultured according to the provider's protocol. On the day of injection, cells were washed in serum-free media, counted, and resuspended in cold serum-free media at a concentration of 2E7 viable H22 cells/100 L. Cell suspensions were kept on ice during transport to the vivarium. Cells were prepared for injection by withdrawing the suspension into a chilled 1 mL slip-tip syringe. The filled syringes were kept on ice.

### 2.2 Tumor Implantation.

100 μL of the cell suspension was subcutaneously injected into the rear flank of the mouse. Animals were undisturbed for up to seven days before observing for tumor growth.

### 2.3 Tumor Measurement.

Animals were monitored weekly for palpable tumors, or any changes in appearance or behavior. Once tumors were palpable, tumors were measured at least twice per week using calipers. Tumor volume was calculated using the following equation: (longest diameter*shortest diameter²)/2. Once tumors were of an appropriate size to begin the study, tumors and body weights were measured twice per week for the duration of the study.

### 2.4 Randomization and Dose Selection.

When the average tumor volume reached 80-120 mm³, mice were randomly assigned to the respective treatment groups and dosed within 24 hours of randomization. Reference Day: Day 0 was defined as dosing start.

### 2.5 Study Termination.

The study was terminated 12 weeks after the first dose.

### 2.6 Body Weight Measurement and Guidelines.

Body weight was measured twice a week following randomization and initiation of treatment. Hydrogel/diet gel and/or dosing holidays were given to animals due to body weight loss; body weight loss was calculated based on the body weight of the mouse on the first day of treatment. Dosing holiday and/or nutritional supplements may have been provided to the animal based on the study director's assessment of animal health. If there were no signs of recovery, the animal was sacrificed for humane reasons.

### 2.6.1 Dosing Holiday for Anti-PD-1 Treatment.

After one measurement of body weight loss greater than 20% of body weight, dosing holidays were given to the individual mouse, while other mice in the same group received dosing as scheduled.

### 2.6.2 Supplemental Gel Recovery Administration.

After body weight loss of greater than 10% was observed in a single animal, a hydrogel/diet gel was supplied to all animals in the same group.

### 2.7 Clinical Observations.

Clinical observations were performed twice a week at the time of tumor and body weight measurements. During daily routine monitoring, the animals were checked for any adverse effects of tumor growth and treatments on behavior such as mobility, food and water consumption, eye/hair matting and any other abnormalities. Mortality and observed abnormal clinical signs were recorded for individual animals in detail. Daily monitoring took place for mice showing any signs of morbidity or mortality.

### 2.8 Tissue Collection.

Tumor samples were collected according to Table 9 (Tumor) and Table 10 (Blood).

TABLE 9
Tumor Tissue Collection for H22

| Groups | N | Time Point | Tissue |
|---|---|---|---|
| 1-4 | 32 | Termination @ 12 weeks/MTV | Tumor |

*Formalin Fixed Paraffin Embedded (FFPE)

TABLE 10
Blood Collection for H22

| Groups | N | Time Point |
|---|---|---|
| 1-4 | 32 | 1 week before Dose 1 |
| | | (1 day before innoculation) |
| 1-4 | 32 | 2 weeks after Dose 1 |
| 1-4 | 32 | 4 weeks after Dose 1 |

### 2.8.1 Meso-Scale Discovery (MSD).

H22 serum samples will be processed for IL-12 quntification by Meso-Scale Discovery (MSD, Rockville, MD, USA). MSD assay will be performed in triplicate. The standard and blank controls will be performed in duplicate.

Samples will be diluted with Diluent 2 (MSD Catalog #R51BB) (a minimum 2-fold dilution). For one plate, 60 μL of each 50× detection antibody stock solution will be combined. Diluent 2 will be added to bring the final volume to 6 mL. For one plate, 10 mL of Read Buffer T (4×) (MSD Catalog #R92TD) and 10 mL of deionized water will be combined to prepare a 2× Read Buffer solution. Excess dilution Read Buffer T was kept in a tightly sealed container at room temperature up to one month.

Seven calibrator samples plus a zero calibrator standard will be prepared by reconstituting calibrator 1 (highest calibrator) (MSD Catalog #C0060-2) by adding 1000 μL of Diluent 2 to the lyophilized calibrator vail and inverting three to five times without vortexing. The reconstituted solution will be equilibrated at room temperature for 15-30 minutes and then vortexed briefly using short pulses. 100 μL of the highest calibrator will be transferred to 300 μL of Diluent 2 and mixed well by vortexing. 4-fold serial dilution will be repeated five additional times to generate seven calibrators. Diluent 2 will be used as the zero calibrator.

The plates will be washed three times with 150 μL wash buffer/well. 50 μL of the calibrator standards or samples will be added to each well in duplicates. The plate will be sealed with a plate seal and incubated at room temperature with shaking for 2 hours. The plate will be washed three times with 150 μL wash buffer/well. 25 μL of detection antibody solution will be added to each well. The plate will be sealed with a plate seal and incubated at room temperature with shaking for 2 hours. The plate will be washed 3 times with at least 150 μL wash buffer/well. 150 μL of 2× Read Buffer T will be added to each well. Next, the plate will be analyzed with the MSD instrument.

The data result will be analyzed by MSD discovery workbench analysis software to determine acceptance criteria of the standards. The regression model will be a 4-parameter logistic model (sigmoidal dose-response) with a 1/Y2 weighting. The correlation coefficients of standard curve will be accepted as if R2>0.98. The bias % of at least 6 non-zero points on the standard curve including upper and lower limits of quantification will not exceed±20.0% (upper and lower limit of quantification is ±30.0%). Bias %=(measured concentration-theoretical concentration)/theoretical concentration×100%. CV % of at least six non-zero points on the standard curve including upper and lower limits of quantification did not exceed±20.0% (upper and lower limit of quantification is ±30.0%). CV %=SD of measured concentration/Average of measured concentration×100%.

### 2.9 Results.

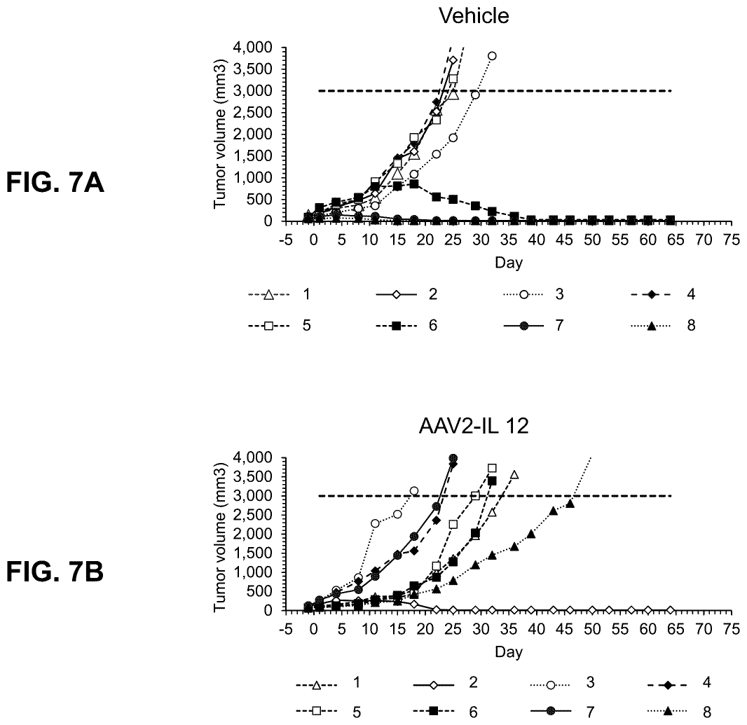

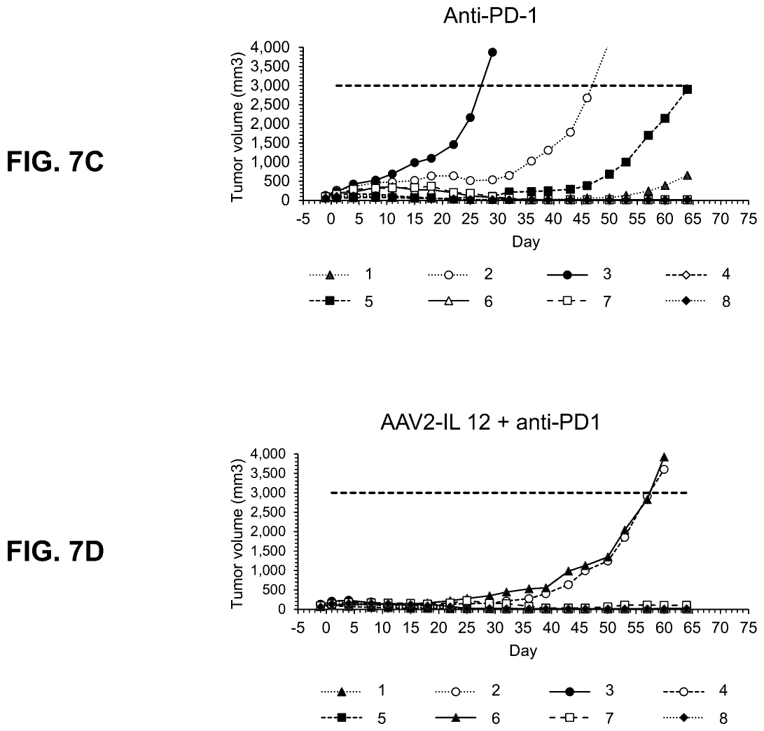

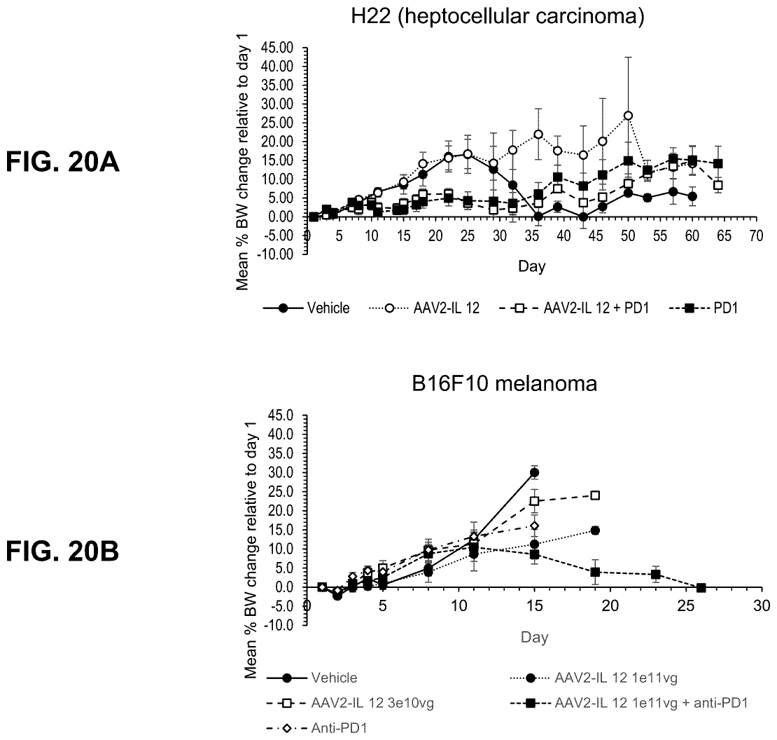

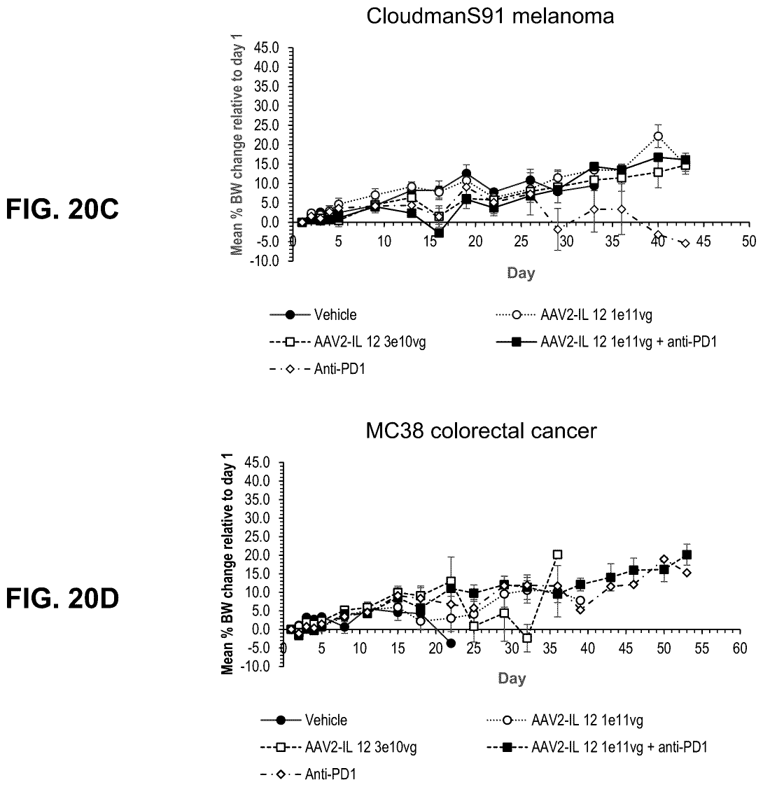

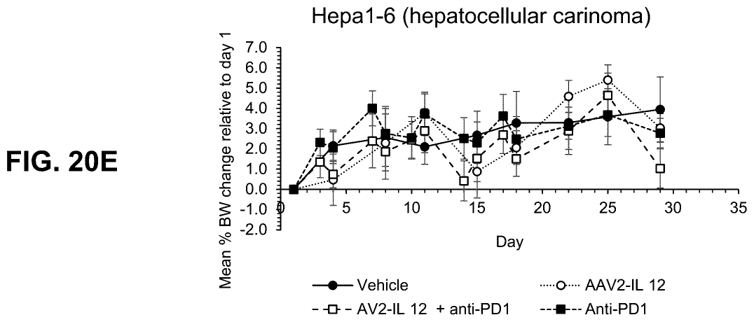

Tumor regression was measured by tumor volume (mm³) in H22 hepatocellular cancer mouse models after treatment with vehicle (FIG. 7A), AAV2-IL12 (FIG. 7B), RMP1-14 (an anti-PD-1 antibody) (FIG. 7C), or AAV-IL12 and RMP1-14 (FIG. 7D). Treatment with vehicle was consistent with historical data, although some tumors failed to grow out. Treatment with AAV-IL12 showed heterogenous response to AAV-IL12 and delayed tumor growth was observed in 5 out of 8 samples. Treatment with RMP1-14 also showed heterogeneous response, with 3 out of 8 samples showing complete response. Treatment with AAV2-IL 12 and RMP1-14 showed improved overall response compared to RMP1-14 alone, with 5 out of 8 samples showing complete response. No meaningful negative impact on body weight was observed with AAV2-IL12 alone or in combination with RMP1-14 (FIG. 20A).

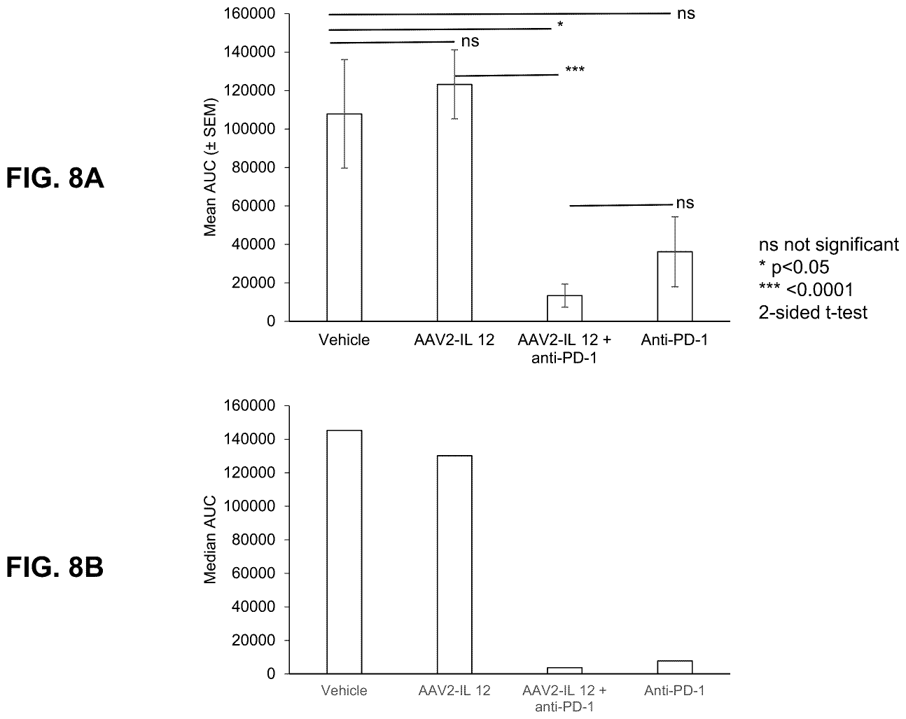

Tumor regression was also measured by both mean area under the curve (AUC) (FIG. 8A) and median area under the curve (AUC) (FIG. 8B) of tumor growth in H22 hepatocellular cancer mouse models after treatment with vehicle, AAV-IL12, the combination of AAV-IL12 and RMP1-14, or RMP1-14 alone. The effect of AAV2-IL12+anti-PD-1 antibody (RMP1-14) and RMP1-14 alone were significant relative to both vehicle and AAV2-IL12 alone (p<0.05). AAV2-IL 12+RMP1-14 resulted in lower AUC relative to RMP1-14 alone, but the effect was not statistically significant (p>0.05) (FIG. 8A).

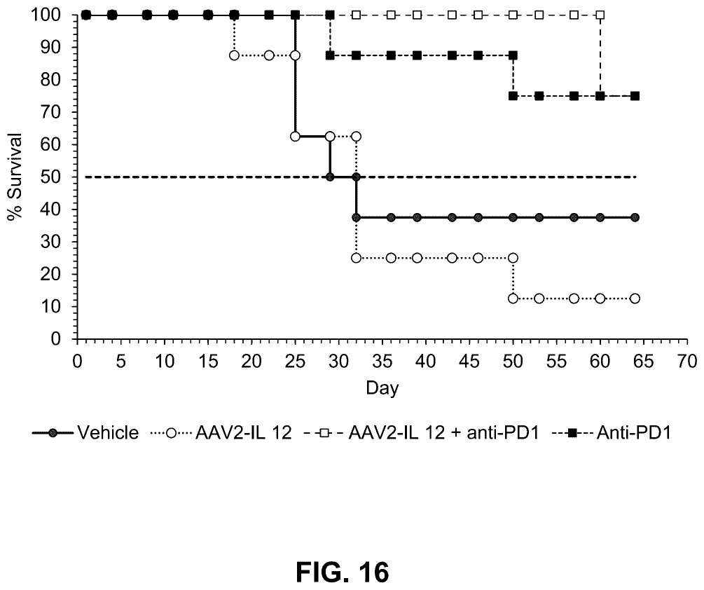

Overall survival was also measured in H22 hepatocellular cancer mouse models after treatment with vehicle, AAV-IL12, AAV-IL12 in combination with RMP1-14, or RMP1-14 alone (FIG. 16). The results showed that the combination treatment of RMP1-14 antibody and AAV2-IL12 led to a significant improvement in overall survival.

### Example 3: In Vivo Evaluation of AAV2-IL12 and an Anti-PD-1 Antibody Therapy in MC38 Murine Colon Carcinoma Mouse Models

The anti-tumor efficacy of AAV2-IL12 (AAV2-CAG-mIL12a-F2A-mIL12b-hgh PA) vectors in MC38 murine colon carcinoma mouse models was demonstrated using female C57BL/6 mice. The study was designed as shown in Table 11.

TABLE 11
Study Design for MC38 murine colon carcinoma

| Gr. | N | Agent | Active dose | Route | Schedule |
|---|---|---|---|---|---|
| 1^{#} | 8 | vehicle | n/a | intra-tumoral | day 1 |
| 2 | 8 | AAV2-IL12 | 1E + 11 vg/mouse | intra-tumoral | day 1 |
| 3 | 8 | AAV2-IL12 | 3E + 10 vg/mouse | intra-tumoral | day 1 |
| 4 | 8 | AAV2-IL12// | 1E + 11 vg/mouse// | intra-tumoral// | day 1// |
| | | anti-PD-1 | 5 mg/kg | intraperitoneal | biwk × 2 |
| | | RMP1-14 | | | |
| 5 | 8 | anti-PD-1 | 5 mg/kg | intraperitoneal | biwk × 2 |
| | | RMP1-14 | | | |

^{#}control group

Mice were anesthetized with isoflurane for the implantation of cells to reduce the ulcerations. Female C57BL/6 mice (Charles River Laboratories, USA) were subcutaneously injected with 5×10⁵ MC38 tumor cells in 0% Matrigel (total cell injection volume=0.1 mL/mouse) in flank. Mice were 8 to 12 weeks old at the start date. A pair match was performed when tumors reached an average size of 80-120 mm³, and treatment began.

Body weight was measured daily for five days, then twice a week until the end of the study. Tumors were measured by calipers twice a week to the end of the study. Any adverse reactions or death were reported immediately. Any individual animal with a single observation of greater than 30% body weight loss or three consecutive measurements of greater than 25% body weight loss was euthanized. Any group with a mean body weight loss of greater than 20% or greater than 10% mortality stopped receiving dosing. The group was not euthanized and recovery was allowed. Within a group with greater than 20% weight loss, animals that hit the individual body weight loss endpoint were euthanized. If the group treatment related body weight loss was recovered to within 10% of the original weights, dosing was resumed at a lower dose or less frequent dosing schedule. Exceptions to non-treatment body weight % recovery were allowed on a case-by-case basis.

Animals were monitored individually. The endpoint of the experiment was a tumor volume of 1500 mm³ or 29 days, whichever came first. Responders were followed longer. When the endpoint was reached, the animals were euthanized.

AAV2-IL12 vectors were provided in 5% Sorbitol+elevated 350 mM NaCl in PBS. Vehicle was provided in 5% Sorbitol+elevated 350 mM NaCl in PBS. The dosing volume for vehicle and the AAV2-IL12 vectors were 0.025 mL/mouse, and volume was not adjusted for body weight. AAV2-IL12 vector dosing syringes were prewet with vehicle buffer prior to drawing up dosing solution for first time each syringe was used. The dosing volume for an anti-mouse PD-1 antibody (RMP1-14)=10 mL/kg (0.200 mL/20 g mouse), and volume was adjusted accordingly for body weight.

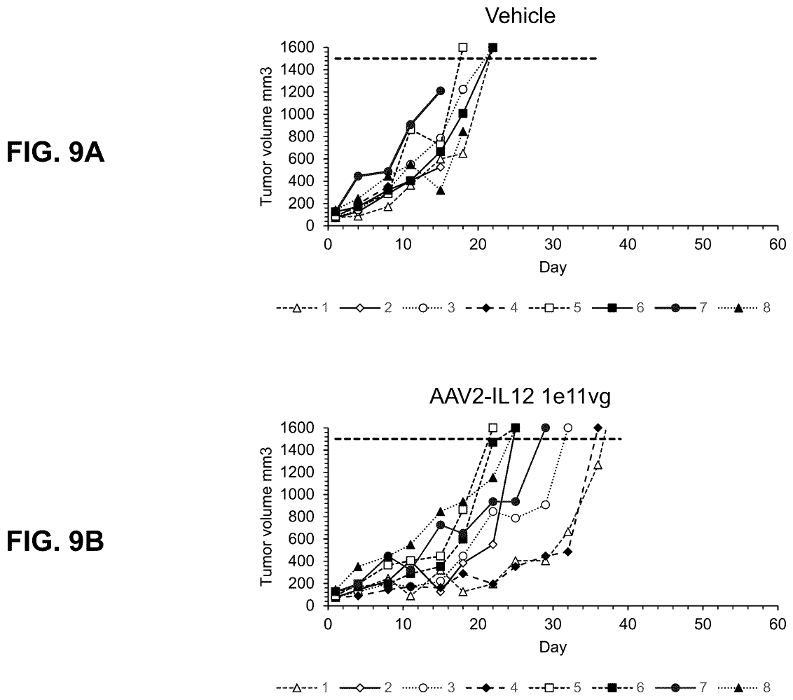

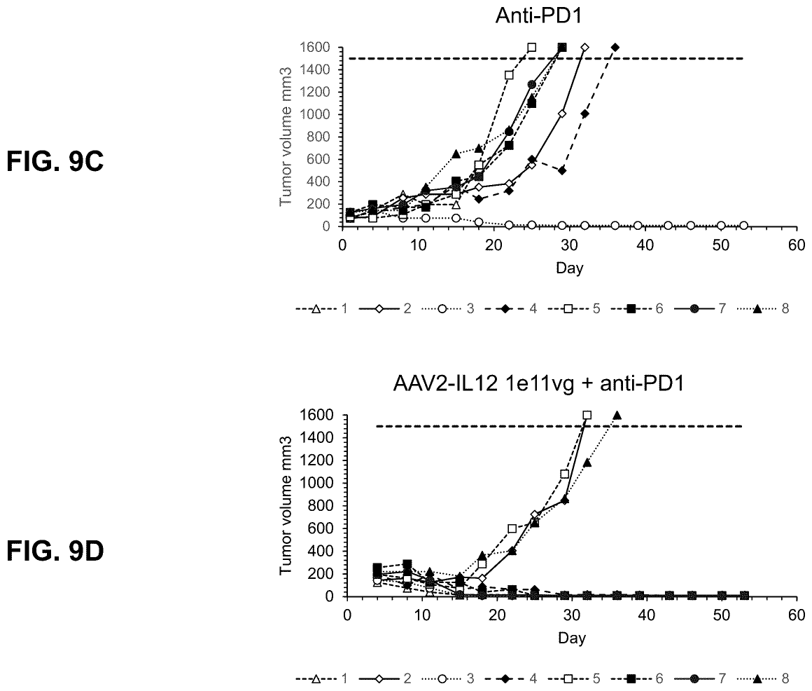

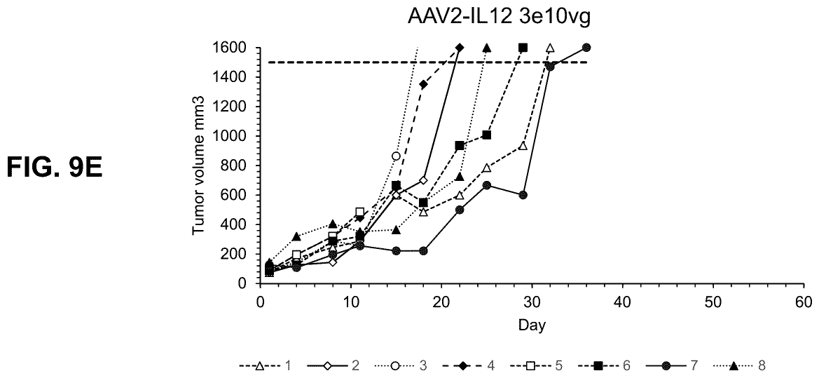

Tumor regression was measured by tumor volume in MC38 (mm³) colorectal cancer mouse models after treatment with vehicle (FIG. 9A), AAV2-IL12 (FIG. 9B and FIG. 9E), RMP1-14 (FIG. 9C), or AAV-IL12 in combination with RMP1-14 (FIG. 9D). Tumor growth in vehicle treated animals was consistent with historical data. Tumor growth in AAV-IL12 treated animals showed a heterogenous response to AAV-IL12 at both doses of 1×10¹¹ viral genomes (FIG. 9B) and 3×10¹⁰ viral genomes (FIG. 9E), with some delayed tumor growth. Tumor growth in an anti-PD-1 antibody treated animals showed a heterogenous response, with 1 out of 8 animals showing a complete response. Tumor growth in animals treated with AAV2-IL12 and RMP1-14 combination showed a superior response to the other treatments, with 5 out of 8 animals showing a complete response. No meaningful negative impact on body weight was observed with AAV2-IL12 alone or in combination with RMP1-14 (FIG. 20D).

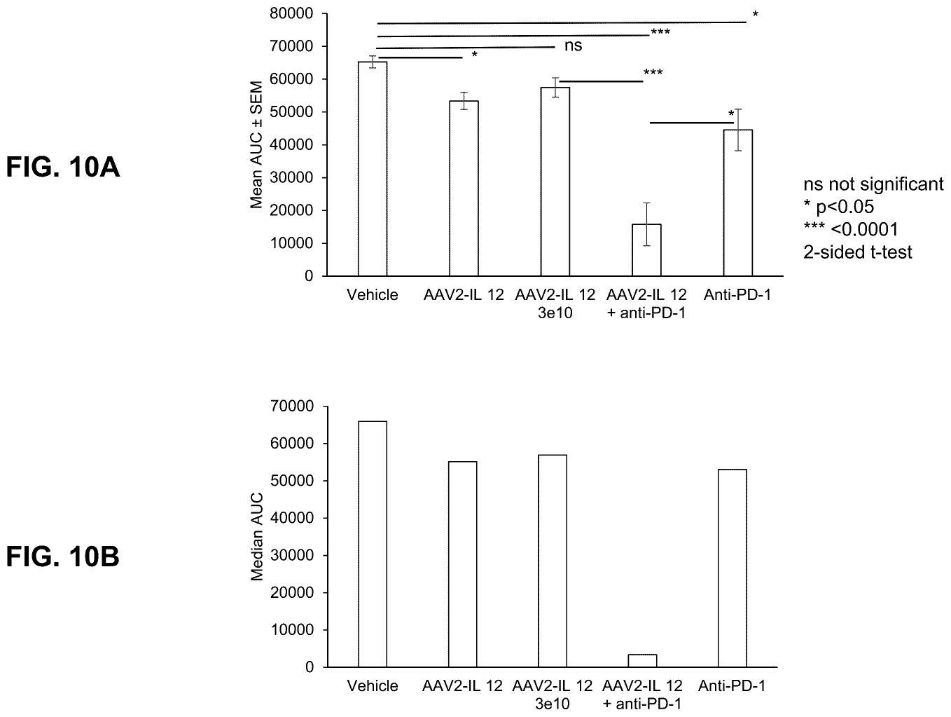

Tumor regression was also measured by both mean area under the curve (FIG. 10A) and median area under the curve (FIG. 10B) of tumor growth in MC38 colorectal cancer mouse models after treatment with vehicle, AAV-IL12, AAV-IL12+RMP1-14 combination, or RMP1-14 alone. The combination therapy AAV2-IL12+RMP1-14 resulted in significantly reduced tumor AUC relative to all other treatments or vehicle (p<0.03) (FIG. 10A).

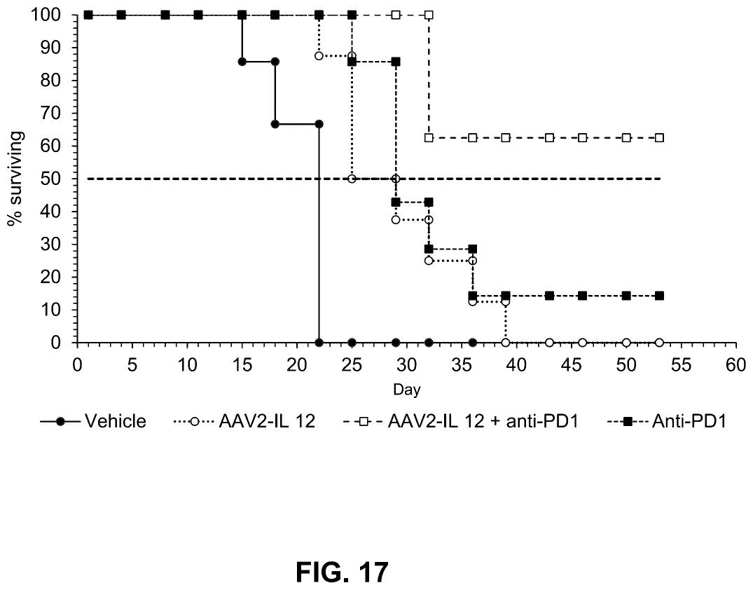

Overall survival was also measured in MC38 colorectal cancer mouse models after treatment with vehicle, AAV-IL12, AAV-IL12 and RMP1-14 combination therapy, or RMP1-14 alone (FIG. 17). Monotherapy with RMP1-14 or AAV2-IL12 had similar improvement on overall survival. However, the combination of AAV-IL12 and an anti-PD1 antibody was superior in terms of an overall survival and response to either single agent alone.

### Example 4: In Vivo Evaluation of AAV2-IL12 and an Anti-PD-1 Antibody Therapy in Cloudman S91 Melanoma Mouse Models

The anti-tumor efficacy of AAV2-IL12 (AAV2-CAG-mIL12a-F2A-mIL12b-hgh PA) vectors in Cloudman S91 melanoma mouse models was demonstrated using female DBA/2 mice. The study was designed as shown in Table 12.

TABLE 12
Study Design for Cloudman S91 melanoma

| Gr. | N | Agent | Active dose | Route | Schedule |
|---|---|---|---|---|---|
| 1^{#} | 8 | vehicle | n/a | intra-tumoral | day 1 |
| 2 | 8 | AAV2-IL12 | 1E + 11 vg/mouse | intra-tumoral | day 1 |
| 3 | 8 | AAV2-IL12 | 3E + 10 vg/mouse | intra-tumoral | day 1 |
| 4 | 8 | AAV2-IL12// | 1E + 11 vg/mouse// | intra-tumoral// | day 1// |
| | | anti-PD-1 | 5 mg/kg | intraperitoneal | biwk × 2 |
| | | RMP1-14 | | | |
| 5 | 8 | anti-PD-1 | 5 mg/kg | intraperitoneal | biwk × 2 |
| | | RMP1-14 | | | |

^{#}control group

Female DBA/2 mice were subcutaneously injected with 5×10⁵ Cloudman S91 tumor cells in 50% Matrigel (total cell injection volume=0.1 mL/mouse) in flank. Mice were 8 to 12 weeks old at the start date. A pair match was performed when tumors reached an average size of 80-120 mm³, and treatment began.

Body weight was measured daily for five days, then twice a week until the end of the study. Tumors were measured by calipers twice a week to the end of the study. Any adverse reactions or deaths were reported immediately. Any individual animal with a single observation of greater than 30% body weight loss or three consecutive measurements of greater than 25% body weight loss was euthanized. Any group with a mean body weight loss of greater than 20% or greater than 10% mortality stopped receiving dosing. The group was not euthanized and recovery was allowed. Within a group with greater than 20% weight loss, individuals that hit the individual body weight loss endpoint were euthanized. If the group treatment related body weight loss was recovered to within 10% of the original weights, dosing was resumed at a lower dose or less frequent dosing schedule. Exceptions to non-treatment body weight % recovery were allowed on a case-by-case basis.

Animals were monitored individually. The endpoint of the experiment was a tumor volume of 2000 mm³ or 29 days, whichever came first. Responders were followed longer. When the endpoint was reached, the animals were euthanized.

AAV2-IL12 vectors were provided in 5% Sorbitol+elevated 350 mM NaCl in PBS. Vehicle was provided in 5% Sorbitol+elevated 350 mM NaCl in PBS. The dosing volume for vehicle and the AAV2-IL12 vectors were 0.025 mL/mouse, and volume was not adjusted for body weight. AAV-IL12 vector dosing syringes were prewet with vehicle buffer prior to drawing up dosing solution for first time each syringe was used. The dosing volume for RMP1−14=10 mL/kg (0.200 mL/20 g mouse), and volume was adjusted accordingly for body weight.

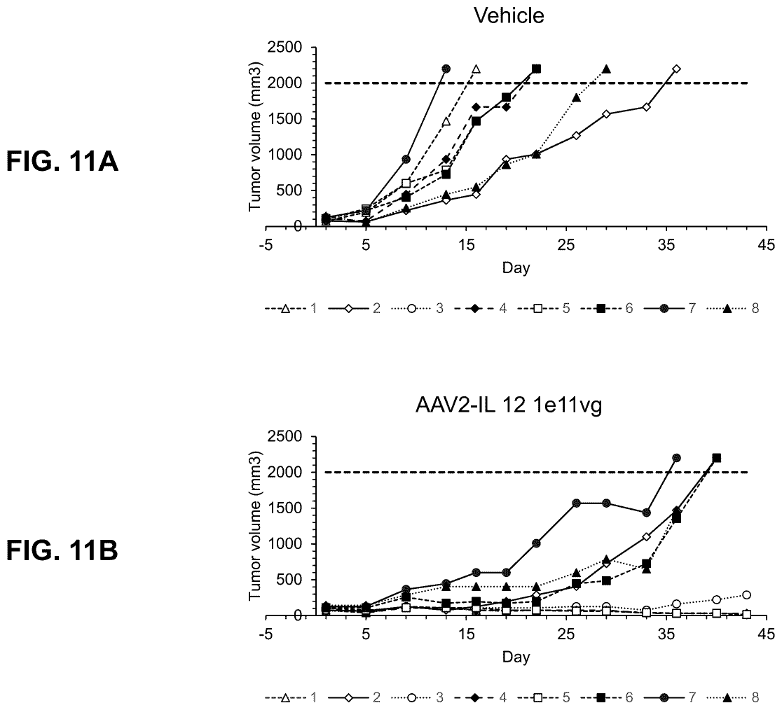

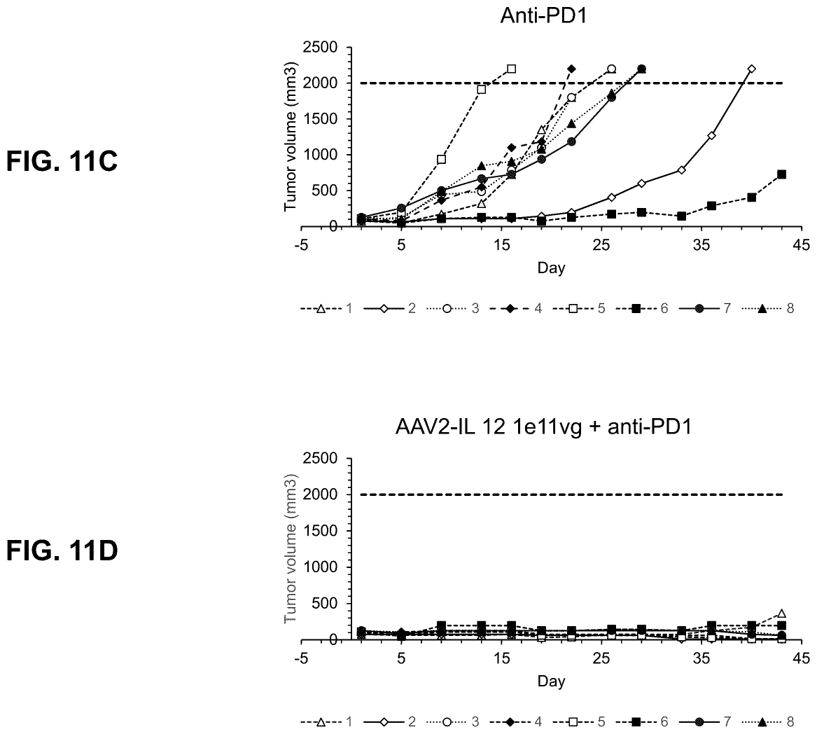

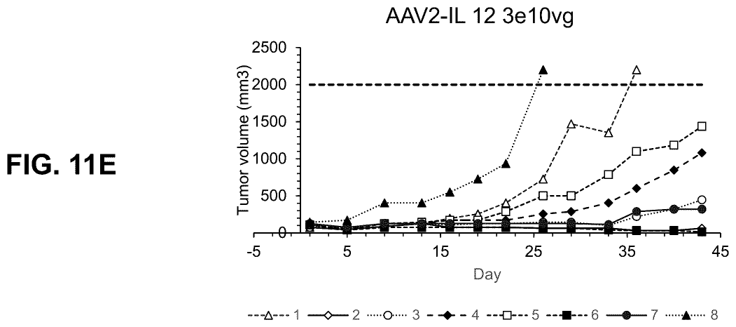

Tumor regression was measured by tumor volume in Cloudman S91 melanoma cancer models after treatment with vehicle (FIG. 11A), AAV2-IL12 (FIG. 11B and FIG. 11E), RMP1-14 (FIG. 11C), or AAV-IL12 and RMP1-14 combination therapy (FIG. 11D). Vehicle treated animals showed tumor growth consistent with historical data. AAV2-IL12 treated animals were sensitive to IL12 alone at both doses of 1×10¹¹ viral genomes (FIG. 111B) and 3×10¹⁰ viral genomes (FIG. 11E), showing decreased tumor volume. Animals treated with RMP1-14 showed a heterogenous response, consistent with historical data. AAV2-IL12 and RMP1-14 treated animals showed the best response, displaying that the combination treatment was superior to a single agent therapy. No meaningful negative impact on body weight was observed with AAV2-IL12 alone or in combination with RMP1-14 (FIG. 20C).

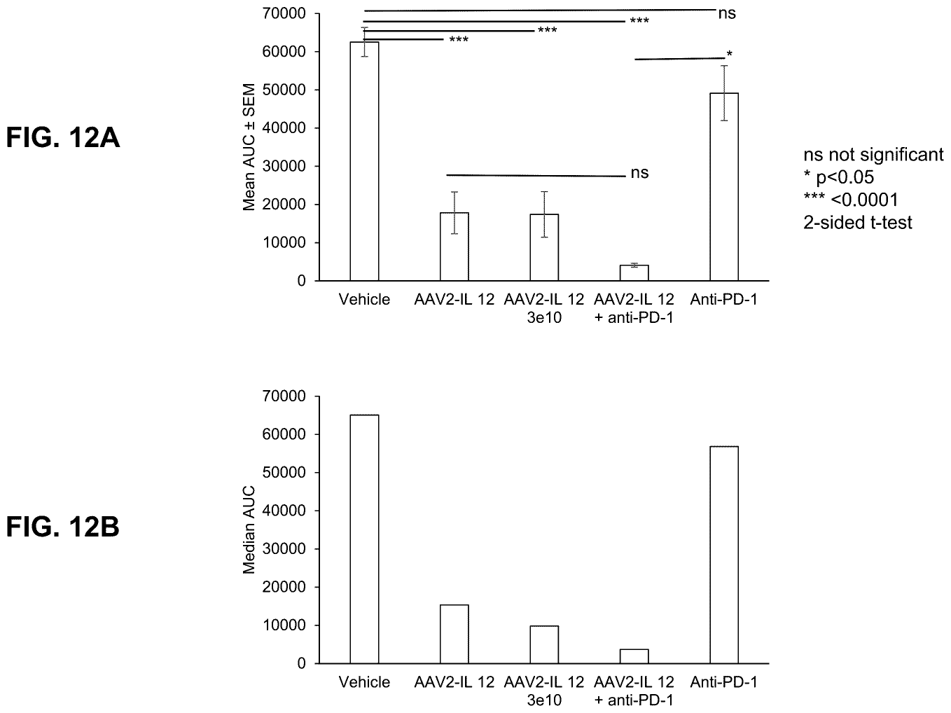

Tumor regression was also measured by both mean area under the curve (FIG. 12A) and median area under the curve (FIG. 12B) of tumor growth in Cloudman S91 melanoma cancer mouse models after treatment with vehicle, AAV-IL12, AAV-IL12 and RMP1-14, or RMP1-14 alone antibody. Treatment with AAV2-IL-12 at both 1E11 and 3E10 doses, as well as the combination of AAV2-IL12+RMP1-14 all resulted in significantly reduced AUC relative to vehicle and RMP1-14 alone, (p=0.01). However, AAV2-IL-12 at both 1E11 and 3E10 doses and AAV2-IL12+RMP1-14 were not significantly different from one another. (FIG. 12A).

A significant difference was observed between animals treated with RMP1-14 and animals treated with AAV2-IL12 alone. Animals treated with AAVL2-IL12 and RMP1-14 combination therapy had the lowest AUC, but the results were not significantly different from AAVL2-IL12 treatment alone.

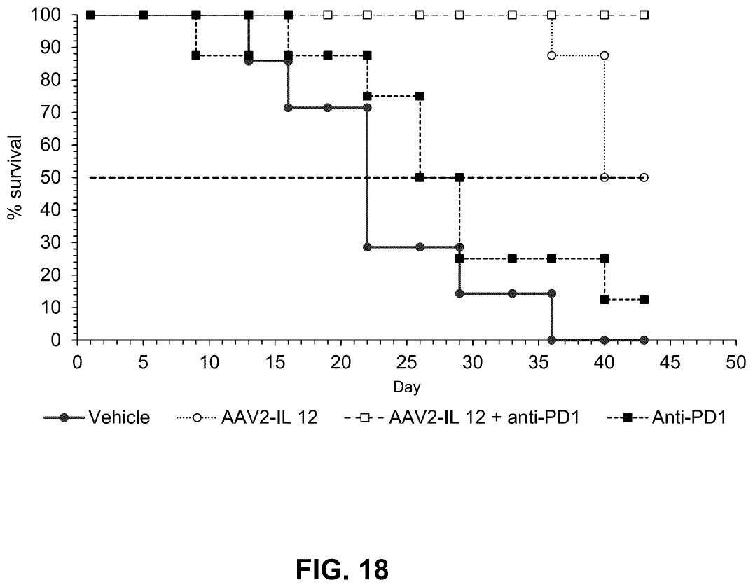

Overall survival was also measured in Cloudman S91 melanoma cancer mouse models after treatment with vehicle, AAV-IL12, AAV-IL12 and RMP1-14, or RMP1-14 alone (FIG. 18). RMP1-14 therapy had a minor improvement in overall survival. 100% of the mice that received AAV2-IL12 alone, and AAV2-IL12 in combination with RMP1-14 remained alive at the end of the study.

### Example 5: In Vivo Evaluation of AAV2-IL12 and an Anti-PD-1 Antibody Therapy in Hepa1-6 Hepatocellular Carcinoma Models

The anti-tumor efficacy of the AAV2-IL2 (AAV2-CAG-mIL12a-F2A-mIL12b-hgh PA) treatment alone and in combination with an anti-PD-1 antibody (RMP1-14) therapy was evaluated in the Hepatocellular Carcinoma (HCC) mouse model Hepa1-6 in female C57BL/6 mice. The study design is shown in Table 13 for vehicle, AAV2-IL12, RMP1-14, or a combination of AAV2-IL12 and RMP1-14.

TABLE 13
Study Design for Hepa1-6

| | |
|---|---|
| Model(s) | Hepa1-6 |
| Strain(s) | C57BL/6 |
| Number of mice on study | 32 |
| Number of groups | 4 |
| N per group | 8 |
| Inoculation | SubQ-Hind Right Flank |
| Dosing regimen | Single (AAV)/BIW × 3 (PD-1) |
| Dosing route | Intratumoral (AAV)/ |
| | Intraperitoneal (RMP1-14) |
| Dosing duration | 3 weeks (PD-1) |
| BW and TV measurement | 2×/week |
| frequency | |
| Randomization range | 80-120 mm3 |
| Dietary supplements | Body Weight Loss of >10%, |
| | all in group |
| Dosing holiday (PD-1) | Body Weight Loss of >20%, |
| | individual mouse |
| Tissue collections | Blood (cheek) Weeks: −1, 2, 4 |
| | Tumor (IHC) |
| Maximal tumor volume (MTV) | 3000 mm³ |

Tumor cell preparation, tumor implantation, tumor measurement, randomization and dose selection, study termination, body weight measurements, clinical observations, dosing holiday for an anti-PD-1 treatment, supplemental gel recovery administration, and tissue collection were preformed as described above in Example 2.

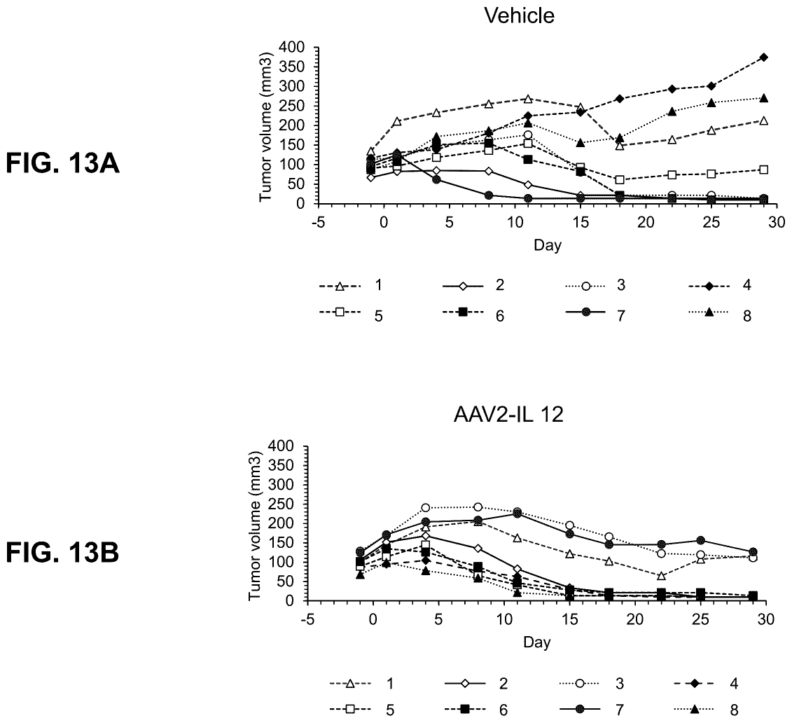

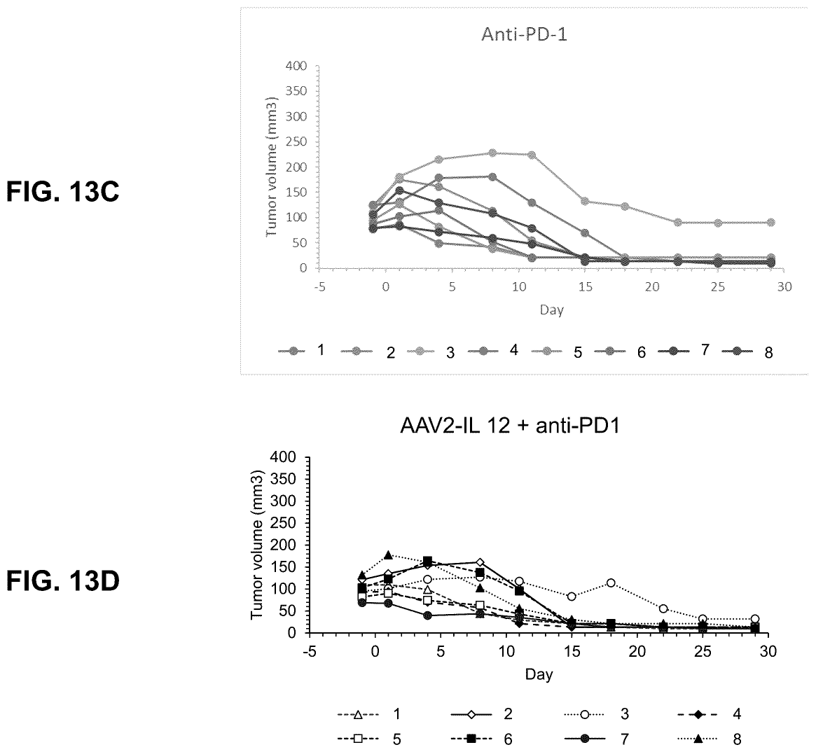

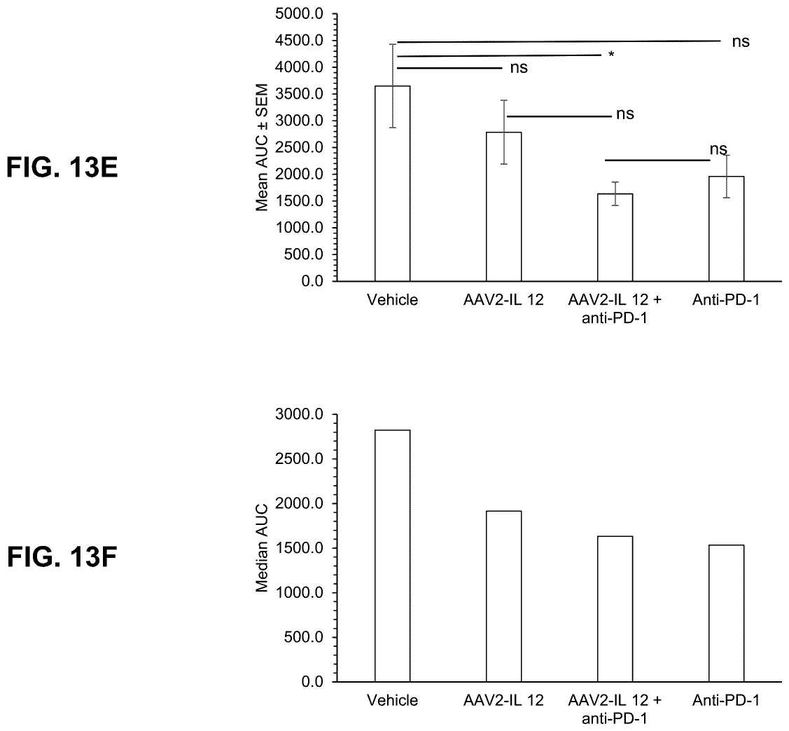

Tumor regression was measured by tumor volume in Hepa1-6 hepatocellular cancer models after treatment with vehicle (FIG. 13A), AAV2-IL12 (FIG. 13B), an anti-PD-1 antibody (FIG. 13C), or AAV-IL12 and an anti-PD-1 antibody (FIG. 13D). Tumor regression was also measured by both mean area under the curve (FIG. 13E) and median area under the curve (FIG. 13F) of tumor growth in Hepa1-6 heptacellular mouse models after treatment with vehicle, AAV-IL12, AAV-IL12 and RMP1-14, or RMP1-14 alone. AAV2-IL 12+RMP1-14 resulted in lower AUC relative to anti-PD-1 antibody alone, but the effect was not statistically significant. (FIG. 13E).

No meaningful impact on body weight was observed with AAV2-IL12 alone or in combination with RMP1-14 (FIG. 20E).

### Example 6: In Vivo Evaluation of AAV2-IL12 and an Anti-PD-1 Antibody Therapy in B16F10 Melanoma Mouse Models

The anti-tumor efficacy of AAV2-IL12 (AAV2-CAG-mIL12a-F2A-mIL12b-hgh PA) vectors in B16F10 melanoma cancer models was demonstrated using female B6D2F1 mice. The study was designed as shown in Table 14.

TABLE 14
Study Design for B16F10 melanoma.

| Gr. | N | Agent | Active dose | Route | Schedule |
|---|---|---|---|---|---|
| 1^{#} | 8 | vehicle | n/a | intra-tumoral | day 1 |
| 2 | 8 | AAV2-IL12 | 1E + 11 vg/mouse | intra-tumoral | day 1 |
| 3 | 8 | AAV2-IL12 | 3E + 10 vg/mouse | intra-tumoral | day 1 |
| 4 | 8 | AAV2-IL12// | 1E + 11 vg/mouse// | intra-tumoral// | day 1// |
| | | anti-PD-1 | 5 mg/kg | intraperitoneal | biwk × 2 |
| | | RMP1-14 | | | |
| 5 | 8 | anti-PD-1 | 5 mg/kg | intraperitoneal | biwk × 2 |
| | | RMP1-14 | | | |

^{#}control group

CR female B6D2F1 mice were subcutaneously injected with 5×10⁵ B16F10 tumor cells in 0% Matrigel (total cell injection volume=0.1 mL/mouse) in flank. Mice were 8 to 12 weeks old at the start date. A pair match was performed when tumors reached an average size of 60-90 mm³, and treatment began.

Body weight was measured daily for five days, then twice a week until the end of the study. Tumors were measured by calipers twice a week to the end of the study. Any adverse reactions or deaths were reported immediately. Any individual animal with a single observation of greater than 30% body weight loss or three consecutive measurements of greater than 25% body weight loss was euthanized. Any group with a mean body weight loss of greater than 20% or greater than 10% mortality stopped receiving dosing. The group was not euthanized and recovery was allowed. Within a group with greater than 20% weight loss, individuals that hit the individual body weight loss endpoint were euthanized. If the group treatment related body weight loss was recovered to within 10% of the original weights, dosing was resumed at a lower dose or less frequent dosing schedule. Exceptions to non-treatment body weight percent recovery were allowed on a case-by-case basis.

Animals were monitored individually. The endpoint of the experiment was a tumor volume of 2000 mm³ or 29 days, whichever came first. Responders were followed longer. When the endpoint was reached, the animals were euthanized.

AAV2-IL12 vectors were provided in 5% Sorbitol+elevated 350 mM NaCl in PBS. Vehicle was provided in 5% Sorbitol+elevated 350 mM NaCl in PBS. The dosing volume for vehicle and the AAV2-IL12 vectors were 0.025 mL/mouse, and volume was not adjusted for body weight. AAV-IL12 vector dosing syringes were prewet with vehicle buffer prior to drawing up dosing solution for first time each syringe was used. The dosing volume for anti-PD-1 antibody (RMP1-14)=10 mL/kg (0.200 mL/20 g mouse), and volume was adjusted accordingly for body weight.

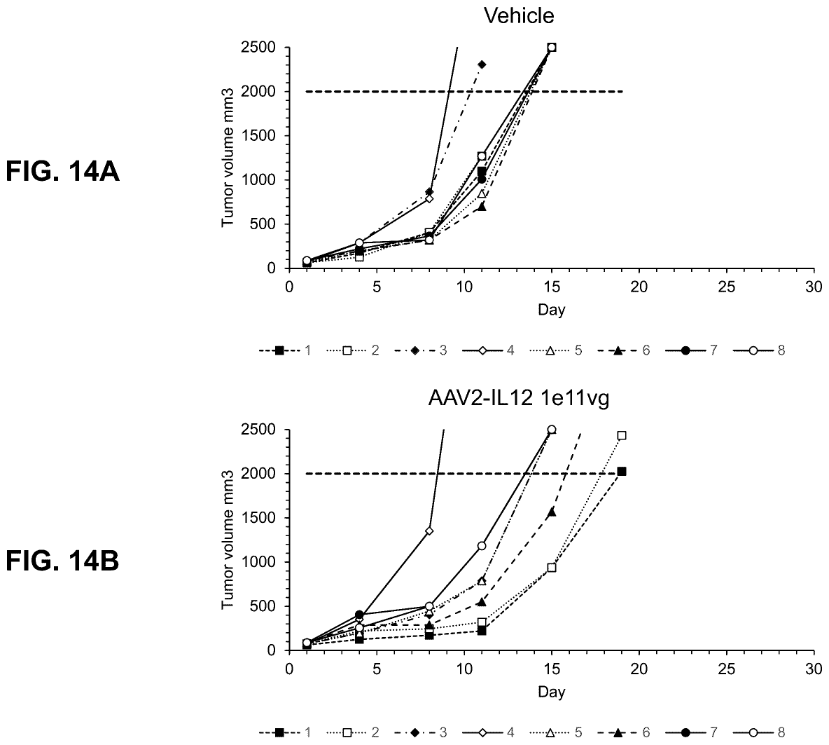

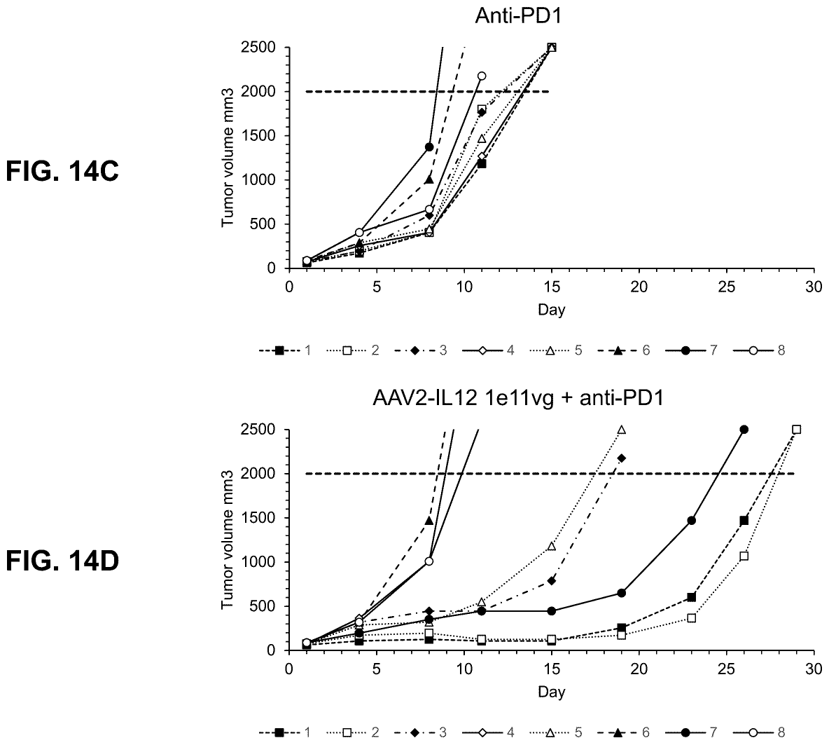

Tumor regression was measured by tumor volume (mm³) in B16F10 melanoma cancer models after treatment with vehicle (FIG. 14A), AAV2-IL12 (FIG. 14B), RMP1-14 (FIG. 14C), or AAV-IL12 and RMP1-14 (FIG. 14D). Vehicle treated animals showed tumor growth consistent with historical data. AAV2-IL12 treated animals showed modest delayed tumor growth. RMP1-14 treated animals showed that B16F10 was refractory to RMP1-14, which was consistent with literature. Animals treated with a combination of AAV2-IL12 and RMP1-14 showed heterogenous response, with 5 out of 8 samples showing delayed tumor growth. No meaningful impact on body weight was observed with AAV2-IL12 alone or in combination with RMP1-14 (FIG. 20B).

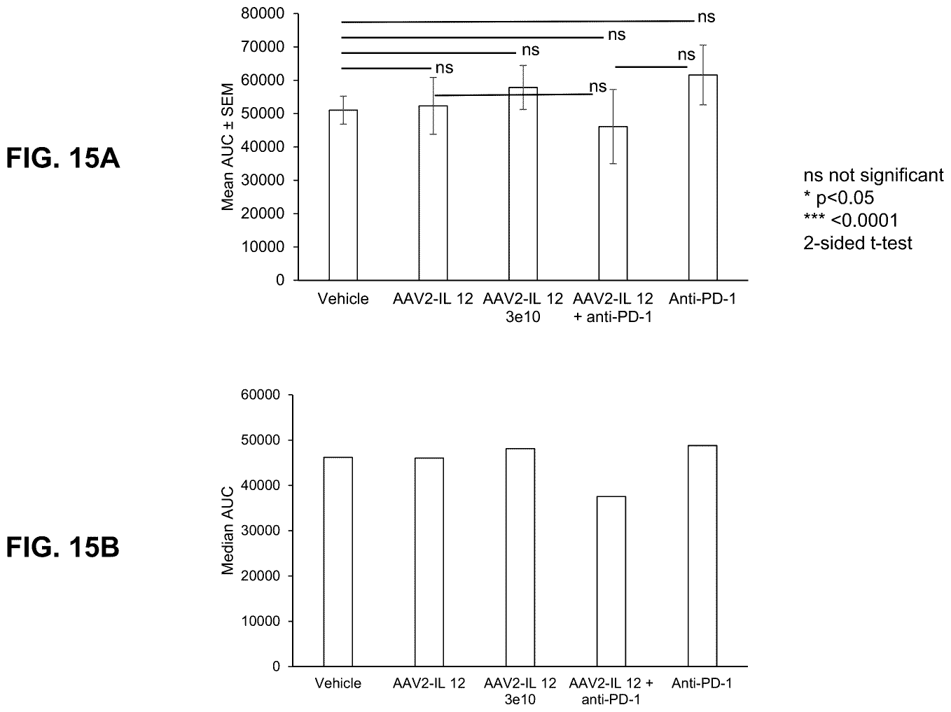

Tumor regression was also measured by both mean area under the curve (FIG. 15A) and median area under the curve (FIG. 15B) of tumor growth in B16F10 melanoma cancer models after treatment with vehicle, AAV-IL12, AAV-IL12 and RMP1-14, or RMP1-14. AAV2-IL-12+an anti-PD-1 antibody combination treatment resulted in slightly decreased AUC relative to control and all other treatments, but the effect was not statistically significant. (FIG. 15A).

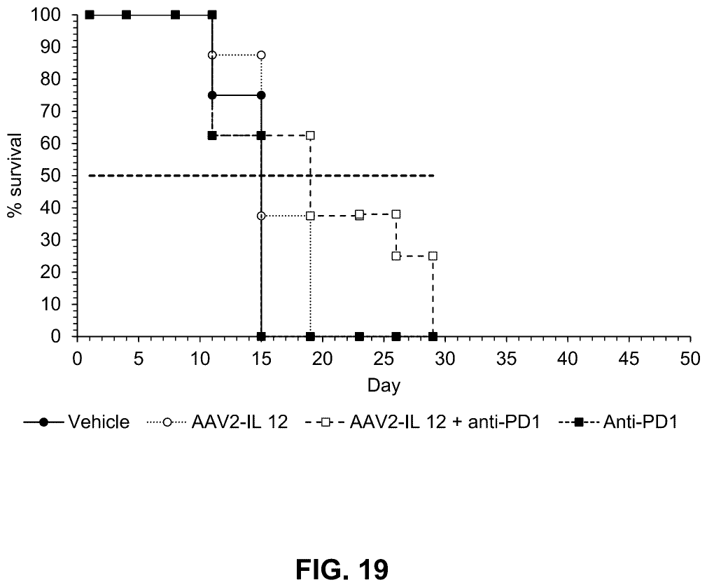

Overall survival of B16F10 melanoma cancer models after treatment with vehicle, AAV-IL12, AAV-IL12 and RMP1-14, or RMP1-14 alone was analyzed (FIG. 19). B16F10 melanoma was refractory to treatment with RMP1-14 alone, which is consistent with historical data in this melanoma model. Finally, AAV2-IL12 alone had an effect on overall survival and the combination of AAV2-IL12 and an anti-PD1 antibody therapy led to an improvement in overall survival.

### Example 7. In Vivo Evaluation of AAV2-IL12 Tolerability after Intravenous Injection

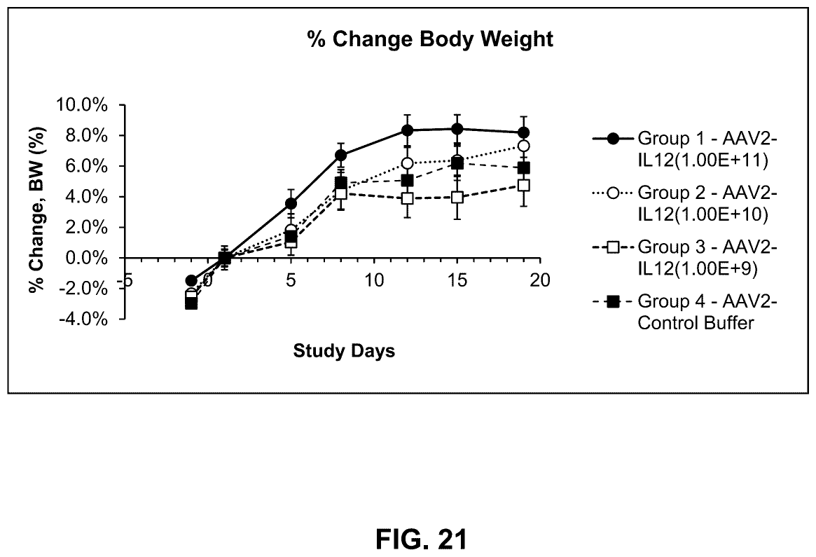

In order to determine tolerability of AAV2-IL12 (AAV2-CAG-mIL12a-F2A-mIL12b-hgh PA) in normal mice, mice were intravenously administered AAV-IL12 in escalated doses (1× 10⁹ viral genomes, 1×10¹⁰ viral genomes, or 1×10¹¹ viral genomes) or control. The escalated doses represented escape of the vector after intratumoral injection. The 1×10⁹ viral genome dose represents 1% of the dose used in efficacy studies, the 1×10¹⁰ viral genome dose represents 10% of the dose used in efficacy studies, and the 1×10¹¹ viral genome dose is equivalent to the dose used in the efficacy studies. Two weeks after AAV2-IL12 was administered to the normal mice, there was no notable adverse clinical observations and no change in body weight (FIG. 21).

### Example 8: In Vitro Evaluation of IL-12 Expression

HEK-293 cells were transduced at an MOI of either 3.0E4, 1.0E5, or 3.0E5 GC/cell of either AAV2 vector #1 (AAV2-CAG-mIL12a-IRES-mIL12b-bgh PA) or AAV2 vector #2 (AAV2-CAG-mIL12a-F2A-mIL12b-hgh PA). 48 hours post transduction media was collected from each well for supernatant evaluation. IL-12 ELISA assay was performed using 48 hour supernatant with the IL-12 ELISA Kit (Abcam, Cat #ab119531). IL-12 concentration was measured according to the manufacturer's instructions.

In vitro IL-12 Functional Assay.

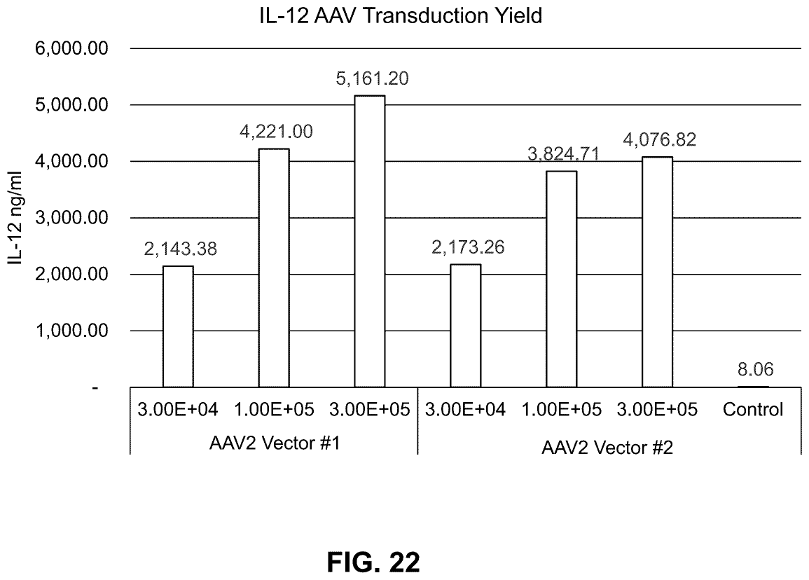

48-hour supernatant was diluted to 100 ng/ml IL-12 based on ELISA quantification results. HEK-Blue IL-12 Reporter Cells (InvivoGen, Cat #hkb-il12) were cultured according to the manufacturer's instructions. Cells were dispensed into wells preloaded with positive control (0.5 ng, 1 ng, 2 ng and 4 ng), 20 ul of diluted 48 hour supernatant, or 20 ul negative control (supernatant from untransduced cells). Cells were cultured for 24 hours. Following 24-hour culture, Quanti-Blue medium was prepared using Quanti-Blue Solution (InvivoGen, Cat #rep-qbs) according to the manufacturer's instruction. Supernatant from HEK-Blue IL-12 cells was added to wells preloaded with Quanti-Blue medium and incubated for up to 6 hours at 37 C. Optical density (OD) at 650 nm was measured using a microplate reader. Both AAV2 vector #1 (AAV2-CAG-mIL12a-IRES-mIL12b-bgh PA) or AAV2 vector #2 (AAV2-CAG-mIL12a-F2A-mIL12b-hgh PA) produced functional IL-12 (see FIG. 22).

### Example 9. Construction of an AAV2-hIL12 Vector

An AAV2 vector was designed to drive expression of a human IL12A subunit and a human IL12B subunit. The AAV2 vector was engineered to include a construct including AAV2 ITRs flanking an expression cassette including a CMV enhancer, CBA promoter, CAG intron, a human IL 12A wild-type coding sequence without an in-frame stop codon (SEQ ID NO: 77), a furin cleavage sequence, a 2A self-processing peptide sequence, a human IL12B wild-type coding sequence with a stop codon (SEQ ID NO: 8), and a human growth hormone PolyA site (AAV2-CAG-hIL12a-F2A-hIL12b-hgh PA).

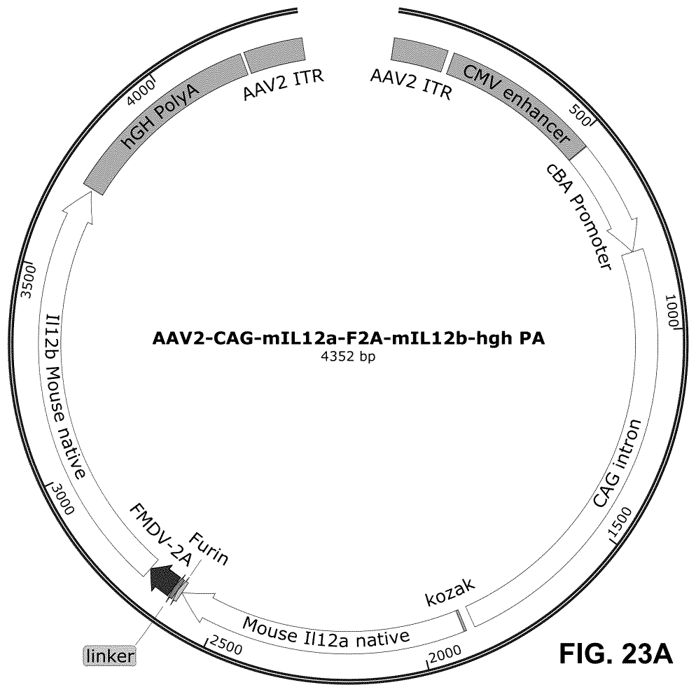

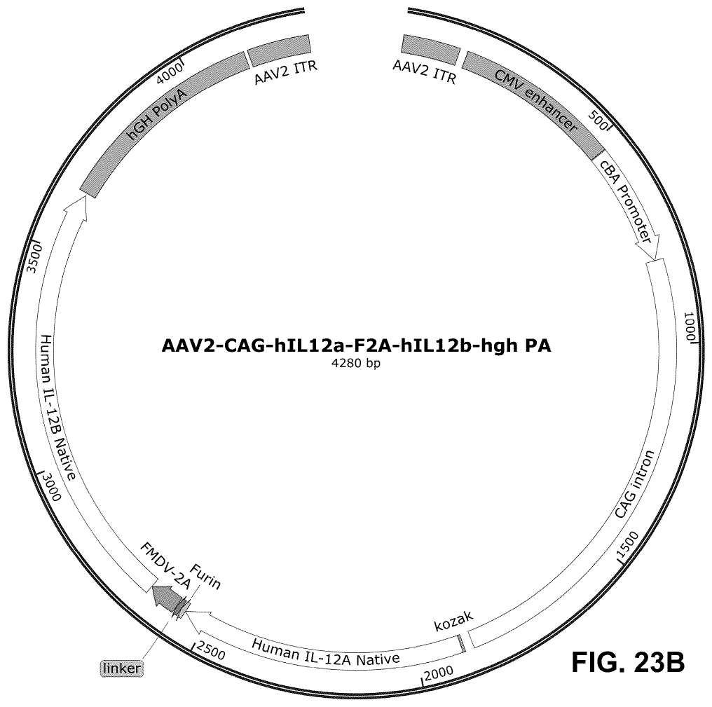

The construct for human studies, based on the mouse study construct (AAV2-CAG-mIL12a-F2A-mIL12b-hgh PA) described above (see also FIG. 23A), was prepared. The resulting human study construct (AAV2-CAG-hIL12a-F2A-hIL12b-hgh PA) is shown in FIG. 23B and Table 5, and the construct has a nucleic acid sequence that corresponds to SEQ ID NO: 82.

1-14. (canceled)

15. A method of treating a subject suffering from a tumor comprising administering a combination therapy to the subject, wherein the combination therapy comprises: (a) a first composition comprising an adeno-associated virus (AAV) vector comprising a polynucleotide comprising (i) a promoter, (ii) a first nucleic acid encoding a first Interleukin-12 (IL-12) subunit or functional fragment thereof, (iii) a translation modification sequence, and (iv) a second nucleic acid encoding a second IL-12 subunit or functional fragment thereof; and (b) a second composition comprising a checkpoint inhibitor.

16. The method of claim 15, wherein the first composition is administered intratumorally.

17. The method of claim 16, wherein the first composition is administered to multiple sites of the tumor.

18. The method of claim 15, wherein the first composition is administered prior to, at the same time, or after the administration of the second composition.

19. The method of claim 15, wherein tumor is derived from a cancer selected from the group consisting of a skin cancer; a breast cancer; a brain cancer; a bone cancer; a head and neck cancer; a salivary gland cancer; a gynecologic cancer; a urologic cancer; a gastrointestinal cancer; an ocular cancer; a thoracic cancer; a blood cancer; a cancer of the endocrine system; a sarcoma of soft tissue; a neoplasm of the central nervous system; and any combination thereof.

20. The method of claim 15, wherein tumor is derived from a cancer selected from the group consisting of a hepatocellular carcinoma, a colon carcinoma, and a melanoma.

21-78. (canceled)

79. A method of expressing one or more immunomodulatory proteins or functional fragments thereof in a subject in need thereof comprising administering an effective amount of the combination therapy of claim 15 to the subject.

80-116. (canceled)

117. A method for inducing tumor regression in a subject comprising administering to a subject in need the combination therapy of claim 15.

118-132. (canceled)

133. The method of claim 15, wherein the AAV vector is an AAV serotype 2 (AAV2) vector.

134. The method of claim 15, wherein the translation modification sequence comprises a furin cleavage sequence, a 2A self-processing peptide sequence, an internal ribosomal entry site (IRES) sequence, or any combination thereof.

135. The method of claim 134, wherein the translation modification sequence comprises a furin cleavage sequence and a 2A self-processing peptide sequence (F2A).

136. The method of claim 15, wherein the promoter comprises a CBA promoter, a CMV promoter, an EF-1a (Elongation Factor 1a) promoter, a RSV (Rous Sarcoma Virus) promoter, an Ubiquitin (UbC) promoter, a CAG promoter, or any combination thereof.

137. The method of claim 15, wherein the polynucleotide further comprises a poly(A) (pA) sequence.

138. The method of claim 15, wherein the polynucleotide further comprises two inverted terminal repeat (ITR) sequences.

139. The method of claim 15, wherein the checkpoint inhibitor comprises an inhibitor for programmed cell death protein 1 (PD-1), programmed death-ligand 1 (PD-L1), cytotoxic T-lymphocyte-associated protein 4 (CTLA-4), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, inducible T-cell costimulatory (ICOS), B7-H3, or any combination thereof.

140. The method of claim 15, wherein the checkpoint inhibitor comprises a programmed cell death protein 1 (PD-1) inhibitor.

141. The method of claim 140, wherein the PD-1 inhibitor is an anti-PD-1 antibody.

142. The method of claim 15, wherein the first IL-12 subunit comprises an amino acid sequence having at least 85%, 90%, 95%, 96%, 97%, 98%, 99%, or 100% identity to SEQ ID NO: 80 and the second IL-12 subunit comprises an amino acid sequence having at least 85%, 90%, 95%, 96%, 97%, 98%, 99%, or 100% identity to SEQ ID NO: 81.

143. The method of claim 15, wherein the first nucleic acid comprises a nucleic acid sequence having at least 85%, 90%, 95%, 96%, 97%, 98%, 99%, or 100% identity to SEQ ID NO: 5, SEQ ID NO: 6, SEQ ID NO: 7, or SEQ ID NO: 77, and the second nucleic acid comprises a nucleic acid sequence having at least 85%, 90%, 95%, 96%, 97%, 98%, 99%, or 100% identity to SEQ ID NO: 8, SEQ ID NO: 9, SEQ ID NO: 10, or SEQ ID NO: 92.

144. The method of claim 15, wherein the polynucleotide comprises a nucleic acid sequence having at least 85%, 90%, 95%, 96%, 97%, 98%, 99%, or 100% identity to SEQ ID NO: 82.
